(19) United States
(12) Patent Application Publication
(10) Pub. No.: US 2021/0348167 A1
(43) Pub. Date: Nov. 11, 2021

(54) siNA MOLECULES, METHODS OF PRODUCTION AND USES THEREOF

(71) Applicant: PHYZAT BIOPHARMACEUTICALS, LDA, Porto (PT)

(72) Inventor: PATRICIO MANUEL VIEIRA ARAUJO SOARES DA SILVA, PORTO (PT)

(21) Appl. No.: 17/245,682

(22) Filed: Apr. 30, 2021

(30) Foreign Application Priority Data
May 9, 2020 (PT) 116354

(51) Int. Cl. C12N 15/113; A61K 45/06; A61K 31/713; A61P 31/14

(52) U.S. Cl. CPC C12N 15/1131; A61K 45/06; C12N 2320/31; A61P 31/14; C12N 2310/14; A61K 31/713

(57) ABSTRACT

The present disclosure relates to method of producing and using short interfering nucleic acids (siNAs) for preventing and treating coronavirus-inflicted infectious conditions. In particular, this disclosure relates to the method of producing and using siNAs for preventing and treating infections by the coronavirus SARS-CoV-2, the causative viral agent of the novel coronavirus disease COVID-19, to mediate gene silencing of viral proteins. The present disclosure is also directed to interfering RNA duplexes and vectors encoding such interfering RNA duplexes.

### CROSS REFERENCE TO RELATED APPLICATION

This patent application claims the benefit and priority of Portugal Patent Application No. 116354 filed on May 9, 2020, the disclosure of which is incorporated by reference herein in its entirety as part of the present application.

### TECHNICAL FIELD

The present disclosure relates to method of producing and using short interfering nucleic acids (siNAs) for preventing and treating coronavirus-inflicted infectious conditions. In particular, this disclosure relates to the method of producing and using siNAs for preventing and treating infections by the coronavirus SARS-CoV-2, the causative viral agent of the novel coronavirus disease COVID-19, to mediate gene silencing of viral proteins. The present disclosure is also directed to interfering RNA duplexes and vectors encoding such interfering RNA duplexes.

### BACKGROUND

Six strains of coronaviruses (CoVs) that are able to infect humans have been identified until 2019. HCoV-OC43, HCoV-229E, HCoV-NL63, and HCoVHKU1 are not highly pathogenic and only cause mild respiratory diseases. SARS-CoV (severe acute respiratory syndrome coronavirus) and MERS-CoV (Middle-East respiratory syndrome coronavirus) have caused two severe epidemics in 2002 and 2012, respectively.

Before efficient antiviral drugs or vaccines were developed for SARS-CoV or MERS-CoV, another outbreak of pneumonia caused by a new coronavirus (SARS-CoV-2) has emerged in Wuhan (China), the virus that causes the disease COVID-19 (Guan et al, 2020; Liu et al., 2020), encompassing asymptomatic infection, mild upper respiratory tract illness, severe viral pneumonia with respiratory failure and even death, and since then spread to multiple continents, leading to WHO's declaration of a Public Health Emergency of International Concern (PHEIC) on 30 Jan. 2020.

No drug or vaccine has yet been approved to treat human coronaviruses. Several options can be envisaged to control or prevent emerging infections by the new coronavirus SARS-CoV-2, including vaccines, monoclonal antibodies, oligonucleotide-based therapies, peptides, interferon therapies and small-molecule drugs (Li & De Clerq, 2020).

SARS-CoV-2 is an enveloped, positive-sense, single-stranded RNA beta-coronavirus. Similar to SARS-CoV or MERS-CoV, the SARS-CoV-2 genome encodes non-structural proteins (NSPs; such as 3-chymotrypsin-like protease, papain-like protease, helicase, and RNA-dependent RNA polymerase), structural proteins (such as spike glycoprotein) and accessory proteins (Zumla et al., 2016).

The spike (S) glycoprotein is critical for virus-cell receptor interactions during viral entry (Hoffmann et al., 2020) and the four non-structural proteins mentioned above are key enzymes in the viral life cycle (Perlman & Netland, 2009; Fehr et al., 2015).

RNA interference (“RNAi”) is a recently discovered mechanism of post-transcriptional gene silencing in which double-stranded RNA corresponding to a gene (or coding region) of interest is introduced into an organism, resulting in degradation of the corresponding mRNA. The phenomenon was originally discovered in Caenorhabditis elegans (Fire et al., 1998).

Unlike antisense technology, the RNAi phenomenon persists for multiple cell divisions before gene expression is regained. The process occurs in at least two steps: an endogenous ribonuclease cleaves the longer dsRNA into shorter, 21-22- or 23-nucleotide-long RNAs, termed “small interfering RNAs” or siRNAs (Hannon, 2002). The siRNA segments then mediate the degradation of the target mRNA. RNAi has been used for gene function determination in a manner similar to but more efficient than antisense oligonucleotides. By making targeted knockouts at the RNA level by RNAi, rather than at the DNA level using conventional gene knockout technology, a vast number of genes can be assayed quickly and efficiently. RNAi is therefore an extremely powerful, simple method for assaying gene function.

RNAi has been shown to be effective in cultured mammalian cells. In most methods described to date, RNAi is carried out by introducing double-stranded RNA into cells by microinjection or by soaking cultured cells in a solution of double-stranded RNA, as well as transfecting the cells with a plasmid carrying a hairpin-structured siRNA expressing cassette under the control of suitable promoters, such as the U6, H1 or cytomegalovirus (“CMV”) promoter (Elbashir et al., 2001; Harborth et al., 2001; Lee et al., 2001; Brummelkamp et al., 2002; Miyagishi et al., 2002; Paddison et al., 2002; Paul et al., 2002; Sui et al., 2002; Xia et al., 2002; Yu et al., 2002). The gene-specific inhibition of gene expression by double-stranded ribonucleic acid is generally described in U.S. Pat. No. 6,506,559, which is incorporated herein by reference. Exemplary use of siRNA technology is further described in Published U.S. Patent Application N. 2003/01090635 and Published U.S. Patent Application N. 20040248174, which are incorporated herein by reference. Davis (Davis, 2009) describes the targeted delivery of siRNA to humans using nanoparticle technology.

Compared with clinically used nonspecific antiviral drugs, a siRNA against non-structural proteins (siRNA-NSPs) from SARS-CoV-2 has more advantages for treatment and prevention of SARS-CoV-2 infection. Firstly, the sequence of the target, the non-structural proteins, is highly conserved. Therefore, a siRNA-NSPs from SARS-CoV-2 possesses a high genetic barrier to resistance and cannot easily induce drug-resistant mutations. Secondly, a siRNA-NSPs from SARS-CoV-2 can be used in an intranasal formulation to prevent coronavirus infection. The small containers can be carried easily by persons who will have close contact with infected patients or high-risk populations. Thirdly, a siRNA-NSPs from SARS-CoV-2 can be used in inhalation formulation for treatment of patients to reduce the viral loads in their lungs, thus attenuating the acute lung injury caused by viral infection and reducing the chance of spreading the virions to the closely contacted persons. The inhalation equipment can be used at home or hotel room, reducing the expense of staying in hospitals. Fourthly, a siRNA-NSPs from SARS-CoV-2 is expected to be safe to humans because it will be used locally, not systemically, and siRNA drugs are generally safer than chemical drugs.

These facts are disclosed in order to illustrate the technical problem addressed by the present disclosure.

### GENERAL DESCRIPTION

The present disclosure relates to method of producing and using short interfering nucleic acids (siNAs) for preventing and treating coronavirus-inflicted infectious conditions. In particular, it relates to the method of producing and using siNAs for preventing and treating infections by the coronavirus SARS-CoV-2, the causative viral agent of the novel coronavirus disease COVID-19, to mediate gene silencing of viral proteins. The present disclosure is also directed to interfering RNA duplexes and vectors encoding such interfering RNA duplexes.

An object of the present disclosure is to use an RNA interference technique to down regulate the expression of the gene encoding nonstructural proteins (NSPs) from SARS-CoV-2 in order to treat or prevent the coronavirus SARS-CoV-2 inflicted infectious conditions. The compositions (or molecules) of the disclosure comprises or consists of short interfering nucleic acid molecules (siNA) and related compounds including, but not limited to, siRNA. The present disclosure encompasses compositions and methods of use of siNA including, but not limited to short interfering RNA (siRNA), double-stranded RNA (dsRNA), micro-RNA (miRNA), antagomirs and short hairpin RNA (shRNA) capable of mediating RNA interference. In one embodiment, the siNA molecule of the disclosure can be incorporated into RISC (RNA-induced silencing complex).

A further object of the present disclosure is to provide a siRNA molecule that efficiently down-regulates the expression of NSPs from SARS-CoV-2 gene.

Accordingly, in a first aspect, the disclosure relates to a siNA molecule, wherein said molecule specifically targets at least one sequence selected from SEQ ID No 1 to SEQ ID No 599 or a variant thereof. In an alternative embodiment, the disclosure relates to an siNA molecule wherein said molecule specifically targets at least one sequence complementary to at least one sequence selected from SEQ ID No 600 to SEQ ID No 1797 or a variant thereof. In one embodiment, the disclosure relates to an isolated siNA molecule, preferably an isolated siRNA molecule.

In one embodiment, the siNA molecule specifically targets at least one sequence selected from SEQ ID No 58, SEQ ID No 59, SEQ ID No 60, SEQ ID No 61, SEQ ID No 62, SEQ ID No 86, SEQ ID No 152, SEQ ID No 153, SEQ ID No 210, SEQ ID No 250, SEQ ID No 263, SEQ ID No 314, SEQ ID No 324, SEQ ID No 325, SEQ ID No 338, SEQ ID No 339, SEQ ID No 345, SEQ ID No 346, SEQ ID No 347, SEQ ID No 352, SEQ ID No 353, SEQ ID No 354, SEQ ID No 367, SEQ ID No 368, SEQ ID No 373, SEQ ID No 374, SEQ ID No 375, SEQ ID No 376, SEQ ID No 377, SEQ ID No 384, SEQ ID No 484, SEQ ID No 485, SEQ ID No 495, SEQ ID No 496, SEQ ID No 497, SEQ ID No 498, SEQ ID No 506, SEQ ID No 517, SEQ ID No 524, SEQ ID No 542, SEQ ID No 545, SEQ ID No 546, SEQ ID No 548, SEQ ID No 558, SEQ ID No 559, SEQ ID No 565, SEQ ID No 567, SEQ ID No 570, SEQ ID No 574, SEQ ID No 579, SEQ ID No 580 and SEQ ID No 582, or a variant thereof. Preferably, the siNA molecule targets a sequence selected from SEQ ID No 58, SEQ ID No 59, SEQ ID No 86, SEQ ID No 152, SEQ ID No 153, SEQ ID No 210, SEQ ID No 250, SEQ ID No 263, SEQ ID No 314, SEQ ID No 324, SEQ ID No 325, SEQ ID No 338, SEQ ID No 339, SEQ ID No 345, SEQ ID No 346, SEQ ID No 347, SEQ ID No 352, SEQ ID No 353, SEQ ID No 354, SEQ ID No 367, SEQ ID No 368, SEQ ID No 373, SEQ ID No 374, SEQ ID No 375, SEQ ID No 376, SEQ ID No 377, SEQ ID No 384, SEQ ID No 484, SEQ ID No 485, SEQ ID No 495, SEQ ID No 506, SEQ ID No 517, SEQ ID No 524, SEQ ID No 542, SEQ ID No 545, SEQ ID No 546, SEQ ID No 548, SEQ ID No 558, SEQ ID No 559, SEQ ID No 565, SEQ ID No 567, SEQ ID No 570, SEQ ID No 574, SEQ ID No 579, SEQ ID No 580 and SEQ ID No 582 or a variant thereof. Preferably, the siNA molecule reduces expression of the non-structural proteins (NSPs) from SARS-CoV-2 gene when expressed into a cell.

In a further embodiment, the siNA preferably comprises a double-stranded RNA molecule, whose antisense strand is substantially complementary to any of SEQ ID No 1 to SEQ ID No 599, more preferably SEQ ID No 58, SEQ ID No 59, SEQ ID No 60, SEQ ID No 61, SEQ ID No 62, SEQ ID No 86, SEQ ID No 152, SEQ ID No 153, SEQ ID No 210, SEQ ID No 250, SEQ ID No 263, SEQ ID No 314, SEQ ID No 324, SEQ ID No 325, SEQ ID No 338, SEQ ID No 339, SEQ ID No 345, SEQ ID No 346, SEQ ID No 347, SEQ ID No 352, SEQ ID No 353, SEQ ID No 354, SEQ ID No 367, SEQ ID No 368, SEQ ID No 373, SEQ ID No 374, SEQ ID No 375, SEQ ID No 376, SEQ ID No 377, SEQ ID No 384, SEQ ID No 484, SEQ ID No 485, SEQ ID No 495, SEQ ID No 496, SEQ ID No 497, SEQ ID No 498, SEQ ID No 506, SEQ ID No 517, SEQ ID No 524, SEQ ID No 542, SEQ ID No 545, SEQ ID No 546, SEQ ID No 548, SEQ ID No 558, SEQ ID No 559, SEQ ID No 565, SEQ ID No 567, SEQ ID No 570, SEQ ID No 574, SEQ ID No 579, SEQ ID No 580 and SEQ ID No 582 or a variant thereof, even more preferably SEQ ID No 58, SEQ ID No 59, SEQ ID No 86, SEQ ID No 152, SEQ ID No 153, SEQ ID No 210, SEQ ID No 250, SEQ ID No 263, SEQ ID No 314, SEQ ID No 324, SEQ ID No 325, SEQ ID No 338, SEQ ID No 339, SEQ ID No 345, SEQ ID No 346, SEQ ID No 347, SEQ ID No 352, SEQ ID No 353, SEQ ID No 354, SEQ ID No 367, SEQ ID No 368, SEQ ID No 373, SEQ ID No 374, SEQ ID No 375, SEQ ID No 376, SEQ ID No 377, SEQ ID No 384, SEQ ID No 484, SEQ ID No 485, SEQ ID No 495, SEQ ID No 506, SEQ ID No 517, SEQ ID No 524, SEQ ID No 542, SEQ ID No 545, SEQ ID No 546, SEQ ID No 548, SEQ ID No 558, SEQ ID No 559, SEQ ID No 565, SEQ ID No 567, SEQ ID No 570, SEQ ID No 574, SEQ ID No 579, SEQ ID No 580 and SEQ ID No 582, and its sense strand will comprise an RNA sequence complementary to the sense strand, wherein both strands are hybridised by standard base pairing between nucleotides.

In a further embodiment, said sense stand comprises or consists of a sequence selected from SEQ ID No 600 to SEQ ID No 1198, preferably SEQ ID No 657, SEQ ID No 658, SEQ ID No 659, SEQ ID No 660, SEQ ID No 661, SEQ ID No 685, SEQ ID No 751, SEQ ID No 752, SEQ ID No 809, SEQ ID No 849, SEQ ID No 862, SEQ ID No 913, SEQ ID No 923, SEQ ID No 924, SEQ ID No 937, SEQ ID No 938, SEQ ID No 944, SEQ ID No 945, SEQ ID No 946, SEQ ID No 951, SEQ ID No 952, SEQ ID No 953, SEQ ID No 966, SEQ ID No 967, SEQ ID No 972, SEQ ID No 974, SEQ ID No 975, SEQ ID No 976, SEQ ID No 1083, SEQ ID No 1084, SEQ ID No 1094, SEQ ID No 1095, SEQ ID No 1096, SEQ ID No 1097, SEQ ID No 1105, SEQ ID No 1116, SEQ ID No 1123, SEQ ID No 1141, SEQ ID No 1144, SEQ ID No 1145, SEQ ID No 1147, SEQ ID No 1157, SEQ ID No 1158, SEQ ID No 1164, SEQ ID No 1166, SEQ ID No 1169, SEQ ID No 1173, SEQ ID No 1178, SEQ ID No 1179 and SEQ ID No 1181, more preferably SEQ ID No 657, SEQ ID No 658, SEQ ID No 685, SEQ ID No 751, SEQ ID No 752, SEQ ID No 809, SEQ ID No 849, SEQ ID No 862, SEQ ID No 913, SEQ ID No 923, SEQ ID No 924, SEQ ID No 937, SEQ ID No 938, SEQ ID No 944, SEQ ID No 945, SEQ ID No 946, SEQ ID No 951, SEQ ID No 952, SEQ ID No 953, SEQ ID No 966, SEQ ID No 967, SEQ ID No 972, SEQ ID No 974, SEQ ID No 975, SEQ ID No 976, SEQ ID No 1083, SEQ ID No 1084, SEQ ID No 1094, SEQ ID No 1105, SEQ ID No 1116, SEQ ID No 1123, SEQ ID No 1141, SEQ ID No 1144, SEQ ID No 1145, SEQ ID No 1147, SEQ ID No 1157, SEQ ID No 1158, SEQ ID No 1164, SEQ ID No 1166, SEQ ID No 1169, SEQ ID No 1173, SEQ ID No 1178, SEQ ID No 1179 and SEQ ID No 1181 or a variant thereof.

In a further embodiment, said antisense strand comprises or consists of a sequence selected from SEQ ID No 1199 to SEQ ID No 1797, preferably SEQ ID No 1256, SEQ ID No 1257, SEQ ID No 1258, SEQ ID No 1259, SEQ ID No 1260, SEQ ID No 1284, SEQ ID No 1350, SEQ ID No 1351, SEQ ID No 1408, SEQ ID No 1448, SEQ ID No 1461, SEQ ID No 1512, SEQ ID No 1522, SEQ ID No 1523, SEQ ID No 1536, SEQ ID No 1537, SEQ ID No 1543, SEQ ID No 1544, SEQ ID No 1545, SEQ ID No 1550, SEQ ID No 1551, SEQ ID No 1552, SEQ ID No 1565, SEQ ID No 1566, SEQ ID No 1571, SEQ ID No 1572, SEQ ID No 1573, SEQ ID No 1574, SEQ ID No 1575, SEQ ID No 1582, SEQ ID No 1682, SEQ ID No 1683, SEQ ID No 1693, SEQ ID No 1694, SEQ ID No 1695, SEQ ID No 1696, SEQ ID No 1704, SEQ ID No 1715, SEQ ID No 1722, SEQ ID No 1740, SEQ ID No 1743, SEQ ID No 1744, SEQ ID No 1746, SEQ ID No 1756, SEQ ID No 1757, SEQ ID No 1763, SEQ ID No 1765, SEQ ID No 1768, SEQ ID No 1772, SEQ ID No 1777, SEQ ID No 1778, and SEQ ID No 1780, more preferably SEQ ID No 1256, SEQ ID No 1257, SEQ ID No 1284, SEQ ID No 1350, SEQ ID No 1351, SEQ ID No 1408, SEQ ID No 1448, SEQ ID No 1461, SEQ ID No 1512, SEQ ID No 1522, SEQ ID No 1523, SEQ ID No 1536, SEQ ID No 1537, SEQ ID No 1543, SEQ ID No 1544, SEQ ID No 1545, SEQ ID No 1550, SEQ ID No 1551, SEQ ID No 1552, SEQ ID No 1565, SEQ ID No 1566, SEQ ID No 1571, SEQ ID No 1572, SEQ ID No 1573, SEQ ID No 1574, SEQ ID No 1575, SEQ ID No 1582, SEQ ID No 1682, SEQ ID No 1683, SEQ ID No 1693, SEQ ID No 1704, SEQ ID No 1715, SEQ ID No 1722, SEQ ID No 1740, SEQ ID No 1743, SEQ ID No 1744, SEQ ID No 1746, SEQ ID No 1756, SEQ ID No 1757, SEQ ID No 1763, SEQ ID No 1765, SEQ ID No 1768, SEQ ID No 1772, SEQ ID No 1777, SEQ ID No 1778, and SEQ ID No 1780 or a variant thereof.

Within the meaning of the present disclosure “substantially complementary” to a target mRNA sequence, may also be understood as “substantially identical” to said target sequence. “Identity” as is known by one of ordinary skill in the art, is the degree of sequence relatedness between nucleotide sequences as determined by matching the order and identity of nucleotides between sequences. In one embodiment the antisense strand of an siRNA having 80%, and between 80% up to 100% complementarity, for example, 85%, 86%, 87%, 88%, 89%, 90%, 91%, 92%, 93%, 94%, 95%, 96%, 97% or 99% complementarity, to the target mRNA sequence are considered substantially complementary and may be used in the present disclosure. The percentage of complementarity describes the percentage of contiguous nucleotides in a first nucleic acid molecule that can base pair in the Watson-Crick sense with a set of contiguous nucleotides in a second nucleic acid molecule.

A gene is “targeted” by a siNA according to the present disclosure when, for example, the siNA molecule selectively decreases or inhibits the expression of the gene. The phrase “selectively decrease or inhibit” as used herein encompasses siNAs that affect expression of the non-structural proteins (NSPs) from SARS-CoV-2. Alternatively, a siNA targets a gene when the siNA hybridizes under stringent conditions to the gene transcript, i.e. its mRNA. Capable of hybridizing “under stringent conditions” means annealing to the target mRNA region, under standard conditions, e.g., high temperature and/or low salt content which tend to disfavor hybridization. A suitable protocol (involving 0.1×SSC, 68° C. for 2 hours) is described in Maniatis, T., et al., Molecular Cloning: A Laboratory Manual, Cold Spring Harbor Laboratory, 1982, at pages 387-389.

Nucleic acid sequences cited herein are written in a 5′ to 3′ direction unless indicated otherwise. The term “nucleic acid” refers to either DNA or RNA or a modified form thereof comprising the purine or pyrimidine bases present in DNA (adenine “A”, cytosine “C”, guanine “G”, thymine “T”) or in RNA (adenine “A”, cytosine “C”, guanine “G”, uracil “U”). Interfering RNAs provided herein may comprise “T” bases, for example at 3′ ends, even though “T” bases do not naturally occur in RNA. In some cases, these bases may appear as “dT” to differentiate deoxyribonucleotides present in a chain of ribonucleotides.

In one embodiment of the disclosure, the siNA molecule is 40 base pairs or fewer in length. Preferably, the siNA molecule is 19 to 25 base pairs in length. In one embodiment, the siNA comprises or consists of a 21 nucleotide double-stranded region. Preferably, the siNA has a sense and an anti-sense strand. In an alternative embodiment, the siNA molecule comprises or consists of a 19 nucleotide double-stranded region. In one embodiment, the siNA has blunt ends. In an alternative embodiment, the siNA has 5′ and/or 3′ overhangs. Preferably the overhangs are between 1 to 5 nucleotides, more preferably, 2 nucleotide overhangs. The overhangs may be ribonucleic acids, or deoxyribonucleic acids.

In one embodiment, the siNA molecule according to the disclosure comprises a chemical modification. Preferably, the chemical modification is on the sense strand, the antisense strand or both. Phosphorothioate (PS)- or boranophosphate (BS)-modified siRNAs have substantial nuclease resistance. Silencing by siRNA duplexes is also compatible with some types of 2′-sugar modifications: 2′-H, 2′-O-methyl, 2′-O-methoxyethyl, 2′-fluoro (2′-F), locked nucleic acid (LNA) and ethylene-bridge nucleic acid (ENA).

In one embodiment, the 5′ or 3′ overhangs are dinucleotides, preferably thymidine dinucleotide. In a embodiment, the 5′ or 3′ overhangs are deoxythymidines. In one embodiment, the sense strand comprises at least one, preferably two 3′ overhangs. Preferably, said sense strand comprises at least one, preferably two 3′ deoxythymidines. In an alternative embodiment, the antisense strand comprises at least one, preferably two 3′ overhangs. Preferably, said sense strand comprises at least one, preferably two 3′ deoxythymidines. In a further preferred embodiment, both the sense and antisense strands comprise 3′ overhangs as described herein.

By “variant” as used herein is meant a sequence with 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic or ribonucleic acid sequence.

By “down-regulating” is meant a decrease in the expression non-structural proteins (NSPs) from SARS-CoV-2 mRNA by up to or more than 10%, 15% 20%, 25%, 30%, 35%, 40%, 45% 50%, 55% 60%, 65%, 70%, 75%, 80%, 85%, 90%, 95% when compared to the level in a control. Alternatively, the siNA molecule described herein may abolish SARS-CoV-2 non-structural proteins (NSPs) expression. The term “abolish” means that no expression of SARS-CoV-2 non-structural proteins (NSPs) is detectable or that no functional SARS-CoV-2 non-structural proteins (NSPs) is produced. For example, a reduction in the expression and/or protein levels of at least SARS-CoV-2 non-structural proteins (NSPs) expression may be a measure of protein and/or nucleic acid levels and can be measured by any technique known to the skilled person, such as, but not limited to, any form of gel electrophoresis or chromatography (e.g. HPLC).

Notably, in some embodiments, the siNA molecule (either the 5′ or 3′ strand or both) may begin with at least one, preferably two alanine nucleotides. Alternatively, if the target sequence starts with one or two alanine sequences, these may not be included (targeted) in the siNA molecule.

In one embodiment, the target sequence may be characterised by at least one, preferably two alanine nucleotides at the 3′ end of the sequence, and/or the target sequence lacks at least one, preferably two alanine nucleotides at the 5′ end of the sequence, and/or the target sequence lacks two consecutive alanine nucleotides within the sequence. In a preferred embodiment, the siNA molecules of the disclosure are characterised in that they target sequences with the above properties.

In one embodiment a plurality of species of siNA molecule are used, wherein said plurality of siNA molecules are targeted to the same or a different mRNA species.

In one embodiment, the siNA is selected from dsRNA, siRNA or shRNA. Preferably, the siNA is siRNA.

In one embodiment, an isolated or synthetic siNA molecule comprising at least a sequence 88% identical to SEQ ID No 396, SEQ ID No 439, SEQ ID No 440, SEQ ID No 442, SEQ ID No 443, SEQ ID No 511, SEQ ID No 530, SEQ ID No 546, SEQ ID No 548, SEQ ID No 549, SEQ ID No 555, SEQ ID No 556, SEQ ID No 596, SEQ ID No 614, SEQ ID No 624, SEQ ID No 625, SEQ ID No 630, SEQ ID No 631, SEQ ID No 633, SEQ ID No 642, SEQ ID No 650, SEQ ID No 654, SEQ ID No 655, SEQ ID No 656, SEQ ID No 657, SEQ ID No 658, SEQ ID No 659, SEQ ID No 660, SEQ ID No 661, SEQ ID No 692, SEQ ID No 693, SEQ ID No 694. Preferably at least 89% identical, or at least 90% identical, or at least 91% identical, or at least 92% identical, or at least 93% identical, or at least 94% identical, or at least 95% identical, or at least 96% identical, or at least 97% identical, or at least 98% identical, or at least 99% identical, or 100% identical to SEQ ID No 396, SEQ ID No 439, SEQ ID No 440, SEQ ID No 442, SEQ ID No 443, SEQ ID No 511, SEQ ID No 530, SEQ ID No 546, SEQ ID No 548, SEQ ID No 549, SEQ ID No 555, SEQ ID No 556, SEQ ID No 596, SEQ ID No 614, SEQ ID No 624, SEQ ID No 625, SEQ ID No 630, SEQ ID No 631, SEQ ID No 633, SEQ ID No 642, SEQ ID No 650, SEQ ID No 654, SEQ ID No 655, SEQ ID No 656, SEQ ID No 657, SEQ ID No 658, SEQ ID No 659, SEQ ID No 660, SEQ ID No 661, SEQ ID No 692, SEQ ID No 693, SEQ ID No 694.

In one embodiment, an isolated or synthetic siNA molecule comprising at least a sequence 88% identical SEQ ID No 743, SEQ ID No 786, SEQ ID No 787, SEQ ID No 789, SEQ ID No 790, SEQ ID No 858, SEQ ID No 877, SEQ ID No 893, SEQ ID No 895, SEQ ID No 896, SEQ ID No 902, SEQ ID No 903, SEQ ID No 943, SEQ ID No 961, SEQ ID No 971, SEQ ID No 972, SEQ ID No 977, SEQ ID No 978, SEQ ID No 980, SEQ ID No 989, SEQ ID No 997, SEQ ID No 1001, SEQ ID No 1002, SEQ ID No 1003, SEQ ID No 1004, SEQ ID No 1005, SEQ ID No 1006, SEQ ID No 1007, SEQ ID No 1008, SEQ ID No 1039, SEQ ID No 1040 and SEQ ID No 104. Preferably at least 89% identical, or at least 90% identical, or at least 91% identical, or at least 92% identical, or at least 93% identical, or at least 94% identical, or at least 95% identical, or at least 96% identical, or at least 97% identical, or at least 98% identical, or at least 99% identical, or 100% identical to SEQ ID No 743, SEQ ID No 786, SEQ ID No 787, SEQ ID No 789, SEQ ID No 790, SEQ ID No 858, SEQ ID No 877, SEQ ID No 893, SEQ ID No 895, SEQ ID No 896, SEQ ID No 902, SEQ ID No 903, SEQ ID No 943, SEQ ID No 961, SEQ ID No 971, SEQ ID No 972, SEQ ID No 977, SEQ ID No 978, SEQ ID No 980, SEQ ID No 989, SEQ ID No 997, SEQ ID No 1001, SEQ ID No 1002, SEQ ID No 1003, SEQ ID No 1004, SEQ ID No 1005, SEQ ID No 1006, SEQ ID No 1007, SEQ ID No 1008, SEQ ID No 1039, SEQ ID No 1040 and SEQ ID No 104.

Methods for the alignment of sequences for comparison are well known in the art, such methods include GAP, BESTFIT, BLAST, FASTA and TFASTA. GAP uses the algorithm of Needleman and Wunsch ((1970) J Mol Biol 48: 443-453) to find the global (over the whole the sequence) alignment of two sequences that maximizes the number of matches and minimizes the number of gaps. The BLAST algorithm (Altschul et al. (1990) J Mol Biol 215: 403-10) calculates percent sequence identity and performs a statistical analysis of the similarity between the two sequences. The software for performing BLAST analysis is publicly available through the National Centre for Biotechnology Information (NCBI). Global percentages of similarity and identity may also be determined using one of the methods available in the MatGAT software package (Campanella et al., BMC Bioinformatics. 2003 Jul. 10; 4:29. MatGAT: an application that generates similarity/identity matrices using protein or DNA sequences). Minor manual editing may be performed to optimise alignment between conserved motifs, as would be apparent to a person skilled in the art. The sequence identity values, which are indicated in the present subject matter as a percentage were determined over the entire amino acid sequence, using BLAST with the default parameters.

In a further embodiment, the disclosure relates to a siNA molecule, as herein described for use as a medicament. In one embodiment, the disclosure relates to a siNA for use in the treatment of a disorder characterised by increased expression levels (compared to the levels in a healthy subject) of SARS-CoV-2 non-structural proteins (NSPs).

In another aspect of the disclosure, there is provided a siNA molecule, as described herein for preventing and treating infections by the coronavirus SARS-CoV-2.

In a further aspect, the disclosure relates to the use of at least one siNA molecule, as described herein in the preparation of a medicament for preventing and treating infections by the coronavirus SARS-CoV-2.

In another aspect, the disclosure relates to a method for preventing and treating infections by the coronavirus SARS-CoV-2, the method comprising administering at least one siNA molecule, as described herein, to a patient or subject in need thereof.

In one embodiment, infection by the coronavirus SARS-CoV-2 is selected from asymptomatic infection, mild upper respiratory tract illness, severe viral pneumonia and with respiratory failure.

In another aspect of the disclosure there is provided a pharmaceutical composition comprising at least one siNA molecule as described herein and a pharmaceutically acceptable carrier.

In a further aspect of the disclosure there is provided a method, preferably an in vitro method of inhibiting non-structural proteins (NSPs) expression for virus-cell interactions during viral life cycle, the method comprising administering a siNA as defined herein to a cell. Preferably, the viral life cycle is promoted by non-structural proteins (NSPs). In one embodiment, non-structural proteins (NSPs) expression in a cell is inhibited by up to or more than 10%, 20%, 30%, 40%, 50%, 60%, 70%, 80% or 90% when compared to the level in a control.

In a further aspect of the disclosure there is provided a method, preferably an in vitro method of inhibiting non-structural proteins (NSPs) for virus-cell interactions during viral life cycle, the method comprising administering a siNA as defined herein to a cell. Preferably, the viral life cycle is promoted by the non-structural proteins (NSPs). In one embodiment, viral life cycle is inhibited by up to or more than 10%, 20%, 30%, 40%, 50%, 60%, 70%, 80% or 90% when compared to the level in a control

In a yet further aspect of the disclosure, there is provided a method of reducing viral infection, preferably in a patient, the method comprising administering at least one siNA as described herein. In one embodiment, said decrease in viral infection may be up to or more than 10%, 20%, 30%, 40%, 50%, 60%, 70%, 80% or 90% when compared to the level in a control.

In another embodiment, the disclosure relates to methods of reducing viral life cycle comprising treating the cells with an siNA of the disclosure in combination with one or more anti-viral agents known in the art, preferably wherein the anti-viral agent comprises a nucleoside analogue antiviral agent and most preferably favipiravir, ribavirin, remdesivir and galidesivir.

The disclosure also relates to methods of treating viral infection comprising administrating an siNA of the disclosure in combination with one or more anti-viral agents known in the art, preferably to a patient in need thereof, preferably wherein the anti-viral agent comprises an anti-nucleoside agent, more preferably an antiviral agent and most preferably favipiravir, ribavirin, remdesivir and galidesivir. The disclosure further relates to pharmaceutical compositions comprising the siNA of the disclosure and the one or more anti-viral agent.

In another embodiment the disclosure relates to methods for increasing the efficacy of an anti-viral therapy given to a patient comprising administering an siNA of the disclosure in combination with the therapy. Said increase in efficacy may be up to or more than 10%, 20%, 30%, 40%, 50%, 60%, 70%, 80% or 90% when compared to the efficacy of either administration of siNA or the anti-viral agent alone.

The disclosure also relates to methods of treating viral infection comprising administrating an siNA of the disclosure in combination with one or more transmembrane protease serine 2 (TMPRSS2) inhibitors known in the art, preferably to a patient in need thereof, preferably wherein the anti-TMPRSS2 agent comprises an, more preferably an anti-TMPRSS2 agent and most preferably camostat or nafamostat. The disclosure further relates to pharmaceutical compositions comprising the siNA of the disclosure and the one or more anti-TMPRSS2 agent.

In another embodiment the disclosure relates to methods for increasing the efficacy of TMPRSS2 inhibition therapy given to a patient comprising administering an siNA of the disclosure in combination with the therapy. Said increase in efficacy may be up to or more than 10%, 20%, 30%, 40%, 50%, 60%, 70%, 80% or 90% when compared to the efficacy of either administration of siNA or the TMPRSS2 inhibition therapy alone.

### DETAILED DESCRIPTION

The present disclosure relates to method of producing and using siNAs for preventing and treating coronavirus-inflicted infectious conditions. siNAs for preventing and treating infections by the coronavirus SARS-CoV-2, the causative viral agent of the novel coronavirus disease COVID-19, to mediate gene silencing of viral proteins.

According to a second aspect of the present disclosure, there is provided a method of treating or preventing by the coronavirus SARS-CoV-2, the causative viral agent of the novel coronavirus disease COVID-19, comprising administering to an individual an effective amount of a siRNA that inhibits non-structural proteins (NSPs) gene expression, wherein the siRNA comprises a sense non-structural proteins (NSPs) nucleic acid and an antisense spike (S) glycoprotein non-structural proteins (NSPs) nucleic acid. The present disclosure also provides a method of treating or preventing coronavirus-inflicted infectious conditions comprising administering to an individual an effective amount of a vector encoding the siRNA that inhibits non-structural proteins (NSPs) gene expression.

The non-structural proteins (NSPs) of coronaviruses, namely the SARS-CoV-2 non-structural proteins (NSPs), are key enzymes in the viral life cycle. The present disclosure is based on the surprising discovery that small interfering RNAs (siRNAs) selective for SARS-CoV-non-structural proteins (NSPs) are effective preventing and treating the coronavirus SARS-CoV-2 inflicted infectious conditions. In particular, infections by the coronavirus SARS-CoV-2 selected from asymptomatic infection, mild upper respiratory tract illness, severe viral pneumonia and with respiratory failure.

The siRNA or vector encoding the siRNA, or the medicament comprising the siRNA or vector encoding the siRNA, may be administered to an individual by topical application, nasal application, inhalation administration, subcutaneous injection or deposition, subcutaneous infusion, intravenous injection, intravenous infusion.

According to a third aspect of the present disclosure there is provided an in vitro method of inhibiting the expression of the non-structural proteins (NSPs) gene in a cell comprising contacting the cell with siNA that inhibits non-structural proteins (NSPs) gene expression as described herein. In one embodiment, said siRNA comprises a sense non-structural proteins (NSPs) nucleic acid and an anti-non-structural proteins (NSPs) nucleic acid, wherein the non-structural proteins (NSPs) nucleic acid is substantially identical to a target sequence contained within non-structural proteins (NSPs) mRNA and the anti-sense non-structural proteins (NSPs) nucleic acid is complementary to the sense non-structural proteins (NSPs) nucleic acid. The present disclosure also provides an in vitro method of inhibiting the expression of the non-structural proteins (NSPs) gene in a cell comprising contacting the cell with a vector encoding a siRNA that inhibits non-structural proteins (NSPs) gene expression, said siRNA comprises a sense non-structural proteins (NSPs) nucleic acid and an anti-sense non-structural proteins (NSPs) nucleic acid, wherein the sense non-structural proteins (NSPs) nucleic acid is substantially identical to a target sequence contained within non-structural proteins (NSPs) mRNA and the anti-sense non-structural proteins (NSPs) nucleic acid is complementary to the sense non-structural proteins (NSPs) nucleic acid.

Expression of the gene may be inhibited by introduction of a double stranded ribonucleic acid (dsRNA) molecule into the cell in an amount sufficient to inhibit expression of the non-structural proteins (NSPs) gene.

The siRNAs used in the disclosure are believed to cause the RNAi-mediated degradation of non-structural proteins (NSPs) from SARS-CoV-2 mRNA so that the protein products of the non-structural proteins (NSPs) from SARS-CoV-2 gene is not produced or is produced in reduced amounts. The siRNAs used in the disclosure can be used to alter gene expression in a cell in which expression of non-structural proteins (NSPs) from SARS-CoV-2 is initiated, e.g., as a result of SARS-CoV-2-inflicted infectious conditions such as in asymptomatic infection, mild upper respiratory tract illness, severe viral pneumonia and with respiratory failure. Binding of the siRNA to the non-structural proteins (NSPs) mRNA transcript in a cell results in a reduction in non-structural proteins (NSPs) production by the infected cell.

The term “siRNA” is used to mean a double stranded RNA molecule which prevents translation of a target mRNA. Standard techniques of introducing siRNA into the cell are used, including those in which DNA is a template from which RNA is transcribed. The siRNA that inhibits non-structural proteins (NSPs) from SARS-CoV-2 gene expression includes a sense non-structural proteins (NSPs) from SARS-CoV-2 nucleic acid sequence and an antisense non-structural proteins (NSPs) from SARS-CoV-2 nucleic acid sequence. The siRNA may be constructed such that a single transcript has both the sense and complementary antisense sequences from the target gene, e.g., in the form of a hairpin.

The siRNA preferably comprises short double-stranded RNA that is targeted to the target mRNA, i.e., non-structural proteins (NSPs) from SARS-CoV-2 mRNA. The siRNA comprises a sense RNA strand and a complementary antisense RNA strand annealed together by standard Watson-Crick base-pairing interactions (hereinafter “base-paired”). The sense strand comprises a nucleic acid sequence which is substantially identical to a target sequence contained within the non-structural proteins (NSPs) from SARS-CoV-2 mRNA.

The terms “sense/antisense sequences” and “sense/antisense strands” are used interchangeable herein to refer to the parts of the siRNA of the present disclosure that are substantially identical (sense) to the target SARS-CoV-2 mRNA sequence or substantially complementary (antisense) to the target non-structural proteins (NSPs) from SARS-CoV-2 mRNA sequence.

As used herein, a nucleic acid sequence “substantially identical” to a target sequence contained within the target mRNA is a nucleic acid sequence which is identical to the target sequence, or which differs from the target sequence by one or more nucleotides. Preferably, the substantially identical sequence is identical to the target sequence or differs from the target sequence by one, two or three nucleotides, more preferably by one or two nucleotides and most preferably by only 1 nucleotide. Sense strands which comprise nucleic acid sequences substantially identical to a target sequence are characterized in that siRNA comprising such a sense strand induces RNAi-mediated degradation of mRNA containing the target sequence. For example, an siRNA of the disclosure can comprise a sense strand comprising a nucleic acid sequence which differs from a target sequence by one, two, three or more nucleotides, as long as RNAi-mediated degradation of the target mRNA is induced by the siRNA.

The sense and antisense strands of the siRNA can comprise two complementary, single-stranded RNA molecules or can comprise a single molecule in which two complementary portions are base-paired and are covalently linked by a single-stranded “hairpin” area. That is, the sense region and antisense region can be covalently connected via a linker molecule. The linker molecule can be a polynucleotide or non-nucleotide linker. The siRNA can also contain alterations, substitutions or modifications of one or more ribonucleotide bases. For example, the present siRNA can be altered, substituted or modified to contain one or more, preferably 0, 1, 2 or 3, deoxyribonucleotide bases. Preferably, the siRNA does not contain any deoxyribonucleotide bases.

The siRNA can comprise partially purified RNA, substantially pure RNA, synthetic RNA, or recombinantly produced RNA, as well as altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA; modifications that make the siRNA resistant to nuclease digestion (e.g., the use of 2′-substituted ribonucleotides or modifications to the sugar-phosphate backbone); or the substitution of one or more, preferably 0, 1, 2 or 3, nucleotides in the siRNA with deoxyribonucleotides.

Degradation can be delayed or avoided by a wide variety of chemical modifications that include alterations in the nucleobases, sugars and the phosphate ester backbone of the siRNAs. All of these chemically modified siRNAs are still able to induce siRNA-mediated gene silencing provided that the modifications were absent in specific regions of the siRNA and included to a limited extent. In general, backbone modifications cause a small loss in binding affinity, but offer nuclease resistance. Phosphorothioate (PS)- or boranophosphate (BS)-modified siRNAs have substantial nuclease resistance. Silencing by siRNA duplexes is also compatible with some types of 2′-sugar modifications: 2′-H, 2′-O-methyl, 2′-O-methoxyethyl, 2′-fluoro (2′-F), locked nucleic acid (LNA) and ethylene-bridge nucleic acid (ENA). Suitable chemical modifications are well known to those skilled in the art.

The siRNA used in the present disclosure is a double-stranded molecule comprising a sense strand and an antisense strand, wherein the sense strand comprises or consists of a ribonucleotide sequence corresponding to non-structural proteins (NSPs) from SARS-CoV-2 target sequence, and wherein the antisense strand comprises a ribonucleotide sequence which is complementary to said sense strand, wherein said sense strand and said antisense strand hybridize to each other to form said double-stranded molecule, and wherein said double-stranded molecule, when introduced into a cell expressing the non-structural proteins (NSPs) from SARS-CoV-2 gene, inhibits expression of the said gene. As indicated further below, said non-structural proteins (NSPs) from SARS-CoV-2 target sequence preferably comprises at least about 15 contiguous, more preferably 19 to 25, and most preferably about 19 to 21 contiguous nucleotides selected from the group consisting of from SEQ ID No 58, SEQ ID No 59, SEQ ID No 60, SEQ ID No 61, SEQ ID No 62, SEQ ID No 86, SEQ ID No 152, SEQ ID No 153, SEQ ID No 210, SEQ ID No 250, SEQ ID No 263, SEQ ID No 314, SEQ ID No 324, SEQ ID No 325, SEQ ID No 338, SEQ ID No 339, SEQ ID No 345, SEQ ID No 346, SEQ ID No 347, SEQ ID No 352, SEQ ID No 353, SEQ ID No 354, SEQ ID No 367, SEQ ID No 368, SEQ ID No 373, SEQ ID No 374, SEQ ID No 375, SEQ ID No 376, SEQ ID No 377, SEQ ID No 384, SEQ ID No 484, SEQ ID No 485, SEQ ID No 495, SEQ ID No 496, SEQ ID No 497, SEQ ID No 498, SEQ ID No 506, SEQ ID No 517, SEQ ID No 524, SEQ ID No 542, SEQ ID No 545, SEQ ID No 546, SEQ ID No 548, SEQ ID No 558, SEQ ID No 559, SEQ ID No 565, SEQ ID No 567, SEQ ID No 570, SEQ ID No 574, SEQ ID No 579, SEQ ID No 580 and SEQ ID No 582.

The siRNA used in the present disclosure can be obtained using a number of techniques known to those of skill in the art. For example, the siRNA can be chemically synthesized or recombinantly produced using methods known in the art, such as the Drosophila in vitro system described in U.S. published application 2002/0086356, the entire disclosure of which is herein incorporated by reference. The siRNA may be chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Biospring (Frankfurt, Germany), ChemGenes (Ashland, Mass., USA), Dharmacon Research (Lafayette, Colo., USA), Glen Research (Sterling, Va., USA), Proligo (Hamburg, Germany), Sigma-Aldrich (St. Louis, Mo. USA) and Thermo Fisher Scientific (Waltham, Mass. USA).

The siRNA can also be expressed from recombinant circular or linear DNA vectors using any suitable promoter. Suitable promoters for expressing siRNA from a vector include, for example, the U6 or H1 RNA pol III promoter sequences and the cytomegalovirus promoter. Selection of other suitable promoters is within the skill in the art. The vector can also comprise inducible or regulable promoters for expression of the siRNA in a particular tissue or in a particular intracellular environment.

The siRNA expressed from a vector can either be isolated from cultured cell expression systems by standard techniques or can be expressed intracellularly. The vector can be used to deliver the siRNA to cells in vivo, e.g., by intracellularly expressing the siRNA in vivo. siRNA can be expressed from a vector either as two separate, complementary RNA molecules, or as a single RNA molecule with two complementary regions. Selection of vectors suitable for expressing the siRNA, methods for inserting nucleic acid sequences for expressing the siRNA into the vector, and methods of delivering the vector to the cells of interest are well known to those skilled in the art.

The siRNA can also be expressed from a vector intracellularly in vivo. As used herein, the term “vector” means any nucleic acid- and/or viral-based technique used to deliver a desired nucleic acid. Any vector capable of accepting the coding sequences for the siRNA molecule(s) to be expressed can be used, including plasmids, cosmids, naked DNA, optionally condensed with a condensing agent, and viral vectors. Suitable viral vectors include vectors derived from adenovirus (AV); adeno-associated virus (AAV); retroviruses (e.g., lentiviruses (LV), Rhabdoviruses, murine leukemia virus); herpes virus, and the like. The tropism of viral vectors can be modified by pseudotyping the vectors with envelope proteins or other surface antigens from other viruses, or by substituting different viral capsid proteins, as appropriate. When the vector is a lentiviral vector it is preferably pseudotyped with surface proteins from vesicular stomatitis virus, rabies virus, Ebola virus or Mokola virus.

Vectors are produced for example by cloning the non-structural proteins (NSPs) from SARS-CoV-2 target sequence into an expression vector so that operatively-linked regulatory sequences flank the non-structural proteins (NSPs) sequence in a manner that allows for expression (by transcription of the DNA molecule) of both strands (Lee et al., 2002). An RNA molecule that is antisense to non-structural proteins (NSPs) mRNA is transcribed by a first promoter (e.g., a promoter sequence 3′ of the cloned DNA) and an RNA molecule that is the sense strand for the non-structural proteins (NSPs) mRNA is transcribed by a second promoter (e. g., a promoter sequence 5′ of the cloned DNA). The sense and antisense strands hybridize in vivo to generate siRNA constructs for silencing of the non-structural proteins (NSPs) gene. Alternatively, two vectors are utilized to create the sense and anti-sense strands of a siRNA construct. Cloned non-structural proteins (NSPs) can encode a construct having secondary structure, e. g., hairpins, wherein a single transcript has both the sense and complementary antisense sequences from the target gene. Such a transcript encoding a construct having secondary structure, will preferably comprises a single-stranded ribonucleotide sequence (loop sequence) linking said sense strand and said antisense strand.

The siRNA is preferably isolated. As used herein, “isolated” means synthetic, or altered or removed from the natural state through human intervention. For example, a siRNA naturally present in a living animal is not “isolated,” but a synthetic siRNA, or a siRNA partially or completely separated from the coexisting materials of its natural state is “isolated.” An isolated siRNA can exist in substantially purified form, or can exist in a non-native environment such as, for example, a cell into which the siRNA has been delivered. By way of example, siRNA which are produced inside a cell by natural processes, but which are produced from an “isolated” precursor molecule, are themselves “isolated” molecules. Thus, an isolated dsRNA can be introduced into a target cell, where it is processed by the Dicer protein (or its equivalent) into isolated siRNA.

As used herein, “inhibit” means that the activity of the non-structural proteins (NSPs) gene expression product or level of the non-structural proteins (NSPs) gene expression product is reduced below that observed in the absence of the siRNA molecule of the disclosure. The inhibition with a siRNA molecule preferably is significantly below that level observed in the presence of an inactive or attenuated molecule that is unable to mediate an RNAi response. Inhibition of gene expression with the siRNA molecule is preferably significantly greater in the presence of the siRNA molecule than in its absence. Preferably, the siRNA inhibits the level of non-structural proteins (NSPs) gene expression by at least 10%, more preferably at least 50% and most preferably at least 75%.

Preferably the siRNA molecule inhibits non-structural proteins (NSPs) gene expression so that the protein product of the non-structural proteins (NSPs) from SARS-CoV-2 gene is not produced or is produced in reduced amounts. By inhibiting non-structural proteins (NSPs) expression during viral life cycle is meant that the treated cell produces at a lower rate or has decreased the viral proteins that allows viral replication than an untreated cell. The non-structural proteins (NSPs) from SARS-CoV-2 is measured by mRNA or protein assays known in the art.

As used herein, an “isolated nucleic acid” is a nucleic acid removed from its original environment (e. g., the natural environment if naturally occurring) and thus, synthetically altered from its natural state. In the present disclosure, isolated nucleic acid includes DNA, RNA, and derivatives thereof. When the isolated nucleic acid is RNA or derivatives thereof, base “t” should be replaced with “u” in the nucleotide sequences.

As used herein, the term “complementary” refers to Watson-Crick or Hoogsteen base pairing between nucleotides units of a polynucleotide, and the term “binding” means the physical or chemical interaction between two polypeptides or compounds or associated polypeptides or compounds or combinations thereof.

As used herein, the phrase “highly conserved sequence region” means a nucleotide sequence of one or more regions in a target gene does not vary significantly from one generation to the other or from one biological system to the other.

As used herein, the term “complementarity” or “complementary” means that a nucleic acid can form hydrogen bond(s) with another nucleic acid sequence by either traditional Watson-Crick or other non-traditional types of interaction. In reference to the present disclosure, the binding free energy for a siRNA molecule with its complementary sequence is sufficient to allow the relevant function of the nucleic acid to proceed, e.g., RNAi activity. For example, the degree of complementarity between the sense and antisense strand of the siRNA molecule can be the same or different from the degree of complementarity between the antisense strand of the siRNA and the target RNA sequence.

A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. Preferably the term “complementarity” or “complementary” means that at least 90%, more preferably at least 95% and most preferably 100% of residues in a first nucleic acid sense can form hydrogen binds with a second nucleic acid sequence.

Complementary nucleic acid sequences hybridize under appropriate conditions to form stable duplexes containing few (one or two) or no mismatches. Furthermore, the sense strand and antisense strand of the siRNA can form a double stranded nucleotide or hairpin loop structure by the hybridization. In a preferred embodiment, such duplexes contain no more than 1 mismatch for every 10 matches. In an especially preferred embodiment, the sense and antisense strands of the duplex are fully complementary, i.e., the duplexes contain no mismatches.

As used herein, the term “cell” is defined using its usual biological sense. The cell can be present in an organism, e.g., mammals such as humans, cows, sheep, apes, monkeys, swine, dogs, and cats. The cell can be eukaryotic (e.g., a mammalian cell). The cell can be of somatic or germ line origin, totipotent or pluripotent, dividing or non-dividing. The cell can also be derived from or can comprise a gamete or embryo, a stem cell, or a fully differentiated cell. Preferably the cell is in the upper respiratory tract, pulmonary parenchyma, brain, colon, head and neck, kidney, liver, lung, or lymph.

As used herein, the term “RNA” means a molecule comprising at least one ribonucleotide residue. By “ribonucleotide” is meant a nucleotide with a hydroxyl group at the 2′ position of a beta-D-ribo-furanose moiety. The term includes double stranded RNA, single stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or internally, for example at one or more nucleotides of the RNA. Nucleotides in the RNA molecules of the instant disclosure can also comprise non-standard nucleotides, such as non-naturally occurring nucleotides or chemically synthesized nucleotides or deoxynucleotides. These altered RNAs can be referred to as analogues of naturally-occurring RNA. Preferably the term “RNA” consists of ribonucleotide residues only.

As used herein, the term “organism” refers to any living entity comprised of at least one cell. A living organism can be as simple as, for example, a single eukaryotic cell or as complex as a mammal, including a human being.

As used herein, the term “subject” means an organism, which is a donor or recipient of explanted cells or the cells themselves. “Subject” also refers to an organism to which the nucleic acid molecules of the disclosure can be administered. The subject is preferably a mammal, e.g., a human, non-human primate, mouse, rat, dog, cat, horse, or cow. Most preferably the subject is a human.

As used herein, the term “biological sample” refers to any sample containing polynucleotides. The sample may be a tissue or cell sample, or a body fluid containing polynucleotides (e.g., blood, mucus, lymphatic fluid, synovial fluid, cerebrospinal fluid, saliva, amniotic fluid, amniotic cord blood, urine, vaginal fluid and semen). The sample may be a homogenate, lysate, extract, cell culture or tissue culture prepared from a whole organism or a subset of its cells, tissues or component parts, or a fraction or portion thereof. Lastly, the sample may be a medium, such as a nutrient broth or gel in which an organism, or cells of an organism, have been propagated, wherein the sample contains polynucleotides.

The disclosure relates to methods of inhibiting non-structural proteins (NSPs) gene expression so that the protein product of the non-structural proteins (NSPs) from SARS-CoV-2 gene is not produced or is produced in reduced amounts. In particular, the disclosure provides a method for can be used to alter gene expression in a cell in which expression of non-structural proteins (NSPs) from SARS-CoV-2 is initiated, e.g., as a result of SARS-CoV-2-inflicted infectious conditions such as in asymptomatic infection, mild upper respiratory tract illness, severe viral pneumonia and with respiratory failure. Binding of the siRNA to non-structural proteins (NSPs) mRNA transcript in a cell results in a reduction in non-structural proteins (NSPs) production by the infected cell. The cell may be further contacted with a transfection-enhancing agent to enhance delivery of the siRNA or siRNA encoding vector to the cell. Depending on the specific method of the present disclosure, the cell may be provided in vitro, in vivo or ex vivo.

Sequence information regarding the coronavirus SARS-CoV-2 non-structural proteins (NSPs) gene (GenBank accession NM_908947) was extracted from the NCBI Entrez nucleotide database. Up to 399 mRNA segments were identified. See for example, U.S. Pat. No. 6,506,559, and Elbashir et al., 2001, herein incorporated by reference in its entirety.

Selection of siRNA target sites can be performed as follows:
i) Beginning with the ATG start codon of the transcript, scan downstream for AA dinucleotide sequences. Record the occurrence of each AA and the 3′ adjacent 19 nucleotides as potential siRNA target sites. Tuschl et al. recommend against designing siRNA to the 5′ and 3′ untranslated regions (UTRs) and regions near the start codon (within 75 bases) as these may be richer in regulatory protein binding sites. UTR-binding proteins and/or translation initiation complexes may interfere with binding of the siRNA endonuclease complex.
ii) Compare the potential target sites to the appropriate genome database (human, mouse, rat, etc.) and eliminate from consideration any target sequences with significant homology to other coding sequences. We suggest using BLAST, which can be found on the NCBI server at: www.ncbi.nlm.nih.gov/BLAST/iii)
iii) Select qualifying target sequences (i.e., sequences having over 45% GC content) for synthesis.

In one aspect of the disclosure, the length of the sense nucleic acid is at least 10 nucleotides and may be as long as the naturally-occurring non-structural proteins (NSPs) transcript. Preferably, the sense nucleic acid is less than 75, 50, or 25 nucleotides in length. It is further preferred that the sense nucleic acid comprises at least 19 nucleotides. Most preferably, the sense nucleic acid is 19-25 nucleotides in length. Examples of non-structural proteins (NSPs) from SARS-CoV-2 target siRNA sense nucleic acids of the present disclosure which inhibit non-structural proteins (NSPs) expression in mammalian cells include oligonucleotides comprising any one of the following target sequences of non-structural proteins (NSPs) gene: SEQ ID 58, 59, 60, 61, 62, 86, 152, 153, 210, 250, 263, 314, 324, 325, 338, 339, 345, 346, 347, 352, 353, 354, 367, 368, 373, 374, 375, 376, 377, 384, 484, 485, 495, 496, 497, 498, 506, 517, 524, 542, 545, 546, 548, 558, 559, 565, 567, 570, 574, 579, 580 and 582.

Three hundred and forty-seven sequences, which set forth the sequence for one strand of the double stranded is RNA, were identified and isolated for non-structural proteins (NSPs) from SARS-CoV-2 (Table 1).

TABLE 1
5′ sense SARS-CoV-2 DNA target
non-structural proteins (NSPs).

| SEQ ID No | 5′ DNA sense |
|---|---|
| SEQ ID No 1 | AGCTGATGTTACTAAAATAAA |
| SEQ ID No 2 | GCTGATGTTACTAAAATAAAA |
| SEQ ID No 3 | ATGGAGCTGATGTTACTAAAA |
| SEQ ID No 4 | GAGCTGATGTTACTAAAATAA |
| SEQ ID No 5 | GTGGTCACTATAAACATATAA |
| SEQ ID No 6 | AAATAAAACCTCATAATTCAC |
| SEQ ID No 7 | AAACAAGCTACAAAATATCTA |
| SEQ ID No 8 | AACAAGCTACAAAATATCTAG |
| SEQ ID No 9 | GGATGGAGCTGATGTTACTAA |
| SEQ ID No 10 | GATGGAGCTGATGTTACTAAA |
| SEQ ID No 11 | ATAATTCACATGAAGGTAAAA |
| SEQ ID No 12 | TAGGTACATGTCAGCATTAAA |
| SEQ ID No 13 | CAGCATTAAATCACACTAAAA |
| SEQ ID No 14 | AGCATTAAATCACACTAAAAA |
| SEQ ID No 15 | ATCTTGCCACTGCATTGTTAA |
| SEQ ID No 16 | TATCTTAGCCTACTGTAATAA |
| SEQ ID No 17 | AGTTAGGTGATGTTAGAGAAA |
| SEQ ID No 18 | TCTTGAACGTGGTGTGTAAAA |
| SEQ ID No 19 | ACCTGCTCAGTATGAACTTAA |
| SEQ ID No 20 | TTACCAGTGTGGTCACTATAA |
| SEQ ID No 21 | TACCAGTGTGGTCACTATAAA |
| SEQ ID No 22 | CTATAAACATATAACTTCTAA |
| SEQ ID No 23 | TATAAACATATAACTTCTAAA |
| SEQ ID No 24 | AAACATATAACTTCTAAAGAA |
| SEQ ID No 25 | AACATATAACTTCTAAAGAAA |
| SEQ ID No 26 | TACAAAGTCCTCAGAATACAA |
| SEQ ID No 27 | ACAAAGTCCTCAGAATACAAA |
| SEQ ID No 28 | AAAATAAAACCTCATAATTCA |
| SEQ ID No 29 | AATAAAACCTCATAATTCACA |
| SEQ ID No 30 | AACACTCCAACAAATAGAGTT |
| SEQ ID No 31 | AACGTGGTGTGTAAAACTTGT |
| SEQ ID No 32 | AAGCTACAAAATATCTAGTAC |
| SEQ ID No 33 | AAACATATAACTTCTAAAGAA |
| SEQ ID No 34 | AACATATAACTTCTAAAGAAA |
| SEQ ID No 35 | AAAGTCCTCAGAATACAAAGG |
| SEQ ID No 36 | TACTAAAATAAAACCTCATAA |
| SEQ ID No 37 | AAACCTCATAATTCACATGAA |
| SEQ ID No 38 | TCATAATTCACATGAAGGTAA |
| SEQ ID No 39 | CATAATTCACATGAAGGTAAA |
| SEQ ID No 40 | GTAGGTACATGTCAGCATTAA |
| SEQ ID No 41 | GTCAGCATTAAATCACACTAA |
| SEQ ID No 42 | TCAGCATTAAATCACACTAAA |
| SEQ ID No 43 | AAATCACACTAAAAAGTGGAA |
| SEQ ID No 44 | AATCACACTAAAAAGTGGAAA |
| SEQ ID No 45 | CATTGTTAACACTCCAACAAA |
| SEQ ID No 46 | ACTCCAACAAATAGAGTTGAA |
| SEQ ID No 47 | ACTTATCTTAGCCTACTGTAA |
| SEQ ID No 48 | GAGTTAGGTGATGTTAGAGAA |
| SEQ ID No 49 | TAGGTGATGTTAGAGAAACAA |
| SEQ ID No 50 | TTCTTGCAAAAGAGTCTTGAA |
| SEQ ID No 51 | GTCTTGAACGTGGTGTGTAAA |
| SEQ ID No 52 | ATACCTTGTACGTGTGGTAAA |
| SEQ ID No 53 | CCTTGTACGTGTGGTAAACAA |
| SEQ ID No 54 | CGTGTGGTAAACAAGCTACAA |
| SEQ ID No 55 | GTGTGGTAAACAAGCTACAAA |
| SEQ ID No 56 | TGTGGTAAACAAGCTACAAAA |
| SEQ ID No 57 | GCTACAAAATATCTAGTACAA |
| SEQ ID No 58 | GCACCACCTGCTCAGTATGAA |
| SEQ ID No 59 | GCACCACCTGCTGAGTATAAA |
| SEQ ID No 60 | ACCACCTGCTCAGTATGAA |
| SEQ ID No 61 | CAGCACCACCTGCTCAGTATGAA |
| SEQ ID No 62 | GTCAGCACCACCTGCTCAGTATGAA |
| SEQ ID No 63 | ACAAAGAAAACAGTTACACAA |
| SEQ ID No 64 | AAGAAAACAGTTACACAACAA |
| SEQ ID No 65 | AATGACATATGGACAACAGTT |
| SEQ ID No 66 | AAAACCTCATAATTCACATGA |
| SEQ ID No 67 | AAACCTCATAATTCACATGAA |
| SEQ ID No 68 | AACCTCATAATTCACATGAAG |
| SEQ ID No 69 | AATTCACATGAAGGTAAAACA |
| SEQ ID No 70 | AAATCACACTAAAAAGTGGAA |
| SEQ ID No 71 | AATCACACTAAAAAGTGGAAA |
| SEQ ID No 72 | AATCCACCTGCTCTACAAGAT |
| SEQ ID No 73 | AAAGGTGTTCAGATACCTTGT |
| SEQ ID No 74 | AAGGTGTTCAGATACCTTGTA |
| SEQ ID No 75 | AAGTCCTCAGAATACAAAGGT |
| SEQ ID No 76 | AATACAAAGGTCCTATTACGG |
| SEQ ID No 77 | AAAGAAAACAGTTACACAACA |
| SEQ ID No 78 | AAGAAAACAGTTACACAACAA |
| SEQ ID No 79 | AAACAGTTACACAACAACCAT |
| SEQ ID No 80 | AACAGTTACACAACAACCATT |
| SEQ ID No 81 | ACTGCATTGTTAACACTCCAA |
| SEQ ID No 82 | GCATTGTTAACACTCCAACAA |
| SEQ ID No 83 | AAGATGCTTATTACAGAGCAA |
| SEQ ID No 84 | AGTCTTGAACGTGGTGTGTAA |
| SEQ ID No 85 | GTGTGTAAAACTTGTGGACAA |
| SEQ ID No 86 | CTTGTGGACAACAGCAGACAA |
| SEQ ID No 87 | GATACCTTGTACGTGTGGTAA |
| SEQ ID No 88 | TGCTAGTGAGTACACTGGTAA |
| SEQ ID No 89 | TTACTTACAAAGTCCTCAGAA |
| SEQ ID No 90 | AATGATGACACTCTACGTGTT |
| SEQ ID No 91 | AAGATGCTTATTACAGAGCAA |
| SEQ ID No 92 | AATAAGACAGTAGGTGAGTTA |
| SEQ ID No 93 | AAGACAGTAGGTGAGTTAGGT |
| SEQ ID No 94 | AAAAGAGTCTTGAACGTGGTG |
| SEQ ID No 95 | AAAACTTGTGGACAACAGCAG |
| SEQ ID No 96 | AAACTTGTGGACAACAGCAGA |
| SEQ ID No 97 | AACTTGTGGACAACAGCAGAC |
| SEQ ID No 98 | AAGAAAGGTGTTCAGATACCT |
| SEQ ID No 99 | AAAATATCTAGTACAACAGGA |
| SEQ ID No 100 | AAATATCTAGTACAACAGGAG |
| SEQ ID No 101 | AATATCTAGTACAACAGGAGT |
| SEQ ID No 102 | AATTACCAGTGTGGTCACTAT |
| SEQ ID No 103 | AAAGGTCCTATTACGGATGTT |
| SEQ ID No 104 | AAAACAGTTACACAACAACCA |
| SEQ ID No 105 | AACAACTGTTATCTTGCCACT |
| SEQ ID No 106 | AAAGAGTCTTGAACGTGGTGT |
| SEQ ID No 107 | AAGAGTCTTGAACGTGGTGTG |
| SEQ ID No 108 | AACTGTTATCTTGCCACTGCA |
| SEQ ID No 109 | CATTCGTAAGTCTAATCATAA |
| SEQ ID No 110 | GTTGTACGCTGCTGTTATAAA |
| SEQ ID No 111 | GCTATGAAGTACAATTATGAA |
| SEQ ID No 112 | TTGTGGTACAACTACACTTAA |
| SEQ ID No 113 | GTACAGGCTGGTAATGTTCAA |
| SEQ ID No 114 | TATTGGACATTCTATGCAAAA |
| SEQ ID No 115 | TATGCAAAATTGTGTACTTAA |
| SEQ ID No 116 | AAATTGTGTACTTAAGCTTAA |
| SEQ ID No 117 | ATCCTAAGACACCTAAGATAA |
| SEQ ID No 118 | TTCAGTGTTAGCTTGTTACAA |
| SEQ ID No 119 | TGCACCATATGGAATTACCAA |
| SEQ ID No 120 | GGACACAACTATTACAGTTAA |
| SEQ ID No 121 | GGTTGTACGCTGCTGTTATAA |
| SEQ ID No 122 | AAAATTGTGTACTTAAGCTTA |
| SEQ ID No 123 | AAATTGTGTACTTAAGCTTAA |
| SEQ ID No 124 | AATTGTGTACTTAAGCTTAAG |
| SEQ ID No 125 | AACCTTGTGGCTATGAAGTAC |
| SEQ ID No 126 | AAGACCATGTTGACATACTAG |
| SEQ ID No 127 | GCACCTCTGAAGACATGCTAA |
| SEQ ID No 128 | GTTATTGGACATTCTATGCAA |
| SEQ ID No 129 | TTATTGGACATTCTATGCAAA |
| SEQ ID No 130 | GGTTGATACAGCCAATCCTAA |
| SEQ ID No 131 | TGTTACATGCACCATATGGAA |
| SEQ ID No 132 | CATGCTGGCACAGACTTAGAA |
| SEQ ID No 133 | TGGCACAGACTTAGAAGGTAA |
| SEQ ID No 134 | CCTTGTGGCTATGAAGTACAA |
| SEQ ID No 135 | AGTACAATTATGAACCTCTAA |
| SEQ ID No 136 | AATTATGAACCTCTAACACAA |
| SEQ ID No 137 | AAGTAACTTGTGGTACAACTA |
| SEQ ID No 138 | AACTTGTGGTACAACTACACT |
| SEQ ID No 139 | AATCCTAAGACACCTAAGATA |
| SEQ ID No 140 | AATTACCAACTGGAGTTCATG |
| SEQ ID No 141 | AATTATGAACCTCTAACACAA |
| SEQ ID No 142 | AACCTCTAACACAAGACCATG |
| SEQ ID No 143 | AACACAAGACCATGTTGACAT |
| SEQ ID No 144 | TACAAGTAACTTGTGGTACAA |
| SEQ ID No 145 | CATGTGATCTGCACCTCTGAA |
| SEQ ID No 146 | GCTTAAGGTTGATACAGCCAA |
| SEQ ID No 147 | AGCCAATCCTAAGACACCTAA |
| SEQ ID No 148 | GTTGACAGGCAAACAGCACAA |
| SEQ ID No 149 | AAGCTTAAGGTTGATACAGCC |
| SEQ ID No 150 | AATGGTTCACCATCTGGTGTT |
| SEQ ID No 151 | AATGGTTCATGTGGTAGTGTT |
| SEQ ID No 152 | AACAGCACAAGCAGCTGGTAC |
| SEQ ID No 153 | AACTGCACAGGCTGCAGGTAC |
| SEQ ID No 154 | AAGTACAATTATGAACCTCTA |
| SEQ ID No 155 | AAGCAGCTGGTACGGACACAA |
| SEQ ID No 156 | AAGACATGTGATCTGCACCTC |
| SEQ ID No 157 | AAGGTTGATACAGCCAATCCT |
| SEQ ID No 158 | AACTGGAGTTCATGCTGGCAC |
| SEQ ID No 159 | AAACAGCACAAGCAGCTGGTA |
| SEQ ID No 160 | AAGCAGCTGGTACGGACACAA |
| SEQ ID No 161 | TGCTAAATTCCTAAAAACTAA |
| SEQ ID No 162 | GGTAATTGTGACACATTAAAA |
| SEQ ID No 163 | AATTGTGACACATTAAAAGAA |
| SEQ ID No 164 | ATTGTGACACATTAAAAGAAA |
| SEQ ID No 165 | TGGTGTACTGACATTAGATAA |
| SEQ ID No 166 | CATTGTTAATGCCTATATTAA |
| SEQ ID No 167 | CTTAACAAAGCCTTACATTAA |
| SEQ ID No 168 | AAGGAAGTTCTGTTGAATTAA |
| SEQ ID No 169 | AGGAAGTTCTGTTGAATTAAA |
| SEQ ID No 170 | GGAAGTTCTGTTGAATTAAAA |
| SEQ ID No 171 | GGTTGGCACAACATGTTAAAA |
| SEQ ID No 172 | GTTGGCACAACATGTTAAAAA |
| SEQ ID No 173 | TGGCGGTTCACTATATGTTAA |
| SEQ ID No 174 | GGCGGTTCACTATATGTTAAA |
| SEQ ID No 175 | AATTGTGACACATTAAAAGAA |
| SEQ ID No 176 | AATACTTGTCACATACAATTG |
| SEQ ID No 177 | AAGGAAGTTCTGTTGAATTAA |
| SEQ ID No 178 | AAGTTCTGTTGAATTAAAACA |
| SEQ ID No 179 | AAAAATTATTGAAATCAATAG |
| SEQ ID No 180 | AACATACAATGCTAGTTAAAC |
| SEQ ID No 181 | CTAACTACCAACATGAAGAAA |
| SEQ ID No 182 | ACTACCAACATGAAGAAACAA |
| SEQ ID No 183 | ATCACGTCAACGTCTTACTAA |
| SEQ ID No 184 | TCACGTCAACGTCTTACTAAA |
| SEQ ID No 185 | AACGTCTTACTAAATACACAA |
| SEQ ID No 186 | AAGGTAATTGTGACACATTAA |
| SEQ ID No 187 | AGGTAATTGTGACACATTAAA |
| SEQ ID No 188 | GTACTGACATTAGATAATCAA |
| SEQ ID No 189 | ATTCTTATTATTCATTGTTAA |
| SEQ ID No 190 | ATGTTGACACTGACTTAACAA |
| SEQ ID No 191 | TGTTGACACTGACTTAACAAA |
| SEQ ID No 192 | TTCACGGAAGAGAGGTTAAAA |
| SEQ ID No 193 | TTGGACCACTAGTGAGAAAAA |
| SEQ ID No 194 | AGAGCTAGGTGTTGTACATAA |
| SEQ ID No 195 | TGGTAATCTATTACTAGATAA |
| SEQ ID No 196 | GGTAATCTATTACTAGATAAA |
| SEQ ID No 197 | TTCAGTAGCTGCACTTACTAA |
| SEQ ID No 198 | AGTAGCTGCACTTACTAACAA |
| SEQ ID No 199 | TTATGACTACTATCGTTATAA |
| SEQ ID No 200 | TGTAGTTGAAGTTGTTGATAA |
| SEQ ID No 201 | TTACGATGGTGGCTGTATTAA |
| SEQ ID No 202 | TGGTGGCTGTATTAATGCTAA |
| SEQ ID No 203 | TATAACTCAAATGAATCTTAA |
| SEQ ID No 204 | ATCAAAAATTATTGAAATCAA |
| SEQ ID No 205 | GAGCTACTGTAGTAATTGGAA |
| SEQ ID No 206 | GTGGTTGGCACAACATGTTAA |
| SEQ ID No 207 | TGGTTGGCACAACATGTTAAA |
| SEQ ID No 208 | TTCTTGCTCGCAAACATACAA |
| SEQ ID No 209 | TGCCACAACTGCTTATGCTAA |
| SEQ ID No 210 | AGCTGTCACGGCCAATGTTAA |
| SEQ ID No 211 | TATCTACTGATGGTAACAAAA |
| SEQ ID No 212 | TTATGAGTGTCTCTATAGAAA |
| SEQ ID No 213 | AGGTCTAGTGGCTAGCATAAA |
| SEQ ID No 214 | TCTAGTGGCTAGCATAAAGAA |
| SEQ ID No 215 | ACTGAGACTGACCTTACTAAA |
| SEQ ID No 216 | CAACATACAATGCTAGTTAAA |
| SEQ ID No 217 | TTGTAGATGATATCGTAAAAA |
| SEQ ID No 218 | GATGGTACACTTATGATTGAA |
| SEQ ID No 219 | TGTACTTACAATACATAAGAA |
| SEQ ID No 220 | GTACTTACAATACATAAGAAA |
| SEQ ID No 221 | GAAAGCTACATGATGAGTTAA |
| SEQ ID No 222 | AAATTCCTAAAAACTAATTGT |
| SEQ ID No 223 | AATTCCTAAAAACTAATTGTT |
| SEQ ID No 224 | AAAAGGACGAAGATGACAATT |
| SEQ ID No 225 | AACGTCTTACTAAATACACAA |
| SEQ ID No 226 | AAGGTAATTGTGACACATTAA |
| SEQ ID No 227 | AAATACTTGTCACATACAATT |
| SEQ ID No 228 | AATTGTTGTGATGATGATTAT |
| SEQ ID No 229 | AATCAAGATCTCAATGGTAAC |
| SEQ ID No 230 | AACAAAGCCTTACATTAAGTG |
| SEQ ID No 231 | AATCAGGATGTAAACTTACAT |
| SEQ ID No 232 | AACTCAAATGAATCTTAAGTA |
| SEQ ID No 233 | AAAATTATTGAAATCAATAGC |
| SEQ ID No 234 | AATTGGAACAAGCAAATTCTA |
| SEQ ID No 235 | AATGAGTGTGCTCAAGTATTG |
| SEQ ID No 236 | AACTGCTTATGCTAATAGTGT |
| SEQ ID No 237 | AACAAAATTGCCGATAAGTAT |
| SEQ ID No 238 | AAAATTGCCGATAAGTATGTC |
| SEQ ID No 239 | AAAAACAGATGGTACACTTAT |
| SEQ ID No 240 | AAAACAGATGGTACACTTATG |
| SEQ ID No 241 | AACAGATGGTACACTTATGAT |
| SEQ ID No 242 | AAAGCTACATGATGAGTTAAC |
| SEQ ID No 243 | TATGGCTGTAGTTGTGATCAA |
| SEQ ID No 244 | AATTGTTGTCGCTTCCAAGAA |
| SEQ ID No 245 | ATTGTTGTCGCTTCCAAGAAA |
| SEQ ID No 246 | TTGTTGTCGCTTCCAAGAAAA |
| SEQ ID No 247 | AGAAAAGGACGAAGATGACAA |
| SEQ ID No 248 | TTCTCTAACTACCAACATGAA |
| SEQ ID No 249 | TCTAACTACCAACATGAAGAA |
| SEQ ID No 250 | GGATTGTCCAGCTGTTGCTAA |
| SEQ ID No 251 | GATTGTCCAGCTGTTGCTAAA |
| SEQ ID No 252 | AGAAATACTTGTCACATACAA |
| SEQ ID No 253 | GTATACGCCAACTTAGGTGAA |
| SEQ ID No 254 | ATTAGATAATCAAGATCTCAA |
| SEQ ID No 255 | TAATCAAGATCTCAATGGTAA |
| SEQ ID No 256 | CACATGTTGACACTGACTTAA |
| SEQ ID No 257 | ACTTCACGGAAGAGAGGTTAA |
| SEQ ID No 258 | CTTCACGGAAGAGAGGTTAAA |
| SEQ ID No 259 | GATGCATTCTGCATTGTGCAA |
| SEQ ID No 260 | ATGCATTCTGCATTGTGCAAA |
| SEQ ID No 261 | TTGTACATAATCAGGATGTAA |
| SEQ ID No 262 | TGTACATAATCAGGATGTAAA |
| SEQ ID No 263 | TATGCACGCTGCTTCTGGTAA |
| SEQ ID No 264 | AAGGAAGGAAGTTCTGTTGAA |
| SEQ ID No 265 | ACTATCGTTATAATCTACCAA |
| SEQ ID No 266 | ATCGTTATAATCTACCAACAA |
| SEQ ID No 267 | ACAATGTGTGATATCAGACAA |
| SEQ ID No 268 | ATCGTCAACAACCTAGACAAA |
| SEQ ID No 269 | TTAAGTATGCCATTAGTGCAA |
| SEQ ID No 270 | TAAGTATGCCATTAGTGCAAA |
| SEQ ID No 271 | GTATGCCATTAGTGCAAAGAA |
| SEQ ID No 272 | TATCTGTAGTACTATGACCAA |
| SEQ ID No 273 | CTAGAGGAGCTACTGTAGTAA |
| SEQ ID No 274 | CTACTGTAGTAATTGGAACAA |
| SEQ ID No 275 | TGTAGTAATTGGAACAAGCAA |
| SEQ ID No 276 | GTAGTAATTGGAACAAGCAAA |
| SEQ ID No 277 | CCATGCCTAACATGCTTAGAA |
| SEQ ID No 278 | TGTGCTCAAGTATTGAGTGAA |
| SEQ ID No 279 | GTGCTCAAGTATTGAGTGAAA |
| SEQ ID No 280 | TTATCTACTGATGGTAACAAA |
| SEQ ID No 281 | AAGGTCTAGTGGCTAGCATAA |
| SEQ ID No 282 | GACTGAGACTGACCTTACTAA |
| SEQ ID No 283 | TCAACATACAATGCTAGTTAA |
| SEQ ID No 284 | GTATTCTGTTATGCTTACTAA |
| SEQ ID No 285 | AAAGGTTATGGCTGTAGTTGT |
| SEQ ID No 286 | AAGGTTATGGCTGTAGTTGTG |
| SEQ ID No 287 | AATTGTTGTCGCTTCCAAGAA |
| SEQ ID No 288 | AAGAAAAGGACGAAGATGACA |
| SEQ ID No 289 | AACTACCAACATGAAGAAACA |
| SEQ ID No 290 | AAAAGAAATACTTGTCACATA |
| SEQ ID No 291 | AAAGAAATACTTGTCACATAC |
| SEQ ID No 292 | AAGAAATACTTGTCACATACA |
| SEQ ID No 293 | AATAAAAAGGACTGGTATGAT |
| SEQ ID No 294 | AAAAACAGTACAATTCTGTGA |
| SEQ ID No 295 | AAAACAGTACAATTCTGTGAT |
| SEQ ID No 296 | AAACAGTACAATTCTGTGATG |
| SEQ ID No 297 | AATGCCTATATTAACCTTGAC |
| SEQ ID No 298 | AACTGCAGAGTCACATGTTGA |
| SEQ ID No 299 | AACTTACATAGCTCTAGACTT |
| SEQ ID No 300 | AATCTATTACTAGATAAACGC |
| SEQ ID No 301 | AAGGAAGGAAGTTCTGTTGAA |
| SEQ ID No 302 | AATGCTGCTATCAGCGATTAT |
| SEQ ID No 303 | AATCTACCAACAATGTGTGAT |
| SEQ ID No 304 | AACAATGTGTGATATCAGACA |
| SEQ ID No 305 | AACCAAGTCATCGTCAACAAC |
| SEQ ID No 306 | AAGTCATCGTCAACAACCTAG |
| SEQ ID No 307 | AACAACCTAGACAAATCAGCT |
| SEQ ID No 308 | AAGTATGCCATTAGTGCAAAG |
| SEQ ID No 309 | AAATTATTGAAATCAATAGCC |
| SEQ ID No 310 | AACATGCTTAGAATTATGGCC |
| SEQ ID No 311 | AATTATGGCCTCACTTGTTCT |
| SEQ ID No 312 | AAACATACAACGTGTTGTAGC |
| SEQ ID No 313 | AAGTATTGAGTGAAATGGTCA |
| SEQ ID No 314 | AAGCTGTCACGGCCAATGTTA |
| SEQ ID No 315 | AAATTGCCGATAAGTATGTCC |
| SEQ ID No 316 | AAGGTCTAGTGGCTAGCATAA |
| SEQ ID No 317 | AAACAGATGGTACACTTATGA |
| SEQ ID No 318 | AACATCCTAATCAGGAGTATG |
| SEQ ID No 319 | AATACATAAGAAAGCTACATG |
| SEQ ID No 320 | AAGAAAGCTACATGATGAGTT |
| SEQ ID No 321 | AAGCTACATGATGAGTTAACA |
| SEQ ID No 322 | AACAGGACACATGTTAGACAT |
| SEQ ID No 323 | AATGATAACACTTCAAGGTAT |
| SEQ ID No 324 | CTTCAGTCAGCTGATGCACAA |
| SEQ ID No 325 | CGCTTCCAAGAAAAGGACGAA |
| SEQ ID No 326 | GTACCACATATATCACGTCAA |
| SEQ ID No 327 | TATATTACGCGTATACGCCAA |
| SEQ ID No 328 | ATTCTGTGATGCCATGCGAAA |
| SEQ ID No 329 | TTAAAATATGACTTCACGGAA |
| SEQ ID No 330 | GGCTGTATTAATGCTAACCAA |
| SEQ ID No 331 | TGCTAACCAAGTCATCGTCAA |
| SEQ ID No 332 | TAACCAAGTCATCGTCAACAA |
| SEQ ID No 333 | CATCGTCAACAACCTAGACAA |
| SEQ ID No 334 | TCAATGAGTTATGAGGATCAA |
| SEQ ID No 335 | ATTCTATGGTGGTTGGCACAA |
| SEQ ID No 336 | ATGTGATAGAGCCATGCCTAA |
| SEQ ID No 337 | TTAGCTAATGAGTGTGCTCAA |
| SEQ ID No 338 | CCTCATCAGGAGATGCCACAA |
| SEQ ID No 339 | CATCATCCGGTGATGCTACAA |
| SEQ ID No 340 | TGGTAACAAAATTGCCGATAA |
| SEQ ID No 341 | AATAGCACTTATGCATCTCAA |
| SEQ ID No 342 | CTTACTAAAGGACCTCATGAA |
| SEQ ID No 343 | AAAAACTAATTGTTGTCGCTT |
| SEQ ID No 344 | AAAACTAATTGTTGTCGCTTC |
| SEQ ID No 345 | AATAGACGGTGACATGGTACC |
| SEQ ID No 346 | AATGGCAGACCTCGTCTATGC |
| SEQ ID No 347 | AATGGCTGATTTAGTCTATGC |
| SEQ ID No 348 | AACAGTACAATTCTGTGATGC |
| SEQ ID No 349 | AAATGCTGGTATTGTTGGTGT |
| SEQ ID No 350 | AATGCTGGTATTGTTGGTGTA |
| SEQ ID No 351 | AAGATCTCAATGGTAACTGGT |
| SEQ ID No 352 | AAACCACGCCAGGTAGTGGAG |
| SEQ ID No 353 | AAGTAGCACCAGGCTGCGGAG |
| SEQ ID No 354 | AACCACGCCAGGTAGTGGAGT |
| SEQ ID No 355 | AAATATGACTTCACGGAAGAG |
| SEQ ID No 356 | AACTGGATACCACTTCAGAGA |
| SEQ ID No 357 | AAACTTACATAGCTCTAGACT |
| SEQ ID No 358 | AAGGAATTACTTGTGTATGCT |
| SEQ ID No 359 | AATTACTTGTGTATGCTGCTG |
| SEQ ID No 360 | AATGTGTGATATCAGACAACT |
| SEQ ID No 361 | AATGCTAACCAAGTCATCGTC |
| SEQ ID No 362 | AATGAGTTATGAGGATCAAGA |
| SEQ ID No 363 | AAATGAATCTTAAGTATGCCA |
| SEQ ID No 364 | AATGAATCTTAAGTATGCCAT |
| SEQ ID No 365 | AATCTTAAGTATGCCATTAGT |
| SEQ ID No 366 | AATTATTGAAATCAATAGCCG |
| SEQ ID No 367 | AATCAATAGCCGCCACTAGAG |
| SEQ ID No 368 | AATAGCCGCCACTAGAGGAGC |
| SEQ ID No 369 | AACAAGCAAATTCTATGGTGG |
| SEQ ID No 370 | AAGCAAATTCTATGGTGGTTG |
| SEQ ID No 371 | AACATACAACGTGTTGTAGCT |
| SEQ ID No 372 | AACGTGTTGTAGCTTGTCACA |
| SEQ ID No 373 | AAACCAGGTGGAACCTCATCA |
| SEQ ID No 374 | AACCAGGTGGAACCTCATCAG |
| SEQ ID No 375 | AACCAGGTGGAACATCATCCG |
| SEQ ID No 376 | AACCTCATCAGGAGATGCCAC |
| SEQ ID No 377 | AACATCATCCGGTGATGCTAC |
| SEQ ID No 378 | AATTGCCGATAAGTATGTCCG |
| SEQ ID No 379 | AAATAGAGATGTTGACACAGA |
| SEQ ID No 380 | AATAGAGATGTTGACACAGAC |
| SEQ ID No 381 | AATAGCACTTATGCATCTCAA |
| SEQ ID No 382 | AAAATGTTGGACTGAGACTGA |
| SEQ ID No 383 | AAATGTTGGACTGAGACTGAC |
| SEQ ID No 384 | AATGTTGGACTGAGACTGACC |
| SEQ ID No 385 | AAACATCCTAATCAGGAGTAT |
| SEQ ID No 386 | AATCAGGAGTATGCTGATGTC |
| SEQ ID No 387 | ACTAATTGTTGTCGCTTCCAA |
| SEQ ID No 388 | TTAGGTGAACGTGTACGCCAA |
| SEQ ID No 389 | AATTCTGTGATGCCATGCGAA |
| SEQ ID No 390 | CTCACTTGTTCTTGCTCGCAA |
| SEQ ID No 391 | TCACTTGTTCTTGCTCGCAAA |
| SEQ ID No 392 | TATATGTTAAACCAGGTGGAA |
| SEQ ID No 393 | TGCCGATAAGTATGTCCGCAA |
| SEQ ID No 394 | AACACCGTGCGGCACAGGCAC |
| SEQ ID No 395 | AAACTAATTGTTGTCGCTTCC |
| SEQ ID No 396 | AACTAATTGTTGTCGCTTCCA |
| SEQ ID No 397 | AAGGATTGTCCAGCTGTTGCT |
| SEQ ID No 398 | AAATACACAATGGCAGACCTC |
| SEQ ID No 399 | AATACACAATGGCAGACCTCG |
| SEQ ID No 400 | AACTTAGGTGAACGTGTACGC |
| SEQ ID No 401 | AATTCTGTGATGCCATGCGAA |
| SEQ ID No 402 | AAAATATGACTTCACGGAAGA |
| SEQ ID No 403 | AATATGACTTCACGGAAGAGA |
| SEQ ID No 404 | AACCTAGACAAATCAGCTGGT |
| SEQ ID No 405 | AATAGAGCTCGCACCGTAGCT |
| SEQ ID No 406 | AAATCAATAGCCGCCACTAGA |
| SEQ ID No 407 | AAATTCTATGGTGGTTGGCAC |
| SEQ ID No 408 | AATTCTATGGTGGTTGGCACA |
| SEQ ID No 409 | AATGTGATAGAGCCATGCCTA |
| SEQ ID No 410 | AATGGTCATGTGTGGCGGTTC |
| SEQ ID No 411 | AATGATGATACTCTCTGACGA |
| SEQ ID No 412 | AAGCAAAATGTTGGACTGAGA |
| SEQ ID No 413 | AGTTGTGATCAACTCCGCGAA |
| SEQ ID No 414 | TTGTCAAGCTGTCACGGCCAA |
| SEQ ID No 415 | AAACACCGTGCGGCACAGGCA |
| SEQ ID No 416 | AAAGAATAGAGCTCGCACCGT |
| SEQ ID No 417 | AAGAATAGAGCTCGCACCGTA |
| SEQ ID No 418 | AAATGTGATAGAGCCATGCCT |
| SEQ ID No 419 | AAATGGTCATGTGTGGCGGTT |
| SEQ ID No 420 | AGGTATGAGCTATTATTGTAA |
| SEQ ID No 421 | GGTATGAGCTATTATTGTAAA |
| SEQ ID No 422 | CTGGTTATCGTGTAACTAAAA |
| SEQ ID No 423 | TGGTTATCGTGTAACTAAAAA |
| SEQ ID No 424 | GTACAACAACTTACAAATTAA |
| SEQ ID No 425 | TACAACAACTTACAAATTAAA |
| SEQ ID No 426 | AGCAATGTTGCAAATTATCAA |
| SEQ ID No 427 | GCAATGTTGCAAATTATCAAA |
| SEQ ID No 428 | CAATGTTGCAAATTATCAAAA |
| SEQ ID No 429 | CAGTGTGTAGACTTATGAAAA |
| SEQ ID No 430 | AGCTCACTCTTGTAATGTAAA |
| SEQ ID No 431 | AAAACAGTAAAGTACAAATAG |
| SEQ ID No 432 | AACAACTTACAAATTAAATGT |
| SEQ ID No 433 | AAGAGCACTATGTTAGAATTA |
| SEQ ID No 434 | AATGTTGCAAATTATCAAAAG |
| SEQ ID No 435 | TAGACCATTCTTATGTTGTAA |
| SEQ ID No 436 | AGACCATTCTTATGTTGTAAA |
| SEQ ID No 437 | GTTACGACCATGTCATATCAA |
| SEQ ID No 438 | TGTCATATCAACATCACATAA |
| SEQ ID No 439 | GTCATATCAACATCACATAAA |
| SEQ ID No 440 | CTATTATTGTAAATCACATAA |
| SEQ ID No 441 | TATTATTGTAAATCACATAAA |
| SEQ ID No 442 | TACTGGTTATCGTGTAACTAA |
| SEQ ID No 443 | ACTGGTTATCGTGTAACTAAA |
| SEQ ID No 444 | TCGTGTAACTAAAAACAGTAA |
| SEQ ID No 445 | CGTGTAACTAAAAACAGTAAA |
| SEQ ID No 446 | ACTAAAAACAGTAAAGTACAA |
| SEQ ID No 447 | CTAAAAACAGTAAAGTACAAA |
| SEQ ID No 448 | CCGAGGTACAACAACTTACAA |
| SEQ ID No 449 | CGAGGTACAACAACTTACAAA |
| SEQ ID No 450 | TGCTGACATCACATACAGTAA |
| SEQ ID No 451 | CACAAGAGCACTATGTTAGAA |
| SEQ ID No 452 | TCAAAAGGTTGGTATGCAAAA |
| SEQ ID No 453 | CTATGTGAGAAGGCATTAAAA |
| SEQ ID No 454 | ATAAATTCAAAGTGAATTCAA |
| SEQ ID No 455 | AAAGTGAATTCAACATTAGAA |
| SEQ ID No 456 | TTCAGTGTGTAGACTTATGAA |
| SEQ ID No 457 | TCAGTGTGTAGACTTATGAAA |
| SEQ ID No 458 | GCTTAAAGCACATAAAGACAA |
| SEQ ID No 459 | CTTAAAGCACATAAAGACAAA |
| SEQ ID No 460 | CAGCTCACTCTTGTAATGTAA |
| SEQ ID No 461 | AACATCACATAAATTAGTCTT |
| SEQ ID No 462 | AAATTAGTCTTGTCTGTTAAT |
| SEQ ID No 463 | AATTAGTCTTGTCTGTTAATC |
| SEQ ID No 464 | AACTAAAAACAGTAAAGTACA |
| SEQ ID No 465 | AAAAACAGTAAAGTACAAATA |
| SEQ ID No 466 | AAACAGTAAAGTACAAATAGG |
| SEQ ID No 467 | AACTTACAAATTAAATGTTGG |
| SEQ ID No 468 | AATTAAATGTTGGTGATTATT |
| SEQ ID No 469 | AAGGTTGGTATGCAAAAGTAT |
| SEQ ID No 470 | AAATGTAGTAGAATTATACCT |
| SEQ ID No 471 | AATGTAGTAGAATTATACCTG |
| SEQ ID No 472 | AAATTCAAAGTGAATTCAACA |
| SEQ ID No 473 | AATTCAAAGTGAATTCAACAT |
| SEQ ID No 474 | AAAGTGAATTCAACATTAGAA |
| SEQ ID No 475 | AAGTGAATTCAACATTAGAAC |
| SEQ ID No 476 | AATTCAGTGTGTAGACTTATG |
| SEQ ID No 477 | AAGCTTAAAGCACATAAAGAC |
| SEQ ID No 478 | AAGCACATAAAGACAAATCAG |
| SEQ ID No 479 | AATTAACAGGCCACAAATAGG |
| SEQ ID No 480 | AAATAGGCGTGGTAAGAGAAT |
| SEQ ID No 481 | AATAGGCGTGGTAAGAGAATT |
| SEQ ID No 482 | AATGCTGTAGCCTCAAAGATT |
| SEQ ID No 483 | AACTCAAACTGTTGATTCATC |
| SEQ ID No 484 | AATATGACTATGTCATATTCA |
| SEQ ID No 485 | AATGTGACTATGTCATATTCA |
| SEQ ID No 486 | AAACAGCTCACTCTTGTAATG |
| SEQ ID No 487 | AACAGCTCACTCTTGTAATGT |
| SEQ ID No 488 | TAAATTAGTCTTGTCTGTTAA |
| SEQ ID No 489 | CCATTGTGTGCTAATGGACAA |
| SEQ ID No 490 | TACATGTGTTGGTAGCGATAA |
| SEQ ID No 491 | TTAGCTAACACCTGTACTGAA |
| SEQ ID No 492 | TAGCTAACACCTGTACTGAAA |
| SEQ ID No 493 | CACCTGTACTGAAAGACTCAA |
| SEQ ID No 494 | TAAACCTAGACCACCACTTAA |
| SEQ ID No 495 | CTAGACCACCACTTAACCGAA |
| SEQ ID No 496 | GCACCACGCACATTGCTAA |
| SEQ ID No 497 | ACCTGCACCACGCACATTGCTAA |
| SEQ ID No 498 | TTACCTGCACCACGCACATTGCTAA |
| SEQ ID No 499 | TAGACCACCACTTAACCGAAA |
| SEQ ID No 500 | CACATACAGTAATGCCATTAA |
| SEQ ID No 501 | ATCAAAAGGTTGGTATGCAAA |
| SEQ ID No 502 | TGTTGATGCACTATGTGAGAA |
| SEQ ID No 503 | CACTATGTGAGAAGGCATTAA |
| SEQ ID No 504 | ACTATGTGAGAAGGCATTAAA |
| SEQ ID No 505 | CTATAGATAAATGTAGTAGAA |
| SEQ ID No 506 | CTGCACCACGCACATTGCTAA |
| SEQ ID No 507 | ACCACGCACATTGCTAACTAA |
| SEQ ID No 508 | TAATAAGCTTAAAGCACATAA |
| SEQ ID No 509 | AATAAGCTTAAAGCACATAAA |
| SEQ ID No 510 | CAAATAGGCGTGGTAAGAGAA |
| SEQ ID No 511 | AAGAGAATTCCTTACACGTAA |
| SEQ ID No 512 | TTCACCTTATAATTCACAGAA |
| SEQ ID No 513 | GACTATGTCATATTCACTCAA |
| SEQ ID No 514 | ACTATGTCATATTCACTCAAA |
| SEQ ID No 515 | TGAAACAGCTCACTCTTGTAA |
| SEQ ID No 516 | GTTGCTATTACCAGAGCAAAA |
| SEQ ID No 517 | AATGCTCCAGGTTGTGATGTC |
| SEQ ID No 518 | AATGCAATTGCAACATGTGAC |
| SEQ ID No 519 | AACATGTGACTGGACAAATGC |
| SEQ ID No 520 | AACACCTGTACTGAAAGACTC |
| SEQ ID No 521 | AACTGTCTTATGGTATTGCTA |
| SEQ ID No 522 | AAGTGCTGTCTGACAGAGAAT |
| SEQ ID No 523 | AAACCTAGACCACCACTTAAC |
| SEQ ID No 524 | AACCTAGACCACCACTTAACC |
| SEQ ID No 525 | AACAGTAAAGTACAAATAGGA |
| SEQ ID No 526 | AAAAAGGTGACTATGGTGATG |
| SEQ ID No 527 | AAATTAAATGTTGGTGATTAT |
| SEQ ID No 528 | AATTATCAAAAGGTTGGTATG |
| SEQ ID No 529 | AAAAGGTTGGTATGCAAAAGT |
| SEQ ID No 530 | AAAGGTTGGTATGCAAAAGTA |
| SEQ ID No 531 | AATTCAACATTAGAACAGTAT |
| SEQ ID No 532 | AACATTAGAACAGTATGTCTT |
| SEQ ID No 533 | AAGCACTATGTGTACATTGGC |
| SEQ ID No 534 | AACTATAGGTCCAGACATGTT |
| SEQ ID No 535 | AATAAGCTTAAAGCACATAAA |
| SEQ ID No 536 | AAAGCACATAAAGACAAATCA |
| SEQ ID No 537 | AAGAGAATTCCTTACACGTAA |
| SEQ ID No 538 | GATGTCACAGATGTGACTCAA |
| SEQ ID No 539 | TTGCAACATGTGACTGGACAA |
| SEQ ID No 540 | TGCAACATGTGACTGGACAAA |
| SEQ ID No 541 | GGTATTGCTACTGTACGTGAA |
| SEQ ID No 542 | GAAGTGCTGTCTGACAGAGAA |
| SEQ ID No 543 | TATCAAAAGGTTGGTATGCAA |
| SEQ ID No 544 | CAATGCCAGATTACGTGCTAA |
| SEQ ID No 545 | GTCGGCGTTGTCCTGCTGAAA |
| SEQ ID No 546 | GTCGCCGTTGTCCTGCTGAAA |
| SEQ ID No 547 | CATAAAGACAAATCAGCTCAA |
| SEQ ID No 548 | CACAGAATGCTGTAGCCTCAA |
| SEQ ID No 549 | ACAGAATGCTGTAGCCTCAAA |
| SEQ ID No 550 | ATATTCACTCAAACCACTGAA |
| SEQ ID No 551 | TATTCACTCAAACCACTGAAA |
| SEQ ID No 552 | TGTTGCTATTACCAGAGCAAA |
| SEQ ID No 553 | AAATGCTGTTACGACCATGTC |
| SEQ ID No 554 | AATGCTGTTACGACCATGTCA |
| SEQ ID No 555 | AAAAATACATGTGTTGGTAGC |
| SEQ ID No 556 | AAAATACATGTGTTGGTAGCG |
| SEQ ID No 557 | AATTGCAACATGTGACTGGAC |
| SEQ ID No 558 | AAACGCTCAAAGCTACTGAGG |
| SEQ ID No 559 | AACGCTCAAAGCTACTGAGGA |
| SEQ ID No 560 | AAACTGTCTTATGGTATTGCT |
| SEQ ID No 561 | AAGTTGGTAAACCTAGACCAC |
| SEQ ID No 562 | AAAGTACAAATAGGAGAGTAC |
| SEQ ID No 563 | AAGTACAAATAGGAGAGTACA |
| SEQ ID No 564 | AAAAGGTGACTATGGTGATGC |
| SEQ ID No 565 | AAGGTGACTATGGTGATGCTG |
| SEQ ID No 566 | AATGCCATTAAGTGCACCTAC |
| SEQ ID No 567 | AAGTGCACCTACACTAGTGCC |
| SEQ ID No 568 | AACACTCAATATCTCAGATGA |
| SEQ ID No 569 | AAATTATCAAAAGGTTGGTAT |
| SEQ ID No 570 | AAATGCATTGCCTGAGACGAC |
| SEQ ID No 571 | AATGCCAGATTACGTGCTAAG |
| SEQ ID No 572 | AAAACTATAGGTCCAGACATG |
| SEQ ID No 573 | AAACTATAGGTCCAGACATGT |
| SEQ ID No 574 | AACTTGTCGGCGTTGTCCTGC |
| SEQ ID No 575 | AAATTGTTGACACTGTGAGTG |
| SEQ ID No 576 | AATTGTTGACACTGTGAGTGC |
| SEQ ID No 577 | AAAGACAAATCAGCTCAATGC |
| SEQ ID No 578 | AAGACAAATCAGCTCAATGCT |
| SEQ ID No 579 | AACAGGCCACAAATAGGCGTG |
| SEQ ID No 580 | AACAGACCTCAAATAGGCGTT |
| SEQ ID No 581 | AAACTGTTGATTCATCACAGG |
| SEQ ID No 582 | AACCACTGAAACAGCTCACTC |
| SEQ ID No 583 | GCACCTACACTAGTGCCACAA |
| SEQ ID No 584 | GTCCAGACATGTTCCTCGGAA |
| SEQ ID No 585 | TGTCGGCGTTGTCCTGCTGAA |
| SEQ ID No 586 | TCTGCAATTAACAGGCCACAA |
| SEQ ID No 587 | CTGCAATTAACAGGCCACAAA |
| SEQ ID No 588 | GGCCACAAATAGGCGTGGTAA |
| SEQ ID No 589 | ATGTTGCTATTACCAGAGCAA |
| SEQ ID No 590 | AAATACATGTGTTGGTAGCGA |
| SEQ ID No 591 | AATACATGTGTTGGTAGCGAT |
| SEQ ID No 592 | AAAGGTGACTATGGTGATGCT |
| SEQ ID No 593 | AAAAGTATTCTACACTCCAGG |
| SEQ ID No 594 | AATTATACCTGCACGTGCTCG |
| SEQ ID No 595 | AATGCATTGCCTGAGACGACA |
| SEQ ID No 596 | AATTACCTGCACCACGCACAT |
| SEQ ID No 597 | AATTCACAGAATGCTGTAGCC |
| SEQ ID No 598 | AAACCACTGAAACAGCTCACT |
| SEQ ID No 599 | AATGTTGCTATTACCAGAGCA |

The non-structural proteins (NSPs) from SARS-CoV-2 gene specificity was confirmed by searching NCBI BlastN database. The siRNAs were chemically synthesized.

All of the purified siRNA duplexes were complexed with lipofectamine and added to the cells for up to 12 h in serum-free medium. Thereafter, cells were cultured for 72-96 h in serum-supplemented medium, which was replaced by serum-free medium 24 h before the experiments. A scrambled negative siRNA duplex was used as control.

The non-structural proteins (NSPs)-siRNA is directed to a single target non-structural proteins (NSPs) from SARS-CoV-2 gene sequence. Alternatively, the siRNA is directed to multiple target non-structural proteins (NSPs) gene sequences. For example, the composition contains non-structural proteins (NSPs)-siRNA directed to two, three, four, five or more non-structural proteins (NSPs) target sequences. By non-structural proteins (NSPs) target sequence is meant a nucleotide sequence that is identical to a portion of the non-structural proteins (NSPs) gene. The target sequence can include the 5′ untranslated (UT) region, the open reading frame (ORF) or the 3′ untranslated region of the SARS-CoV-2 non-structural proteins (NSPs) gene. Alternatively, the siRNA is a nucleic acid sequence complementary to an upstream or downstream modulator of non-structural proteins (NSPs) gene expression. Examples of upstream and downstream modulators include, a transcription factor that binds the non-structural proteins (NSPs) gene promoter, a kinase or phosphatase that interacts with the non-structural proteins (NSPs) polypeptide, a non-structural proteins (NSPs) promoter or enhance.

SARS-CoV-2 non-structural proteins (NSPs)-siRNA which hybridize to target mRNA decrease or inhibit production of the non-structural proteins (NSPs) polypeptide product encoded by the non-structural proteins (NSPs) gene by associating with the normally single-stranded mRNA transcript, thereby interfering with translation and thus, expression of the protein. Exemplary nucleic acid sequence for the production of non-structural proteins (NSPs)-siRNA include the sequences of nucleotides SEQ ID No 58, SEQ ID No 59, SEQ ID No 60, SEQ ID No 61, SEQ ID No 62, SEQ ID No 86, SEQ ID No 152, SEQ ID No 153, SEQ ID No 210, SEQ ID No 250, SEQ ID No 263, SEQ ID No 314, SEQ ID No 324, SEQ ID No 325, SEQ ID No 338, SEQ ID No 339, SEQ ID No 345, SEQ ID No 346, SEQ ID No 347, SEQ ID No 352, SEQ ID No 353, SEQ ID No 354, SEQ ID No 367, SEQ ID No 368, SEQ ID No 373, SEQ ID No 374, SEQ ID No 375, SEQ ID No 376, SEQ ID No 377, SEQ ID No 384, SEQ ID No 484, SEQ ID No 485, SEQ ID No 495, SEQ ID No 496, SEQ ID No 497, SEQ ID No 498, SEQ ID No 506, SEQ ID No 517, SEQ ID No 524, SEQ ID No 542, SEQ ID No 545, SEQ ID No 546, SEQ ID No 548, SEQ ID No 558, SEQ ID No 559, SEQ ID No 565, SEQ ID No 567, SEQ ID No 570, SEQ ID No 574, SEQ ID No 579, SEQ ID No 580 and SEQ ID No 582 as the target sequence. In a further embodiment, in order to enhance the inhibition activity of the siRNA, nucleotide “u” can be added to 3′ end of the antisense strand of the target sequence. Preferably at least 2, more preferably 2 to 10, and most preferably 2 to 5 u's are added. The added u's form single strand at the 3′ end of the antisense strand of the siRNA.

The non-structural proteins (NSPs)-siRNA can be directly introduced into the cells in a form that is capable of binding to the mRNA transcripts. Alternatively, a vector encoding the non-structural proteins (NSPs)-siRNA can be introduced into the cells.

A loop sequence consisting of an arbitrary nucleotide sequence can be located between the sense and antisense sequence in order to form a hairpin loop structure. Thus, the present disclosure also provides siRNA having the general formula 5′-[A]-[B]-[A′]-3′, wherein [A] is a ribonucleotide sequence corresponding to a target sequence of the s non-structural proteins (NSPs) gene. Preferably [A] is a sequence selected from the group consisting of SEQ ID No 58, SEQ ID No 59, SEQ ID No 60, SEQ ID No 61, SEQ ID No 62, SEQ ID No 86, SEQ ID No 152, SEQ ID No 153, SEQ ID No 210, SEQ ID No 250, SEQ ID No 263, SEQ ID No 314, SEQ ID No 324, SEQ ID No 325, SEQ ID No 338, SEQ ID No 339, SEQ ID No 345, SEQ ID No 346, SEQ ID No 347, SEQ ID No 352, SEQ ID No 353, SEQ ID No 354, SEQ ID No 367, SEQ ID No 368, SEQ ID No 373, SEQ ID No 374, SEQ ID No 375, SEQ ID No 376, SEQ ID No 377, SEQ ID No 384, SEQ ID No 484, SEQ ID No 485, SEQ ID No 495, SEQ ID No 496, SEQ ID No 497, SEQ ID No 498, SEQ ID No 506, SEQ ID No 517, SEQ ID No 524, SEQ ID No 542, SEQ ID No 545, SEQ ID No 546, SEQ ID No 548, SEQ ID No 558, SEQ ID No 559, SEQ ID No 565, SEQ ID No 567, SEQ ID No 570, SEQ ID No 574, SEQ ID No 579, SEQ ID No 580 and SEQ ID No 582; [B] is a ribonucleotide sequence consisting of 3 to 23 nucleotides; and [A′] is a ribonucleotide sequence consisting of the complementary sequence of [A]. The region [A] hybridizes to [A′], and then a loop consisting of region [B] is formed. The loop sequence may be preferably 3 to nucleotide in length. Suitable loop sequences are described at http://www.ambion.com/techlib/tb/tb_506. html. Furthermore, loop sequence consisting of 23 nucleotides also provides active siRNA (Jacque et al., 2002).

In an embodiment, 5′ sense siRNA sequences against non-structural proteins (NSPs) from SARS-CoV-2 target sequences were identified. The 5′ anti-sense siRNA sequences against non-structural proteins (NSPs) from SARS-CoV-2 were then designed and produced. Sense and anti-sense siRNA sequences have a length of 19 to 25 nucleotides. Table 2 shows 5′ sense and anti-sense siRNA sequences against non-structural proteins (NSPs) from SARS-CoV-2. siRNA sequences have a length of 19 to 25 nucleotides.

TABLE 2
5′ sense and anti-sense siRNA sequences non-structural proteins (NSPs)
from SARS-CoV-2 - 19 to 25 nucleotides.

| SEQ ID No | 5′ RNA sense | SEQ ID No | 5′ RNA antisense |
|---|---|---|---|
| SEQ ID No 600 | AGCUGAUGUUACUAAAAUAAA | SEQ ID No 1199 | UUUAUUUUAGUAACAUCAGCU |
| SEQ ID No 601 | GCUGAUGUUACUAAAAUAAAA | SEQ ID No 1200 | UUUUAUUUUAGUAACAUCAGC |
| SEQ ID No 602 | AUGGAGCUGAUGUUACUAAAA | SEQ ID No 1201 | UUUUAGUAACAUCAGCUCCAU |
| SEQ ID No 603 | GAGCUGAUGUUACUAAAAUAA | SEQ ID No 1202 | UUAUUUUAGUAACAUCAGCUC |
| SEQ ID No 604 | GUGGUCACUAUAAACAUAUAA | SEQ ID No 1203 | UUAUAUGUUUAUAGUGACCAC |
| SEQ ID No 605 | AAAUAAAACCUCAUAAUUCAC | SEQ ID No 1204 | GUGAAUUAUGAGGUUUUAUUU |
| SEQ ID No 606 | AAACAAGCUACAAAAUAUCUA | SEQ ID No 1205 | UAGAUAUUUUGUAGCUUGUUU |
| SEQ ID No 607 | AACAAGCUACAAAAUAUCUAG | SEQ ID No 1206 | CUAGAUAUUUUGUAGCUUGUU |
| SEQ ID No 608 | GGAUGGAGCUGAUGUUACUAA | SEQ ID No 1207 | UUAGUAACAUCAGCUCCAUCC |
| SEQ ID No 609 | GAUGGAGCUGAUGUUACUAAA | SEQ ID No 1208 | UUUAGUAACAUCAGCUCCAUC |
| SEQ ID No 610 | AUAAUUCACAUGAAGGUAAAA | SEQ ID No 1209 | UUUUACCUUCAUGUGAAUUAU |
| SEQ ID No 611 | UAGGUACAUGUCAGCAUUAAA | SEQ ID No 1210 | UUUAAUGCUGACAUGUACCUA |
| SEQ ID No 612 | CAGCAUUAAAUCACACUAAAA | SEQ ID No 1211 | UUUUAGUGUGAUUUAAUGCUG |
| SEQ ID No 613 | AGCAUUAAAUCACACUAAAAA | SEQ ID No 1212 | UUUUUAGUGUGAUUUAAUGCU |
| SEQ ID No 614 | AUCUUGCCACUGCAUUGUUAA | SEQ ID No 1213 | UUAACAAUGCAGUGGCAAGAU |
| SEQ ID No 615 | UAUCUUAGCCUACUGUAAUAA | SEQ ID No 1214 | UUAUUACAGUAGGCUAAGAUA |
| SEQ ID No 616 | AGUUAGGUGAUGUUAGAGAAA | SEQ ID No 1215 | UUUCUCUAACAUCACCUAACU |
| SEQ ID No 617 | UCUUGAACGUGGUGUGUAAAA | SEQ ID No 1216 | UUUUACACACCACGUUCAAGA |
| SEQ ID No 618 | ACCUGCUCAGUAUGAACUUAA | SEQ ID No 1217 | UUAAGUUCAUACUGAGCAGGU |
| SEQ ID No 619 | UUACCAGUGUGGUCACUAUAA | SEQ ID No 1218 | UUAUAGUGACCACACUGGUAA |
| SEQ ID No 620 | UACCAGUGUGGUCACUAUAAA | SEQ ID No 1219 | UUUAUAGUGACCACACUGGUA |
| SEQ ID No 621 | CUAUAAACAUAUAACUUCUAA | SEQ ID No 1220 | UUAGAAGUUAUAUGUUUAUAG |
| SEQ ID No 622 | UAUAAACAUAUAACUUCUAAA | SEQ ID No 1221 | UUUAGAAGUUAUAUGUUUAUA |
| SEQ ID No 623 | AAACAUAUAACUUCUAAAGAA | SEQ ID No 1222 | UUCUUUAGAAGUUAUAUGUUU |
| SEQ ID No 624 | AACAUAUAACUUCUAAAGAAA | SEQ ID No 1223 | UUUCUUUAGAAGUUAUAUGUU |
| SEQ ID No 625 | UACAAAGUCCUCAGAAUACAA | SEQ ID No 1224 | UUGUAUUCUGAGGACUUUGUA |
| SEQ ID No 626 | ACAAAGUCCUCAGAAUACAAA | SEQ ID No 1225 | UUUGUAUUCUGAGGACUUUGU |
| SEQ ID No 627 | AAAAUAAAACCUCAUAAUUCA | SEQ ID No 1226 | UGAAUUAUGAGGUUUUAUUUU |
| SEQ ID No 628 | AAUAAAACCUCAUAAUUCACA | SEQ ID No 1227 | UGUGAAUUAUGAGGUUUUAUU |
| SEQ ID No 629 | AACACUCCAACAAAUAGAGUU | SEQ ID No 1228 | AACUCUAUUUGUUGGAGUGUU |
| SEQ ID No 630 | AACGUGGUGUGUAAAACUUGU | SEQ ID No 1229 | ACAAGUUUUACACACCACGUU |
| SEQ ID No 631 | AAGCUACAAAAUAUCUAGUAC | SEQ ID No 1230 | GUACUAGAUAUUUUGUAGCUU |
| SEQ ID No 632 | AAACAUAUAACUUCUAAAGAA | SEQ ID No 1231 | UUCUUUAGAAGUUAUAUGUUU |
| SEQ ID No 633 | AACAUAUAACUUCUAAAGAAA | SEQ ID No 1232 | UUUCUUUAGAAGUUAUAUGUU |
| SEQ ID No 634 | AAAGUCCUCAGAAUACAAAGG | SEQ ID No 1233 | CCUUUGUAUUCUGAGGACUUU |
| SEQ ID No 635 | UACUAAAAUAAAACCUCAUAA | SEQ ID No 1234 | UUAUGAGGUUUUAUUUUAGUA |
| SEQ ID No 636 | AAACCUCAUAAUUCACAUGAA | SEQ ID No 1235 | UUCAUGUGAAUUAUGAGGUUU |
| SEQ ID No 637 | UCAUAAUUCACAUGAAGGUAA | SEQ ID No 1236 | UUACCUUCAUGUGAAUUAUGA |
| SEQ ID No 638 | CAUAAUUCACAUGAAGGUAAA | SEQ ID No 1237 | UUUACCUUCAUGUGAAUUAUG |
| SEQ ID No 639 | GUAGGUACAUGUCAGCAUUAA | SEQ ID No 1238 | UUAAUGCUGACAUGUACCUAC |
| SEQ ID No 640 | GUCAGCAUUAAAUCACACUAA | SEQ ID No 1239 | UUAGUGUGAUUUAAUGCUGAC |
| SEQ ID No 641 | UCAGCAUUAAAUCACACUAAA | SEQ ID No 1240 | UUUAGUGUGAUUUAAUGCUGA |
| SEQ ID No 642 | AAAUCACACUAAAAAGUGGAA | SEQ ID No 1241 | UUCCACUUUUUAGUGUGAUUU |
| SEQ ID No 643 | AAUCACACUAAAAAGUGGAAA | SEQ ID No 1242 | UUUCCACUUUUUAGUGUGAUU |
| SEQ ID No 644 | CAUUGUUAACACUCCAACAAA | SEQ ID No 1243 | UUUGUUGGAGUGUUAACAAUG |
| SEQ ID No 645 | ACUCCAACAAAUAGAGUUGAA | SEQ ID No 1244 | UUCAACUCUAUUUGUUGGAGU |
| SEQ ID No 646 | ACUUAUCUUAGCCUACUGUAA | SEQ ID No 1245 | UUACAGUAGGCUAAGAUAAGU |
| SEQ ID No 647 | GAGUUAGGUGAUGUUAGAGAA | SEQ ID No 1246 | UUCUCUAACAUCACCUAACUC |
| SEQ ID No 648 | UAGGUGAUGUUAGAGAAACAA | SEQ ID No 1247 | UUGUUUCUCUAACAUCACCUA |
| SEQ ID No 649 | UUCUUGCAAAAGAGUCUUGAA | SEQ ID No 1248 | UUCAAGACUCUUUUGCAAGAA |
| SEQ ID No 650 | GUCUUGAACGUGGUGUGUAAA | SEQ ID No 1249 | UUUACACACCACGUUCAAGAC |
| SEQ ID No 651 | AUACCUUGUACGUGUGGUAAA | SEQ ID No 1250 | UUUACCACACGUACAAGGUAU |
| SEQ ID No 652 | CCUUGUACGUGUGGUAAACAA | SEQ ID No 1251 | UUGUUUACCACACGUACAAGG |
| SEQ ID No 653 | CGUGUGGUAAACAAGCUACAA | SEQ ID No 1252 | UUGUAGCUUGUUUACCACACG |
| SEQ ID No 654 | GUGUGGUAAACAAGCUACAAA | SEQ ID No 1253 | UUUGUAGCUUGUUUACCACAC |
| SEQ ID No 655 | UGUGGUAAACAAGCUACAAAA | SEQ ID No 1254 | UUUUGUAGCUUGUUUACCACA |
| SEQ ID No 656 | GCUACAAAAUAUCUAGUACAA | SEQ ID No 1255 | UUGUACUAGAUAUUUUGUAGC |
| SEQ ID No 657 | GCACCACCUGCUCAGUAUGAA | SEQ ID No 1256 | UUCAUACUGAGCAGGUGGUGC |
| SEQ ID No 658 | GCACCACCUGCUGAGUAUAAA | SEQ ID No 1257 | UUUAUACUCAGCAGGUGGUGC |
| SEQ ID No 659 | ACCACCUGCUCAGUAUGAA | SEQ ID No 1258 | UUCAUACUGAGCAGGUGGU |
| SEQ ID No 660 | CAGCACCACCUGCUCAGUAUGAA | SEQ ID No 1259 | UUCAUACUGAGCAGGUGGUGCUG |
| SEQ ID No 661 | GUCAGCACCACCUGCUCAGUAUGAA | SEQ ID No 1260 | UUCAUACUGAGCAGGUGGUGCUGAC |
| SEQ ID No 662 | ACAAAGAAAACAGUUACACAA | SEQ ID No 1261 | UUGUGUAACUGUUUUCUUUGU |
| SEQ ID No 663 | AAGAAAACAGUUACACAACAA | SEQ ID No 1262 | UUGUUGUGUAACUGUUUUCUU |
| SEQ ID No 664 | AAUGACAUAUGGACAACAGUU | SEQ ID No 1263 | AACUGUUGUCCAUAUGUCAUU |
| SEQ ID No 665 | AAAACCUCAUAAUUCACAUGA | SEQ ID No 1264 | UCAUGUGAAUUAUGAGGUUUU |
| SEQ ID No 666 | AAACCUCAUAAUUCACAUGAA | SEQ ID No 1265 | UUCAUGUGAAUUAUGAGGUUU |
| SEQ ID No 667 | AACCUCAUAAUUCACAUGAAG | SEQ ID No 1266 | CUUCAUGUGAAUUAUGAGGUU |
| SEQ ID No 668 | AAUUCACAUGAAGGUAAAACA | SEQ ID No 1267 | UGUUUUACCUUCAUGUGAAUU |
| SEQ ID No 669 | AAAUCACACUAAAAAGUGGAA | SEQ ID No 1268 | UUCCACUUUUUAGUGUGAUUU |
| SEQ ID No 670 | AAUCACACUAAAAAGUGGAAA | SEQ ID No 1269 | UUUCCACUUUUUAGUGUGAUU |
| SEQ ID No 671 | AAUCCACCUGCUCUACAAGAU | SEQ ID No 1270 | AUCUUGUAGAGCAGGUGGAUU |
| SEQ ID No 672 | AAAGGUGUUCAGAUACCUUGU | SEQ ID No 1271 | ACAAGGUAUCUGAACACCUUU |
| SEQ ID No 673 | AAGGUGUUCAGAUACCUUGUA | SEQ ID No 1272 | UACAAGGUAUCUGAACACCUU |
| SEQ ID No 674 | AAGUCCUCAGAAUACAAAGGU | SEQ ID No 1273 | ACCUUUGUAUUCUGAGGACUU |
| SEQ ID No 675 | AAUACAAAGGUCCUAUUACGG | SEQ ID No 1274 | CCGUAAUAGGACCUUUGUAUU |
| SEQ ID No 676 | AAAGAAAACAGUUACACAACA | SEQ ID No 1275 | UGUUGUGUAACUGUUUUCUUU |
| SEQ ID No 677 | AAGAAAACAGUUACACAACAA | SEQ ID No 1276 | UUGUUGUGUAACUGUUUUCUU |
| SEQ ID No 678 | AAACAGUUACACAACAACCAU | SEQ ID No 1277 | AUGGUUGUUGUGUAACUGUUU |
| SEQ ID No 679 | AACAGUUACACAACAACCAUU | SEQ ID No 1278 | AAUGGUUGUUGUGUAACUGUU |
| SEQ ID No 680 | ACUGCAUUGUUAACACUCCAA | SEQ ID No 1279 | UUGGAGUGUUAACAAUGCAGU |
| SEQ ID No 681 | GCAUUGUUAACACUCCAACAA | SEQ ID No 1280 | UUGUUGGAGUGUUAACAAUGC |
| SEQ ID No 682 | AAGAUGCUUAUUACAGAGCAA | SEQ ID No 1281 | UUGCUCUGUAAUAAGCAUCUU |
| SEQ ID No 683 | AGUCUUGAACGUGGUGUGUAA | SEQ ID No 1282 | UUACACACCACGUUCAAGACU |
| SEQ ID No 684 | GUGUGUAAAACUUGUGGACAA | SEQ ID No 1283 | UUGUCCACAAGUUUUACACAC |
| SEQ ID No 685 | CUUGUGGACAACAGCAGACAA | SEQ ID No 1284 | UUGUCUGCUGUUGUCCACAAG |
| SEQ ID No 686 | GAUACCUUGUACGUGUGGUAA | SEQ ID No 1285 | UUACCACACGUACAAGGUAUC |
| SEQ ID No 687 | UGCUAGUGAGUACACUGGUAA | SEQ ID No 1286 | UUACCAGUGUACUCACUAGCA |
| SEQ ID No 688 | UUACUUACAAAGUCCUCAGAA | SEQ ID No 1287 | UUCUGAGGACUUUGUAAGUAA |
| SEQ ID No 689 | AAUGAUGACACUCUACGUGUU | SEQ ID No 1288 | AACACGUAGAGUGUCAUCAUU |
| SEQ ID No 690 | AAGAUGCUUAUUACAGAGCAA | SEQ ID No 1289 | UUGCUCUGUAAUAAGCAUCUU |
| SEQ ID No 691 | AAUAAGACAGUAGGUGAGUUA | SEQ ID No 1290 | UAACUCACCUACUGUCUUAUU |
| SEQ ID No 692 | AAGACAGUAGGUGAGUUAGGU | SEQ ID No 1291 | ACCUAACUCACCUACUGUCUU |
| SEQ ID No 693 | AAAAGAGUCUUGAACGUGGUG | SEQ ID No 1292 | CACCACGUUCAAGACUCUUUU |
| SEQ ID No 694 | AAAACUUGUGGACAACAGCAG | SEQ ID No 1293 | CUGCUGUUGUCCACAAGUUUU |
| SEQ ID No 695 | AAACUUGUGGACAACAGCAGA | SEQ ID No 1294 | UCUGCUGUUGUCCACAAGUUU |
| SEQ ID No 696 | AACUUGUGGACAACAGCAGAC | SEQ ID No 1295 | GUCUGCUGUUGUCCACAAGUU |
| SEQ ID No 697 | AAGAAAGGUGUUCAGAUACCU | SEQ ID No 1296 | AGGUAUCUGAACACCUUUCUU |
| SEQ ID No 698 | AAAAUAUCUAGUACAACAGGA | SEQ ID No 1297 | UCCUGUUGUACUAGAUAUUUU |
| SEQ ID No 699 | AAAUAUCUAGUACAACAGGAG | SEQ ID No 1298 | CUCCUGUUGUACUAGAUAUUU |
| SEQ ID No 700 | AAUAUCUAGUACAACAGGAGU | SEQ ID No 1299 | ACUCCUGUUGUACUAGAUAUU |
| SEQ ID No 701 | AAUUACCAGUGUGGUCACUAU | SEQ ID No 1300 | AUAGUGACCACACUGGUAAUU |
| SEQ ID No 702 | AAAGGUCCUAUUACGGAUGUU | SEQ ID No 1301 | AACAUCCGUAAUAGGACCUUU |
| SEQ ID No 703 | AAAACAGUUACACAACAACCA | SEQ ID No 1302 | UGGUUGUUGUGUAACUGUUUU |
| SEQ ID No 704 | AACAACUGUUAUCUUGCCACU | SEQ ID No 1303 | AGUGGCAAGAUAACAGUUGUU |
| SEQ ID No 705 | AAAGAGUCUUGAACGUGGUGU | SEQ ID No 1304 | ACACCACGUUCAAGACUCUUU |
| SEQ ID No 706 | AAGAGUCUUGAACGUGGUGUG | SEQ ID No 1305 | CACACCACGUUCAAGACUCUU |
| SEQ ID No 707 | AACUGUUAUCUUGCCACUGCA | SEQ ID No 1306 | UGCAGUGGCAAGAUAACAGUU |
| SEQ ID No 708 | CAUUCGUAAGUCUAAUCAUAA | SEQ ID No 1307 | UUAUGAUUAGACUUACGAAUG |
| SEQ ID No 709 | GUUGUACGCUGCUGUUAUAAA | SEQ ID No 1308 | UUUAUAACAGCAGCGUACAAC |
| SEQ ID No 710 | GCUAUGAAGUACAAUUAUGAA | SEQ ID No 1309 | UUCAUAAUUGUACUUCAUAGC |
| SEQ ID No 711 | UUGUGGUACAACUACACUUAA | SEQ ID No 1310 | UUAAGUGUAGUUGUACCACAA |
| SEQ ID No 712 | GUACAGGCUGGUAAUGUUCAA | SEQ ID No 1311 | UUGAACAUUACCAGCCUGUAC |
| SEQ ID No 713 | UAUUGGACAUUCUAUGCAAAA | SEQ ID No 1312 | UUUUGCAUAGAAUGUCCAAUA |
| SEQ ID No 714 | UAUGCAAAAUUGUGUACUUAA | SEQ ID No 1313 | UUAAGUACACAAUUUUGCAUA |
| SEQ ID No 715 | AAAUUGUGUACUUAAGCUUAA | SEQ ID No 1314 | UUAAGCUUAAGUACACAAUUU |
| SEQ ID No 716 | AUCCUAAGACACCUAAGAUAA | SEQ ID No 1315 | UUAUCUUAGGUGUCUUAGGAU |
| SEQ ID No 717 | UUCAGUGUUAGCUUGUUACAA | SEQ ID No 1316 | UUGUAACAAGCUAACACUGAA |
| SEQ ID No 718 | UGCACCAUAUGGAAUUACCAA | SEQ ID No 1317 | UUGGUAAUUCCAUAUGGUGCA |
| SEQ ID No 719 | GGACACAACUAUUACAGUUAA | SEQ ID No 1318 | UUAACUGUAAUAGUUGUGUCC |
| SEQ ID No 720 | GGUUGUACGCUGCUGUUAUAA | SEQ ID No 1319 | UUAUAACAGCAGCGUACAACC |
| SEQ ID No 721 | AAAAUUGUGUACUUAAGCUUA | SEQ ID No 1320 | UAAGCUUAAGUACACAAUUUU |
| SEQ ID No 722 | AAAUUGUGUACUUAAGCUUAA | SEQ ID No 1321 | UUAAGCUUAAGUACACAAUUU |
| SEQ ID No 723 | AAUUGUGUACUUAAGCUUAAG | SEQ ID No 1322 | CUUAAGCUUAAGUACACAAUU |
| SEQ ID No 724 | AACCUUGUGGCUAUGAAGUAC | SEQ ID No 1323 | GUACUUCAUAGCCACAAGGUU |
| SEQ ID No 725 | AAGACCAUGUUGACAUACUAG | SEQ ID No 1324 | CUAGUAUGUCAACAUGGUCUU |
| SEQ ID No 726 | GCACCUCUGAAGACAUGCUAA | SEQ ID No 1325 | UUAGCAUGUCUUCAGAGGUGC |
| SEQ ID No 727 | GUUAUUGGACAUUCUAUGCAA | SEQ ID No 1326 | UUGCAUAGAAUGUCCAAUAAC |
| SEQ ID No 728 | UUAUUGGACAUUCUAUGCAAA | SEQ ID No 1327 | UUUGCAUAGAAUGUCCAAUAA |
| SEQ ID No 729 | GGUUGAUACAGCCAAUCCUAA | SEQ ID No 1328 | UUAGGAUUGGCUGUAUCAACC |
| SEQ ID No 730 | UGUUACAUGCACCAUAUGGAA | SEQ ID No 1329 | UUCCAUAUGGUGCAUGUAACA |
| SEQ ID No 731 | CAUGCUGGCACAGACUUAGAA | SEQ ID No 1330 | UUCUAAGUCUGUGCCAGCAUG |
| SEQ ID No 732 | UGGCACAGACUUAGAAGGUAA | SEQ ID No 1331 | UUACCUUCUAAGUCUGUGCCA |
| SEQ ID No 733 | CCUUGUGGCUAUGAAGUACAA | SEQ ID No 1332 | UUGUACUUCAUAGCCACAAGG |
| SEQ ID No 734 | AGUACAAUUAUGAACCUCUAA | SEQ ID No 1333 | UUAGAGGUUCAUAAUUGUACU |
| SEQ ID No 735 | AAUUAUGAACCUCUAACACAA | SEQ ID No 1334 | UUGUGUUAGAGGUUCAUAAUU |
| SEQ ID No 736 | AAGUAACUUGUGGUACAACUA | SEQ ID No 1335 | UAGUUGUACCACAAGUUACUU |
| SEQ ID No 737 | AACUUGUGGUACAACUACACU | SEQ ID No 1336 | AGUGUAGUUGUACCACAAGUU |
| SEQ ID No 738 | AAUCCUAAGACACCUAAGAUA | SEQ ID No 1337 | UAUCUUAGGUGUCUUAGGAUU |
| SEQ ID No 739 | AAUUACCAACUGGAGUUCAUG | SEQ ID No 1338 | CAUGAACUCCAGUUGGUAAUU |
| SEQ ID No 740 | AAUUAUGAACCUCUAACACAA | SEQ ID No 1339 | UUGUGUUAGAGGUUCAUAAUU |
| SEQ ID No 741 | AACCUCUAACACAAGACCAUG | SEQ ID No 1340 | CAUGGUCUUGUGUUAGAGGUU |
| SEQ ID No 742 | AACACAAGACCAUGUUGACAU | SEQ ID No 1341 | AUGUCAACAUGGUCUUGUGUU |
| SEQ ID No 743 | UACAAGUAACUUGUGGUACAA | SEQ ID No 1342 | UUGUACCACAAGUUACUUGUA |
| SEQ ID No 744 | CAUGUGAUCUGCACCUCUGAA | SEQ ID No 1343 | UUCAGAGGUGCAGAUCACAUG |
| SEQ ID No 745 | GCUUAAGGUUGAUACAGCCAA | SEQ ID No 1344 | UUGGCUGUAUCAACCUUAAGC |
| SEQ ID No 746 | AGCCAAUCCUAAGACACCUAA | SEQ ID No 1345 | UUAGGUGUCUUAGGAUUGGCU |
| SEQ ID No 747 | GUUGACAGGCAAACAGCACAA | SEQ ID No 1346 | UUGUGCUGUUUGCCUGUCAAC |
| SEQ ID No 748 | AAGCUUAAGGUUGAUACAGCC | SEQ ID No 1347 | GGCUGUAUCAACCUUAAGCUU |
| SEQ ID No 749 | AAUGGUUCACCAUCUGGUGUU | SEQ ID No 1348 | AACACCAGAUGGUGAACCAUU |
| SEQ ID No 750 | AAUGGUUCAUGUGGUAGUGUU | SEQ ID No 1349 | AACACUACCACAUGAACCAUU |
| SEQ ID No 751 | AACAGCACAAGCAGCUGGUAC | SEQ ID No 1350 | GUACCAGCUGCUUGUGCUGUU |
| SEQ ID No 752 | AACUGCACAGGCUGCAGGUAC | SEQ ID No 1351 | GUACCUGCAGCCUGUGCAGUU |
| SEQ ID No 753 | AAGUACAAUUAUGAACCUCUA | SEQ ID No 1352 | UAGAGGUUCAUAAUUGUACUU |
| SEQ ID No 754 | AAGCAGCUGGUACGGACACAA | SEQ ID No 1353 | UUGUGUCCGUACCAGCUGCUU |
| SEQ ID No 755 | AAGACAUGUGAUCUGCACCUC | SEQ ID No 1354 | GAGGUGCAGAUCACAUGUCUU |
| SEQ ID No 756 | AAGGUUGAUACAGCCAAUCCU | SEQ ID No 1355 | AGGAUUGGCUGUAUCAACCUU |
| SEQ ID No 757 | AACUGGAGUUCAUGCUGGCAC | SEQ ID No 1356 | GUGCCAGCAUGAACUCCAGUU |
| SEQ ID No 758 | AAACAGCACAAGCAGCUGGUA | SEQ ID No 1357 | UACCAGCUGCUUGUGCUGUUU |
| SEQ ID No 759 | AAGCAGCUGGUACGGACACAA | SEQ ID No 1358 | UUGUGUCCGUACCAGCUGCUU |
| SEQ ID No 760 | UGCUAAAUUCCUAAAAACUAA | SEQ ID No 1359 | UUAGUUUUUAGGAAUUUAGCA |
| SEQ ID No 761 | GGUAAUUGUGACACAUUAAAA | SEQ ID No 1360 | UUUUAAUGUGUCACAAUUACC |
| SEQ ID No 762 | AAUUGUGACACAUUAAAAGAA | SEQ ID No 1361 | UUCUUUUAAUGUGUCACAAUU |
| SEQ ID No 763 | AUUGUGACACAUUAAAAGAAA | SEQ ID No 1362 | UUUCUUUUAAUGUGUCACAAU |
| SEQ ID No 764 | UGGUGUACUGACAUUAGAUAA | SEQ ID No 1363 | UUAUCUAAUGUCAGUACACCA |
| SEQ ID No 765 | CAUUGUUAAUGCCUAUAUUAA | SEQ ID No 1364 | UUAAUAUAGGCAUUAACAAUG |
| SEQ ID No 766 | CUUAACAAAGCCUUACAUUAA | SEQ ID No 1365 | UUAAUGUAAGGCUUUGUUAAG |
| SEQ ID No 767 | AAGGAAGUUCUGUUGAAUUAA | SEQ ID No 1366 | UUAAUUCAACAGAACUUCCUU |
| SEQ ID No 768 | AGGAAGUUCUGUUGAAUUAAA | SEQ ID No 1367 | UUUAAUUCAACAGAACUUCCU |
| SEQ ID No 769 | GGAAGUUCUGUUGAAUUAAAA | SEQ ID No 1368 | UUUUAAUUCAACAGAACUUCC |
| SEQ ID No 770 | GGUUGGCACAACAUGUUAAAA | SEQ ID No 1369 | UUUUAACAUGUUGUGCCAACC |
| SEQ ID No 771 | GUUGGCACAACAUGUUAAAAA | SEQ ID No 1370 | UUUUUAACAUGUUGUGCCAAC |
| SEQ ID No 772 | UGGCGGUUCACUAUAUGUUAA | SEQ ID No 1371 | UUAACAUAUAGUGAACCGCCA |
| SEQ ID No 773 | GGCGGUUCACUAUAUGUUAAA | SEQ ID No 1372 | UUUAACAUAUAGUGAACCGCC |
| SEQ ID No 774 | AAUUGUGACACAUUAAAAGAA | SEQ ID No 1373 | UUCUUUUAAUGUGUCACAAUU |
| SEQ ID No 775 | AAUACUUGUCACAUACAAUUG | SEQ ID No 1374 | CAAUUGUAUGUGACAAGUAUU |
| SEQ ID No 776 | AAGGAAGUUCUGUUGAAUUAA | SEQ ID No 1375 | UUAAUUCAACAGAACUUCCUU |
| SEQ ID No 777 | AAGUUCUGUUGAAUUAAAACA | SEQ ID No 1376 | UGUUUUAAUUCAACAGAACUU |
| SEQ ID No 778 | AAAAAUUAUUGAAAUCAAUAG | SEQ ID No 1377 | CUAUUGAUUUCAAUAAUUUUU |
| SEQ ID No 779 | AACAUACAAUGCUAGUUAAAC | SEQ ID No 1378 | GUUUAACUAGCAUUGUAUGUU |
| SEQ ID No 780 | CUAACUACCAACAUGAAGAAA | SEQ ID No 1379 | UUUCUUCAUGUUGGUAGUUAG |
| SEQ ID No 781 | ACUACCAACAUGAAGAAACAA | SEQ ID No 1380 | UUGUUUCUUCAUGUUGGUAGU |
| SEQ ID No 782 | AUCACGUCAACGUCUUACUAA | SEQ ID No 1381 | UUAGUAAGACGUUGACGUGAU |
| SEQ ID No 783 | UCACGUCAACGUCUUACUAAA | SEQ ID No 1382 | UUUAGUAAGACGUUGACGUGA |
| SEQ ID No 784 | AACGUCUUACUAAAUACACAA | SEQ ID No 1383 | UUGUGUAUUUAGUAAGACGUU |
| SEQ ID No 785 | AAGGUAAUUGUGACACAUUAA | SEQ ID No 1384 | UUAAUGUGUCACAAUUACCUU |
| SEQ ID No 786 | AGGUAAUUGUGACACAUUAAA | SEQ ID No 1385 | UUUAAUGUGUCACAAUUACCU |
| SEQ ID No 787 | GUACUGACAUUAGAUAAUCAA | SEQ ID No 1386 | UUGAUUAUCUAAUGUCAGUAC |
| SEQ ID No 788 | AUUCUUAUUAUUCAUUGUUAA | SEQ ID No 1387 | UUAACAAUGAAUAAUAAGAAU |
| SEQ ID No 789 | AUGUUGACACUGACUUAACAA | SEQ ID No 1388 | UUGUUAAGUCAGUGUCAACAU |
| SEQ ID No 790 | UGUUGACACUGACUUAACAAA | SEQ ID No 1389 | UUUGUUAAGUCAGUGUCAACA |
| SEQ ID No 791 | UUCACGGAAGAGAGGUUAAAA | SEQ ID No 1390 | UUUUAACCUCUCUUCCGUGAA |
| SEQ ID No 792 | UUGGACCACUAGUGAGAAAAA | SEQ ID No 1391 | UUUUUCUCACUAGUGGUCCAA |
| SEQ ID No 793 | AGAGCUAGGUGUUGUACAUAA | SEQ ID No 1392 | UUAUGUACAACACCUAGCUCU |
| SEQ ID No 794 | UGGUAAUCUAUUACUAGAUAA | SEQ ID No 1393 | UUAUCUAGUAAUAGAUUACCA |
| SEQ ID No 795 | GGUAAUCUAUUACUAGAUAAA | SEQ ID No 1394 | UUUAUCUAGUAAUAGAUUACC |
| SEQ ID No 796 | UUCAGUAGCUGCACUUACUAA | SEQ ID No 1395 | UUAGUAAGUGCAGCUACUGAA |
| SEQ ID No 797 | AGUAGCUGCACUUACUAACAA | SEQ ID No 1396 | UUGUUAGUAAGUGCAGCUACU |
| SEQ ID No 798 | UUAUGACUACUAUCGUUAUAA | SEQ ID No 1397 | UUAUAACGAUAGUAGUCAUAA |
| SEQ ID No 799 | UGUAGUUGAAGUUGUUGAUAA | SEQ ID No 1398 | UUAUCAACAACUUCAACUACA |
| SEQ ID No 800 | UUACGAUGGUGGCUGUAUUAA | SEQ ID No 1399 | UUAAUACAGCCACCAUCGUAA |
| SEQ ID No 801 | UGGUGGCUGUAUUAAUGCUAA | SEQ ID No 1400 | UUAGCAUUAAUACAGCCACCA |
| SEQ ID No 802 | UAUAACUCAAAUGAAUCUUAA | SEQ ID No 1401 | UUAAGAUUCAUUUGAGUUAUA |
| SEQ ID No 803 | AUCAAAAAUUAUUGAAAUCAA | SEQ ID No 1402 | UUGAUUUCAAUAAUUUUUGAU |
| SEQ ID No 804 | GAGCUACUGUAGUAAUUGGAA | SEQ ID No 1403 | UUCCAAUUACUACAGUAGCUC |
| SEQ ID No 805 | GUGGUUGGCACAACAUGUUAA | SEQ ID No 1404 | UUAACAUGUUGUGCCAACCAC |
| SEQ ID No 806 | UGGUUGGCACAACAUGUUAAA | SEQ ID No 1405 | UUUAACAUGUUGUGCCAACCA |
| SEQ ID No 807 | UUCUUGCUCGCAAACAUACAA | SEQ ID No 1406 | UUGUAUGUUUGCGAGCAAGAA |
| SEQ ID No 808 | UGCCACAACUGCUUAUGCUAA | SEQ ID No 1407 | UUAGCAUAAGCAGUUGUGGCA |
| SEQ ID No 809 | AGCUGUCACGGCCAAUGUUAA | SEQ ID No 1408 | UUAACAUUGGCCGUGACAGCU |
| SEQ ID No 810 | UAUCUACUGAUGGUAACAAAA | SEQ ID No 1409 | UUUUGUUACCAUCAGUAGAUA |
| SEQ ID No 811 | UUAUGAGUGUCUCUAUAGAAA | SEQ ID No 1410 | UUUCUAUAGAGACACUCAUAA |
| SEQ ID No 812 | AGGUCUAGUGGCUAGCAUAAA | SEQ ID No 1411 | UUUAUGCUAGCCACUAGACCU |
| SEQ ID No 813 | UCUAGUGGCUAGCAUAAAGAA | SEQ ID No 1412 | UUCUUUAUGCUAGCCACUAGA |
| SEQ ID No 814 | ACUGAGACUGACCUUACUAAA | SEQ ID No 1413 | UUUAGUAAGGUCAGUCUCAGU |
| SEQ ID No 815 | CAACAUACAAUGCUAGUUAAA | SEQ ID No 1414 | UUUAACUAGCAUUGUAUGUUG |
| SEQ ID No 816 | UUGUAGAUGAUAUCGUAAAAA | SEQ ID No 1415 | UUUUUACGAUAUCAUCUACAA |
| SEQ ID No 817 | GAUGGUACACUUAUGAUUGAA | SEQ ID No 1416 | UUCAAUCAUAAGUGUACCAUC |
| SEQ ID No 818 | UGUACUUACAAUACAUAAGAA | SEQ ID No 1417 | UUCUUAUGUAUUGUAAGUACA |
| SEQ ID No 819 | GUACUUACAAUACAUAAGAAA | SEQ ID No 1418 | UUUCUUAUGUAUUGUAAGUAC |
| SEQ ID No 820 | GAAAGCUACAUGAUGAGUUAA | SEQ ID No 1419 | UUAACUCAUCAUGUAGCUUUC |
| SEQ ID No 821 | AAAUUCCUAAAAACUAAUUGU | SEQ ID No 1420 | ACAAUUAGUUUUUAGGAAUUU |
| SEQ ID No 822 | AAUUCCUAAAAACUAAUUGUU | SEQ ID No 1421 | AACAAUUAGUUUUUAGGAAUU |
| SEQ ID No 823 | AAAAGGACGAAGAUGACAAUU | SEQ ID No 1422 | AAUUGUCAUCUUCGUCCUUUU |
| SEQ ID No 824 | AACGUCUUACUAAAUACACAA | SEQ ID No 1423 | UUGUGUAUUUAGUAAGACGUU |
| SEQ ID No 825 | AAGGUAAUUGUGACACAUUAA | SEQ ID No 1424 | UUAAUGUGUCACAAUUACCUU |
| SEQ ID No 826 | AAAUACUUGUCACAUACAAUU | SEQ ID No 1425 | AAUUGUAUGUGACAAGUAUUU |
| SEQ ID No 827 | AAUUGUUGUGAUGAUGAUUAU | SEQ ID No 1426 | AUAAUCAUCAUCACAACAAUU |
| SEQ ID No 828 | AAUCAAGAUCUCAAUGGUAAC | SEQ ID No 1427 | GUUACCAUUGAGAUCUUGAUU |
| SEQ ID No 829 | AACAAAGCCUUACAUUAAGUG | SEQ ID No 1428 | CACUUAAUGUAAGGCUUUGUU |
| SEQ ID No 830 | AAUCAGGAUGUAAACUUACAU | SEQ ID No 1429 | AUGUAAGUUUACAUCCUGAUU |
| SEQ ID No 831 | AACUCAAAUGAAUCUUAAGUA | SEQ ID No 1430 | UACUUAAGAUUCAUUUGAGUU |
| SEQ ID No 832 | AAAAUUAUUGAAAUCAAUAGC | SEQ ID No 1431 | GCUAUUGAUUUCAAUAAUUUU |
| SEQ ID No 833 | AAUUGGAACAAGCAAAUUCUA | SEQ ID No 1432 | UAGAAUUUGCUUGUUCCAAUU |
| SEQ ID No 834 | AAUGAGUGUGCUCAAGUAUUG | SEQ ID No 1433 | CAAUACUUGAGCACACUCAUU |
| SEQ ID No 835 | AACUGCUUAUGCUAAUAGUGU | SEQ ID No 1434 | ACACUAUUAGCAUAAGCAGUU |
| SEQ ID No 836 | AACAAAAUUGCCGAUAAGUAU | SEQ ID No 1435 | AUACUUAUCGGCAAUUUUGUU |
| SEQ ID No 837 | AAAAUUGCCGAUAAGUAUGUC | SEQ ID No 1436 | GACAUACUUAUCGGCAAUUUU |
| SEQ ID No 838 | AAAAACAGAUGGUACACUUAU | SEQ ID No 1437 | AUAAGUGUACCAUCUGUUUUU |
| SEQ ID No 839 | AAAACAGAUGGUACACUUAUG | SEQ ID No 1438 | CAUAAGUGUACCAUCUGUUUU |
| SEQ ID No 840 | AACAGAUGGUACACUUAUGAU | SEQ ID No 1439 | AUCAUAAGUGUACCAUCUGUU |
| SEQ ID No 841 | AAAGCUACAUGAUGAGUUAAC | SEQ ID No 1440 | GUUAACUCAUCAUGUAGCUUU |
| SEQ ID No 842 | UAUGGCUGUAGUUGUGAUCAA | SEQ ID No 1441 | UUGAUCACAACUACAGCCAUA |
| SEQ ID No 843 | AAUUGUUGUCGCUUCCAAGAA | SEQ ID No 1442 | UUCUUGGAAGCGACAACAAUU |
| SEQ ID No 844 | AUUGUUGUCGCUUCCAAGAAA | SEQ ID No 1443 | UUUCUUGGAAGCGACAACAAU |
| SEQ ID No 845 | UUGUUGUCGCUUCCAAGAAAA | SEQ ID No 1444 | UUUUCUUGGAAGCGACAACAA |
| SEQ ID No 846 | AGAAAAGGACGAAGAUGACAA | SEQ ID No 1445 | UUGUCAUCUUCGUCCUUUUCU |
| SEQ ID No 847 | UUCUCUAACUACCAACAUGAA | SEQ ID No 1446 | UUCAUGUUGGUAGUUAGAGAA |
| SEQ ID No 848 | UCUAACUACCAACAUGAAGAA | SEQ ID No 1447 | UUCUUCAUGUUGGUAGUUAGA |
| SEQ ID No 849 | GGAUUGUCCAGCUGUUGCUAA | SEQ ID No 1448 | UUAGCAACAGCUGGACAAUCC |
| SEQ ID No 850 | GAUUGUCCAGCUGUUGCUAAA | SEQ ID No 1449 | UUUAGCAACAGCUGGACAAUC |
| SEQ ID No 851 | AGAAAUACUUGUCACAUACAA | SEQ ID No 1450 | UUGUAUGUGACAAGUAUUUCU |
| SEQ ID No 852 | GUAUACGCCAACUUAGGUGAA | SEQ ID No 1451 | UUCACCUAAGUUGGCGUAUAC |
| SEQ ID No 853 | AUUAGAUAAUCAAGAUCUCAA | SEQ ID No 1452 | UUGAGAUCUUGAUUAUCUAAU |
| SEQ ID No 854 | UAAUCAAGAUCUCAAUGGUAA | SEQ ID No 1453 | UUACCAUUGAGAUCUUGAUUA |
| SEQ ID No 855 | CACAUGUUGACACUGACUUAA | SEQ ID No 1454 | UUAAGUCAGUGUCAACAUGUG |
| SEQ ID No 856 | ACUUCACGGAAGAGAGGUUAA | SEQ ID No 1455 | UUAACCUCUCUUCCGUGAAGU |
| SEQ ID No 857 | CUUCACGGAAGAGAGGUUAAA | SEQ ID No 1456 | UUUAACCUCUCUUCCGUGAAG |
| SEQ ID No 858 | GAUGCAUUCUGCAUUGUGCAA | SEQ ID No 1457 | UUGCACAAUGCAGAAUGCAUC |
| SEQ ID No 859 | AUGCAUUCUGCAUUGUGCAAA | SEQ ID No 1458 | UUUGCACAAUGCAGAAUGCAU |
| SEQ ID No 860 | UUGUACAUAAUCAGGAUGUAA | SEQ ID No 1459 | UUACAUCCUGAUUAUGUACAA |
| SEQ ID No 861 | UGUACAUAAUCAGGAUGUAAA | SEQ ID No 1460 | UUUACAUCCUGAUUAUGUACA |
| SEQ ID No 862 | UAUGCACGCUGCUUCUGGUAA | SEQ ID No 1461 | UUACCAGAAGCAGCGUGCAUA |
| SEQ ID No 863 | AAGGAAGGAAGUUCUGUUGAA | SEQ ID No 1462 | UUCAACAGAACUUCCUUCCUU |
| SEQ ID No 864 | ACUAUCGUUAUAAUCUACCAA | SEQ ID No 1463 | UUGGUAGAUUAUAACGAUAGU |
| SEQ ID No 865 | AUCGUUAUAAUCUACCAACAA | SEQ ID No 1464 | UUGUUGGUAGAUUAUAACGAU |
| SEQ ID No 866 | ACAAUGUGUGAUAUCAGACAA | SEQ ID No 1465 | UUGUCUGAUAUCACACAUUGU |
| SEQ ID No 867 | AUCGUCAACAACCUAGACAAA | SEQ ID No 1466 | UUUGUCUAGGUUGUUGACGAU |
| SEQ ID No 868 | UUAAGUAUGCCAUUAGUGCAA | SEQ ID No 1467 | UUGCACUAAUGGCAUACUUAA |
| SEQ ID No 869 | UAAGUAUGCCAUUAGUGCAAA | SEQ ID No 1468 | UUUGCACUAAUGGCAUACUUA |
| SEQ ID No 870 | GUAUGCCAUUAGUGCAAAGAA | SEQ ID No 1469 | UUCUUUGCACUAAUGGCAUAC |
| SEQ ID No 871 | UAUCUGUAGUACUAUGACCAA | SEQ ID No 1470 | UUGGUCAUAGUACUACAGAUA |
| SEQ ID No 872 | CUAGAGGAGCUACUGUAGUAA | SEQ ID No 1471 | UUACUACAGUAGCUCCUCUAG |
| SEQ ID No 873 | CUACUGUAGUAAUUGGAACAA | SEQ ID No 1472 | UUGUUCCAAUUACUACAGUAG |
| SEQ ID No 874 | UGUAGUAAUUGGAACAAGCAA | SEQ ID No 1473 | UUGCUUGUUCCAAUUACUACA |
| SEQ ID No 875 | GUAGUAAUUGGAACAAGCAAA | SEQ ID No 1474 | UUUGCUUGUUCCAAUUACUAC |
| SEQ ID No 876 | CCAUGCCUAACAUGCUUAGAA | SEQ ID No 1475 | UUCUAAGCAUGUUAGGCAUGG |
| SEQ ID No 877 | UGUGCUCAAGUAUUGAGUGAA | SEQ ID No 1476 | UUCACUCAAUACUUGAGCACA |
| SEQ ID No 878 | GUGCUCAAGUAUUGAGUGAAA | SEQ ID No 1477 | UUUCACUCAAUACUUGAGCAC |
| SEQ ID No 879 | UUAUCUACUGAUGGUAACAAA | SEQ ID No 1478 | UUUGUUACCAUCAGUAGAUAA |
| SEQ ID No 880 | AAGGUCUAGUGGCUAGCAUAA | SEQ ID No 1479 | UUAUGCUAGCCACUAGACCUU |
| SEQ ID No 881 | GACUGAGACUGACCUUACUAA | SEQ ID No 1480 | UUAGUAAGGUCAGUCUCAGUC |
| SEQ ID No 882 | UCAACAUACAAUGCUAGUUAA | SEQ ID No 1481 | UUAACUAGCAUUGUAUGUUGA |
| SEQ ID No 883 | GUAUUCUGUUAUGCUUACUAA | SEQ ID No 1482 | UUAGUAAGCAUAACAGAAUAC |
| SEQ ID No 884 | AAAGGUUAUGGCUGUAGUUGU | SEQ ID No 1483 | ACAACUACAGCCAUAACCUUU |
| SEQ ID No 885 | AAGGUUAUGGCUGUAGUUGUG | SEQ ID No 1484 | CACAACUACAGCCAUAACCUU |
| SEQ ID No 886 | AAUUGUUGUCGCUUCCAAGAA | SEQ ID No 1485 | UUCUUGGAAGCGACAACAAUU |
| SEQ ID No 887 | AAGAAAAGGACGAAGAUGACA | SEQ ID No 1486 | UGUCAUCUUCGUCCUUUUCUU |
| SEQ ID No 888 | AACUACCAACAUGAAGAAACA | SEQ ID No 1487 | UGUUUCUUCAUGUUGGUAGUU |
| SEQ ID No 889 | AAAAGAAAUACUUGUCACAUA | SEQ ID No 1488 | UAUGUGACAAGUAUUUCUUUU |
| SEQ ID No 890 | AAAGAAAUACUUGUCACAUAC | SEQ ID No 1489 | GUAUGUGACAAGUAUUUCUUU |
| SEQ ID No 891 | AAGAAAUACUUGUCACAUACA | SEQ ID No 1490 | UGUAUGUGACAAGUAUUUCUU |
| SEQ ID No 892 | AAUAAAAAGGACUGGUAUGAU | SEQ ID No 1491 | AUCAUACCAGUCCUUUUUAUU |
| SEQ ID No 893 | AAAAACAGUACAAUUCUGUGA | SEQ ID No 1492 | UCACAGAAUUGUACUGUUUUU |
| SEQ ID No 894 | AAAACAGUACAAUUCUGUGAU | SEQ ID No 1493 | AUCACAGAAUUGUACUGUUUU |
| SEQ ID No 895 | AAACAGUACAAUUCUGUGAUG | SEQ ID No 1494 | CAUCACAGAAUUGUACUGUUU |
| SEQ ID No 896 | AAUGCCUAUAUUAACCUUGAC | SEQ ID No 1495 | GUCAAGGUUAAUAUAGGCAUU |
| SEQ ID No 897 | AACUGCAGAGUCACAUGUUGA | SEQ ID No 1496 | UCAACAUGUGACUCUGCAGUU |
| SEQ ID No 898 | AACUUACAUAGCUCUAGACUU | SEQ ID No 1497 | AAGUCUAGAGCUAUGUAAGUU |
| SEQ ID No 899 | AAUCUAUUACUAGAUAAACGC | SEQ ID No 1498 | GCGUUUAUCUAGUAAUAGAUU |
| SEQ ID No 900 | AAGGAAGGAAGUUCUGUUGAA | SEQ ID No 1499 | UUCAACAGAACUUCCUUCCUU |
| SEQ ID No 901 | AAUGCUGCUAUCAGCGAUUAU | SEQ ID No 1500 | AUAAUCGCUGAUAGCAGCAUU |
| SEQ ID No 902 | AAUCUACCAACAAUGUGUGAU | SEQ ID No 1501 | AUCACACAUUGUUGGUAGAUU |
| SEQ ID No 903 | AACAAUGUGUGAUAUCAGACA | SEQ ID No 1502 | UGUCUGAUAUCACACAUUGUU |
| SEQ ID No 904 | AACCAAGUCAUCGUCAACAAC | SEQ ID No 1503 | GUUGUUGACGAUGACUUGGUU |
| SEQ ID No 905 | AAGUCAUCGUCAACAACCUAG | SEQ ID No 1504 | CUAGGUUGUUGACGAUGACUU |
| SEQ ID No 906 | AACAACCUAGACAAAUCAGCU | SEQ ID No 1505 | AGCUGAUUUGUCUAGGUUGUU |
| SEQ ID No 907 | AAGUAUGCCAUUAGUGCAAAG | SEQ ID No 1506 | CUUUGCACUAAUGGCAUACUU |
| SEQ ID No 908 | AAAUUAUUGAAAUCAAUAGCC | SEQ ID No 1507 | GGCUAUUGAUUUCAAUAAUUU |
| SEQ ID No 909 | AACAUGCUUAGAAUUAUGGCC | SEQ ID No 1508 | GGCCAUAAUUCUAAGCAUGUU |
| SEQ ID No 910 | AAUUAUGGCCUCACUUGUUCU | SEQ ID No 1509 | AGAACAAGUGAGGCCAUAAUU |
| SEQ ID No 911 | AAACAUACAACGUGUUGUAGC | SEQ ID No 1510 | GCUACAACACGUUGUAUGUUU |
| SEQ ID No 912 | AAGUAUUGAGUGAAAUGGUCA | SEQ ID No 1511 | UGACCAUUUCACUCAAUACUU |
| SEQ ID No 913 | AAGCUGUCACGGCCAAUGUUA | SEQ ID No 1512 | UAACAUUGGCCGUGACAGCUU |
| SEQ ID No 914 | AAAUUGCCGAUAAGUAUGUCC | SEQ ID No 1513 | GGACAUACUUAUCGGCAAUUU |
| SEQ ID No 915 | AAGGUCUAGUGGCUAGCAUAA | SEQ ID No 1514 | UUAUGCUAGCCACUAGACCUU |
| SEQ ID No 916 | AAACAGAUGGUACACUUAUGA | SEQ ID No 1515 | UCAUAAGUGUACCAUCUGUUU |
| SEQ ID No 917 | AACAUCCUAAUCAGGAGUAUG | SEQ ID No 1516 | CAUACUCCUGAUUAGGAUGUU |
| SEQ ID No 918 | AAUACAUAAGAAAGCUACAUG | SEQ ID No 1517 | CAUGUAGCUUUCUUAUGUAUU |
| SEQ ID No 919 | AAGAAAGCUACAUGAUGAGUU | SEQ ID No 1518 | AACUCAUCAUGUAGCUUUCUU |
| SEQ ID No 920 | AAGCUACAUGAUGAGUUAACA | SEQ ID No 1519 | UGUUAACUCAUCAUGUAGCUU |
| SEQ ID No 921 | AACAGGACACAUGUUAGACAU | SEQ ID No 1520 | AUGUCUAACAUGUGUCCUGUU |
| SEQ ID No 922 | AAUGAUAACACUUCAAGGUAU | SEQ ID No 1521 | AUACCUUGAAGUGUUAUCAUU |
| SEQ ID No 923 | CUUCAGUCAGCUGAUGCACAA | SEQ ID No 1522 | UUGUGCAUCAGCUGACUGAAG |
| SEQ ID No 924 | CGCUUCCAAGAAAAGGACGAA | SEQ ID No 1523 | UUCGUCCUUUUCUUGGAAGCG |
| SEQ ID No 925 | GUACCACAUAUAUCACGUCAA | SEQ ID No 1524 | UUGACGUGAUAUAUGUGGUAC |
| SEQ ID No 926 | UAUAUUACGCGUAUACGCCAA | SEQ ID No 1525 | UUGGCGUAUACGCGUAAUAUA |
| SEQ ID No 927 | AUUCUGUGAUGCCAUGCGAAA | SEQ ID No 1526 | UUUCGCAUGGCAUCACAGAAU |
| SEQ ID No 928 | UUAAAAUAUGACUUCACGGAA | SEQ ID No 1527 | UUCCGUGAAGUCAUAUUUUAA |
| SEQ ID No 929 | GGCUGUAUUAAUGCUAACCAA | SEQ ID No 1528 | UUGGUUAGCAUUAAUACAGCC |
| SEQ ID No 930 | UGCUAACCAAGUCAUCGUCAA | SEQ ID No 1529 | UUGACGAUGACUUGGUUAGCA |
| SEQ ID No 931 | UAACCAAGUCAUCGUCAACAA | SEQ ID No 1530 | UUGUUGACGAUGACUUGGUUA |
| SEQ ID No 932 | CAUCGUCAACAACCUAGACAA | SEQ ID No 1531 | UUGUCUAGGUUGUUGACGAUG |
| SEQ ID No 933 | UCAAUGAGUUAUGAGGAUCAA | SEQ ID No 1532 | UUGAUCCUCAUAACUCAUUGA |
| SEQ ID No 934 | AUUCUAUGGUGGUUGGCACAA | SEQ ID No 1533 | UUGUGCCAACCACCAUAGAAU |
| SEQ ID No 935 | AUGUGAUAGAGCCAUGCCUAA | SEQ ID No 1534 | UUAGGCAUGGCUCUAUCACAU |
| SEQ ID No 936 | UUAGCUAAUGAGUGUGCUCAA | SEQ ID No 1535 | UUGAGCACACUCAUUAGCUAA |
| SEQ ID No 937 | CCUCAUCAGGAGAUGCCACAA | SEQ ID No 1536 | UUGUGGCAUCUCCUGAUGAGG |
| SEQ ID No 938 | CAUCAUCCGGUGAUGCUACAA | SEQ ID No 1537 | UUGUAGCAUCACCGGAUGAUG |
| SEQ ID No 939 | UGGUAACAAAAUUGCCGAUAA | SEQ ID No 1538 | UUAUCGGCAAUUUUGUUACCA |
| SEQ ID No 940 | AAUAGCACUUAUGCAUCUCAA | SEQ ID No 1539 | UUGAGAUGCAUAAGUGCUAUU |
| SEQ ID No 941 | CUUACUAAAGGACCUCAUGAA | SEQ ID No 1540 | UUCAUGAGGUCCUUUAGUAAG |
| SEQ ID No 942 | AAAAACUAAUUGUUGUCGCUU | SEQ ID No 1541 | AAGCGACAACAAUUAGUUUUU |
| SEQ ID No 943 | AAAACUAAUUGUUGUCGCUUC | SEQ ID No 1542 | GAAGCGACAACAAUUAGUUUU |
| SEQ ID No 944 | AAUAGACGGUGACAUGGUACC | SEQ ID No 1543 | GGUACCAUGUCACCGUCUAUU |
| SEQ ID No 945 | AAUGGCAGACCUCGUCUAUGC | SEQ ID No 1544 | GCAUAGACGAGGUCUGCCAUU |
| SEQ ID No 946 | AAUGGCUGAUUUAGUCUAUGC | SEQ ID No 1545 | GCAUAGACUAAAUCAGCCAUU |
| SEQ ID No 947 | AACAGUACAAUUCUGUGAUGC | SEQ ID No 1546 | GCAUCACAGAAUUGUACUGUU |
| SEQ ID No 948 | AAAUGCUGGUAUUGUUGGUGU | SEQ ID No 1547 | ACACCAACAAUACCAGCAUUU |
| SEQ ID No 949 | AAUGCUGGUAUUGUUGGUGUA | SEQ ID No 1548 | UACACCAACAAUACCAGCAUU |
| SEQ ID No 950 | AAGAUCUCAAUGGUAACUGGU | SEQ ID No 1549 | ACCAGUUACCAUUGAGAUCUU |
| SEQ ID No 951 | AAACCACGCCAGGUAGUGGAG | SEQ ID No 1550 | CUCCACUACCUGGCGUGGUUU |
| SEQ ID No 952 | AAGUAGCACCAGGCUGCGGAG | SEQ ID No 1551 | CUCCGCAGCCUGGUGCUACUU |
| SEQ ID No 953 | AACCACGCCAGGUAGUGGAGU | SEQ ID No 1552 | ACUCCACUACCUGGCGUGGUU |
| SEQ ID No 954 | AAAUAUGACUUCACGGAAGAG | SEQ ID No 1553 | CUCUUCCGUGAAGUCAUAUUU |
| SEQ ID No 955 | AACUGGAUACCACUUCAGAGA | SEQ ID No 1554 | UCUCUGAAGUGGUAUCCAGUU |
| SEQ ID No 956 | AAACUUACAUAGCUCUAGACU | SEQ ID No 1555 | AGUCUAGAGCUAUGUAAGUUU |
| SEQ ID No 957 | AAGGAAUUACUUGUGUAUGCU | SEQ ID No 1556 | AGCAUACACAAGUAAUUCCUU |
| SEQ ID No 958 | AAUUACUUGUGUAUGCUGCUG | SEQ ID No 1557 | CAGCAGCAUACACAAGUAAUU |
| SEQ ID No 959 | AAUGUGUGAUAUCAGACAACU | SEQ ID No 1558 | AGUUGUCUGAUAUCACACAUU |
| SEQ ID No 960 | AAUGCUAACCAAGUCAUCGUC | SEQ ID No 1559 | GACGAUGACUUGGUUAGCAUU |
| SEQ ID No 961 | AAUGAGUUAUGAGGAUCAAGA | SEQ ID No 1560 | UCUUGAUCCUCAUAACUCAUU |
| SEQ ID No 962 | AAAUGAAUCUUAAGUAUGCCA | SEQ ID No 1561 | UGGCAUACUUAAGAUUCAUUU |
| SEQ ID No 963 | AAUGAAUCUUAAGUAUGCCAU | SEQ ID No 1562 | AUGGCAUACUUAAGAUUCAUU |
| SEQ ID No 964 | AAUCUUAAGUAUGCCAUUAGU | SEQ ID No 1563 | ACUAAUGGCAUACUUAAGAUU |
| SEQ ID No 965 | AAUUAUUGAAAUCAAUAGCCG | SEQ ID No 1564 | CGGCUAUUGAUUUCAAUAAUU |
| SEQ ID No 966 | AAUCAAUAGCCGCCACUAGAG | SEQ ID No 1565 | CUCUAGUGGCGGCUAUUGAUU |
| SEQ ID No 967 | AAUAGCCGCCACUAGAGGAGC | SEQ ID No 1566 | GCUCCUCUAGUGGCGGCUAUU |
| SEQ ID No 968 | AACAAGCAAAUUCUAUGGUGG | SEQ ID No 1567 | CCACCAUAGAAUUUGCUUGUU |
| SEQ ID No 969 | AAGCAAAUUCUAUGGUGGUUG | SEQ ID No 1568 | CAACCACCAUAGAAUUUGCUU |
| SEQ ID No 970 | AACAUACAACGUGUUGUAGCU | SEQ ID No 1569 | AGCUACAACACGUUGUAUGUU |
| SEQ ID No 971 | AACGUGUUGUAGCUUGUCACA | SEQ ID No 1570 | UGUGACAAGCUACAACACGUU |
| SEQ ID No 972 | AAACCAGGUGGAACCUCAUCA | SEQ ID No 1571 | UGAUGAGGUUCCACCUGGUUU |
| SEQ ID No 973 | AACCAGGUGGAACCUCAUCAG | SEQ ID No 1572 | CUGAUGAGGUUCCACCUGGUU |
| SEQ ID No 974 | AACCAGGUGGAACAUCAUCCG | SEQ ID No 1573 | CGGAUGAUGUUCCACCUGGUU |
| SEQ ID No 975 | AACCUCAUCAGGAGAUGCCAC | SEQ ID No 1574 | GUGGCAUCUCCUGAUGAGGUU |
| SEQ ID No 976 | AACAUCAUCCGGUGAUGCUAC | SEQ ID No 1575 | GUAGCAUCACCGGAUGAUGUU |
| SEQ ID No 977 | AAUUGCCGAUAAGUAUGUCCG | SEQ ID No 1576 | CGGACAUACUUAUCGGCAAUU |
| SEQ ID No 978 | AAAUAGAGAUGUUGACACAGA | SEQ ID No 1577 | UCUGUGUCAACAUCUCUAUUU |
| SEQ ID No 979 | AAUAGAGAUGUUGACACAGAC | SEQ ID No 1578 | GUCUGUGUCAACAUCUCUAUU |
| SEQ ID No 980 | AAUAGCACUUAUGCAUCUCAA | SEQ ID No 1579 | UUGAGAUGCAUAAGUGCUAUU |
| SEQ ID No 981 | AAAAUGUUGGACUGAGACUGA | SEQ ID No 1580 | UCAGUCUCAGUCCAACAUUUU |
| SEQ ID No 982 | AAAUGUUGGACUGAGACUGAC | SEQ ID No 1581 | GUCAGUCUCAGUCCAACAUUU |
| SEQ ID No 983 | AAUGUUGGACUGAGACUGACC | SEQ ID No 1582 | GGUCAGUCUCAGUCCAACAUU |
| SEQ ID No 984 | AAACAUCCUAAUCAGGAGUAU | SEQ ID No 1583 | AUACUCCUGAUUAGGAUGUUU |
| SEQ ID No 985 | AAUCAGGAGUAUGCUGAUGUC | SEQ ID No 1584 | GACAUCAGCAUACUCCUGAUU |
| SEQ ID No 986 | ACUAAUUGUUGUCGCUUCCAA | SEQ ID No 1585 | UUGGAAGCGACAACAAUUAGU |
| SEQ ID No 987 | UUAGGUGAACGUGUACGCCAA | SEQ ID No 1586 | UUGGCGUACACGUUCACCUAA |
| SEQ ID No 988 | AAUUCUGUGAUGCCAUGCGAA | SEQ ID No 1587 | UUCGCAUGGCAUCACAGAAUU |
| SEQ ID No 989 | CUCACUUGUUCUUGCUCGCAA | SEQ ID No 1588 | UUGCGAGCAAGAACAAGUGAG |
| SEQ ID No 990 | UCACUUGUUCUUGCUCGCAAA | SEQ ID No 1589 | UUUGCGAGCAAGAACAAGUGA |
| SEQ ID No 991 | UAUAUGUUAAACCAGGUGGAA | SEQ ID No 1590 | UUCCACCUGGUUUAACAUAUA |
| SEQ ID No 992 | UGCCGAUAAGUAUGUCCGCAA | SEQ ID No 1591 | UUGCGGACAUACUUAUCGGCA |
| SEQ ID No 993 | AACACCGUGCGGCACAGGCAC | SEQ ID No 1592 | GUGCCUGUGCCGCACGGUGUU |
| SEQ ID No 994 | AAACUAAUUGUUGUCGCUUCC | SEQ ID No 1593 | GGAAGCGACAACAAUUAGUUU |
| SEQ ID No 995 | AACUAAUUGUUGUCGCUUCCA | SEQ ID No 1594 | UGGAAGCGACAACAAUUAGUU |
| SEQ ID No 996 | AAGGAUUGUCCAGCUGUUGCU | SEQ ID No 1595 | AGCAACAGCUGGACAAUCCUU |
| SEQ ID No 997 | AAAUACACAAUGGCAGACCUC | SEQ ID No 1596 | GAGGUCUGCCAUUGUGUAUUU |
| SEQ ID No 998 | AAUACACAAUGGCAGACCUCG | SEQ ID No 1597 | CGAGGUCUGCCAUUGUGUAUU |
| SEQ ID No 999 | AACUUAGGUGAACGUGUACGC | SEQ ID No 1598 | GCGUACACGUUCACCUAAGUU |
| SEQ ID No 1000 | AAUUCUGUGAUGCCAUGCGAA | SEQ ID No 1599 | UUCGCAUGGCAUCACAGAAUU |
| SEQ ID No 1001 | AAAAUAUGACUUCACGGAAGA | SEQ ID No 1600 | UCUUCCGUGAAGUCAUAUUUU |
| SEQ ID No 1002 | AAUAUGACUUCACGGAAGAGA | SEQ ID No 1601 | UCUCUUCCGUGAAGUCAUAUU |
| SEQ ID No 1003 | AACCUAGACAAAUCAGCUGGU | SEQ ID No 1602 | ACCAGCUGAUUUGUCUAGGUU |
| SEQ ID No 1004 | AAUAGAGCUCGCACCGUAGCU | SEQ ID No 1603 | AGCUACGGUGCGAGCUCUAUU |
| SEQ ID No 1005 | AAAUCAAUAGCCGCCACUAGA | SEQ ID No 1604 | UCUAGUGGCGGCUAUUGAUUU |
| SEQ ID No 1006 | AAAUUCUAUGGUGGUUGGCAC | SEQ ID No 1605 | GUGCCAACCACCAUAGAAUUU |
| SEQ ID No 1007 | AAUUCUAUGGUGGUUGGCACA | SEQ ID No 1606 | UGUGCCAACCACCAUAGAAUU |
| SEQ ID No 1008 | AAUGUGAUAGAGCCAUGCCUA | SEQ ID No 1607 | UAGGCAUGGCUCUAUCACAUU |
| SEQ ID No 1009 | AAUGGUCAUGUGUGGCGGUUC | SEQ ID No 1608 | GAACCGCCACACAUGACCAUU |
| SEQ ID No 1010 | AAUGAUGAUACUCUCUGACGA | SEQ ID No 1609 | UCGUCAGAGAGUAUCAUCAUU |
| SEQ ID No 1011 | AAGCAAAAUGUUGGACUGAGA | SEQ ID No 1610 | UCUCAGUCCAACAUUUUGCUU |
| SEQ ID No 1012 | AGUUGUGAUCAACUCCGCGAA | SEQ ID No 1611 | UUCGCGGAGUUGAUCACAACU |
| SEQ ID No 1013 | UUGUCAAGCUGUCACGGCCAA | SEQ ID No 1612 | UUGGCCGUGACAGCUUGACAA |
| SEQ ID No 1014 | AAACACCGUGCGGCACAGGCA | SEQ ID No 1613 | UGCCUGUGCCGCACGGUGUUU |
| SEQ ID No 1015 | AAAGAAUAGAGCUCGCACCGU | SEQ ID No 1614 | ACGGUGCGAGCUCUAUUCUUU |
| SEQ ID No 1016 | AAGAAUAGAGCUCGCACCGUA | SEQ ID No 1615 | UACGGUGCGAGCUCUAUUCUU |
| SEQ ID No 1017 | AAAUGUGAUAGAGCCAUGCCU | SEQ ID No 1616 | AGGCAUGGCUCUAUCACAUUU |
| SEQ ID No 1018 | AAAUGGUCAUGUGUGGCGGUU | SEQ ID No 1617 | AACCGCCACACAUGACCAUUU |
| SEQ ID No 1019 | AGGUAUGAGCUAUUAUUGUAA | SEQ ID No 1618 | UUACAAUAAUAGCUCAUACCU |
| SEQ ID No 1020 | GGUAUGAGCUAUUAUUGUAAA | SEQ ID No 1619 | UUUACAAUAAUAGCUCAUACC |
| SEQ ID No 1021 | CUGGUUAUCGUGUAACUAAAA | SEQ ID No 1620 | UUUUAGUUACACGAUAACCAG |
| SEQ ID No 1022 | UGGUUAUCGUGUAACUAAAAA | SEQ ID No 1621 | UUUUUAGUUACACGAUAACCA |
| SEQ ID No 1023 | GUACAACAACUUACAAAUUAA | SEQ ID No 1622 | UUAAUUUGUAAGUUGUUGUAC |
| SEQ ID No 1024 | UACAACAACUUACAAAUUAAA | SEQ ID No 1623 | UUUAAUUUGUAAGUUGUUGUA |
| SEQ ID No 1025 | AGCAAUGUUGCAAAUUAUCAA | SEQ ID No 1624 | UUGAUAAUUUGCAACAUUGCU |
| SEQ ID No 1026 | GCAAUGUUGCAAAUUAUCAAA | SEQ ID No 1625 | UUUGAUAAUUUGCAACAUUGC |
| SEQ ID No 1027 | CAAUGUUGCAAAUUAUCAAAA | SEQ ID No 1626 | UUUUGAUAAUUUGCAACAUUG |
| SEQ ID No 1028 | CAGUGUGUAGACUUAUGAAAA | SEQ ID No 1627 | UUUUCAUAAGUCUACACACUG |
| SEQ ID No 1029 | AGCUCACUCUUGUAAUGUAAA | SEQ ID No 1628 | UUUACAUUACAAGAGUGAGCU |
| SEQ ID No 1030 | AAAACAGUAAAGUACAAAUAG | SEQ ID No 1629 | CUAUUUGUACUUUACUGUUUU |
| SEQ ID No 1031 | AACAACUUACAAAUUAAAUGU | SEQ ID No 1630 | ACAUUUAAUUUGUAAGUUGUU |
| SEQ ID No 1032 | AAGAGCACUAUGUUAGAAUUA | SEQ ID No 1631 | UAAUUCUAACAUAGUGCUCUU |
| SEQ ID No 1033 | AAUGUUGCAAAUUAUCAAAAG | SEQ ID No 1632 | CUUUUGAUAAUUUGCAACAUU |
| SEQ ID No 1034 | UAGACCAUUCUUAUGUUGUAA | SEQ ID No 1633 | UUACAACAUAAGAAUGGUCUA |
| SEQ ID No 1035 | AGACCAUUCUUAUGUUGUAAA | SEQ ID No 1634 | UUUACAACAUAAGAAUGGUCU |
| SEQ ID No 1036 | GUUACGACCAUGUCAUAUCAA | SEQ ID No 1635 | UUGAUAUGACAUGGUCGUAAC |
| SEQ ID No 1037 | UGUCAUAUCAACAUCACAUAA | SEQ ID No 1636 | UUAUGUGAUGUUGAUAUGACA |
| SEQ ID No 1038 | GUCAUAUCAACAUCACAUAAA | SEQ ID No 1637 | UUUAUGUGAUGUUGAUAUGAC |
| SEQ ID No 1039 | CUAUUAUUGUAAAUCACAUAA | SEQ ID No 1638 | UUAUGUGAUUUACAAUAAUAG |
| SEQ ID No 1040 | UAUUAUUGUAAAUCACAUAAA | SEQ ID No 1639 | UUUAUGUGAUUUACAAUAAUA |
| SEQ ID No 1041 | UACUGGUUAUCGUGUAACUAA | SEQ ID No 1640 | UUAGUUACACGAUAACCAGUA |
| SEQ ID No 1042 | ACUGGUUAUCGUGUAACUAAA | SEQ ID No 1641 | UUUAGUUACACGAUAACCAGU |
| SEQ ID No 1043 | UCGUGUAACUAAAAACAGUAA | SEQ ID No 1642 | UUACUGUUUUUAGUUACACGA |
| SEQ ID No 1044 | CGUGUAACUAAAAACAGUAAA | SEQ ID No 1643 | UUUACUGUUUUUAGUUACACG |
| SEQ ID No 1045 | ACUAAAAACAGUAAAGUACAA | SEQ ID No 1644 | UUGUACUUUACUGUUUUUAGU |
| SEQ ID No 1046 | CUAAAAACAGUAAAGUACAAA | SEQ ID No 1645 | UUUGUACUUUACUGUUUUUAG |
| SEQ ID No 1047 | CCGAGGUACAACAACUUACAA | SEQ ID No 1646 | UUGUAAGUUGUUGUACCUCGG |
| SEQ ID No 1048 | CGAGGUACAACAACUUACAAA | SEQ ID No 1647 | UUUGUAAGUUGUUGUACCUCG |
| SEQ ID No 1049 | UGCUGACAUCACAUACAGUAA | SEQ ID No 1648 | UUACUGUAUGUGAUGUCAGCA |
| SEQ ID No 1050 | CACAAGAGCACUAUGUUAGAA | SEQ ID No 1649 | UUCUAACAUAGUGCUCUUGUG |
| SEQ ID No 1051 | UCAAAAGGUUGGUAUGCAAAA | SEQ ID No 1650 | UUUUGCAUACCAACCUUUUGA |
| SEQ ID No 1052 | CUAUGUGAGAAGGCAUUAAAA | SEQ ID No 1651 | UUUUAAUGCCUUCUCACAUAG |
| SEQ ID No 1053 | AUAAAUUCAAAGUGAAUUCAA | SEQ ID No 1652 | UUGAAUUCACUUUGAAUUUAU |
| SEQ ID No 1054 | AAAGUGAAUUCAACAUUAGAA | SEQ ID No 1653 | UUCUAAUGUUGAAUUCACUUU |
| SEQ ID No 1055 | UUCAGUGUGUAGACUUAUGAA | SEQ ID No 1654 | UUCAUAAGUCUACACACUGAA |
| SEQ ID No 1056 | UCAGUGUGUAGACUUAUGAAA | SEQ ID No 1655 | UUUCAUAAGUCUACACACUGA |
| SEQ ID No 1057 | GCUUAAAGCACAUAAAGACAA | SEQ ID No 1656 | UUGUCUUUAUGUGCUUUAAGC |
| SEQ ID No 1058 | CUUAAAGCACAUAAAGACAAA | SEQ ID No 1657 | UUUGUCUUUAUGUGCUUUAAG |
| SEQ ID No 1059 | CAGCUCACUCUUGUAAUGUAA | SEQ ID No 1658 | UUACAUUACAAGAGUGAGCUG |
| SEQ ID No 1060 | AACAUCACAUAAAUUAGUCUU | SEQ ID No 1659 | AAGACUAAUUUAUGUGAUGUU |
| SEQ ID No 1061 | AAAUUAGUCUUGUCUGUUAAU | SEQ ID No 1660 | AUUAACAGACAAGACUAAUUU |
| SEQ ID No 1062 | AAUUAGUCUUGUCUGUUAAUC | SEQ ID No 1661 | GAUUAACAGACAAGACUAAUU |
| SEQ ID No 1063 | AACUAAAAACAGUAAAGUACA | SEQ ID No 1662 | UGUACUUUACUGUUUUUAGUU |
| SEQ ID No 1064 | AAAAACAGUAAAGUACAAAUA | SEQ ID No 1663 | UAUUUGUACUUUACUGUUUUU |
| SEQ ID No 1065 | AAACAGUAAAGUACAAAUAGG | SEQ ID No 1664 | CCUAUUUGUACUUUACUGUUU |
| SEQ ID No 1066 | AACUUACAAAUUAAAUGUUGG | SEQ ID No 1665 | CCAACAUUUAAUUUGUAAGUU |
| SEQ ID No 1067 | AAUUAAAUGUUGGUGAUUAUU | SEQ ID No 1666 | AAUAAUCACCAACAUUUAAUU |
| SEQ ID No 1068 | AAGGUUGGUAUGCAAAAGUAU | SEQ ID No 1667 | AUACUUUUGCAUACCAACCUU |
| SEQ ID No 1069 | AAAUGUAGUAGAAUUAUACCU | SEQ ID No 1668 | AGGUAUAAUUCUACUACAUUU |
| SEQ ID No 1070 | AAUGUAGUAGAAUUAUACCUG | SEQ ID No 1669 | CAGGUAUAAUUCUACUACAUU |
| SEQ ID No 1071 | AAAUUCAAAGUGAAUUCAACA | SEQ ID No 1670 | UGUUGAAUUCACUUUGAAUUU |
| SEQ ID No 1072 | AAUUCAAAGUGAAUUCAACAU | SEQ ID No 1671 | AUGUUGAAUUCACUUUGAAUU |
| SEQ ID No 1073 | AAAGUGAAUUCAACAUUAGAA | SEQ ID No 1672 | UUCUAAUGUUGAAUUCACUUU |
| SEQ ID No 1074 | AAGUGAAUUCAACAUUAGAAC | SEQ ID No 1673 | GUUCUAAUGUUGAAUUCACUU |
| SEQ ID No 1075 | AAUUCAGUGUGUAGACUUAUG | SEQ ID No 1674 | CAUAAGUCUACACACUGAAUU |
| SEQ ID No 1076 | AAGCUUAAAGCACAUAAAGAC | SEQ ID No 1675 | GUCUUUAUGUGCUUUAAGCUU |
| SEQ ID No 1077 | AAGCACAUAAAGACAAAUCAG | SEQ ID No 1676 | CUGAUUUGUCUUUAUGUGCUU |
| SEQ ID No 1078 | AAUUAACAGGCCACAAAUAGG | SEQ ID No 1677 | CCUAUUUGUGGCCUGUUAAUU |
| SEQ ID No 1079 | AAAUAGGCGUGGUAAGAGAAU | SEQ ID No 1678 | AUUCUCUUACCACGCCUAUUU |
| SEQ ID No 1080 | AAUAGGCGUGGUAAGAGAAUU | SEQ ID No 1679 | AAUUCUCUUACCACGCCUAUU |
| SEQ ID No 1081 | AAUGCUGUAGCCUCAAAGAUU | SEQ ID No 1680 | AAUCUUUGAGGCUACAGCAUU |
| SEQ ID No 1082 | AACUCAAACUGUUGAUUCAUC | SEQ ID No 1681 | GAUGAAUCAACAGUUUGAGUU |
| SEQ ID No 1083 | AAUAUGACUAUGUCAUAUUCA | SEQ ID No 1682 | UGAAUAUGACAUAGUCAUAUU |
| SEQ ID No 1084 | AAUGUGACUAUGUCAUAUUCA | SEQ ID No 1683 | UUACACUGAUACAGUAUAAGU |
| SEQ ID No 1085 | AAACAGCUCACUCUUGUAAUG | SEQ ID No 1684 | CAUUACAAGAGUGAGCUGUUU |
| SEQ ID No 1086 | AACAGCUCACUCUUGUAAUGU | SEQ ID No 1685 | ACAUUACAAGAGUGAGCUGUU |
| SEQ ID No 1087 | UAAAUUAGUCUUGUCUGUUAA | SEQ ID No 1686 | UUAACAGACAAGACUAAUUUA |
| SEQ ID No 1088 | CCAUUGUGUGCUAAUGGACAA | SEQ ID No 1687 | UUGUCCAUUAGCACACAAUGG |
| SEQ ID No 1089 | UACAUGUGUUGGUAGCGAUAA | SEQ ID No 1688 | UUAUCGCUACCAACACAUGUA |
| SEQ ID No 1090 | UUAGCUAACACCUGUACUGAA | SEQ ID No 1689 | UUCAGUACAGGUGUUAGCUAA |
| SEQ ID No 1091 | UAGCUAACACCUGUACUGAAA | SEQ ID No 1690 | UUUCAGUACAGGUGUUAGCUA |
| SEQ ID No 1092 | CACCUGUACUGAAAGACUCAA | SEQ ID No 1691 | UUGAGUCUUUCAGUACAGGUG |
| SEQ ID No 1093 | UAAACCUAGACCACCACUUAA | SEQ ID No 1692 | UUAAGUGGUGGUCUAGGUUUA |
| SEQ ID No 1094 | CUAGACCACCACUUAACCGAA | SEQ ID No 1693 | UUCGGUUAAGUGGUGGUCUAG |
| SEQ ID No 1095 | GCACCACGCACAUUGCUAA | SEQ ID No 1694 | UUAGCAAUGUGCGUGGUGC |
| SEQ ID No 1096 | ACCUGCACCACGCACAUUGCUAA | SEQ ID No 1695 | UUAGCAAUGUGCGUGGUGCAGGU |
| SEQ ID No 1097 | UUACCUGCACCACGCACAUUGCUAA | SEQ ID No 1696 | UUAGCAAUGUGCGUGGUGCAGGUAA |
| SEQ ID No 1098 | UAGACCACCACUUAACCGAAA | SEQ ID No 1697 | UUUCGGUUAAGUGGUGGUCUA |
| SEQ ID No 1099 | CACAUACAGUAAUGCCAUUAA | SEQ ID No 1698 | UUAAUGGCAUUACUGUAUGUG |
| SEQ ID No 1100 | AUCAAAAGGUUGGUAUGCAAA | SEQ ID No 1699 | UUUGCAUACCAACCUUUUGAU |
| SEQ ID No 1101 | UGUUGAUGCACUAUGUGAGAA | SEQ ID No 1700 | UUCUCACAUAGUGCAUCAACA |
| SEQ ID No 1102 | CACUAUGUGAGAAGGCAUUAA | SEQ ID No 1701 | UUAAUGCCUUCUCACAUAGUG |
| SEQ ID No 1103 | ACUAUGUGAGAAGGCAUUAAA | SEQ ID No 1702 | UUUAAUGCCUUCUCACAUAGU |
| SEQ ID No 1104 | CUAUAGAUAAAUGUAGUAGAA | SEQ ID No 1703 | UUCUACUACAUUUAUCUAUAG |
| SEQ ID No 1105 | CUGCACCACGCACAUUGCUAA | SEQ ID No 1704 | UUAGCAAUGUGCGUGGUGCAG |
| SEQ ID No 1106 | ACCACGCACAUUGCUAACUAA | SEQ ID No 1705 | UUAGUUAGCAAUGUGCGUGGU |
| SEQ ID No 1107 | UAAUAAGCUUAAAGCACAUAA | SEQ ID No 1706 | UUAUGUGCUUUAAGCUUAUUA |
| SEQ ID No 1108 | AAUAAGCUUAAAGCACAUAAA | SEQ ID No 1707 | UUUAUGUGCUUUAAGCUUAUU |
| SEQ ID No 1109 | CAAAUAGGCGUGGUAAGAGAA | SEQ ID No 1708 | UUCUCUUACCACGCCUAUUUG |
| SEQ ID No 1110 | AAGAGAAUUCCUUACACGUAA | SEQ ID No 1709 | UUACGUGUAAGGAAUUCUCUU |
| SEQ ID No 1111 | UUCACCUUAUAAUUCACAGAA | SEQ ID No 1710 | UUCUGUGAAUUAUAAGGUGAA |
| SEQ ID No 1112 | GACUAUGUCAUAUUCACUCAA | SEQ ID No 1711 | UUGAGUGAAUAUGACAUAGUC |
| SEQ ID No 1113 | ACUAUGUCAUAUUCACUCAAA | SEQ ID No 1712 | UUUGAGUGAAUAUGACAUAGU |
| SEQ ID No 1114 | UGAAACAGCUCACUCUUGUAA | SEQ ID No 1713 | UUACAAGAGUGAGCUGUUUCA |
| SEQ ID No 1115 | GUUGCUAUUACCAGAGCAAAA | SEQ ID No 1714 | UUUUGCUCUGGUAAUAGCAAC |
| SEQ ID No 1116 | AAUGCUCCAGGUUGUGAUGUC | SEQ ID No 1715 | GACAUCACAACCUGGAGCAUU |
| SEQ ID No 1117 | AAUGCAAUUGCAACAUGUGAC | SEQ ID No 1716 | GUCACAUGUUGCAAUUGCAUU |
| SEQ ID No 1118 | AACAUGUGACUGGACAAAUGC | SEQ ID No 1717 | GCAUUUGUCCAGUCACAUGUU |
| SEQ ID No 1119 | AACACCUGUACUGAAAGACUC | SEQ ID No 1718 | GAGUCUUUCAGUACAGGUGUU |
| SEQ ID No 1120 | AACUGUCUUAUGGUAUUGCUA | SEQ ID No 1719 | UAGCAAUACCAUAAGACAGUU |
| SEQ ID No 1121 | AAGUGCUGUCUGACAGAGAAU | SEQ ID No 1720 | AUUCUCUGUCAGACAGCACUU |
| SEQ ID No 1122 | AAACCUAGACCACCACUUAAC | SEQ ID No 1721 | GUUAAGUGGUGGUCUAGGUUU |
| SEQ ID No 1123 | AACCUAGACCACCACUUAACC | SEQ ID No 1722 | GGUUAAGUGGUGGUCUAGGUU |
| SEQ ID No 1124 | AACAGUAAAGUACAAAUAGGA | SEQ ID No 1723 | UCCUAUUUGUACUUUACUGUU |
| SEQ ID No 1125 | AAAAAGGUGACUAUGGUGAUG | SEQ ID No 1724 | CAUCACCAUAGUCACCUUUUU |
| SEQ ID No 1126 | AAAUUAAAUGUUGGUGAUUAU | SEQ ID No 1725 | AUAAUCACCAACAUUUAAUUU |
| SEQ ID No 1127 | AAUUAUCAAAAGGUUGGUAUG | SEQ ID No 1726 | CAUACCAACCUUUUGAUAAUU |
| SEQ ID No 1128 | AAAAGGUUGGUAUGCAAAAGU | SEQ ID No 1727 | ACUUUUGCAUACCAACCUUUU |
| SEQ ID No 1129 | AAAGGUUGGUAUGCAAAAGUA | SEQ ID No 1728 | UACUUUUGCAUACCAACCUUU |
| SEQ ID No 1130 | AAUUCAACAUUAGAACAGUAU | SEQ ID No 1729 | AUACUGUUCUAAUGUUGAAUU |
| SEQ ID No 1131 | AACAUUAGAACAGUAUGUCUU | SEQ ID No 1730 | AAGACAUACUGUUCUAAUGUU |
| SEQ ID No 1132 | AAGCACUAUGUGUACAUUGGC | SEQ ID No 1731 | GCCAAUGUACACAUAGUGCUU |
| SEQ ID No 1133 | AACUAUAGGUCCAGACAUGUU | SEQ ID No 1732 | AACAUGUCUGGACCUAUAGUU |
| SEQ ID No 1134 | AAUAAGCUUAAAGCACAUAAA | SEQ ID No 1733 | UUUAUGUGCUUUAAGCUUAUU |
| SEQ ID No 1135 | AAAGCACAUAAAGACAAAUCA | SEQ ID No 1734 | UGAUUUGUCUUUAUGUGCUUU |
| SEQ ID No 1136 | AAGAGAAUUCCUUACACGUAA | SEQ ID No 1735 | UUACGUGUAAGGAAUUCUCUU |
| SEQ ID No 1137 | GAUGUCACAGAUGUGACUCAA | SEQ ID No 1736 | UUGAGUCACAUCUGUGACAUC |
| SEQ ID No 1138 | UUGCAACAUGUGACUGGACAA | SEQ ID No 1737 | UUGUCCAGUCACAUGUUGCAA |
| SEQ ID No 1139 | UGCAACAUGUGACUGGACAAA | SEQ ID No 1738 | UUUGUCCAGUCACAUGUUGCA |
| SEQ ID No 1140 | GGUAUUGCUACUGUACGUGAA | SEQ ID No 1739 | UUCACGUACAGUAGCAAUACC |
| SEQ ID No 1141 | GAAGUGCUGUCUGACAGAGAA | SEQ ID No 1740 | UUCUCUGUCAGACAGCACUUC |
| SEQ ID No 1142 | UAUCAAAAGGUUGGUAUGCAA | SEQ ID No 1741 | UUGCAUACCAACCUUUUGAUA |
| SEQ ID No 1143 | CAAUGCCAGAUUACGUGCUAA | SEQ ID No 1742 | UUAGCACGUAAUCUGGCAUUG |
| SEQ ID No 1144 | GUCGGCGUUGUCCUGCUGAAA | SEQ ID No 1743 | UUUCAGCAGGACAACGCCGAC |
| SEQ ID No 1145 | GUCGCCGUUGUCCUGCUGAAA | SEQ ID No 1744 | UUUCAGCAGGACAACGGCGAC |
| SEQ ID No 1146 | CAUAAAGACAAAUCAGCUCAA | SEQ ID No 1745 | UUGAGCUGAUUUGUCUUUAUG |
| SEQ ID No 1147 | CACAGAAUGCUGUAGCCUCAA | SEQ ID No 1746 | UUGAGGCUACAGCAUUCUGUG |
| SEQ ID No 1148 | ACAGAAUGCUGUAGCCUCAAA | SEQ ID No 1747 | UUUGAGGCUACAGCAUUCUGU |
| SEQ ID No 1149 | AUAUUCACUCAAACCACUGAA | SEQ ID No 1748 | UUCAGUGGUUUGAGUGAAUAU |
| SEQ ID No 1150 | UAUUCACUCAAACCACUGAAA | SEQ ID No 1749 | UUUCAGUGGUUUGAGUGAAUA |
| SEQ ID No 1151 | UGUUGCUAUUACCAGAGCAAA | SEQ ID No 1750 | UUUGCUCUGGUAAUAGCAACA |
| SEQ ID No 1152 | AAAUGCUGUUACGACCAUGUC | SEQ ID No 1751 | GACAUGGUCGUAACAGCAUUU |
| SEQ ID No 1153 | AAUGCUGUUACGACCAUGUCA | SEQ ID No 1752 | UGACAUGGUCGUAACAGCAUU |
| SEQ ID No 1154 | AAAAAUACAUGUGUUGGUAGC | SEQ ID No 1753 | GCUACCAACACAUGUAUUUUU |
| SEQ ID No 1155 | AAAAUACAUGUGUUGGUAGCG | SEQ ID No 1754 | CGCUACCAACACAUGUAUUUU |
| SEQ ID No 1156 | AAUUGCAACAUGUGACUGGAC | SEQ ID No 1755 | GUCCAGUCACAUGUUGCAAUU |
| SEQ ID No 1157 | AAACGCUCAAAGCUACUGAGG | SEQ ID No 1756 | CCUCAGUAGCUUUGAGCGUUU |
| SEQ ID No 1158 | AACGCUCAAAGCUACUGAGGA | SEQ ID No 1757 | UCCUCAGUAGCUUUGAGCGUU |
| SEQ ID No 1159 | AAACUGUCUUAUGGUAUUGCU | SEQ ID No 1758 | AGCAAUACCAUAAGACAGUUU |
| SEQ ID No 1160 | AAGUUGGUAAACCUAGACCAC | SEQ ID No 1759 | GUGGUCUAGGUUUACCAACUU |
| SEQ ID No 1161 | AAAGUACAAAUAGGAGAGUAC | SEQ ID No 1760 | GUACUCUCCUAUUUGUACUUU |
| SEQ ID No 1162 | AAGUACAAAUAGGAGAGUACA | SEQ ID No 1761 | UGUACUCUCCUAUUUGUACUU |
| SEQ ID No 1163 | AAAAGGUGACUAUGGUGAUGC | SEQ ID No 1762 | GCAUCACCAUAGUCACCUUUU |
| SEQ ID No 1164 | AAGGUGACUAUGGUGAUGCUG | SEQ ID No 1763 | CAGCAUCACCAUAGUCACCUU |
| SEQ ID No 1165 | AAUGCCAUUAAGUGCACCUAC | SEQ ID No 1764 | GUAGGUGCACUUAAUGGCAUU |
| SEQ ID No 1166 | AAGUGCACCUACACUAGUGCC | SEQ ID No 1765 | GGCACUAGUGUAGGUGCACUU |
| SEQ ID No 1167 | AACACUCAAUAUCUCAGAUGA | SEQ ID No 1766 | UCAUCUGAGAUAUUGAGUGUU |
| SEQ ID No 1168 | AAAUUAUCAAAAGGUUGGUAU | SEQ ID No 1767 | AUACCAACCUUUUGAUAAUUU |
| SEQ ID No 1169 | AAAUGCAUUGCCUGAGACGAC | SEQ ID No 1768 | GUCGUCUCAGGCAAUGCAUUU |
| SEQ ID No 1170 | AAUGCCAGAUUACGUGCUAAG | SEQ ID No 1769 | CUUAGCACGUAAUCUGGCAUU |
| SEQ ID No 1171 | AAAACUAUAGGUCCAGACAUG | SEQ ID No 1770 | CAUGUCUGGACCUAUAGUUUU |
| SEQ ID No 1172 | AAACUAUAGGUCCAGACAUGU | SEQ ID No 1771 | ACAUGUCUGGACCUAUAGUUU |
| SEQ ID No 1173 | AACUUGUCGGCGUUGUCCUGC | SEQ ID No 1772 | GCAGGACAACGCCGACAAGUU |
| SEQ ID No 1174 | AAAUUGUUGACACUGUGAGUG | SEQ ID No 1773 | CACUCACAGUGUCAACAAUUU |
| SEQ ID No 1175 | AAUUGUUGACACUGUGAGUGC | SEQ ID No 1774 | GCACUCACAGUGUCAACAAUU |
| SEQ ID No 1176 | AAAGACAAAUCAGCUCAAUGC | SEQ ID No 1775 | GCAUUGAGCUGAUUUGUCUUU |
| SEQ ID No 1177 | AAGACAAAUCAGCUCAAUGCU | SEQ ID No 1776 | AGCAUUGAGCUGAUUUGUCUU |
| SEQ ID No 1178 | AACAGGCCACAAAUAGGCGUG | SEQ ID No 1777 | CACGCCUAUUUGUGGCCUGUU |
| SEQ ID No 1179 | AACAGACCUCAAAUAGGCGUU | SEQ ID No 1778 | AACGCCUAUUUGAGGUCUGUU |
| SEQ ID No 1180 | AAACUGUUGAUUCAUCACAGG | SEQ ID No 1779 | CCUGUGAUGAAUCAACAGUUU |
| SEQ ID No 1181 | AACCACUGAAACAGCUCACUC | SEQ ID No 1780 | GAGUGAGCUGUUUCAGUGGUU |
| SEQ ID No 1182 | GCACCUACACUAGUGCCACAA | SEQ ID No 1781 | UUGUGGCACUAGUGUAGGUGC |
| SEQ ID No 1183 | GUCCAGACAUGUUCCUCGGAA | SEQ ID No 1782 | UUCCGAGGAACAUGUCUGGAC |
| SEQ ID No 1184 | UGUCGGCGUUGUCCUGCUGAA | SEQ ID No 1783 | UUCAGCAGGACAACGCCGACA |
| SEQ ID No 1185 | UCUGCAAUUAACAGGCCACAA | SEQ ID No 1784 | UUGUGGCCUGUUAAUUGCAGA |
| SEQ ID No 1186 | CUGCAAUUAACAGGCCACAAA | SEQ ID No 1785 | UUUGUGGCCUGUUAAUUGCAG |
| SEQ ID No 1187 | GGCCACAAAUAGGCGUGGUAA | SEQ ID No 1786 | UUACCACGCCUAUUUGUGGCC |
| SEQ ID No 1188 | AUGUUGCUAUUACCAGAGCAA | SEQ ID No 1787 | UUGCUCUGGUAAUAGCAACAU |
| SEQ ID No 1189 | AAAUACAUGUGUUGGUAGCGA | SEQ ID No 1788 | UCGCUACCAACACAUGUAUUU |
| SEQ ID No 1190 | AAUACAUGUGUUGGUAGCGAU | SEQ ID No 1789 | AUCGCUACCAACACAUGUAUU |
| SEQ ID No 1191 | AAAGGUGACUAUGGUGAUGCU | SEQ ID No 1790 | AGCAUCACCAUAGUCACCUUU |
| SEQ ID No 1192 | AAAAGUAUUCUACACUCCAGG | SEQ ID No 1791 | CCUGGAGUGUAGAAUACUUUU |
| SEQ ID No 1193 | AAUUAUACCUGCACGUGCUCG | SEQ ID No 1792 | CGAGCACGUGCAGGUAUAAUU |
| SEQ ID No 1194 | AAUGCAUUGCCUGAGACGACA | SEQ ID No 1793 | UGUCGUCUCAGGCAAUGCAUU |
| SEQ ID No 1195 | AAUUACCUGCACCACGCACAU | SEQ ID No 1794 | AUGUGCGUGGUGCAGGUAAUU |
| SEQ ID No 1196 | AAUUCACAGAAUGCUGUAGCC | SEQ ID No 1795 | GGCUACAGCAUUCUGUGAAUU |
| SEQ ID No 1197 | AAACCACUGAAACAGCUCACU | SEQ ID No 1796 | AGUGAGCUGUUUCAGUGGUUU |
| SEQ ID No 1198 | AAUGUUGCUAUUACCAGAGCA | SEQ ID No 1797 | UGCUCUGGUAAUAGCAACAUU |

The inventors have surprisingly found that siRNAs targeted to certain target sequences of the SARS-CoV-2 non-structural proteins (NSPs) gene are particularly effective at inhibiting non-structural proteins (NSPs) mRNA expression, inhibiting non-structural proteins (NSPs) expression for virus-cell interactions during viral life cycle in a cell, SARS-CoV-2 viral life cycle, and increase the survival of SARS-CoV-2 infected mice treated by intranasal administration of siRNAs targeting certain sequences of the SARS-CoV-2 non-structural proteins (NSPs) gene.

In a specific embodiment of the present disclosure, the sense strand of the SARS-CoV-2 non-structural proteins (NSPs) siRNA used in the present disclosure comprises or consists of a sequence selected from the group comprising SEQ ID No 657, SEQ ID No 658, SEQ ID No 659, SEQ ID No 660, SEQ ID No 661, SEQ ID No 685, SEQ ID No 751, SEQ ID No 752, SEQ ID No 809, SEQ ID No 849, SEQ ID No 862, SEQ ID No 913, SEQ ID No 923, SEQ ID No 924, SEQ ID No 937, SEQ ID No 938, SEQ ID No 944, SEQ ID No 945, SEQ ID No 946, SEQ ID No 951, SEQ ID No 952, SEQ ID No 953, SEQ ID No 966, SEQ ID No 967, SEQ ID No 972, SEQ ID No 974, SEQ ID No 975, SEQ ID No 976, SEQ ID No 1083, SEQ ID No 1084, SEQ ID No 1094, SEQ ID No 1095, SEQ ID No 1096, SEQ ID No 1097, SEQ ID No 1105, SEQ ID No 1116, SEQ ID No 1123, SEQ ID No 1141, SEQ ID No 1144, SEQ ID No 1145, SEQ ID No 1147, SEQ ID No 1157, SEQ ID No 1158, SEQ ID No 1164, SEQ ID No 1166, SEQ ID No 1169, SEQ ID No 1173, SEQ ID No 1178, SEQ ID No 1179 and SEQ ID No 1181. The siRNA also comprises a corresponding antisense strand comprising SEQ ID No 1256, SEQ ID No 1257, SEQ ID No 1258, SEQ ID No 1259, SEQ ID No 1260, SEQ ID No 1284, SEQ ID No 1350, SEQ ID No 1351, SEQ ID No 1408, SEQ ID No 1448, SEQ ID No 1461, SEQ ID No 1512, SEQ ID No 1522, SEQ ID No 1523, SEQ ID No 1536, SEQ ID No 1537, SEQ ID No 1543, SEQ ID No 1544, SEQ ID No 1545, SEQ ID No 1550, SEQ ID No 1551, SEQ ID No 1552, SEQ ID No 1565, SEQ ID No 1566, SEQ ID No 1571, SEQ ID No 1572, SEQ ID No 1573, SEQ ID No 1574, SEQ ID No 1575, SEQ ID No 1582, SEQ ID No 1682, SEQ ID No 1683, SEQ ID No 1693, SEQ ID No 1694, SEQ ID No 1695, SEQ ID No 1696, SEQ ID No 1704, SEQ ID No 1715, SEQ ID No 1722, SEQ ID No 1740, SEQ ID No 1743, SEQ ID No 1744, SEQ ID No 1746, SEQ ID No 1756, SEQ ID No 1757, SEQ ID No 1763, SEQ ID No 1765, SEQ ID No 1768, SEQ ID No 1772, SEQ ID No 1777, SEQ ID No 1778, and SEQ ID No 1780. The use of such an siRNA has been found to be particularly effective in inhibiting non-structural proteins (NSPs) mRNA expression, inhibiting non-structural proteins (NSPs) expression for virus-cell interactions during viral life cycle in a cell, SARS-CoV-2 viral replication in a cell, and increase the survival of SARS-CoV-2 infected mice treated by intranasal administration of siRNAs targeting certain sequences of the SARS-CoV-2 non-structural proteins (NSPs) gene.

According to a another aspect of the present disclosure there is provided a siRNA comprising a sense SARS-CoV-2 non-structural proteins (NSPs) nucleic acid and an anti-sense SARS-CoV-2 non-structural proteins (NSPs) nucleic acid, and the sense SARS-CoV-2 non-structural proteins (NSPs) nucleic acid is substantially identical to a target sequence contained within SARS-CoV-2 non-structural proteins (NSPs) mRNA and the anti-sense SARS-CoV-2 non-structural proteins (NSPs) nucleic acid is complementary to the sense SARS-CoV-2 non-structural proteins (NSPs) nucleic acid. The sense and antisense nucleic acids hybridize to each other to form a double-stranded molecule.

The siRNA molecules of the present disclosure have the property to inhibit expression of the SARS-CoV-2 non-structural proteins (NSPs) gene when introduced into a cell expressing said gene.

The siRNA molecules of the present disclosure have the property to inhibit SARS-CoV-2 viral life cycle in a cell when introduced into a cell expressing SARS-CoV-2 non-structural proteins (NSPs) gene.

The siRNA molecules of the present disclosure have the property to increase the survival of SARS-CoV-2 infected mice treated by intranasal administration of siRNAs targeting certain sequences of the SARS-CoV-2 non-structural proteins (NSPs) gene.

Another aspect of the disclosure relates to nucleic acid sequences and vectors encoding the siRNA according to the fourth aspect of the present disclosure, as well as to compositions comprising them, useful, for example, in the methods of the present disclosure. Compositions of the present disclosure may additionally comprise transfection enhancing agents. The nucleic acid sequence may be operably linked to an inducible or regulatable promoter. Suitable vectors are discussed above. Preferably the vector is an adeno-associated viral vector.

The composition of the present disclosure may additionally comprise a pharmaceutical agent for preventing and treating infections by the coronavirus SARS-CoV-2, wherein the agent is different from the siRNA. Preferably the pharmaceutical agent is selected from the group consisting of a nucleoside analogue antiviral agent and most preferably favipiravir, ribavirin, remdesivir and galidesivir.

Non-viral delivery siRNA systems involve the creation of nucleic acid transfection reagents. Nucleic acid transfection reagents have two basic properties. First, they must interact in some manner with the nucleic acid cargo. Most often this involves electrostatic forces, which allow the formation of nucleic acid complexes. Formation of a complex ensures that the nucleic acid and transfection reagents are presented simultaneously to the cell membrane. Complexes can be divided into three classes, based on the nature of the delivery reagent: lipoplexes; polyplexes; and lipopolyplexes. Lipoplexes are formed by the interaction of anionic nucleic acids with cationic lipids, polyplexes by interaction with cationic polymers. Lipopolyplex reagents can combine the action of cationic lipids and polymers to deliver nucleic acids. Addition of histone, poly-L-lysine and protamine to some formulations of cationic lipids results in levels of delivery that are higher than either lipid or polymer alone. The combined formulations might also be less toxic. The biocompatible systems most relevant to this purpose are non-viral biodegradable nanocapsules designed especially according to the physical chemistry of nucleic acids. They have an aqueous core surrounded by a biodegradable polymeric envelope, which provides protection and transport of the siRNA into the cytosol and allow the siRNA to function efficiently in vivo.

The present disclosure also provides a cell containing the siRNA according to the fourth aspect of the present disclosure or the vector of the present disclosure. Preferably the cell is a mammalian cell, more preferably a human cell. It is further preferred that the cell is an isolated cell.

While the foregoing disclosure provides a general description of the subject matter encompassed within the scope of the present disclosure, including methods, as well as the best mode thereof, of making and using this disclosure, the following examples are provided to further enable those skilled in the art to practice this disclosure and to provide a complete written description thereof. However, those skilled in the art will appreciate that the specifics of these examples should not be read as limiting on the disclosure, the scope of which should be apprehended from the claims and equivalents thereof appended to this disclosure. Various further aspects and embodiments of the present disclosure will be apparent to those skilled in the art in view of the present disclosure.

All documents mentioned in this specification, including reference to sequence database identifiers, are incorporated herein by reference in their entirety. Unless otherwise specified, when reference to sequence database identifiers is made, the version number is 1.

Unless context dictates otherwise, the descriptions and definitions of the features set out above are not limited to any particular aspect or embodiment of the disclosure and apply equally to all aspects and embodiments which are described. The disclosure is further described in the following non-limiting examples.

The following examples further illustrate the present disclosure in detail but are not to be construed to limit the scope thereof.

### DESCRIPTION OF THE DRAWINGS

The following figures provide preferred embodiments for illustrating the disclosure and should not be seen as limiting the scope of invention.

FIG. 1. Integrity of a natural (siNACoV-1) or chemically modified (siNACoV-F1) 21 nucleotide siRNA anti-SARS-CoV-2 non-structural proteins (NSPs) when exposed for 30 min in cell culture medium in the absence (0%) and the presence of increasing amounts of serum (10% fetal bovine serum).

FIG. 2. Integrity of natural (siNACoV-1 and siNACoV-2) or chemically modified (siNACoV-F1 and siNACoV-F2) 21 nucleotide siRNA anti-SARS-CoV-2 non-structural proteins (NSPs) when exposed for 30 in cell culture medium in the absence and the presence of RNase I (0.50 Units).

FIG. 3. Relative abundance of SARS-CoV-non-structural proteins (NSPs) mRNA in 293T cells expressing SARS-CoV-2 non-structural proteins (NSPs) by RT-qPCR (relative to GADPH) after exposure (6 h) to transfection agent (0.25% iMax) and 10 nM of a natural (siNACoV-1 and siNACoV-2) or chemically modified (siNACoV-F1 and siNACoV-F2) 21 nucleotide siRNA anti-SARS-CoV-2 non-structural proteins (NSPs) at 24 h after treatment. Significantly different from corresponding control values (****P<0.001).

siNA molecules described in the present disclosure are tested in one or more of these examples and show to have activity and stability.

### EXAMPLE 1

Cell culture: 293T (aka HEK-283T) cell lines expressing SARS-CoV-2 non-structural proteins (NSPs) and wild-type cells were maintained in a humidified atmosphere of 5% CO₂ at 37° C. Cells were grown in RPMI-1640 (Sigma, St. Louis, Mo.) supplemented with 10% fetal bovine serum (FBS) (Gibco, UK), 100 U/mL penicillin G, 0.25 μg/mL amphotericin B, 100 μg/mL streptomycin (Gibco, UK), 25 mM sodium bicarbonate (Merck, Germany) and 25 mM N-2-hydroxyethylpiperazine-N′-2-ethanosulfonic acid (HEPES) (Sigma, St. Louis, Mo.). For all cell lines the medium was changed every 2 days, and cells reached confluence 3-4 days after initial seeding. For subculturing, cells were dissociated with 0.25% trypsin-ethylenediaminetetraacetic acid (EDTA) (Sigma, St. Louis, Mo.), split 1:15 or 1:20 and subcultured in a 21-cm² growth area (Sarstedt, Germany).

### EXAMPLE 2

SARS-CoV-2 non-structural proteins (NSPs) gene silencing: Total RNA was isolated and purified using the SV Total RNA Isolation System (Promega, USA) according to manufacturer's instructions. RNA quality and concentration were verified in the NanoDrop ND1000 Spectrophotometer (Thermo Scientific, USA), and RNA integrity and genomic DNA contamination were evaluated by agarose gel electrophoresis. Total RNA (1 μg) was converted into cDNA using the Maxima Scientific First Strand cDNA Synthesis Kit for RT-qPCR (Thermo Scientific, USA), according to instructions. The following protocol was used: 1^{st} step, 10 min at 25° C.; 2^{nd} step, 15 min at 50° C.; 3^{rd} step, 5 min at 85° C. cDNA was used for qPCR analysis using Maxima SYBR Green qPCR Master Mix (Thermo Scientific, USA) in the StepOnePlus instrument (Applied Biosystems, USA). Primer Assay for SARS-CoV-2 and for the endogenous control gene GAPDH (Quiagen, Germany) were used. The qPCR reaction was performed in 96-well PCR plates (Sarstedt, Germany) as follows: one cycle of 10 min at 95° C., followed by 40 PCR cycles at 95° C. 15 s and 60° C. 60 s. A melting curve was made immediately after the qPCR, to demonstrate the specificity of the amplification. No template controls were always evaluated for each target gene. Quantification cycle (Cq) values were generated automatically by the StepOnePlus 2.3 Software and the ratio of the target gene was expressed in comparison to the endogenous control gene GAPDH. Real-time PCR efficiencies were found to be between 90% and 110%.

### EXAMPLE 3

SARS-CoV-2 non-structural proteins (NSPs) expression: Cells were rinsed twice with cold phosphate-buffered saline (PBS) and incubated with 100 μL RIPA lysis buffer (154 mM NaCl, 65.2 mM TRIZMA base, 1 mM EDTA, 1% NP-40 (IGEPAL), 6 mM sodium deoxycholate) containing protease inhibitors: 1 mM PMSF, 1 μg/mL leupeptine and 1 μg/mL aprotinin; and phosphatase inhibitors: 1 mM Na₃VO₄ and 1 mM NaF. Cells were scraped and briefly sonicated. Equal amounts of total protein (30 μg) were separated on a 10% SDS-polyacrylamide gel and electrotransfered to a nitrocellulose membrane in Tris-Glycine transfer buffer containing 20% methanol. The transblot sheets were blocked in 5% non-fat dry milk in Tris-buffered saline (TBS) for 60 min and then incubated overnight, at 4° C., with the antibodies against SARS-CoV-2 and GAPDH, diluted in 2.5% non-fat dry milk in TBS-Tween 20 (0.1% vol/vol). The immunoblots were subsequently washed and incubated with fluorescently-labelled secondary antibodies (1:20,000; AlexaFluor 680, Molecular Probes) for 60 min at room temperature (RT) and protected from light. Membranes were washed and imaged by scanning at both 700 nm and 800 nm with an Odyssey Infrared Imaging System (LI-COR Biosciences).

### EXAMPLE 4

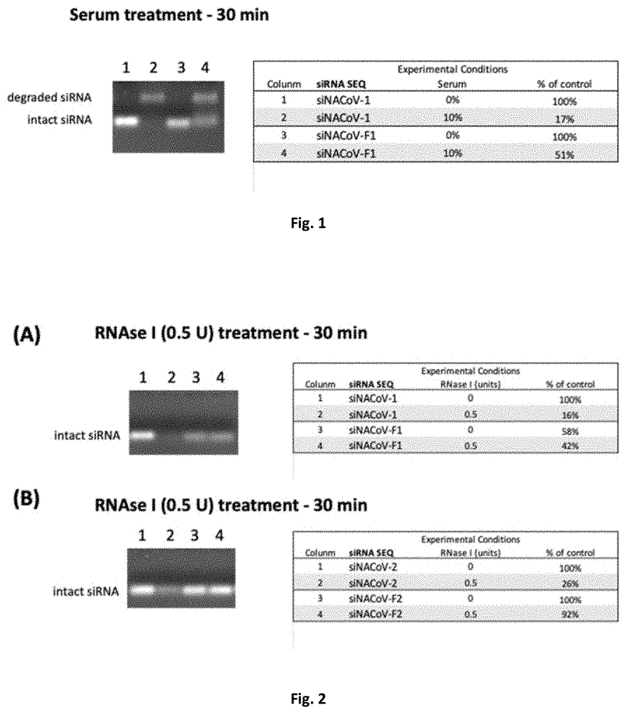

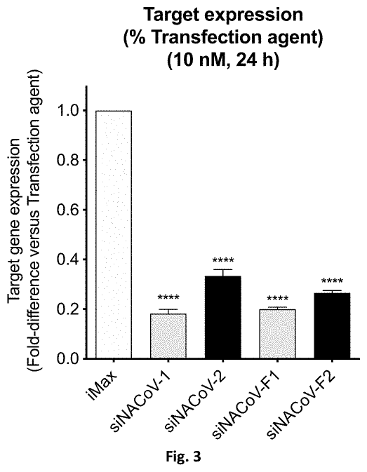

Stability of chemically modified siRNAs against SARS-CoV-2 non-structural proteins (NSPs): siRNA sequences to be used in the study were thaw and incubated at 37° C. during up to 120 min with cell serum-free culture medium added with RNase I (0.25 or 0.50 Units) or with culture medium containing 5% or 10% fetal bovine serum. In contrast to non-modified (natural) siRNAs, chemically modified siRNAs against SARS-CoV-2 non-structural proteins (NSPs) show a significant resistance to degradation in culture medium containing 10% fetal bovine serum (FIG. 1) or RNAse I (0.50 Units) for up to 30 min (FIG. 2). These chemically modified siRNAs against SARS-CoV-2 non-structural proteins (NSPs) retain their capacity in RISC engagement and downregulation of SARS-CoV-2 non-structural proteins (NSPs) mRNA expression (FIG. 3).

### EXAMPLE 5

Mouse infection studies: Pregnant Balb/c mice (18 days) were separated into four groups after delivery of their offspring. Twelve new-born mice were chosen for each group. Mice in the prevention and treatment groups were intranasally administered peptide (5 mg/kg in 2 μl of PBS) 30 min before or after intranasal challenge with a viral dose of 10² TCID₅₀ (in 2 μl DMEM). Mice in the viral control group and the normal control group were intranasally administered with 2 μl of PBS 30 min before viral challenge or without viral challenge. Mouse survival rate and body weight variations were recorded up to 2 weeks after infection. On day 5 after infection, five mice in each group were randomly selected for euthanasia to collect and assess the viral titter in mouse tissues.

The treatment with siRNA-non-structural proteins (NSPs) from SARS-CoV-2 leads to a decrease non-structural proteins (NSPs) expression for virus-cell interactions during viral life cycle in a cell and SARS-CoV-2 viral replication in a cell, and increase the survival of SARS-CoV-2 infected mice treated by intranasal administration of siRNAs targeting certain sequences of the SARS-CoV-2 non-structural proteins (NSPs) gene. This decrease in non-structural proteins (NSPs) expression by the siRNA-non-structural proteins (NSPs) from SARS-CoV-2 is accompanied by increase the survival of SARS-CoV-2 infected mice treated by intranasal administration of siRNAs targeting certain sequences of the SARS-CoV-2 non-structural proteins (NSPs) gene.

Additional aspects of the invention will be apparent to those skilled in the art, or may be learned from the practice of the invention. The objects and advantages of the invention may be realized and attained by means of the instrumentalities and combinations particularly pointed out in the appended claims.

### REFERENCES

1. Davis M E (2009). The first targeted delivery of siRNA in humans via a self-assembling, cyclodextrin polymer-based nanoparticle: from concept to clinic. Mol Pharm 6: 659-668.
2. Elbashir S M, Harborth J, Lendeckel W, Yalcin A, Weber K, & Tuschl T (2001). Duplexes of 21-nucleotide RNAs mediate RNA interference in cultured mammalian cells. Nature 411: 494-498.
3. Fire A, Xu S, Montgomery M K, Kostas S A, Driver S E, & Mello C C (1998). Potent and specific genetic interference by double-stranded RNA in Caenorhabditis elegans. Nature 391: 806-811.
4. Fehr, A. R. and S. Perlman (2015). Coronaviruses: an overview of their replication and pathogenesis. Methods Mol Biol 1282: 1-23.
5. Guan W J, Ni Z Y, Hu Y, Liang W H, Ou C Q, He J X, et al. (2020). Clinical Characteristics of Coronavirus Disease 2019 in China. N Engl J Med.
6. Hannon G J (2002). RNA interference. Nature 418: 244-251.
7. Harborth J, Elbashir S M, Bechert K, Tuschl T, & Weber K (2001). Identification of essential genes in cultured mammalian cells using small interfering RNAs. J Cell Sci 114: 4557-4565.
8. Hoffmann M, Kleine-Weber H, Schroeder S, Kruger N, Herrler T, Erichsen S, et al. (2020). SARS-CoV-2 Cell Entry Depends on ACE2 and TMPRSS2 and Is Blocked by a Clinically Proven Protease Inhibitor. Cell.
9. Jacque J-M, Triques K, & Stevenson M (2002). Modulation of HIV-1 replication by RNA interference. Nature 418: 435-438.
10. Lee N S, Dohjima T, Bauer G, Li H, Li M-J, Ehsani A, et al. (2001). Expression of small interfering RNAs targeted against HIV-1 rev transcripts in human cells. Nature Biotechnology 19: 500-505.
11. Lee N S, Dohjima T, Bauer G, Li H, Li M-J, Ehsani A, et al. (2002). Expression of small interfering RNAs targeted against HIV-1 rev transcripts in human cells. Nature Biotechnology 20: 500-505.
12. Li G, & De Clercq E (2020). Therapeutic options for the 2019 novel coronavirus (2019-nCoV). Nat Rev Drug Discov 19: 149-150.
13. Liu K, Fang Y Y, Deng Y, Liu W, Wang M F, Ma J P, et al. (2020). Clinical characteristics of novel coronavirus cases in tertiary hospitals in Hubei Province. Chin Med J (Engl).
14. Miyagishi M, & Taira K (2002). U6 promoter-driven siRNAs with four uridine 3′ overhangs efficiently suppress targeted gene expression in mammalian cells. Nature Biotechnology 19: 497-500.
15. Paddison P J, Caudy A A, Bernstein E, Hannon G J, & Conklin D S S (2002). hort hairpin RNAs (shRNAs) induce sequence-specific silencing in mammalian cells. Genes Dev 16: 948-958.
16. Paul C P, Good P D, Winer I, & Engelke D R (2002). Effective Expression of Small Interfering RNA in human cells. Nature Biotechnology 19: 505-508.
17. Perlman, S. and J. Netland (2009). Coronaviruses post-SARS: update on replication and pathogenesis. Nat Rev Microbiol 7: 439-450.
18. Sui G, Soohoo C, Affar E B, Gay F, Shi Y, Forrester W C, et al. (2002). A DNA vector-based RNAi technology to suppress gene expression in mammalian cells. Proc Natl Acad Sci USA 99: 5515-5520.
19. Xia H, Mao Q, Paulson H L, & Davidson B L (2002). siRNA-mediated gene silencing in vitro and in vivo. Nat Biotechnol 20: 1006-1010.
20. Yu J Y, DeRuiter S L, & Turner D L (2002). RNA interference by expression of short-interfering RNAs and hairpin RNAs in mammalian cells. Proc Natl Acad Sci USA 99: 6047-6052.
21. Zhou P, Yang X L, Wang X G, Hu B, Zhang L, Zhang W, et al. (2020). A pneumonia outbreak associated with a new coronavirus of probable bat origin. Nature 579: 270-273.
22. Zumla A, Chan J F, Azhar E I, Hui D S, & Yuen K Y (2016). Coronaviruses—drug discovery and therapeutic options. Nat Rev Drug Discov 15: 327-347.

1. An isolated or synthetic siNA (short interfering nucleic acid) molecule, wherein said molecule comprises a nucleic acid sequence selected from the group consisting of: SEQ ID No 657, SEQ ID No 658, SEQ ID No 659, SEQ ID No 660, SEQ ID No 661, SEQ ID No 685, SEQ ID No 751, SEQ ID No 752, SEQ ID No 809, SEQ ID No 849, SEQ ID No 862, SEQ ID No 913, SEQ ID No 923, SEQ ID No 924, SEQ ID No 937, SEQ ID No 938, SEQ ID No 944, SEQ ID No 945, SEQ ID No 946, SEQ ID No 951, SEQ ID No 952, SEQ ID No 953, SEQ ID No 966, SEQ ID No 967, SEQ ID No 972, SEQ ID No 974, SEQ ID No 975, SEQ ID No 976, SEQ ID No 1083, SEQ ID No 1084, SEQ ID No 1094, SEQ ID No 1095, SEQ ID No 1096, SEQ ID No 1097, SEQ ID No 1105, SEQ ID No 1116, SEQ ID No 1123, SEQ ID No 1141, SEQ ID No 1144, SEQ ID No 1145, SEQ ID No 1147, SEQ ID No 1157, SEQ ID No 1158, SEQ ID No 1164, SEQ ID No 1166, SEQ ID No 1169, SEQ ID No 1173, SEQ ID No 1178, SEQ ID No 1179, SEQ ID No 1181, and variants thereof.

2. The siNA molecule of claim 1, wherein said siNA molecule is complementary to a nucleic acid sequence selected from the group consisting of: SEQ ID No 1256, SEQ ID No 1257, SEQ ID No 1258, SEQ ID No 1259, SEQ ID No 1260, SEQ ID No 1284, SEQ ID No 1350, SEQ ID No 1351, SEQ ID No 1408, SEQ ID No 1448, SEQ ID No 1461, SEQ ID No 1512, SEQ ID No 1522, SEQ ID No 1523, SEQ ID No 1536, SEQ ID No 1537, SEQ ID No 1543, SEQ ID No 1544, SEQ ID No 1545, SEQ ID No 1550, SEQ ID No 1551, SEQ ID No 1552, SEQ ID No 1565, SEQ ID No 1566, SEQ ID No 1571, SEQ ID No 1572, SEQ ID No 1573, SEQ ID No 1574, SEQ ID No 1575, SEQ ID No 1582, SEQ ID No 1682, SEQ ID No 1683, SEQ ID No 1693, SEQ ID No 1694, SEQ ID No 1695, SEQ ID No 1696, SEQ ID No 1704, SEQ ID No 1715, SEQ ID No 1722, SEQ ID No 1740, SEQ ID No 1743, SEQ ID No 1744, SEQ ID No 1746, SEQ ID No 1756, SEQ ID No 1757, SEQ ID No 1763, SEQ ID No 1765, SEQ ID No 1768, SEQ ID No 1772, SEQ ID No 1777, SEQ ID No 1778, SEQ ID No 1780, and variants thereof.

3. The siNA molecule of claim 1, wherein said molecule is between 19 and 25 base pairs in length.

4. The siNA molecule of claim 1, wherein said molecule is between 21 and 23 base pairs in length.

5. The siNA molecule of claim 1, wherein said molecule comprises at least a sequence selected from SEQ ID No 600 to SEQ ID No 1797.

6. The siNA molecule of claim 1, wherein siNA is selected from the group consisting of dsRNA, siRNA and shRNA.

7. The siNA molecule of claim 6, wherein siNA is siRNA.

8. The siNA molecule of claim 1, wherein siNA comprises 5′ and/or 3′ overhangs.

9. The siNA molecule of claim 1, wherein siNA comprises at least one chemical modification.

10. The siNA molecule of claim 1, wherein the siNA molecule reduces the expression of the gene for non-structural proteins (NSPs) from SARS-CoV-2.

11. The siNA molecule of claim 1, for use in preventing and treating infectious diseases, preferably a virus infection.

12. The siNA molecule of claim 1, for use in preventing and treating the coronavirus SARS-CoV-2 inflicted infectious conditions.

13. The siNA molecule of claim 1, wherein the siRNA molecule comprises at least one sequence selected from the group consisting of: SEQ ID No 657, SEQ ID No 658, SEQ ID No 659, SEQ ID No 660, SEQ ID No 661, SEQ ID No 685, SEQ ID No 751, SEQ ID No 752, SEQ ID No 809, SEQ ID No 849, SEQ ID No 862, SEQ ID No 913, SEQ ID No 923, SEQ ID No 924, SEQ ID No 937, SEQ ID No 938, SEQ ID No 944, SEQ ID No 945, SEQ ID No 946, SEQ ID No 951, SEQ ID No 952, SEQ ID No 953, SEQ ID No 966, SEQ ID No 967, SEQ ID No 972, SEQ ID No 974, SEQ ID No 975, SEQ ID No 976, SEQ ID No 1083, SEQ ID No 1084, SEQ ID No 1094, SEQ ID No 1095, SEQ ID No 1096, SEQ ID No 1097, SEQ ID No 1105, SEQ ID No 1116, SEQ ID No 1123, SEQ ID No 1141, SEQ ID No 1144, SEQ ID No 1145, SEQ ID No 1147, SEQ ID No 1157, SEQ ID No 1158, SEQ ID No 1164, SEQ ID No 1166, SEQ ID No 1169, SEQ ID No 1173, SEQ ID No 1178, SEQ ID No 1179 and SEQ ID No 1181, preferably, said molecule reduces the expression of the gene for non-structural proteins (NSPs) from SARS-CoV-2.

14. The siNA molecule of claim 1, for use in preventing and treating coronavirus-inflicted infectious conditions.

15. The siNA molecule of claim 1, wherein the coronavirus-inflicted infectious conditions is selected from the following list: SARS-CoV-2, SARS-CoV and MERS-CoV, encompassing asymptomatic infection, mild upper respiratory tract illness, severe viral pneumonia and with respiratory failure.

16. A vector, liposome, microsphere, nanoparticle or capsule comprising a molecule described in claim 1.

17. A pharmaceutical composition comprising at least one siRNA molecule of claim 1 and a pharmaceutically acceptable carrier.

18. The composition of claim 17, further comprising a second active ingredient for the treatment of infections by the coronavirus SARS-CoV-2.

19. The composition of claim 17, further comprising an active ingredient wherein said further active ingredient is selected from the group consisting of: anti-HIV agent; anti-malarial agent, anti-tuberculosis agent, and mixtures thereof.

20. The composition of claim 17, wherein the route of administration is selected from the group consisting of: topical application, nasal application, inhalation administration, subcutaneous injection or deposition, subcutaneous infusion, intravenous injection, and intravenous infusion.


SEQUENCE LISTING (1797 sequences)

SEQ ID NO: 1 - DNA, 21 bp, SARS coronavirus 2
  5′ sense SARS-CoV-2 DNA target non-structural proteins (NSPs)
agctgatgttactaaaataaa

SEQ ID NO: 2 - DNA, 21 bp, SARS coronavirus 2
  5′ sense SARS-CoV-2 DNA target non-structural proteins (NSPs)
gctgatgttactaaaataaaa

SEQ ID NO: 3 - DNA, 21 bp, SARS coronavirus 2
  5′ sense SARS-CoV-2 DNA target non-structural proteins (NSPs)
atggagctgatgttactaaaa

SEQ ID NO: 4 - DNA, 21 bp, SARS coronavirus 2
  5′ sense SARS-CoV-2 DNA target non-structural proteins (NSPs)
gagctgatgttactaaaataa

SEQ ID NO: 5 - DNA, 21 bp, SARS coronavirus 2
  5′ sense SARS-CoV-2 DNA target non-structural proteins (NSPs)
gtggtcactataaacatataa

SEQ ID NO: 6 - DNA, 21 bp, SARS coronavirus 2
  5′ sense SARS-CoV-2 DNA target non-structural proteins (NSPs)
aaataaaacctcataattcac

SEQ ID NO: 7 - DNA, 21 bp, SARS coronavirus 2
  5′ sense SARS-CoV-2 DNA target non-structural proteins (NSPs)
aaacaagctacaaaatatcta

SEQ ID NO: 8 - DNA, 21 bp, SARS coronavirus 2
  5′ sense SARS-CoV-2 DNA target non-structural proteins (NSPs)
aacaagctacaaaatatctag

SEQ ID NO: 9 - DNA, 21 bp, SARS coronavirus 2
  5′ sense SARS-CoV-2 DNA target non-structural proteins (NSPs)
ggatggagctgatgttactaa

SEQ ID NO: 10 - DNA, 21 bp, SARS coronavirus 2
  5′ sense SARS-CoV-2 DNA target non-structural proteins (NSPs)
gatggagctgatgttactaaa

SEQ ID NO: 11 - DNA, 21 bp, SARS coronavirus 2
  5′ sense SARS-CoV-2 DNA target non-structural proteins (NSPs)
ataattcacatgaaggtaaaa

SEQ ID NO: 12 - DNA, 21 bp, SARS coronavirus 2
  5′ sense SARS-CoV-2 DNA target non-structural proteins (NSPs)
taggtacatgtcagcattaaa

SEQ ID NO: 13 - DNA, 21 bp, SARS coronavirus 2
  5′ sense SARS-CoV-2 DNA target non-structural proteins (NSPs)
cagcattaaatcacactaaaa

SEQ ID NO: 14 - DNA, 21 bp, SARS coronavirus 2
  5′ sense SARS-CoV-2 DNA target non-structural proteins (NSPs)
agcattaaatcacactaaaaa

SEQ ID NO: 15 - DNA, 21 bp, SARS coronavirus 2
  5′ sense SARS-CoV-2 DNA target non-structural proteins (NSPs)
atcttgccactgcattgttaa

SEQ ID NO: 16 - DNA, 21 bp, SARS coronavirus 2
  5′ sense SARS-CoV-2 DNA target non-structural proteins (NSPs)
tatcttagcctactgtaataa

SEQ ID NO: 17 - DNA, 21 bp, SARS coronavirus 2
  5′ sense SARS-CoV-2 DNA target non-structural proteins (NSPs)
agttaggtgatgttagagaaa

SEQ ID NO: 18 - DNA, 21 bp, SARS coronavirus 2
  5′ sense SARS-CoV-2 DNA target non-structural proteins (NSPs)
tcttgaacgtggtgtgtaaaa

SEQ ID NO: 19 - DNA, 21 bp, SARS coronavirus 2
  5′ sense SARS-CoV-2 DNA target non-structural proteins (NSPs)
acctgctcagtatgaacttaa

SEQ ID NO: 20 - DNA, 21 bp, SARS coronavirus 2
  5′ sense SARS-CoV-2 DNA target non-structural proteins (NSPs)
ttaccagtgtggtcactataa

SEQ ID NO: 21 - DNA, 21 bp, SARS coronavirus 2
  5′ sense SARS-CoV-2 DNA target non-structural proteins (NSPs)
taccagtgtggtcactataaa

SEQ ID NO: 22 - DNA, 21 bp, SARS coronavirus 2
  5′ sense SARS-CoV-2 DNA target non-structural proteins (NSPs)
ctataaacatataacttctaa

SEQ ID NO: 23 - DNA, 21 bp, SARS coronavirus 2
  5′ sense SARS-CoV-2 DNA target non-structural proteins (NSPs)
tataaacatataacttctaaa

SEQ ID NO: 24 - DNA, 21 bp, SARS coronavirus 2
  5′ sense SARS-CoV-2 DNA target non-structural proteins (NSPs)
aaacatataacttctaaagaa

SEQ ID NO: 25 - DNA, 21 bp, SARS coronavirus 2
  5′ sense SARS-CoV-2 DNA target non-structural proteins (NSPs)
aacatataacttctaaagaaa

SEQ ID NO: 26 - DNA, 21 bp, SARS coronavirus 2
  5′ sense SARS-CoV-2 DNA target non-structural proteins (NSPs)
tacaaagtcctcagaatacaa

SEQ ID NO: 27 - DNA, 21 bp, SARS coronavirus 2
  5′ sense SARS-CoV-2 DNA target non-structural proteins (NSPs)
acaaagtcctcagaatacaaa

SEQ ID NO: 28 - DNA, 21 bp, SARS coronavirus 2
  5′ sense SARS-CoV-2 DNA target non-structural proteins (NSPs)
aaaataaaacctcataattca

SEQ ID NO: 29 - DNA, 21 bp, SARS coronavirus 2
  5′ sense SARS-CoV-2 DNA target non-structural proteins (NSPs)
aataaaacctcataattcaca

SEQ ID NO: 30 - DNA, 21 bp, SARS coronavirus 2
  5′ sense SARS-CoV-2 DNA target non-structural proteins (NSPs)
aacactccaacaaatagagtt

SEQ ID NO: 31 - DNA, 21 bp, SARS coronavirus 2
  5′ sense SARS-CoV-2 DNA target non-structural proteins (NSPs)
aacgtggtgtgtaaaacttgt

SEQ ID NO: 32 - DNA, 21 bp, SARS coronavirus 2
  5′ sense SARS-CoV-2 DNA target non-structural proteins (NSPs)
aagctacaaaatatctagtac

SEQ ID NO: 33 - DNA, 21 bp, SARS coronavirus 2
  5′ sense SARS-CoV-2 DNA target non-structural proteins (NSPs)
aaacatataacttctaaagaa

SEQ ID NO: 34 - DNA, 21 bp, SARS coronavirus 2
  5′ sense SARS-CoV-2 DNA target non-structural proteins (NSPs)
aacatataacttctaaagaaa

SEQ ID NO: 35 - DNA, 21 bp, SARS coronavirus 2
  5′ sense SARS-CoV-2 DNA target non-structural proteins (NSPs)
aaagtcctcagaatacaaagg

SEQ ID NO: 36 - DNA, 21 bp, SARS coronavirus 2
  5′ sense SARS-CoV-2 DNA target non-structural proteins (NSPs)
tactaaaataaaacctcataa

SEQ ID NO: 37 - DNA, 21 bp, SARS coronavirus 2
  5′ sense SARS-CoV-2 DNA target non-structural proteins (NSPs)
aaacctcataattcacatgaa

SEQ ID NO: 38 - DNA, 21 bp, SARS coronavirus 2
  5′ sense SARS-CoV-2 DNA target non-structural proteins (NSPs)
tcataattcacatgaaggtaa

SEQ ID NO: 39 - DNA, 21 bp, SARS coronavirus 2
  5′ sense SARS-CoV-2 DNA target non-structural proteins (NSPs)
cataattcacatgaaggtaaa

SEQ ID NO: 40 - DNA, 21 bp, SARS coronavirus 2
  5′ sense SARS-CoV-2 DNA target non-structural proteins (NSPs)
gtaggtacatgtcagcattaa

SEQ ID NO: 41 - DNA, 21 bp, SARS coronavirus 2
  5′ sense SARS-CoV-2 DNA target non-structural proteins (NSPs)
gtcagcattaaatcacactaa

SEQ ID NO: 42 - DNA, 21 bp, SARS coronavirus 2
  5′ sense SARS-CoV-2 DNA target non-structural proteins (NSPs)
tcagcattaaatcacactaaa

SEQ ID NO: 43 - DNA, 21 bp, SARS coronavirus 2
  5′ sense SARS-CoV-2 DNA target non-structural proteins (NSPs)
aaatcacactaaaaagtggaa

SEQ ID NO: 44 - DNA, 21 bp, SARS coronavirus 2
  5′ sense SARS-CoV-2 DNA target non-structural proteins (NSPs)
aatcacactaaaaagtggaaa

SEQ ID NO: 45 - DNA, 21 bp, SARS coronavirus 2
  5′ sense SARS-CoV-2 DNA target non-structural proteins (NSPs)
cattgttaacactccaacaaa

SEQ ID NO: 46 - DNA, 21 bp, SARS coronavirus 2
  5′ sense SARS-CoV-2 DNA target non-structural proteins (NSPs)
actccaacaaatagagttgaa

SEQ ID NO: 47 - DNA, 21 bp, SARS coronavirus 2
  5′ sense SARS-CoV-2 DNA target non-structural proteins (NSPs)
acttatcttagcctactgtaa

SEQ ID NO: 48 - DNA, 21 bp, SARS coronavirus 2
  5′ sense SARS-CoV-2 DNA target non-structural proteins (NSPs)
gagttaggtgatgttagagaa

SEQ ID NO: 49 - DNA, 21 bp, SARS coronavirus 2
  5′ sense SARS-CoV-2 DNA target non-structural proteins (NSPs)
taggtgatgttagagaaacaa

SEQ ID NO: 50 - DNA, 21 bp, SARS coronavirus 2
  5′ sense SARS-CoV-2 DNA target non-structural proteins (NSPs)
ttcttgcaaaagagtcttgaa

SEQ ID NO: 51 - DNA, 21 bp, SARS coronavirus 2
  5′ sense SARS-CoV-2 DNA target non-structural proteins (NSPs)
gtcttgaacgtggtgtgtaaa

SEQ ID NO: 52 - DNA, 21 bp, SARS coronavirus 2
  5′ sense SARS-CoV-2 DNA target non-structural proteins (NSPs)
ataccttgtacgtgtggtaaa

SEQ ID NO: 53 - DNA, 21 bp, SARS coronavirus 2
  5′ sense SARS-CoV-2 DNA target non-structural proteins (NSPs)
ccttgtacgtgtggtaaacaa

SEQ ID NO: 54 - DNA, 21 bp, SARS coronavirus 2
  5′ sense SARS-CoV-2 DNA target non-structural proteins (NSPs)
cgtgtggtaaacaagctacaa

SEQ ID NO: 55 - DNA, 21 bp, SARS coronavirus 2
  5′ sense SARS-CoV-2 DNA target non-structural proteins (NSPs)
gtgtggtaaacaagctacaaa

SEQ ID NO: 56 - DNA, 21 bp, SARS coronavirus 2
  5′ sense SARS-CoV-2 DNA target non-structural proteins (NSPs)
tgtggtaaacaagctacaaaa

SEQ ID NO: 57 - DNA, 21 bp, SARS coronavirus 2
  5′ sense SARS-CoV-2 DNA target non-structural proteins (NSPs)
gctacaaaatatctagtacaa

SEQ ID NO: 58 - DNA, 21 bp, SARS coronavirus 2
  5′ sense SARS-CoV-2 DNA target non-structural proteins (NSPs)
gcaccacctgctcagtatgaa

SEQ ID NO: 59 - DNA, 21 bp, SARS coronavirus 2
  5′ sense SARS-CoV-2 DNA target non-structural proteins (NSPs)
gcaccacctgctgagtataaa

SEQ ID NO: 60 - DNA, 19 bp, SARS coronavirus 2
  5′ sense SARS-CoV-2 DNA target non-structural proteins (NSPs)
accacctgctcagtatgaa

SEQ ID NO: 61 - DNA, 23 bp, SARS coronavirus 2
  5′ sense SARS-CoV-2 DNA target non-structural proteins (NSPs)
cagcaccacctgctcagtatgaa

SEQ ID NO: 62 - DNA, 25 bp, SARS coronavirus 2
  5′ sense SARS-CoV-2 DNA target non-structural proteins (NSPs)
gtcagcaccacctgctcagtatgaa

SEQ ID NO: 63 - DNA, 21 bp, SARS coronavirus 2
  5′ sense SARS-CoV-2 DNA target non-structural proteins (NSPs)
acaaagaaaacagttacacaa

SEQ ID NO: 64 - DNA, 21 bp, SARS coronavirus 2
  5′ sense SARS-CoV-2 DNA target non-structural proteins (NSPs)
aagaaaacagttacacaacaa

SEQ ID NO: 65 - DNA, 21 bp, SARS coronavirus 2
  5′ sense SARS-CoV-2 DNA target non-structural proteins (NSPs)
aatgacatatggacaacagtt

SEQ ID NO: 66 - DNA, 21 bp, SARS coronavirus 2
  5′ sense SARS-CoV-2 DNA target non-structural proteins (NSPs)
aaaacctcataattcacatga

SEQ ID NO: 67 - DNA, 21 bp, SARS coronavirus 2
  5′ sense SARS-CoV-2 DNA target non-structural proteins (NSPs)
aaacctcataattcacatgaa

SEQ ID NO: 68 - DNA, 21 bp, SARS coronavirus 2
  5′ sense SARS-CoV-2 DNA target non-structural proteins (NSPs)
aacctcataattcacatgaag

SEQ ID NO: 69 - DNA, 21 bp, SARS coronavirus 2
  5′ sense SARS-CoV-2 DNA target non-structural proteins (NSPs)
aattcacatgaaggtaaaaca

SEQ ID NO: 70 - DNA, 21 bp, SARS coronavirus 2
  5′ sense SARS-CoV-2 DNA target non-structural proteins (NSPs)
aaatcacactaaaaagtggaa

SEQ ID NO: 71 - DNA, 21 bp, SARS coronavirus 2
  5′ sense SARS-CoV-2 DNA target non-structural proteins (NSPs)
aatcacactaaaaagtggaaa

SEQ ID NO: 72 - DNA, 21 bp, SARS coronavirus 2
  5′ sense SARS-CoV-2 DNA target non-structural proteins (NSPs)
aatccacctgctctacaagat

SEQ ID NO: 73 - DNA, 21 bp, SARS coronavirus 2
  5′ sense SARS-CoV-2 DNA target non-structural proteins (NSPs)
aaaggtgttcagataccttgt

SEQ ID NO: 74 - DNA, 21 bp, SARS coronavirus 2
  5′ sense SARS-CoV-2 DNA target non-structural proteins (NSPs)
aaggtgttcagataccttgta

SEQ ID NO: 75 - DNA, 21 bp, SARS coronavirus 2
  5′ sense SARS-CoV-2 DNA target non-structural proteins (NSPs)
aagtcctcagaatacaaaggt

SEQ ID NO: 76 - DNA, 21 bp, SARS coronavirus 2
  5′ sense SARS-CoV-2 DNA target non-structural proteins (NSPs)
aatacaaaggtcctattacgg

SEQ ID NO: 77 - DNA, 21 bp, SARS coronavirus 2
  5′ sense SARS-CoV-2 DNA target non-structural proteins (NSPs)
aaagaaaacagttacacaaca

SEQ ID NO: 78 - DNA, 21 bp, SARS coronavirus 2
  5′ sense SARS-CoV-2 DNA target non-structural proteins (NSPs)
aagaaaacagttacacaacaa

SEQ ID NO: 79 - DNA, 21 bp, SARS coronavirus 2
  5′ sense SARS-CoV-2 DNA target non-structural proteins (NSPs)
aaacagttacacaacaaccat

SEQ ID NO: 80 - DNA, 21 bp, SARS coronavirus 2
  5′ sense SARS-CoV-2 DNA target non-structural proteins (NSPs)
aacagttacacaacaaccatt

SEQ ID NO: 81 - DNA, 21 bp, SARS coronavirus 2
  5′ sense SARS-CoV-2 DNA target non-structural proteins (NSPs)
actgcattgttaacactccaa

SEQ ID NO: 82 - DNA, 21 bp, SARS coronavirus 2
  5′ sense SARS-CoV-2 DNA target non-structural proteins (NSPs)
gcattgttaacactccaacaa

SEQ ID NO: 83 - DNA, 21 bp, SARS coronavirus 2
  5′ sense SARS-CoV-2 DNA target non-structural proteins (NSPs)
aagatgcttattacagagcaa

SEQ ID NO: 84 - DNA, 21 bp, SARS coronavirus 2
  5′ sense SARS-CoV-2 DNA target non-structural proteins (NSPs)
agtcttgaacgtggtgtgtaa

SEQ ID NO: 85 - DNA, 21 bp, SARS coronavirus 2
  5′ sense SARS-CoV-2 DNA target non-structural proteins (NSPs)
gtgtgtaaaacttgtggacaa

SEQ ID NO: 86 - DNA, 21 bp, SARS coronavirus 2
  5′ sense SARS-CoV-2 DNA target non-structural proteins (NSPs)
cttgtggacaacagcagacaa

SEQ ID NO: 87 - DNA, 21 bp, SARS coronavirus 2
  5′ sense SARS-CoV-2 DNA target non-structural proteins (NSPs)
gataccttgtacgtgtggtaa

SEQ ID NO: 88 - DNA, 21 bp, SARS coronavirus 2
  5′ sense SARS-CoV-2 DNA target non-structural proteins (NSPs)
tgctagtgagtacactggtaa

SEQ ID NO: 89 - DNA, 21 bp, SARS coronavirus 2
  5′ sense SARS-CoV-2 DNA target non-structural proteins (NSPs)
ttacttacaaagtcctcagaa

SEQ ID NO: 90 - DNA, 21 bp, SARS coronavirus 2
  5′ sense SARS-CoV-2 DNA target non-structural proteins (NSPs)
aatgatgacactctacgtgtt

SEQ ID NO: 91 - DNA, 21 bp, SARS coronavirus 2
  5′ sense SARS-CoV-2 DNA target non-structural proteins (NSPs)
aagatgcttattacagagcaa

SEQ ID NO: 92 - DNA, 21 bp, SARS coronavirus 2
  5′ sense SARS-CoV-2 DNA target non-structural proteins (NSPs)
aataagacagtaggtgagtta

SEQ ID NO: 93 - DNA, 21 bp, SARS coronavirus 2
  5′ sense SARS-CoV-2 DNA target non-structural proteins (NSPs)
aagacagtaggtgagttaggt

SEQ ID NO: 94 - DNA, 21 bp, SARS coronavirus 2
  5′ sense SARS-CoV-2 DNA target non-structural proteins (NSPs)
aaaagagtcttgaacgtggtg

SEQ ID NO: 95 - DNA, 21 bp, SARS coronavirus 2
  5′ sense SARS-CoV-2 DNA target non-structural proteins (NSPs)
aaaacttgtggacaacagcag

SEQ ID NO: 96 - DNA, 21 bp, SARS coronavirus 2
  5′ sense SARS-CoV-2 DNA target non-structural proteins (NSPs)
aaacttgtggacaacagcaga

SEQ ID NO: 97 - DNA, 21 bp, SARS coronavirus 2
  5′ sense SARS-CoV-2 DNA target non-structural proteins (NSPs)
aacttgtggacaacagcagac

SEQ ID NO: 98 - DNA, 21 bp, SARS coronavirus 2
  5′ sense SARS-CoV-2 DNA target non-structural proteins (NSPs)
aagaaaggtgttcagatacct

SEQ ID NO: 99 - DNA, 21 bp, SARS coronavirus 2
  5′ sense SARS-CoV-2 DNA target non-structural proteins (NSPs)
aaaatatctagtacaacagga

SEQ ID NO: 100 - DNA, 21 bp, SARS coronavirus 2
  5′ sense SARS-CoV-2 DNA target non-structural proteins (NSPs)
aaatatctagtacaacaggag

SEQ ID NO: 101 - DNA, 21 bp, SARS coronavirus 2
  5′ sense SARS-CoV-2 DNA target non-structural proteins (NSPs)
aatatctagtacaacaggagt

SEQ ID NO: 102 - DNA, 21 bp, SARS coronavirus 2
  5′ sense SARS-CoV-2 DNA target non-structural proteins (NSPs)
aattaccagtgtggtcactat

SEQ ID NO: 103 - DNA, 21 bp, SARS coronavirus 2
  5′ sense SARS-CoV-2 DNA target non-structural proteins (NSPs)
aaaggtcctattacggatgtt

SEQ ID NO: 104 - DNA, 21 bp, SARS coronavirus 2
  5′ sense SARS-CoV-2 DNA target non-structural proteins (NSPs)
aaaacagttacacaacaacca

SEQ ID NO: 105 - DNA, 21 bp, SARS coronavirus 2
  5′ sense SARS-CoV-2 DNA target non-structural proteins (NSPs)
aacaactgttatcttgccact

SEQ ID NO: 106 - DNA, 21 bp, SARS coronavirus 2
  5′ sense SARS-CoV-2 DNA target non-structural proteins (NSPs)
aaagagtcttgaacgtggtgt

SEQ ID NO: 107 - DNA, 21 bp, SARS coronavirus 2
  5′ sense SARS-CoV-2 DNA target non-structural proteins (NSPs)
aagagtcttgaacgtggtgtg

SEQ ID NO: 108 - DNA, 21 bp, SARS coronavirus 2
  5′ sense SARS-CoV-2 DNA target non-structural proteins (NSPs)
aactgttatcttgccactgca

SEQ ID NO: 109 - DNA, 21 bp, SARS coronavirus 2
  5′ sense SARS-CoV-2 DNA target non-structural proteins (NSPs)
cattcgtaagtctaatcataa

SEQ ID NO: 110 - DNA, 21 bp, SARS coronavirus 2
  5′ sense SARS-CoV-2 DNA target non-structural proteins (NSPs)
gttgtacgctgctgttataaa

SEQ ID NO: 111 - DNA, 21 bp, SARS coronavirus 2
  5′ sense SARS-CoV-2 DNA target non-structural proteins (NSPs)
gctatgaagtacaattatgaa

SEQ ID NO: 112 - DNA, 21 bp, SARS coronavirus 2
  5′ sense SARS-CoV-2 DNA target non-structural proteins (NSPs)
ttgtggtacaactacacttaa

SEQ ID NO: 113 - DNA, 21 bp, SARS coronavirus 2
  5′ sense SARS-CoV-2 DNA target non-structural proteins (NSPs)
gtacaggctggtaatgttcaa

SEQ ID NO: 114 - DNA, 21 bp, SARS coronavirus 2
  5′ sense SARS-CoV-2 DNA target non-structural proteins (NSPs)
tattggacattctatgcaaaa

SEQ ID NO: 115 - DNA, 21 bp, SARS coronavirus 2
  5′ sense SARS-CoV-2 DNA target non-structural proteins (NSPs)
tatgcaaaattgtgtacttaa

SEQ ID NO: 116 - DNA, 21 bp, SARS coronavirus 2
  5′ sense SARS-CoV-2 DNA target non-structural proteins (NSPs)
aaattgtgtacttaagcttaa

SEQ ID NO: 117 - DNA, 21 bp, SARS coronavirus 2
  5′ sense SARS-CoV-2 DNA target non-structural proteins (NSPs)
atcctaagacacctaagataa

SEQ ID NO: 118 - DNA, 21 bp, SARS coronavirus 2
  5′ sense SARS-CoV-2 DNA target non-structural proteins (NSPs)
ttcagtgttagcttgttacaa

SEQ ID NO: 119 - DNA, 21 bp, SARS coronavirus 2
  5′ sense SARS-CoV-2 DNA target non-structural proteins (NSPs)
tgcaccatatggaattaccaa

SEQ ID NO: 120 - DNA, 21 bp, SARS coronavirus 2
  5′ sense SARS-CoV-2 DNA target non-structural proteins (NSPs)
ggacacaactattacagttaa

SEQ ID NO: 121 - DNA, 21 bp, SARS coronavirus 2
  5′ sense SARS-CoV-2 DNA target non-structural proteins (NSPs)
ggttgtacgctgctgttataa

SEQ ID NO: 122 - DNA, 21 bp, SARS coronavirus 2
  5′ sense SARS-CoV-2 DNA target non-structural proteins (NSPs)
aaaattgtgtacttaagctta

SEQ ID NO: 123 - DNA, 21 bp, SARS coronavirus 2
  5′ sense SARS-CoV-2 DNA target non-structural proteins (NSPs)
aaattgtgtacttaagcttaa

SEQ ID NO: 124 - DNA, 21 bp, SARS coronavirus 2
  5′ sense SARS-CoV-2 DNA target non-structural proteins (NSPs)
aattgtgtacttaagcttaag

SEQ ID NO: 125 - DNA, 21 bp, SARS coronavirus 2
  5′ sense SARS-CoV-2 DNA target non-structural proteins (NSPs)
aaccttgtggctatgaagtac

SEQ ID NO: 126 - DNA, 21 bp, SARS coronavirus 2
  5′ sense SARS-CoV-2 DNA target non-structural proteins (NSPs)
aagaccatgttgacatactag

SEQ ID NO: 127 - DNA, 21 bp, SARS coronavirus 2
  5′ sense SARS-CoV-2 DNA target non-structural proteins (NSPs)
gcacctctgaagacatgctaa

SEQ ID NO: 128 - DNA, 21 bp, SARS coronavirus 2
  5′ sense SARS-CoV-2 DNA target non-structural proteins (NSPs)
gttattggacattctatgcaa

SEQ ID NO: 129 - DNA, 21 bp, SARS coronavirus 2
  5′ sense SARS-CoV-2 DNA target non-structural proteins (NSPs)
ttattggacattctatgcaaa

SEQ ID NO: 130 - DNA, 21 bp, SARS coronavirus 2
  5′ sense SARS-CoV-2 DNA target non-structural proteins (NSPs)
ggttgatacagccaatcctaa

SEQ ID NO: 131 - DNA, 21 bp, SARS coronavirus 2
  5′ sense SARS-CoV-2 DNA target non-structural proteins (NSPs)
tgttacatgcaccatatggaa

SEQ ID NO: 132 - DNA, 21 bp, SARS coronavirus 2
  5′ sense SARS-CoV-2 DNA target non-structural proteins (NSPs)
catgctggcacagacttagaa

SEQ ID NO: 133 - DNA, 21 bp, SARS coronavirus 2
  5′ sense SARS-CoV-2 DNA target non-structural proteins (NSPs)
tggcacagacttagaaggtaa

SEQ ID NO: 134 - DNA, 21 bp, SARS coronavirus 2
  5′ sense SARS-CoV-2 DNA target non-structural proteins (NSPs)
ccttgtggctatgaagtacaa

SEQ ID NO: 135 - DNA, 21 bp, SARS coronavirus 2
  5′ sense SARS-CoV-2 DNA target non-structural proteins (NSPs)
agtacaattatgaacctctaa

SEQ ID NO: 136 - DNA, 21 bp, SARS coronavirus 2
  5′ sense SARS-CoV-2 DNA target non-structural proteins (NSPs)
aattatgaacctctaacacaa

SEQ ID NO: 137 - DNA, 21 bp, SARS coronavirus 2
  5′ sense SARS-CoV-2 DNA target non-structural proteins (NSPs)
aagtaacttgtggtacaacta

SEQ ID NO: 138 - DNA, 21 bp, SARS coronavirus 2
  5′ sense SARS-CoV-2 DNA target non-structural proteins (NSPs)
aacttgtggtacaactacact

SEQ ID NO: 139 - DNA, 21 bp, SARS coronavirus 2
  5′ sense SARS-CoV-2 DNA target non-structural proteins (NSPs)
aatcctaagacacctaagata

SEQ ID NO: 140 - DNA, 21 bp, SARS coronavirus 2
  5′ sense SARS-CoV-2 DNA target non-structural proteins (NSPs)
aattaccaactggagttcatg

SEQ ID NO: 141 - DNA, 21 bp, SARS coronavirus 2
  5′ sense SARS-CoV-2 DNA target non-structural proteins (NSPs)
aattatgaacctctaacacaa

SEQ ID NO: 142 - DNA, 21 bp, SARS coronavirus 2
  5′ sense SARS-CoV-2 DNA target non-structural proteins (NSPs)
aacctctaacacaagaccatg

SEQ ID NO: 143 - DNA, 21 bp, SARS coronavirus 2
  5′ sense SARS-CoV-2 DNA target non-structural proteins (NSPs)
aacacaagaccatgttgacat

SEQ ID NO: 144 - DNA, 21 bp, SARS coronavirus 2
  5′ sense SARS-CoV-2 DNA target non-structural proteins (NSPs)
tacaagtaacttgtggtacaa

SEQ ID NO: 145 - DNA, 21 bp, SARS coronavirus 2
  5′ sense SARS-CoV-2 DNA target non-structural proteins (NSPs)
catgtgatctgcacctctgaa

SEQ ID NO: 146 - DNA, 21 bp, SARS coronavirus 2
  5′ sense SARS-CoV-2 DNA target non-structural proteins (NSPs)
gcttaaggttgatacagccaa

SEQ ID NO: 147 - DNA, 21 bp, SARS coronavirus 2
  5′ sense SARS-CoV-2 DNA target non-structural proteins (NSPs)
agccaatcctaagacacctaa

SEQ ID NO: 148 - DNA, 21 bp, SARS coronavirus 2
  5′ sense SARS-CoV-2 DNA target non-structural proteins (NSPs)
gttgacaggcaaacagcacaa

SEQ ID NO: 149 - DNA, 21 bp, SARS coronavirus 2
  5′ sense SARS-CoV-2 DNA target non-structural proteins (NSPs)
aagcttaaggttgatacagcc

SEQ ID NO: 150 - DNA, 21 bp, SARS coronavirus 2
  5′ sense SARS-CoV-2 DNA target non-structural proteins (NSPs)
aatggttcaccatctggtgtt

SEQ ID NO: 151 - DNA, 21 bp, SARS coronavirus 2
  5′ sense SARS-CoV-2 DNA target non-structural proteins (NSPs)
aatggttcatgtggtagtgtt

SEQ ID NO: 152 - DNA, 21 bp, SARS coronavirus 2
  5′ sense SARS-CoV-2 DNA target non-structural proteins (NSPs)
aacagcacaagcagctggtac

SEQ ID NO: 153 - DNA, 21 bp, SARS coronavirus 2
  5′ sense SARS-CoV-2 DNA target non-structural proteins (NSPs)
aactgcacaggctgcaggtac

SEQ ID NO: 154 - DNA, 21 bp, SARS coronavirus 2
  5′ sense SARS-CoV-2 DNA target non-structural proteins (NSPs)
aagtacaattatgaacctcta

SEQ ID NO: 155 - DNA, 21 bp, SARS coronavirus 2
  5′ sense SARS-CoV-2 DNA target non-structural proteins (NSPs)
aagcagctggtacggacacaa

SEQ ID NO: 156 - DNA, 21 bp, SARS coronavirus 2
  5′ sense SARS-CoV-2 DNA target non-structural proteins (NSPs)
aagacatgtgatctgcacctc

SEQ ID NO: 157 - DNA, 21 bp, SARS coronavirus 2
  5′ sense SARS-CoV-2 DNA target non-structural proteins (NSPs)
aaggttgatacagccaatcct

SEQ ID NO: 158 - DNA, 21 bp, SARS coronavirus 2
  5′ sense SARS-CoV-2 DNA target non-structural proteins (NSPs)
aactggagttcatgctggcac

SEQ ID NO: 159 - DNA, 21 bp, SARS coronavirus 2
  5′ sense SARS-CoV-2 DNA target non-structural proteins (NSPs)
aaacagcacaagcagctggta

SEQ ID NO: 160 - DNA, 21 bp, SARS coronavirus 2
  5′ sense SARS-CoV-2 DNA target non-structural proteins (NSPs)
aagcagctggtacggacacaa

SEQ ID NO: 161 - DNA, 21 bp, SARS coronavirus 2
  5′ sense SARS-CoV-2 DNA target non-structural proteins (NSPs)
tgctaaattcctaaaaactaa

SEQ ID NO: 162 - DNA, 21 bp, SARS coronavirus 2
  5′ sense SARS-CoV-2 DNA target non-structural proteins (NSPs)
ggtaattgtgacacattaaaa

SEQ ID NO: 163 - DNA, 21 bp, SARS coronavirus 2
  5′ sense SARS-CoV-2 DNA target non-structural proteins (NSPs)
aattgtgacacattaaaagaa

SEQ ID NO: 164 - DNA, 21 bp, SARS coronavirus 2
  5′ sense SARS-CoV-2 DNA target non-structural proteins (NSPs)
attgtgacacattaaaagaaa

SEQ ID NO: 165 - DNA, 21 bp, SARS coronavirus 2
  5′ sense SARS-CoV-2 DNA target non-structural proteins (NSPs)
tggtgtactgacattagataa

SEQ ID NO: 166 - DNA, 21 bp, SARS coronavirus 2
  5′ sense SARS-CoV-2 DNA target non-structural proteins (NSPs)
cattgttaatgcctatattaa

SEQ ID NO: 167 - DNA, 21 bp, SARS coronavirus 2
  5′ sense SARS-CoV-2 DNA target non-structural proteins (NSPs)
cttaacaaagccttacattaa

SEQ ID NO: 168 - DNA, 21 bp, SARS coronavirus 2
  5′ sense SARS-CoV-2 DNA target non-structural proteins (NSPs)
aaggaagttctgttgaattaa

SEQ ID NO: 169 - DNA, 21 bp, SARS coronavirus 2
  5′ sense SARS-CoV-2 DNA target non-structural proteins (NSPs)
aggaagttctgttgaattaaa

SEQ ID NO: 170 - DNA, 21 bp, SARS coronavirus 2
  5′ sense SARS-CoV-2 DNA target non-structural proteins (NSPs)
ggaagttctgttgaattaaaa

SEQ ID NO: 171 - DNA, 21 bp, SARS coronavirus 2
  5′ sense SARS-CoV-2 DNA target non-structural proteins (NSPs)
ggttggcacaacatgttaaaa

SEQ ID NO: 172 - DNA, 21 bp, SARS coronavirus 2
  5′ sense SARS-CoV-2 DNA target non-structural proteins (NSPs)
gttggcacaacatgttaaaaa

SEQ ID NO: 173 - DNA, 21 bp, SARS coronavirus 2
  5′ sense SARS-CoV-2 DNA target non-structural proteins (NSPs)
tggcggttcactatatgttaa

SEQ ID NO: 174 - DNA, 21 bp, SARS coronavirus 2
  5′ sense SARS-CoV-2 DNA target non-structural proteins (NSPs)
ggcggttcactatatgttaaa

SEQ ID NO: 175 - DNA, 21 bp, SARS coronavirus 2
  5′ sense SARS-CoV-2 DNA target non-structural proteins (NSPs)
aattgtgacacattaaaagaa

SEQ ID NO: 176 - DNA, 21 bp, SARS coronavirus 2
  5′ sense SARS-CoV-2 DNA target non-structural proteins (NSPs)
aatacttgtcacatacaattg

SEQ ID NO: 177 - DNA, 21 bp, SARS coronavirus 2
  5′ sense SARS-CoV-2 DNA target non-structural proteins (NSPs)
aaggaagttctgttgaattaa

SEQ ID NO: 178 - DNA, 21 bp, SARS coronavirus 2
  5′ sense SARS-CoV-2 DNA target non-structural proteins (NSPs)
aagttctgttgaattaaaaca

SEQ ID NO: 179 - DNA, 21 bp, SARS coronavirus 2
  5′ sense SARS-CoV-2 DNA target non-structural proteins (NSPs)
aaaaattattgaaatcaatag

SEQ ID NO: 180 - DNA, 21 bp, SARS coronavirus 2
  5′ sense SARS-CoV-2 DNA target non-structural proteins (NSPs)
aacatacaatgctagttaaac

SEQ ID NO: 181 - DNA, 21 bp, SARS coronavirus 2
  5′ sense SARS-CoV-2 DNA target non-structural proteins (NSPs)
ctaactaccaacatgaagaaa

SEQ ID NO: 182 - DNA, 21 bp, SARS coronavirus 2
  5′ sense SARS-CoV-2 DNA target non-structural proteins (NSPs)
actaccaacatgaagaaacaa

SEQ ID NO: 183 - DNA, 21 bp, SARS coronavirus 2
  5′ sense SARS-CoV-2 DNA target non-structural proteins (NSPs)
atcacgtcaacgtcttactaa

SEQ ID NO: 184 - DNA, 21 bp, SARS coronavirus 2
  5′ sense SARS-CoV-2 DNA target non-structural proteins (NSPs)
tcacgtcaacgtcttactaaa

SEQ ID NO: 185 - DNA, 21 bp, SARS coronavirus 2
  5′ sense SARS-CoV-2 DNA target non-structural proteins (NSPs)
aacgtcttactaaatacacaa

SEQ ID NO: 186 - DNA, 21 bp, SARS coronavirus 2
  5′ sense SARS-CoV-2 DNA target non-structural proteins (NSPs)
aaggtaattgtgacacattaa

SEQ ID NO: 187 - DNA, 21 bp, SARS coronavirus 2
  5′ sense SARS-CoV-2 DNA target non-structural proteins (NSPs)
aggtaattgtgacacattaaa

SEQ ID NO: 188 - DNA, 21 bp, SARS coronavirus 2
  5′ sense SARS-CoV-2 DNA target non-structural proteins (NSPs)
gtactgacattagataatcaa

SEQ ID NO: 189 - DNA, 21 bp, SARS coronavirus 2
  5′ sense SARS-CoV-2 DNA target non-structural proteins (NSPs)
attcttattattcattgttaa

SEQ ID NO: 190 - DNA, 21 bp, SARS coronavirus 2
  5′ sense SARS-CoV-2 DNA target non-structural proteins (NSPs)
atgttgacactgacttaacaa

SEQ ID NO: 191 - DNA, 21 bp, SARS coronavirus 2
  5′ sense SARS-CoV-2 DNA target non-structural proteins (NSPs)
tgttgacactgacttaacaaa

SEQ ID NO: 192 - DNA, 21 bp, SARS coronavirus 2
  5′ sense SARS-CoV-2 DNA target non-structural proteins (NSPs)
ttcacggaagagaggttaaaa

SEQ ID NO: 193 - DNA, 21 bp, SARS coronavirus 2
  5′ sense SARS-CoV-2 DNA target non-structural proteins (NSPs)
ttggaccactagtgagaaaaa

SEQ ID NO: 194 - DNA, 21 bp, SARS coronavirus 2
  5′ sense SARS-CoV-2 DNA target non-structural proteins (NSPs)
agagctaggtgttgtacataa

SEQ ID NO: 195 - DNA, 21 bp, SARS coronavirus 2
  5′ sense SARS-CoV-2 DNA target non-structural proteins (NSPs)
tggtaatctattactagataa

SEQ ID NO: 196 - DNA, 21 bp, SARS coronavirus 2
  5′ sense SARS-CoV-2 DNA target non-structural proteins (NSPs)
ggtaatctattactagataaa

SEQ ID NO: 197 - DNA, 21 bp, SARS coronavirus 2
  5′ sense SARS-CoV-2 DNA target non-structural proteins (NSPs)
ttcagtagctgcacttactaa

SEQ ID NO: 198 - DNA, 21 bp, SARS coronavirus 2
  5′ sense SARS-CoV-2 DNA target non-structural proteins (NSPs)
agtagctgcacttactaacaa

SEQ ID NO: 199 - DNA, 21 bp, SARS coronavirus 2
  5′ sense SARS-CoV-2 DNA target non-structural proteins (NSPs)
ttatgactactatcgttataa

SEQ ID NO: 200 - DNA, 21 bp, SARS coronavirus 2
  5′ sense SARS-CoV-2 DNA target non-structural proteins (NSPs)
tgtagttgaagttgttgataa

SEQ ID NO: 201 - DNA, 21 bp, SARS coronavirus 2
  5′ sense SARS-CoV-2 DNA target non-structural proteins (NSPs)
ttacgatggtggctgtattaa

SEQ ID NO: 202 - DNA, 21 bp, SARS coronavirus 2
  5′ sense SARS-CoV-2 DNA target non-structural proteins (NSPs)
tggtggctgtattaatgctaa

SEQ ID NO: 203 - DNA, 21 bp, SARS coronavirus 2
  5′ sense SARS-CoV-2 DNA target non-structural proteins (NSPs)
tataactcaaatgaatcttaa

SEQ ID NO: 204 - DNA, 21 bp, SARS coronavirus 2
  5′ sense SARS-CoV-2 DNA target non-structural proteins (NSPs)
atcaaaaattattgaaatcaa

SEQ ID NO: 205 - DNA, 21 bp, SARS coronavirus 2
  5′ sense SARS-CoV-2 DNA target non-structural proteins (NSPs)
gagctactgtagtaattggaa

SEQ ID NO: 206 - DNA, 21 bp, SARS coronavirus 2
  5′ sense SARS-CoV-2 DNA target non-structural proteins (NSPs)
gtggttggcacaacatgttaa

SEQ ID NO: 207 - DNA, 21 bp, SARS coronavirus 2
  5′ sense SARS-CoV-2 DNA target non-structural proteins (NSPs)
tggttggcacaacatgttaaa

SEQ ID NO: 208 - DNA, 21 bp, SARS coronavirus 2
  5′ sense SARS-CoV-2 DNA target non-structural proteins (NSPs)
ttcttgctcgcaaacatacaa

SEQ ID NO: 209 - DNA, 21 bp, SARS coronavirus 2
  5′ sense SARS-CoV-2 DNA target non-structural proteins (NSPs)
tgccacaactgcttatgctaa

SEQ ID NO: 210 - DNA, 21 bp, SARS coronavirus 2
  5′ sense SARS-CoV-2 DNA target non-structural proteins (NSPs)
agctgtcacggccaatgttaa

SEQ ID NO: 211 - DNA, 21 bp, SARS coronavirus 2
  5′ sense SARS-CoV-2 DNA target non-structural proteins (NSPs)
tatctactgatggtaacaaaa

SEQ ID NO: 212 - DNA, 21 bp, SARS coronavirus 2
  5′ sense SARS-CoV-2 DNA target non-structural proteins (NSPs)
ttatgagtgtctctatagaaa

SEQ ID NO: 213 - DNA, 21 bp, SARS coronavirus 2
  5′ sense SARS-CoV-2 DNA target non-structural proteins (NSPs)
aggtctagtggctagcataaa

SEQ ID NO: 214 - DNA, 21 bp, SARS coronavirus 2
  5′ sense SARS-CoV-2 DNA target non-structural proteins (NSPs)
tctagtggctagcataaagaa

SEQ ID NO: 215 - DNA, 21 bp, SARS coronavirus 2
  5′ sense SARS-CoV-2 DNA target non-structural proteins (NSPs)
actgagactgaccttactaaa

SEQ ID NO: 216 - DNA, 21 bp, SARS coronavirus 2
  5′ sense SARS-CoV-2 DNA target non-structural proteins (NSPs)
caacatacaatgctagttaaa

SEQ ID NO: 217 - DNA, 21 bp, SARS coronavirus 2
  5′ sense SARS-CoV-2 DNA target non-structural proteins (NSPs)
ttgtagatgatatcgtaaaaa

SEQ ID NO: 218 - DNA, 21 bp, SARS coronavirus 2
  5′ sense SARS-CoV-2 DNA target non-structural proteins (NSPs)
gatggtacacttatgattgaa

SEQ ID NO: 219 - DNA, 21 bp, SARS coronavirus 2
  5′ sense SARS-CoV-2 DNA target non-structural proteins (NSPs)
tgtacttacaatacataagaa

SEQ ID NO: 220 - DNA, 21 bp, SARS coronavirus 2
  5′ sense SARS-CoV-2 DNA target non-structural proteins (NSPs)
gtacttacaatacataagaaa

SEQ ID NO: 221 - DNA, 21 bp, SARS coronavirus 2
  5′ sense SARS-CoV-2 DNA target non-structural proteins (NSPs)
gaaagctacatgatgagttaa

SEQ ID NO: 222 - DNA, 21 bp, SARS coronavirus 2
  5′ sense SARS-CoV-2 DNA target non-structural proteins (NSPs)
aaattcctaaaaactaattgt

SEQ ID NO: 223 - DNA, 21 bp, SARS coronavirus 2
  5′ sense SARS-CoV-2 DNA target non-structural proteins (NSPs)
aattcctaaaaactaattgtt

SEQ ID NO: 224 - DNA, 21 bp, SARS coronavirus 2
  5′ sense SARS-CoV-2 DNA target non-structural proteins (NSPs)
aaaaggacgaagatgacaatt

SEQ ID NO: 225 - DNA, 21 bp, SARS coronavirus 2
  5′ sense SARS-CoV-2 DNA target non-structural proteins (NSPs)
aacgtcttactaaatacacaa

SEQ ID NO: 226 - DNA, 21 bp, SARS coronavirus 2
  5′ sense SARS-CoV-2 DNA target non-structural proteins (NSPs)
aaggtaattgtgacacattaa

SEQ ID NO: 227 - DNA, 21 bp, SARS coronavirus 2
  5′ sense SARS-CoV-2 DNA target non-structural proteins (NSPs)
aaatacttgtcacatacaatt

SEQ ID NO: 228 - DNA, 21 bp, SARS coronavirus 2
  5′ sense SARS-CoV-2 DNA target non-structural proteins (NSPs)
aattgttgtgatgatgattat

SEQ ID NO: 229 - DNA, 21 bp, SARS coronavirus 2
  5′ sense SARS-CoV-2 DNA target non-structural proteins (NSPs)
aatcaagatctcaatggtaac

SEQ ID NO: 230 - DNA, 21 bp, SARS coronavirus 2
  5′ sense SARS-CoV-2 DNA target non-structural proteins (NSPs)
aacaaagccttacattaagtg

SEQ ID NO: 231 - DNA, 21 bp, SARS coronavirus 2
  5′ sense SARS-CoV-2 DNA target non-structural proteins (NSPs)
aatcaggatgtaaacttacat

SEQ ID NO: 232 - DNA, 21 bp, SARS coronavirus 2
  5′ sense SARS-CoV-2 DNA target non-structural proteins (NSPs)
aactcaaatgaatcttaagta

SEQ ID NO: 233 - DNA, 21 bp, SARS coronavirus 2
  5′ sense SARS-CoV-2 DNA target non-structural proteins (NSPs)
aaaattattgaaatcaatagc

SEQ ID NO: 234 - DNA, 21 bp, SARS coronavirus 2
  5′ sense SARS-CoV-2 DNA target non-structural proteins (NSPs)
aattggaacaagcaaattcta

SEQ ID NO: 235 - DNA, 21 bp, SARS coronavirus 2
  5′ sense SARS-CoV-2 DNA target non-structural proteins (NSPs)
aatgagtgtgctcaagtattg

SEQ ID NO: 236 - DNA, 21 bp, SARS coronavirus 2
  5′ sense SARS-CoV-2 DNA target non-structural proteins (NSPs)
aactgcttatgctaatagtgt

SEQ ID NO: 237 - DNA, 21 bp, SARS coronavirus 2
  5′ sense SARS-CoV-2 DNA target non-structural proteins (NSPs)
aacaaaattgccgataagtat

SEQ ID NO: 238 - DNA, 21 bp, SARS coronavirus 2
  5′ sense SARS-CoV-2 DNA target non-structural proteins (NSPs)
aaaattgccgataagtatgtc

SEQ ID NO: 239 - DNA, 21 bp, SARS coronavirus 2
  5′ sense SARS-CoV-2 DNA target non-structural proteins (NSPs)
aaaaacagatggtacacttat

SEQ ID NO: 240 - DNA, 21 bp, SARS coronavirus 2
  5′ sense SARS-CoV-2 DNA target non-structural proteins (NSPs)
aaaacagatggtacacttatg

SEQ ID NO: 241 - DNA, 21 bp, SARS coronavirus 2
  5′ sense SARS-CoV-2 DNA target non-structural proteins (NSPs)
aacagatggtacacttatgat

SEQ ID NO: 242 - DNA, 21 bp, SARS coronavirus 2
  5′ sense SARS-CoV-2 DNA target non-structural proteins (NSPs)
aaagctacatgatgagttaac

SEQ ID NO: 243 - DNA, 21 bp, SARS coronavirus 2
  5′ sense SARS-CoV-2 DNA target non-structural proteins (NSPs)
tatggctgtagttgtgatcaa

SEQ ID NO: 244 - DNA, 21 bp, SARS coronavirus 2
  5′ sense SARS-CoV-2 DNA target non-structural proteins (NSPs)
aattgttgtcgcttccaagaa

SEQ ID NO: 245 - DNA, 21 bp, SARS coronavirus 2
  5′ sense SARS-CoV-2 DNA target non-structural proteins (NSPs)
attgttgtcgcttccaagaaa

SEQ ID NO: 246 - DNA, 21 bp, SARS coronavirus 2
  5′ sense SARS-CoV-2 DNA target non-structural proteins (NSPs)
ttgttgtcgcttccaagaaaa

SEQ ID NO: 247 - DNA, 21 bp, SARS coronavirus 2
  5′ sense SARS-CoV-2 DNA target non-structural proteins (NSPs)
agaaaaggacgaagatgacaa

SEQ ID NO: 248 - DNA, 21 bp, SARS coronavirus 2
  5′ sense SARS-CoV-2 DNA target non-structural proteins (NSPs)
ttctctaactaccaacatgaa

SEQ ID NO: 249 - DNA, 21 bp, SARS coronavirus 2
  5′ sense SARS-CoV-2 DNA target non-structural proteins (NSPs)
tctaactaccaacatgaagaa

SEQ ID NO: 250 - DNA, 21 bp, SARS coronavirus 2
  5′ sense SARS-CoV-2 DNA target non-structural proteins (NSPs)
ggattgtccagctgttgctaa

SEQ ID NO: 251 - DNA, 21 bp, SARS coronavirus 2
  5′ sense SARS-CoV-2 DNA target non-structural proteins (NSPs)
gattgtccagctgttgctaaa

SEQ ID NO: 252 - DNA, 21 bp, SARS coronavirus 2
  5′ sense SARS-CoV-2 DNA target non-structural proteins (NSPs)
agaaatacttgtcacatacaa

SEQ ID NO: 253 - DNA, 21 bp, SARS coronavirus 2
  5′ sense SARS-CoV-2 DNA target non-structural proteins (NSPs)
gtatacgccaacttaggtgaa

SEQ ID NO: 254 - DNA, 21 bp, SARS coronavirus 2
  5′ sense SARS-CoV-2 DNA target non-structural proteins (NSPs)
attagataatcaagatctcaa

SEQ ID NO: 255 - DNA, 21 bp, SARS coronavirus 2
  5′ sense SARS-CoV-2 DNA target non-structural proteins (NSPs)
taatcaagatctcaatggtaa

SEQ ID NO: 256 - DNA, 21 bp, SARS coronavirus 2
  5′ sense SARS-CoV-2 DNA target non-structural proteins (NSPs)
cacatgttgacactgacttaa

SEQ ID NO: 257 - DNA, 21 bp, SARS coronavirus 2
  5′ sense SARS-CoV-2 DNA target non-structural proteins (NSPs)
acttcacggaagagaggttaa

SEQ ID NO: 258 - DNA, 21 bp, SARS coronavirus 2
  5′ sense SARS-CoV-2 DNA target non-structural proteins (NSPs)
cttcacggaagagaggttaaa

SEQ ID NO: 259 - DNA, 21 bp, SARS coronavirus 2
  5′ sense SARS-CoV-2 DNA target non-structural proteins (NSPs)
gatgcattctgcattgtgcaa

SEQ ID NO: 260 - DNA, 21 bp, SARS coronavirus 2
  5′ sense SARS-CoV-2 DNA target non-structural proteins (NSPs)
atgcattctgcattgtgcaaa

SEQ ID NO: 261 - DNA, 21 bp, SARS coronavirus 2
  5′ sense SARS-CoV-2 DNA target non-structural proteins (NSPs)
ttgtacataatcaggatgtaa

SEQ ID NO: 262 - DNA, 21 bp, SARS coronavirus 2
  5′ sense SARS-CoV-2 DNA target non-structural proteins (NSPs)
tgtacataatcaggatgtaaa

SEQ ID NO: 263 - DNA, 21 bp, SARS coronavirus 2
  5′ sense SARS-CoV-2 DNA target non-structural proteins (NSPs)
tatgcacgctgcttctggtaa

SEQ ID NO: 264 - DNA, 21 bp, SARS coronavirus 2
  5′ sense SARS-CoV-2 DNA target non-structural proteins (NSPs)
aaggaaggaagttctgttgaa

SEQ ID NO: 265 - DNA, 21 bp, SARS coronavirus 2
  5′ sense SARS-CoV-2 DNA target non-structural proteins (NSPs)
actatcgttataatctaccaa

SEQ ID NO: 266 - DNA, 21 bp, SARS coronavirus 2
  5′ sense SARS-CoV-2 DNA target non-structural proteins (NSPs)
atcgttataatctaccaacaa

SEQ ID NO: 267 - DNA, 21 bp, SARS coronavirus 2
  5′ sense SARS-CoV-2 DNA target non-structural proteins (NSPs)
acaatgtgtgatatcagacaa

SEQ ID NO: 268 - DNA, 21 bp, SARS coronavirus 2
  5′ sense SARS-CoV-2 DNA target non-structural proteins (NSPs)
atcgtcaacaacctagacaaa

SEQ ID NO: 269 - DNA, 21 bp, SARS coronavirus 2
  5′ sense SARS-CoV-2 DNA target non-structural proteins (NSPs)
ttaagtatgccattagtgcaa

SEQ ID NO: 270 - DNA, 21 bp, SARS coronavirus 2
  5′ sense SARS-CoV-2 DNA target non-structural proteins (NSPs)
taagtatgccattagtgcaaa

SEQ ID NO: 271 - DNA, 21 bp, SARS coronavirus 2
  5′ sense SARS-CoV-2 DNA target non-structural proteins (NSPs)
gtatgccattagtgcaaagaa

SEQ ID NO: 272 - DNA, 21 bp, SARS coronavirus 2
  5′ sense SARS-CoV-2 DNA target non-structural proteins (NSPs)
tatctgtagtactatgaccaa

SEQ ID NO: 273 - DNA, 21 bp, SARS coronavirus 2
  5′ sense SARS-CoV-2 DNA target non-structural proteins (NSPs)
ctagaggagctactgtagtaa

SEQ ID NO: 274 - DNA, 21 bp, SARS coronavirus 2
  5′ sense SARS-CoV-2 DNA target non-structural proteins (NSPs)
ctactgtagtaattggaacaa

SEQ ID NO: 275 - DNA, 21 bp, SARS coronavirus 2
  5′ sense SARS-CoV-2 DNA target non-structural proteins (NSPs)
tgtagtaattggaacaagcaa

SEQ ID NO: 276 - DNA, 21 bp, SARS coronavirus 2
  5′ sense SARS-CoV-2 DNA target non-structural proteins (NSPs)
gtagtaattggaacaagcaaa

SEQ ID NO: 277 - DNA, 21 bp, SARS coronavirus 2
  5′ sense SARS-CoV-2 DNA target non-structural proteins (NSPs)
ccatgcctaacatgcttagaa

SEQ ID NO: 278 - DNA, 21 bp, SARS coronavirus 2
  5′ sense SARS-CoV-2 DNA target non-structural proteins (NSPs)
tgtgctcaagtattgagtgaa

SEQ ID NO: 279 - DNA, 21 bp, SARS coronavirus 2
  5′ sense SARS-CoV-2 DNA target non-structural proteins (NSPs)
gtgctcaagtattgagtgaaa

SEQ ID NO: 280 - DNA, 21 bp, SARS coronavirus 2
  5′ sense SARS-CoV-2 DNA target non-structural proteins (NSPs)
ttatctactgatggtaacaaa

SEQ ID NO: 281 - DNA, 21 bp, SARS coronavirus 2
  5′ sense SARS-CoV-2 DNA target non-structural proteins (NSPs)
aaggtctagtggctagcataa

SEQ ID NO: 282 - DNA, 21 bp, SARS coronavirus 2
  5′ sense SARS-CoV-2 DNA target non-structural proteins (NSPs)
gactgagactgaccttactaa

SEQ ID NO: 283 - DNA, 21 bp, SARS coronavirus 2
  5′ sense SARS-CoV-2 DNA target non-structural proteins (NSPs)
tcaacatacaatgctagttaa

SEQ ID NO: 284 - DNA, 21 bp, SARS coronavirus 2
  5′ sense SARS-CoV-2 DNA target non-structural proteins (NSPs)
gtattctgttatgcttactaa

SEQ ID NO: 285 - DNA, 21 bp, SARS coronavirus 2
  5′ sense SARS-CoV-2 DNA target non-structural proteins (NSPs)
aaaggttatggctgtagttgt

SEQ ID NO: 286 - DNA, 21 bp, SARS coronavirus 2
  5′ sense SARS-CoV-2 DNA target non-structural proteins (NSPs)
aaggttatggctgtagttgtg

SEQ ID NO: 287 - DNA, 21 bp, SARS coronavirus 2
  5′ sense SARS-CoV-2 DNA target non-structural proteins (NSPs)
aattgttgtcgcttccaagaa

SEQ ID NO: 288 - DNA, 21 bp, SARS coronavirus 2
  5′ sense SARS-CoV-2 DNA target non-structural proteins (NSPs)
aagaaaaggacgaagatgaca

SEQ ID NO: 289 - DNA, 21 bp, SARS coronavirus 2
  5′ sense SARS-CoV-2 DNA target non-structural proteins (NSPs)
aactaccaacatgaagaaaca

SEQ ID NO: 290 - DNA, 21 bp, SARS coronavirus 2
  5′ sense SARS-CoV-2 DNA target non-structural proteins (NSPs)
aaaagaaatacttgtcacata

SEQ ID NO: 291 - DNA, 21 bp, SARS coronavirus 2
  5′ sense SARS-CoV-2 DNA target non-structural proteins (NSPs)
aaagaaatacttgtcacatac

SEQ ID NO: 292 - DNA, 21 bp, SARS coronavirus 2
  5′ sense SARS-CoV-2 DNA target non-structural proteins (NSPs)
aagaaatacttgtcacataca

SEQ ID NO: 293 - DNA, 21 bp, SARS coronavirus 2
  5′ sense SARS-CoV-2 DNA target non-structural proteins (NSPs)
aataaaaaggactggtatgat

SEQ ID NO: 294 - DNA, 21 bp, SARS coronavirus 2
  5′ sense SARS-CoV-2 DNA target non-structural proteins (NSPs)
aaaaacagtacaattctgtga

SEQ ID NO: 295 - DNA, 21 bp, SARS coronavirus 2
  5′ sense SARS-CoV-2 DNA target non-structural proteins (NSPs)
aaaacagtacaattctgtgat

SEQ ID NO: 296 - DNA, 21 bp, SARS coronavirus 2
  5′ sense SARS-CoV-2 DNA target non-structural proteins (NSPs)
aaacagtacaattctgtgatg

SEQ ID NO: 297 - DNA, 21 bp, SARS coronavirus 2
  5′ sense SARS-CoV-2 DNA target non-structural proteins (NSPs)
aatgcctatattaaccttgac

SEQ ID NO: 298 - DNA, 21 bp, SARS coronavirus 2
  5′ sense SARS-CoV-2 DNA target non-structural proteins (NSPs)
aactgcagagtcacatgttga

SEQ ID NO: 299 - DNA, 21 bp, SARS coronavirus 2
  5′ sense SARS-CoV-2 DNA target non-structural proteins (NSPs)
aacttacatagctctagactt

SEQ ID NO: 300 - DNA, 21 bp, SARS coronavirus 2
  5′ sense SARS-CoV-2 DNA target non-structural proteins (NSPs)
aatctattactagataaacgc

SEQ ID NO: 301 - DNA, 21 bp, SARS coronavirus 2
  5′ sense SARS-CoV-2 DNA target non-structural proteins (NSPs)
aaggaaggaagttctgttgaa

SEQ ID NO: 302 - DNA, 21 bp, SARS coronavirus 2
  5′ sense SARS-CoV-2 DNA target non-structural proteins (NSPs)
aatgctgctatcagcgattat

SEQ ID NO: 303 - DNA, 21 bp, SARS coronavirus 2
  5′ sense SARS-CoV-2 DNA target non-structural proteins (NSPs)
aatctaccaacaatgtgtgat

SEQ ID NO: 304 - DNA, 21 bp, SARS coronavirus 2
  5′ sense SARS-CoV-2 DNA target non-structural proteins (NSPs)
aacaatgtgtgatatcagaca

SEQ ID NO: 305 - DNA, 21 bp, SARS coronavirus 2
  5′ sense SARS-CoV-2 DNA target non-structural proteins (NSPs)
aaccaagtcatcgtcaacaac

SEQ ID NO: 306 - DNA, 21 bp, SARS coronavirus 2
  5′ sense SARS-CoV-2 DNA target non-structural proteins (NSPs)
aagtcatcgtcaacaacctag

SEQ ID NO: 307 - DNA, 21 bp, SARS coronavirus 2
  5′ sense SARS-CoV-2 DNA target non-structural proteins (NSPs)
aacaacctagacaaatcagct

SEQ ID NO: 308 - DNA, 21 bp, SARS coronavirus 2
  5′ sense SARS-CoV-2 DNA target non-structural proteins (NSPs)
aagtatgccattagtgcaaag

SEQ ID NO: 309 - DNA, 21 bp, SARS coronavirus 2
  5′ sense SARS-CoV-2 DNA target non-structural proteins (NSPs)
aaattattgaaatcaatagcc

SEQ ID NO: 310 - DNA, 21 bp, SARS coronavirus 2
  5′ sense SARS-CoV-2 DNA target non-structural proteins (NSPs)
aacatgcttagaattatggcc

SEQ ID NO: 311 - DNA, 21 bp, SARS coronavirus 2
  5′ sense SARS-CoV-2 DNA target non-structural proteins (NSPs)
aattatggcctcacttgttct

SEQ ID NO: 312 - DNA, 21 bp, SARS coronavirus 2
  5′ sense SARS-CoV-2 DNA target non-structural proteins (NSPs)
aaacatacaacgtgttgtagc

SEQ ID NO: 313 - DNA, 21 bp, SARS coronavirus 2
  5′ sense SARS-CoV-2 DNA target non-structural proteins (NSPs)
aagtattgagtgaaatggtca

SEQ ID NO: 314 - DNA, 21 bp, SARS coronavirus 2
  5′ sense SARS-CoV-2 DNA target non-structural proteins (NSPs)
aagctgtcacggccaatgtta

SEQ ID NO: 315 - DNA, 21 bp, SARS coronavirus 2
  5′ sense SARS-CoV-2 DNA target non-structural proteins (NSPs)
aaattgccgataagtatgtcc

SEQ ID NO: 316 - DNA, 21 bp, SARS coronavirus 2
  5′ sense SARS-CoV-2 DNA target non-structural proteins (NSPs)
aaggtctagtggctagcataa

SEQ ID NO: 317 - DNA, 21 bp, SARS coronavirus 2
  5′ sense SARS-CoV-2 DNA target non-structural proteins (NSPs)
aaacagatggtacacttatga

SEQ ID NO: 318 - DNA, 21 bp, SARS coronavirus 2
  5′ sense SARS-CoV-2 DNA target non-structural proteins (NSPs)
aacatcctaatcaggagtatg

SEQ ID NO: 319 - DNA, 21 bp, SARS coronavirus 2
  5′ sense SARS-CoV-2 DNA target non-structural proteins (NSPs)
aatacataagaaagctacatg

SEQ ID NO: 320 - DNA, 21 bp, SARS coronavirus 2
  5′ sense SARS-CoV-2 DNA target non-structural proteins (NSPs)
aagaaagctacatgatgagtt

SEQ ID NO: 321 - DNA, 21 bp, SARS coronavirus 2
  5′ sense SARS-CoV-2 DNA target non-structural proteins (NSPs)
aagctacatgatgagttaaca

SEQ ID NO: 322 - DNA, 21 bp, SARS coronavirus 2
  5′ sense SARS-CoV-2 DNA target non-structural proteins (NSPs)
aacaggacacatgttagacat

SEQ ID NO: 323 - DNA, 21 bp, SARS coronavirus 2
  5′ sense SARS-CoV-2 DNA target non-structural proteins (NSPs)
aatgataacacttcaaggtat

SEQ ID NO: 324 - DNA, 21 bp, SARS coronavirus 2
  5′ sense SARS-CoV-2 DNA target non-structural proteins (NSPs)
cttcagtcagctgatgcacaa

SEQ ID NO: 325 - DNA, 21 bp, SARS coronavirus 2
  5′ sense SARS-CoV-2 DNA target non-structural proteins (NSPs)
cgcttccaagaaaaggacgaa

SEQ ID NO: 326 - DNA, 21 bp, SARS coronavirus 2
  5′ sense SARS-CoV-2 DNA target non-structural proteins (NSPs)
gtaccacatatatcacgtcaa

SEQ ID NO: 327 - DNA, 21 bp, SARS coronavirus 2
  5′ sense SARS-CoV-2 DNA target non-structural proteins (NSPs)
tatattacgcgtatacgccaa

SEQ ID NO: 328 - DNA, 21 bp, SARS coronavirus 2
  5′ sense SARS-CoV-2 DNA target non-structural proteins (NSPs)
attctgtgatgccatgcgaaa

SEQ ID NO: 329 - DNA, 21 bp, SARS coronavirus 2
  5′ sense SARS-CoV-2 DNA target non-structural proteins (NSPs)
ttaaaatatgacttcacggaa

SEQ ID NO: 330 - DNA, 21 bp, SARS coronavirus 2
  5′ sense SARS-CoV-2 DNA target non-structural proteins (NSPs)
ggctgtattaatgctaaccaa

SEQ ID NO: 331 - DNA, 21 bp, SARS coronavirus 2
  5′ sense SARS-CoV-2 DNA target non-structural proteins (NSPs)
tgctaaccaagtcatcgtcaa

SEQ ID NO: 332 - DNA, 21 bp, SARS coronavirus 2
  5′ sense SARS-CoV-2 DNA target non-structural proteins (NSPs)
taaccaagtcatcgtcaacaa

SEQ ID NO: 333 - DNA, 21 bp, SARS coronavirus 2
  5′ sense SARS-CoV-2 DNA target non-structural proteins (NSPs)
catcgtcaacaacctagacaa

SEQ ID NO: 334 - DNA, 21 bp, SARS coronavirus 2
  5′ sense SARS-CoV-2 DNA target non-structural proteins (NSPs)
tcaatgagttatgaggatcaa

SEQ ID NO: 335 - DNA, 21 bp, SARS coronavirus 2
  5′ sense SARS-CoV-2 DNA target non-structural proteins (NSPs)
attctatggtggttggcacaa

SEQ ID NO: 336 - DNA, 21 bp, SARS coronavirus 2
  5′ sense SARS-CoV-2 DNA target non-structural proteins (NSPs)
atgtgatagagccatgcctaa

SEQ ID NO: 337 - DNA, 21 bp, SARS coronavirus 2
  5′ sense SARS-CoV-2 DNA target non-structural proteins (NSPs)
ttagctaatgagtgtgctcaa

SEQ ID NO: 338 - DNA, 21 bp, SARS coronavirus 2
  5′ sense SARS-CoV-2 DNA target non-structural proteins (NSPs)
cctcatcaggagatgccacaa

SEQ ID NO: 339 - DNA, 21 bp, SARS coronavirus 2
  5′ sense SARS-CoV-2 DNA target non-structural proteins (NSPs)
catcatccggtgatgctacaa

SEQ ID NO: 340 - DNA, 21 bp, SARS coronavirus 2
  5′ sense SARS-CoV-2 DNA target non-structural proteins (NSPs)
tggtaacaaaattgccgataa

SEQ ID NO: 341 - DNA, 21 bp, SARS coronavirus 2
  5′ sense SARS-CoV-2 DNA target non-structural proteins (NSPs)
aatagcacttatgcatctcaa

SEQ ID NO: 342 - DNA, 21 bp, SARS coronavirus 2
  5′ sense SARS-CoV-2 DNA target non-structural proteins (NSPs)
cttactaaaggacctcatgaa

SEQ ID NO: 343 - DNA, 21 bp, SARS coronavirus 2
  5′ sense SARS-CoV-2 DNA target non-structural proteins (NSPs)
aaaaactaattgttgtcgctt

SEQ ID NO: 344 - DNA, 21 bp, SARS coronavirus 2
  5′ sense SARS-CoV-2 DNA target non-structural proteins (NSPs)
aaaactaattgttgtcgcttc

SEQ ID NO: 345 - DNA, 21 bp, SARS coronavirus 2
  5′ sense SARS-CoV-2 DNA target non-structural proteins (NSPs)
aatagacggtgacatggtacc

SEQ ID NO: 346 - DNA, 21 bp, SARS coronavirus 2
  5′ sense SARS-CoV-2 DNA target non-structural proteins (NSPs)
aatggcagacctcgtctatgc

SEQ ID NO: 347 - DNA, 21 bp, SARS coronavirus 2
  5′ sense SARS-CoV-2 DNA target non-structural proteins (NSPs)
aatggctgatttagtctatgc

SEQ ID NO: 348 - DNA, 21 bp, SARS coronavirus 2
  5′ sense SARS-CoV-2 DNA target non-structural proteins (NSPs)
aacagtacaattctgtgatgc

SEQ ID NO: 349 - DNA, 21 bp, SARS coronavirus 2
  5′ sense SARS-CoV-2 DNA target non-structural proteins (NSPs)
aaatgctggtattgttggtgt

SEQ ID NO: 350 - DNA, 21 bp, SARS coronavirus 2
  5′ sense SARS-CoV-2 DNA target non-structural proteins (NSPs)
aatgctggtattgttggtgta

SEQ ID NO: 351 - DNA, 21 bp, SARS coronavirus 2
  5′ sense SARS-CoV-2 DNA target non-structural proteins (NSPs)
aagatctcaatggtaactggt

SEQ ID NO: 352 - DNA, 21 bp, SARS coronavirus 2
  5′ sense SARS-CoV-2 DNA target non-structural proteins (NSPs)
aaaccacgccaggtagtggag

SEQ ID NO: 353 - DNA, 21 bp, SARS coronavirus 2
  5′ sense SARS-CoV-2 DNA target non-structural proteins (NSPs)
aagtagcaccaggctgcggag

SEQ ID NO: 354 - DNA, 21 bp, SARS coronavirus 2
  5′ sense SARS-CoV-2 DNA target non-structural proteins (NSPs)
aaccacgccaggtagtggagt

SEQ ID NO: 355 - DNA, 21 bp, SARS coronavirus 2
  5′ sense SARS-CoV-2 DNA target non-structural proteins (NSPs)
aaatatgacttcacggaagag

SEQ ID NO: 356 - DNA, 21 bp, SARS coronavirus 2
  5′ sense SARS-CoV-2 DNA target non-structural proteins (NSPs)
aactggataccacttcagaga

SEQ ID NO: 357 - DNA, 21 bp, SARS coronavirus 2
  5′ sense SARS-CoV-2 DNA target non-structural proteins (NSPs)
aaacttacatagctctagact

SEQ ID NO: 358 - DNA, 21 bp, SARS coronavirus 2
  5′ sense SARS-CoV-2 DNA target non-structural proteins (NSPs)
aaggaattacttgtgtatgct

SEQ ID NO: 359 - DNA, 21 bp, SARS coronavirus 2
  5′ sense SARS-CoV-2 DNA target non-structural proteins (NSPs)
aattacttgtgtatgctgctg

SEQ ID NO: 360 - DNA, 21 bp, SARS coronavirus 2
  5′ sense SARS-CoV-2 DNA target non-structural proteins (NSPs)
aatgtgtgatatcagacaact

SEQ ID NO: 361 - DNA, 21 bp, SARS coronavirus 2
  5′ sense SARS-CoV-2 DNA target non-structural proteins (NSPs)
aatgctaaccaagtcatcgtc

SEQ ID NO: 362 - DNA, 21 bp, SARS coronavirus 2
  5′ sense SARS-CoV-2 DNA target non-structural proteins (NSPs)
aatgagttatgaggatcaaga

SEQ ID NO: 363 - DNA, 21 bp, SARS coronavirus 2
  5′ sense SARS-CoV-2 DNA target non-structural proteins (NSPs)
aaatgaatcttaagtatgcca

SEQ ID NO: 364 - DNA, 21 bp, SARS coronavirus 2
  5′ sense SARS-CoV-2 DNA target non-structural proteins (NSPs)
aatgaatcttaagtatgccat

SEQ ID NO: 365 - DNA, 21 bp, SARS coronavirus 2
  5′ sense SARS-CoV-2 DNA target non-structural proteins (NSPs)
aatcttaagtatgccattagt

SEQ ID NO: 366 - DNA, 21 bp, SARS coronavirus 2
  5′ sense SARS-CoV-2 DNA target non-structural proteins (NSPs)
aattattgaaatcaatagccg

SEQ ID NO: 367 - DNA, 21 bp, SARS coronavirus 2
  5′ sense SARS-CoV-2 DNA target non-structural proteins (NSPs)
aatcaatagccgccactagag

SEQ ID NO: 368 - DNA, 21 bp, SARS coronavirus 2
  5′ sense SARS-CoV-2 DNA target non-structural proteins (NSPs)
aatagccgccactagaggagc

SEQ ID NO: 369 - DNA, 21 bp, SARS coronavirus 2
  5′ sense SARS-CoV-2 DNA target non-structural proteins (NSPs)
aacaagcaaattctatggtgg

SEQ ID NO: 370 - DNA, 21 bp, SARS coronavirus 2
  5′ sense SARS-CoV-2 DNA target non-structural proteins (NSPs)
aagcaaattctatggtggttg

SEQ ID NO: 371 - DNA, 21 bp, SARS coronavirus 2
  5′ sense SARS-CoV-2 DNA target non-structural proteins (NSPs)
aacatacaacgtgttgtagct

SEQ ID NO: 372 - DNA, 21 bp, SARS coronavirus 2
  5′ sense SARS-CoV-2 DNA target non-structural proteins (NSPs)
aacgtgttgtagcttgtcaca

SEQ ID NO: 373 - DNA, 21 bp, SARS coronavirus 2
  5′ sense SARS-CoV-2 DNA target non-structural proteins (NSPs)
aaaccaggtggaacctcatca

SEQ ID NO: 374 - DNA, 21 bp, SARS coronavirus 2
  5′ sense SARS-CoV-2 DNA target non-structural proteins (NSPs)
aaccaggtggaacctcatcag

SEQ ID NO: 375 - DNA, 21 bp, SARS coronavirus 2
  5′ sense SARS-CoV-2 DNA target non-structural proteins (NSPs)
aaccaggtggaacatcatccg

SEQ ID NO: 376 - DNA, 21 bp, SARS coronavirus 2
  5′ sense SARS-CoV-2 DNA target non-structural proteins (NSPs)
aacctcatcaggagatgccac

SEQ ID NO: 377 - DNA, 21 bp, SARS coronavirus 2
  5′ sense SARS-CoV-2 DNA target non-structural proteins (NSPs)
aacatcatccggtgatgctac

SEQ ID NO: 378 - DNA, 21 bp, SARS coronavirus 2
  5′ sense SARS-CoV-2 DNA target non-structural proteins (NSPs)
aattgccgataagtatgtccg

SEQ ID NO: 379 - DNA, 21 bp, SARS coronavirus 2
  5′ sense SARS-CoV-2 DNA target non-structural proteins (NSPs)
aaatagagatgttgacacaga

SEQ ID NO: 380 - DNA, 21 bp, SARS coronavirus 2
  5′ sense SARS-CoV-2 DNA target non-structural proteins (NSPs)
aatagagatgttgacacagac

SEQ ID NO: 381 - DNA, 21 bp, SARS coronavirus 2
  5′ sense SARS-CoV-2 DNA target non-structural proteins (NSPs)
aatagcacttatgcatctcaa

SEQ ID NO: 382 - DNA, 21 bp, SARS coronavirus 2
  5′ sense SARS-CoV-2 DNA target non-structural proteins (NSPs)
aaaatgttggactgagactga

SEQ ID NO: 383 - DNA, 21 bp, SARS coronavirus 2
  5′ sense SARS-CoV-2 DNA target non-structural proteins (NSPs)
aaatgttggactgagactgac

SEQ ID NO: 384 - DNA, 21 bp, SARS coronavirus 2
  5′ sense SARS-CoV-2 DNA target non-structural proteins (NSPs)
aatgttggactgagactgacc

SEQ ID NO: 385 - DNA, 21 bp, SARS coronavirus 2
  5′ sense SARS-CoV-2 DNA target non-structural proteins (NSPs)
aaacatcctaatcaggagtat

SEQ ID NO: 386 - DNA, 21 bp, SARS coronavirus 2
  5′ sense SARS-CoV-2 DNA target non-structural proteins (NSPs)
aatcaggagtatgctgatgtc

SEQ ID NO: 387 - DNA, 21 bp, SARS coronavirus 2
  5′ sense SARS-CoV-2 DNA target non-structural proteins (NSPs)
actaattgttgtcgcttccaa

SEQ ID NO: 388 - DNA, 21 bp, SARS coronavirus 2
  5′ sense SARS-CoV-2 DNA target non-structural proteins (NSPs)
ttaggtgaacgtgtacgccaa

SEQ ID NO: 389 - DNA, 21 bp, SARS coronavirus 2
  5′ sense SARS-CoV-2 DNA target non-structural proteins (NSPs)
aattctgtgatgccatgcgaa

SEQ ID NO: 390 - DNA, 21 bp, SARS coronavirus 2
  5′ sense SARS-CoV-2 DNA target non-structural proteins (NSPs)
ctcacttgttcttgctcgcaa

SEQ ID NO: 391 - DNA, 21 bp, SARS coronavirus 2
  5′ sense SARS-CoV-2 DNA target non-structural proteins (NSPs)
tcacttgttcttgctcgcaaa

SEQ ID NO: 392 - DNA, 21 bp, SARS coronavirus 2
  5′ sense SARS-CoV-2 DNA target non-structural proteins (NSPs)
tatatgttaaaccaggtggaa

SEQ ID NO: 393 - DNA, 21 bp, SARS coronavirus 2
  5′ sense SARS-CoV-2 DNA target non-structural proteins (NSPs)
tgccgataagtatgtccgcaa

SEQ ID NO: 394 - DNA, 21 bp, SARS coronavirus 2
  5′ sense SARS-CoV-2 DNA target non-structural proteins (NSPs)
aacaccgtgcggcacaggcac

SEQ ID NO: 395 - DNA, 21 bp, SARS coronavirus 2
  5′ sense SARS-CoV-2 DNA target non-structural proteins (NSPs)
aaactaattgttgtcgcttcc

SEQ ID NO: 396 - DNA, 21 bp, SARS coronavirus 2
  5′ sense SARS-CoV-2 DNA target non-structural proteins (NSPs)
aactaattgttgtcgcttcca

SEQ ID NO: 397 - DNA, 21 bp, SARS coronavirus 2
  5′ sense SARS-CoV-2 DNA target non-structural proteins (NSPs)
aaggattgtccagctgttgct

SEQ ID NO: 398 - DNA, 21 bp, SARS coronavirus 2
  5′ sense SARS-CoV-2 DNA target non-structural proteins (NSPs)
aaatacacaatggcagacctc

SEQ ID NO: 399 - DNA, 21 bp, SARS coronavirus 2
  5′ sense SARS-CoV-2 DNA target non-structural proteins (NSPs)
aatacacaatggcagacctcg

SEQ ID NO: 400 - DNA, 21 bp, SARS coronavirus 2
  5′ sense SARS-CoV-2 DNA target non-structural proteins (NSPs)
aacttaggtgaacgtgtacgc

SEQ ID NO: 401 - DNA, 21 bp, SARS coronavirus 2
  5′ sense SARS-CoV-2 DNA target non-structural proteins (NSPs)
aattctgtgatgccatgcgaa

SEQ ID NO: 402 - DNA, 21 bp, SARS coronavirus 2
  5′ sense SARS-CoV-2 DNA target non-structural proteins (NSPs)
aaaatatgacttcacggaaga

SEQ ID NO: 403 - DNA, 21 bp, SARS coronavirus 2
  5′ sense SARS-CoV-2 DNA target non-structural proteins (NSPs)
aatatgacttcacggaagaga

SEQ ID NO: 404 - DNA, 21 bp, SARS coronavirus 2
  5′ sense SARS-CoV-2 DNA target non-structural proteins (NSPs)
aacctagacaaatcagctggt

SEQ ID NO: 405 - DNA, 21 bp, SARS coronavirus 2
  5′ sense SARS-CoV-2 DNA target non-structural proteins (NSPs)
aatagagctcgcaccgtagct

SEQ ID NO: 406 - DNA, 21 bp, SARS coronavirus 2
  5′ sense SARS-CoV-2 DNA target non-structural proteins (NSPs)
aaatcaatagccgccactaga

SEQ ID NO: 407 - DNA, 21 bp, SARS coronavirus 2
  5′ sense SARS-CoV-2 DNA target non-structural proteins (NSPs)
aaattctatggtggttggcac

SEQ ID NO: 408 - DNA, 21 bp, SARS coronavirus 2
  5′ sense SARS-CoV-2 DNA target non-structural proteins (NSPs)
aattctatggtggttggcaca

SEQ ID NO: 409 - DNA, 21 bp, SARS coronavirus 2
  5′ sense SARS-CoV-2 DNA target non-structural proteins (NSPs)
aatgtgatagagccatgccta

SEQ ID NO: 410 - DNA, 21 bp, SARS coronavirus 2
  5′ sense SARS-CoV-2 DNA target non-structural proteins (NSPs)
aatggtcatgtgtggcggttc

SEQ ID NO: 411 - DNA, 21 bp, SARS coronavirus 2
  5′ sense SARS-CoV-2 DNA target non-structural proteins (NSPs)
aatgatgatactctctgacga

SEQ ID NO: 412 - DNA, 21 bp, SARS coronavirus 2
  5′ sense SARS-CoV-2 DNA target non-structural proteins (NSPs)
aagcaaaatgttggactgaga

SEQ ID NO: 413 - DNA, 21 bp, SARS coronavirus 2
  5′ sense SARS-CoV-2 DNA target non-structural proteins (NSPs)
agttgtgatcaactccgcgaa

SEQ ID NO: 414 - DNA, 21 bp, SARS coronavirus 2
  5′ sense SARS-CoV-2 DNA target non-structural proteins (NSPs)
ttgtcaagctgtcacggccaa

SEQ ID NO: 415 - DNA, 21 bp, SARS coronavirus 2
  5′ sense SARS-CoV-2 DNA target non-structural proteins (NSPs)
aaacaccgtgcggcacaggca

SEQ ID NO: 416 - DNA, 21 bp, SARS coronavirus 2
  5′ sense SARS-CoV-2 DNA target non-structural proteins (NSPs)
aaagaatagagctcgcaccgt

SEQ ID NO: 417 - DNA, 21 bp, SARS coronavirus 2
  5′ sense SARS-CoV-2 DNA target non-structural proteins (NSPs)
aagaatagagctcgcaccgta

SEQ ID NO: 418 - DNA, 21 bp, SARS coronavirus 2
  5′ sense SARS-CoV-2 DNA target non-structural proteins (NSPs)
aaatgtgatagagccatgcct

SEQ ID NO: 419 - DNA, 21 bp, SARS coronavirus 2
  5′ sense SARS-CoV-2 DNA target non-structural proteins (NSPs)
aaatggtcatgtgtggcggtt

SEQ ID NO: 420 - DNA, 21 bp, SARS coronavirus 2
  5′ sense SARS-CoV-2 DNA target non-structural proteins (NSPs)
aggtatgagctattattgtaa

SEQ ID NO: 421 - DNA, 21 bp, SARS coronavirus 2
  5′ sense SARS-CoV-2 DNA target non-structural proteins (NSPs)
ggtatgagctattattgtaaa

SEQ ID NO: 422 - DNA, 21 bp, SARS coronavirus 2
  5′ sense SARS-CoV-2 DNA target non-structural proteins (NSPs)
ctggttatcgtgtaactaaaa

SEQ ID NO: 423 - DNA, 21 bp, SARS coronavirus 2
  5′ sense SARS-CoV-2 DNA target non-structural proteins (NSPs)
tggttatcgtgtaactaaaaa

SEQ ID NO: 424 - DNA, 21 bp, SARS coronavirus 2
  5′ sense SARS-CoV-2 DNA target non-structural proteins (NSPs)
gtacaacaacttacaaattaa

SEQ ID NO: 425 - DNA, 21 bp, SARS coronavirus 2
  5′ sense SARS-CoV-2 DNA target non-structural proteins (NSPs)
tacaacaacttacaaattaaa

SEQ ID NO: 426 - DNA, 21 bp, SARS coronavirus 2
  5′ sense SARS-CoV-2 DNA target non-structural proteins (NSPs)
agcaatgttgcaaattatcaa

SEQ ID NO: 427 - DNA, 21 bp, SARS coronavirus 2
  5′ sense SARS-CoV-2 DNA target non-structural proteins (NSPs)
gcaatgttgcaaattatcaaa

SEQ ID NO: 428 - DNA, 21 bp, SARS coronavirus 2
  5′ sense SARS-CoV-2 DNA target non-structural proteins (NSPs)
caatgttgcaaattatcaaaa

SEQ ID NO: 429 - DNA, 21 bp, SARS coronavirus 2
  5′ sense SARS-CoV-2 DNA target non-structural proteins (NSPs)
cagtgtgtagacttatgaaaa

SEQ ID NO: 430 - DNA, 21 bp, SARS coronavirus 2
  5′ sense SARS-CoV-2 DNA target non-structural proteins (NSPs)
agctcactcttgtaatgtaaa

SEQ ID NO: 431 - DNA, 21 bp, SARS coronavirus 2
  5′ sense SARS-CoV-2 DNA target non-structural proteins (NSPs)
aaaacagtaaagtacaaatag

SEQ ID NO: 432 - DNA, 21 bp, SARS coronavirus 2
  5′ sense SARS-CoV-2 DNA target non-structural proteins (NSPs)
aacaacttacaaattaaatgt

SEQ ID NO: 433 - DNA, 21 bp, SARS coronavirus 2
  5′ sense SARS-CoV-2 DNA target non-structural proteins (NSPs)
aagagcactatgttagaatta

SEQ ID NO: 434 - DNA, 21 bp, SARS coronavirus 2
  5′ sense SARS-CoV-2 DNA target non-structural proteins (NSPs)
aatgttgcaaattatcaaaag

SEQ ID NO: 435 - DNA, 21 bp, SARS coronavirus 2
  5′ sense SARS-CoV-2 DNA target non-structural proteins (NSPs)
tagaccattcttatgttgtaa

SEQ ID NO: 436 - DNA, 21 bp, SARS coronavirus 2
  5′ sense SARS-CoV-2 DNA target non-structural proteins (NSPs)
agaccattcttatgttgtaaa

SEQ ID NO: 437 - DNA, 21 bp, SARS coronavirus 2
  5′ sense SARS-CoV-2 DNA target non-structural proteins (NSPs)
gttacgaccatgtcatatcaa

SEQ ID NO: 438 - DNA, 21 bp, SARS coronavirus 2
  5′ sense SARS-CoV-2 DNA target non-structural proteins (NSPs)
tgtcatatcaacatcacataa

SEQ ID NO: 439 - DNA, 21 bp, SARS coronavirus 2
  5′ sense SARS-CoV-2 DNA target non-structural proteins (NSPs)
gtcatatcaacatcacataaa

SEQ ID NO: 440 - DNA, 21 bp, SARS coronavirus 2
  5′ sense SARS-CoV-2 DNA target non-structural proteins (NSPs)
ctattattgtaaatcacataa

SEQ ID NO: 441 - DNA, 21 bp, SARS coronavirus 2
  5′ sense SARS-CoV-2 DNA target non-structural proteins (NSPs)
tattattgtaaatcacataaa

SEQ ID NO: 442 - DNA, 21 bp, SARS coronavirus 2
  5′ sense SARS-CoV-2 DNA target non-structural proteins (NSPs)
tactggttatcgtgtaactaa

SEQ ID NO: 443 - DNA, 21 bp, SARS coronavirus 2
  5′ sense SARS-CoV-2 DNA target non-structural proteins (NSPs)
actggttatcgtgtaactaaa

SEQ ID NO: 444 - DNA, 21 bp, SARS coronavirus 2
  5′ sense SARS-CoV-2 DNA target non-structural proteins (NSPs)
tcgtgtaactaaaaacagtaa

SEQ ID NO: 445 - DNA, 21 bp, SARS coronavirus 2
  5′ sense SARS-CoV-2 DNA target non-structural proteins (NSPs)
cgtgtaactaaaaacagtaaa

SEQ ID NO: 446 - DNA, 21 bp, SARS coronavirus 2
  5′ sense SARS-CoV-2 DNA target non-structural proteins (NSPs)
actaaaaacagtaaagtacaa

SEQ ID NO: 447 - DNA, 21 bp, SARS coronavirus 2
  5′ sense SARS-CoV-2 DNA target non-structural proteins (NSPs)
ctaaaaacagtaaagtacaaa

SEQ ID NO: 448 - DNA, 21 bp, SARS coronavirus 2
  5′ sense SARS-CoV-2 DNA target non-structural proteins (NSPs)
ccgaggtacaacaacttacaa

SEQ ID NO: 449 - DNA, 21 bp, SARS coronavirus 2
  5′ sense SARS-CoV-2 DNA target non-structural proteins (NSPs)
cgaggtacaacaacttacaaa

SEQ ID NO: 450 - DNA, 21 bp, SARS coronavirus 2
  5′ sense SARS-CoV-2 DNA target non-structural proteins (NSPs)
tgctgacatcacatacagtaa

SEQ ID NO: 451 - DNA, 21 bp, SARS coronavirus 2
  5′ sense SARS-CoV-2 DNA target non-structural proteins (NSPs)
cacaagagcactatgttagaa

SEQ ID NO: 452 - DNA, 21 bp, SARS coronavirus 2
  5′ sense SARS-CoV-2 DNA target non-structural proteins (NSPs)
tcaaaaggttggtatgcaaaa

SEQ ID NO: 453 - DNA, 21 bp, SARS coronavirus 2
  5′ sense SARS-CoV-2 DNA target non-structural proteins (NSPs)
ctatgtgagaaggcattaaaa

SEQ ID NO: 454 - DNA, 21 bp, SARS coronavirus 2
  5′ sense SARS-CoV-2 DNA target non-structural proteins (NSPs)
ataaattcaaagtgaattcaa

SEQ ID NO: 455 - DNA, 21 bp, SARS coronavirus 2
  5′ sense SARS-CoV-2 DNA target non-structural proteins (NSPs)
aaagtgaattcaacattagaa

SEQ ID NO: 456 - DNA, 21 bp, SARS coronavirus 2
  5′ sense SARS-CoV-2 DNA target non-structural proteins (NSPs)
ttcagtgtgtagacttatgaa

SEQ ID NO: 457 - DNA, 21 bp, SARS coronavirus 2
  5′ sense SARS-CoV-2 DNA target non-structural proteins (NSPs)
tcagtgtgtagacttatgaaa

SEQ ID NO: 458 - DNA, 21 bp, SARS coronavirus 2
  5′ sense SARS-CoV-2 DNA target non-structural proteins (NSPs)
gcttaaagcacataaagacaa

SEQ ID NO: 459 - DNA, 21 bp, SARS coronavirus 2
  5′ sense SARS-CoV-2 DNA target non-structural proteins (NSPs)
cttaaagcacataaagacaaa

SEQ ID NO: 460 - DNA, 21 bp, SARS coronavirus 2
  5′ sense SARS-CoV-2 DNA target non-structural proteins (NSPs)
cagctcactcttgtaatgtaa

SEQ ID NO: 461 - DNA, 21 bp, SARS coronavirus 2
  5′ sense SARS-CoV-2 DNA target non-structural proteins (NSPs)
aacatcacataaattagtctt

SEQ ID NO: 462 - DNA, 21 bp, SARS coronavirus 2
  5′ sense SARS-CoV-2 DNA target non-structural proteins (NSPs)
aaattagtcttgtctgttaat

SEQ ID NO: 463 - DNA, 21 bp, SARS coronavirus 2
  5′ sense SARS-CoV-2 DNA target non-structural proteins (NSPs)
aattagtcttgtctgttaatc

SEQ ID NO: 464 - DNA, 21 bp, SARS coronavirus 2
  5′ sense SARS-CoV-2 DNA target non-structural proteins (NSPs)
aactaaaaacagtaaagtaca

SEQ ID NO: 465 - DNA, 21 bp, SARS coronavirus 2
  5′ sense SARS-CoV-2 DNA target non-structural proteins (NSPs)
aaaaacagtaaagtacaaata

SEQ ID NO: 466 - DNA, 21 bp, SARS coronavirus 2
  5′ sense SARS-CoV-2 DNA target non-structural proteins (NSPs)
aaacagtaaagtacaaatagg

SEQ ID NO: 467 - DNA, 21 bp, SARS coronavirus 2
  5′ sense SARS-CoV-2 DNA target non-structural proteins (NSPs)
aacttacaaattaaatgttgg

SEQ ID NO: 468 - DNA, 21 bp, SARS coronavirus 2
  5′ sense SARS-CoV-2 DNA target non-structural proteins (NSPs)
aattaaatgttggtgattatt

SEQ ID NO: 469 - DNA, 21 bp, SARS coronavirus 2
  5′ sense SARS-CoV-2 DNA target non-structural proteins (NSPs)
aaggttggtatgcaaaagtat

SEQ ID NO: 470 - DNA, 21 bp, SARS coronavirus 2
  5′ sense SARS-CoV-2 DNA target non-structural proteins (NSPs)
aaatgtagtagaattatacct

SEQ ID NO: 471 - DNA, 21 bp, SARS coronavirus 2
  5′ sense SARS-CoV-2 DNA target non-structural proteins (NSPs)
aatgtagtagaattatacctg

SEQ ID NO: 472 - DNA, 21 bp, SARS coronavirus 2
  5′ sense SARS-CoV-2 DNA target non-structural proteins (NSPs)
aaattcaaagtgaattcaaca

SEQ ID NO: 473 - DNA, 21 bp, SARS coronavirus 2
  5′ sense SARS-CoV-2 DNA target non-structural proteins (NSPs)
aattcaaagtgaattcaacat

SEQ ID NO: 474 - DNA, 21 bp, SARS coronavirus 2
  5′ sense SARS-CoV-2 DNA target non-structural proteins (NSPs)
aaagtgaattcaacattagaa

SEQ ID NO: 475 - DNA, 21 bp, SARS coronavirus 2
  5′ sense SARS-CoV-2 DNA target non-structural proteins (NSPs)
aagtgaattcaacattagaac

SEQ ID NO: 476 - DNA, 21 bp, SARS coronavirus 2
  5′ sense SARS-CoV-2 DNA target non-structural proteins (NSPs)
aattcagtgtgtagacttatg

SEQ ID NO: 477 - DNA, 21 bp, SARS coronavirus 2
  5′ sense SARS-CoV-2 DNA target non-structural proteins (NSPs)
aagcttaaagcacataaagac

SEQ ID NO: 478 - DNA, 21 bp, SARS coronavirus 2
  5′ sense SARS-CoV-2 DNA target non-structural proteins (NSPs)
aagcacataaagacaaatcag

SEQ ID NO: 479 - DNA, 21 bp, SARS coronavirus 2
  5′ sense SARS-CoV-2 DNA target non-structural proteins (NSPs)
aattaacaggccacaaatagg

SEQ ID NO: 480 - DNA, 21 bp, SARS coronavirus 2
  5′ sense SARS-CoV-2 DNA target non-structural proteins (NSPs)
aaataggcgtggtaagagaat

SEQ ID NO: 481 - DNA, 21 bp, SARS coronavirus 2
  5′ sense SARS-CoV-2 DNA target non-structural proteins (NSPs)
aataggcgtggtaagagaatt

SEQ ID NO: 482 - DNA, 21 bp, SARS coronavirus 2
  5′ sense SARS-CoV-2 DNA target non-structural proteins (NSPs)
aatgctgtagcctcaaagatt

SEQ ID NO: 483 - DNA, 21 bp, SARS coronavirus 2
  5′ sense SARS-CoV-2 DNA target non-structural proteins (NSPs)
aactcaaactgttgattcatc

SEQ ID NO: 484 - DNA, 21 bp, SARS coronavirus 2
  5′ sense SARS-CoV-2 DNA target non-structural proteins (NSPs)
aatatgactatgtcatattca

SEQ ID NO: 485 - DNA, 21 bp, SARS coronavirus 2
  5′ sense SARS-CoV-2 DNA target non-structural proteins (NSPs)
aatgtgactatgtcatattca

SEQ ID NO: 486 - DNA, 21 bp, SARS coronavirus 2
  5′ sense SARS-CoV-2 DNA target non-structural proteins (NSPs)
aaacagctcactcttgtaatg

SEQ ID NO: 487 - DNA, 21 bp, SARS coronavirus 2
  5′ sense SARS-CoV-2 DNA target non-structural proteins (NSPs)
aacagctcactcttgtaatgt

SEQ ID NO: 488 - DNA, 21 bp, SARS coronavirus 2
  5′ sense SARS-CoV-2 DNA target non-structural proteins (NSPs)
taaattagtcttgtctgttaa

SEQ ID NO: 489 - DNA, 21 bp, SARS coronavirus 2
  5′ sense SARS-CoV-2 DNA target non-structural proteins (NSPs)
ccattgtgtgctaatggacaa

SEQ ID NO: 490 - DNA, 21 bp, SARS coronavirus 2
  5′ sense SARS-CoV-2 DNA target non-structural proteins (NSPs)
tacatgtgttggtagcgataa

SEQ ID NO: 491 - DNA, 21 bp, SARS coronavirus 2
  5′ sense SARS-CoV-2 DNA target non-structural proteins (NSPs)
ttagctaacacctgtactgaa

SEQ ID NO: 492 - DNA, 21 bp, SARS coronavirus 2
  5′ sense SARS-CoV-2 DNA target non-structural proteins (NSPs)
tagctaacacctgtactgaaa

SEQ ID NO: 493 - DNA, 21 bp, SARS coronavirus 2
  5′ sense SARS-CoV-2 DNA target non-structural proteins (NSPs)
cacctgtactgaaagactcaa

SEQ ID NO: 494 - DNA, 21 bp, SARS coronavirus 2
  5′ sense SARS-CoV-2 DNA target non-structural proteins (NSPs)
taaacctagaccaccacttaa

SEQ ID NO: 495 - DNA, 21 bp, SARS coronavirus 2
  5′ sense SARS-CoV-2 DNA target non-structural proteins (NSPs)
ctagaccaccacttaaccgaa

SEQ ID NO: 496 - DNA, 19 bp, SARS coronavirus 2
  5′ sense SARS-CoV-2 DNA target non-structural proteins (NSPs)
gcaccacgcacattgctaa

SEQ ID NO: 497 - DNA, 23 bp, SARS coronavirus 2
  5′ sense SARS-CoV-2 DNA target non-structural proteins (NSPs)
acctgcaccacgcacattgctaa

SEQ ID NO: 498 - DNA, 25 bp, SARS coronavirus 2
  5′ sense SARS-CoV-2 DNA target non-structural proteins (NSPs)
ttacctgcaccacgcacattgctaa

SEQ ID NO: 499 - DNA, 21 bp, SARS coronavirus 2
  5′ sense SARS-CoV-2 DNA target non-structural proteins (NSPs)
tagaccaccacttaaccgaaa

SEQ ID NO: 500 - DNA, 21 bp, SARS coronavirus 2
  5′ sense SARS-CoV-2 DNA target non-structural proteins (NSPs)
cacatacagtaatgccattaa

SEQ ID NO: 501 - DNA, 21 bp, SARS coronavirus 2
  5′ sense SARS-CoV-2 DNA target non-structural proteins (NSPs)
atcaaaaggttggtatgcaaa

SEQ ID NO: 502 - DNA, 21 bp, SARS coronavirus 2
  5′ sense SARS-CoV-2 DNA target non-structural proteins (NSPs)
tgttgatgcactatgtgagaa

SEQ ID NO: 503 - DNA, 21 bp, SARS coronavirus 2
  5′ sense SARS-CoV-2 DNA target non-structural proteins (NSPs)
cactatgtgagaaggcattaa

SEQ ID NO: 504 - DNA, 21 bp, SARS coronavirus 2
  5′ sense SARS-CoV-2 DNA target non-structural proteins (NSPs)
actatgtgagaaggcattaaa

SEQ ID NO: 505 - DNA, 21 bp, SARS coronavirus 2
  5′ sense SARS-CoV-2 DNA target non-structural proteins (NSPs)
ctatagataaatgtagtagaa

SEQ ID NO: 506 - DNA, 21 bp, SARS coronavirus 2
  5′ sense SARS-CoV-2 DNA target non-structural proteins (NSPs)
ctgcaccacgcacattgctaa

SEQ ID NO: 507 - DNA, 21 bp, SARS coronavirus 2
  5′ sense SARS-CoV-2 DNA target non-structural proteins (NSPs)
accacgcacattgctaactaa

SEQ ID NO: 508 - DNA, 21 bp, SARS coronavirus 2
  5′ sense SARS-CoV-2 DNA target non-structural proteins (NSPs)
taataagcttaaagcacataa

SEQ ID NO: 509 - DNA, 21 bp, SARS coronavirus 2
  5′ sense SARS-CoV-2 DNA target non-structural proteins (NSPs)
aataagcttaaagcacataaa

SEQ ID NO: 510 - DNA, 21 bp, SARS coronavirus 2
  5′ sense SARS-CoV-2 DNA target non-structural proteins (NSPs)
caaataggcgtggtaagagaa

SEQ ID NO: 511 - DNA, 21 bp, SARS coronavirus 2
  5′ sense SARS-CoV-2 DNA target non-structural proteins (NSPs)
aagagaattccttacacgtaa

SEQ ID NO: 512 - DNA, 21 bp, SARS coronavirus 2
  5′ sense SARS-CoV-2 DNA target non-structural proteins (NSPs)
ttcaccttataattcacagaa

SEQ ID NO: 513 - DNA, 21 bp, SARS coronavirus 2
  5′ sense SARS-CoV-2 DNA target non-structural proteins (NSPs)
gactatgtcatattcactcaa

SEQ ID NO: 514 - DNA, 21 bp, SARS coronavirus 2
  5′ sense SARS-CoV-2 DNA target non-structural proteins (NSPs)
actatgtcatattcactcaaa

SEQ ID NO: 515 - DNA, 21 bp, SARS coronavirus 2
  5′ sense SARS-CoV-2 DNA target non-structural proteins (NSPs)
tgaaacagctcactcttgtaa

SEQ ID NO: 516 - DNA, 21 bp, SARS coronavirus 2
  5′ sense SARS-CoV-2 DNA target non-structural proteins (NSPs)
gttgctattaccagagcaaaa

SEQ ID NO: 517 - DNA, 21 bp, SARS coronavirus 2
  5′ sense SARS-CoV-2 DNA target non-structural proteins (NSPs)
aatgctccaggttgtgatgtc

SEQ ID NO: 518 - DNA, 21 bp, SARS coronavirus 2
  5′ sense SARS-CoV-2 DNA target non-structural proteins (NSPs)
aatgcaattgcaacatgtgac

SEQ ID NO: 519 - DNA, 21 bp, SARS coronavirus 2
  5′ sense SARS-CoV-2 DNA target non-structural proteins (NSPs)
aacatgtgactggacaaatgc

SEQ ID NO: 520 - DNA, 21 bp, SARS coronavirus 2
  5′ sense SARS-CoV-2 DNA target non-structural proteins (NSPs)
aacacctgtactgaaagactc

SEQ ID NO: 521 - DNA, 21 bp, SARS coronavirus 2
  5′ sense SARS-CoV-2 DNA target non-structural proteins (NSPs)
aactgtcttatggtattgcta

SEQ ID NO: 522 - DNA, 21 bp, SARS coronavirus 2
  5′ sense SARS-CoV-2 DNA target non-structural proteins (NSPs)
aagtgctgtctgacagagaat

SEQ ID NO: 523 - DNA, 21 bp, SARS coronavirus 2
  5′ sense SARS-CoV-2 DNA target non-structural proteins (NSPs)
aaacctagaccaccacttaac

SEQ ID NO: 524 - DNA, 21 bp, SARS coronavirus 2
  5′ sense SARS-CoV-2 DNA target non-structural proteins (NSPs)
aacctagaccaccacttaacc

SEQ ID NO: 525 - DNA, 21 bp, SARS coronavirus 2
  5′ sense SARS-CoV-2 DNA target non-structural proteins (NSPs)
aacagtaaagtacaaatagga

SEQ ID NO: 526 - DNA, 21 bp, SARS coronavirus 2
  5′ sense SARS-CoV-2 DNA target non-structural proteins (NSPs)
aaaaaggtgactatggtgatg

SEQ ID NO: 527 - DNA, 21 bp, SARS coronavirus 2
  5′ sense SARS-CoV-2 DNA target non-structural proteins (NSPs)
aaattaaatgttggtgattat

SEQ ID NO: 528 - DNA, 21 bp, SARS coronavirus 2
  5′ sense SARS-CoV-2 DNA target non-structural proteins (NSPs)
aattatcaaaaggttggtatg

SEQ ID NO: 529 - DNA, 21 bp, SARS coronavirus 2
  5′ sense SARS-CoV-2 DNA target non-structural proteins (NSPs)
aaaaggttggtatgcaaaagt

SEQ ID NO: 530 - DNA, 21 bp, SARS coronavirus 2
  5′ sense SARS-CoV-2 DNA target non-structural proteins (NSPs)
aaaggttggtatgcaaaagta

SEQ ID NO: 531 - DNA, 21 bp, SARS coronavirus 2
  5′ sense SARS-CoV-2 DNA target non-structural proteins (NSPs)
aattcaacattagaacagtat

SEQ ID NO: 532 - DNA, 21 bp, SARS coronavirus 2
  5′ sense SARS-CoV-2 DNA target non-structural proteins (NSPs)
aacattagaacagtatgtctt

SEQ ID NO: 533 - DNA, 21 bp, SARS coronavirus 2
  5′ sense SARS-CoV-2 DNA target non-structural proteins (NSPs)
aagcactatgtgtacattggc

SEQ ID NO: 534 - DNA, 21 bp, SARS coronavirus 2
  5′ sense SARS-CoV-2 DNA target non-structural proteins (NSPs)
aactataggtccagacatgtt

SEQ ID NO: 535 - DNA, 21 bp, SARS coronavirus 2
  5′ sense SARS-CoV-2 DNA target non-structural proteins (NSPs)
aataagcttaaagcacataaa

SEQ ID NO: 536 - DNA, 21 bp, SARS coronavirus 2
  5′ sense SARS-CoV-2 DNA target non-structural proteins (NSPs)
aaagcacataaagacaaatca

SEQ ID NO: 537 - DNA, 21 bp, SARS coronavirus 2
  5′ sense SARS-CoV-2 DNA target non-structural proteins (NSPs)
aagagaattccttacacgtaa

SEQ ID NO: 538 - DNA, 21 bp, SARS coronavirus 2
  5′ sense SARS-CoV-2 DNA target non-structural proteins (NSPs)
gatgtcacagatgtgactcaa

SEQ ID NO: 539 - DNA, 21 bp, SARS coronavirus 2
  5′ sense SARS-CoV-2 DNA target non-structural proteins (NSPs)
ttgcaacatgtgactggacaa

SEQ ID NO: 540 - DNA, 21 bp, SARS coronavirus 2
  5′ sense SARS-CoV-2 DNA target non-structural proteins (NSPs)
tgcaacatgtgactggacaaa

SEQ ID NO: 541 - DNA, 21 bp, SARS coronavirus 2
  5′ sense SARS-CoV-2 DNA target non-structural proteins (NSPs)
ggtattgctactgtacgtgaa

SEQ ID NO: 542 - DNA, 21 bp, SARS coronavirus 2
  5′ sense SARS-CoV-2 DNA target non-structural proteins (NSPs)
gaagtgctgtctgacagagaa

SEQ ID NO: 543 - DNA, 21 bp, SARS coronavirus 2
  5′ sense SARS-CoV-2 DNA target non-structural proteins (NSPs)
tatcaaaaggttggtatgcaa

SEQ ID NO: 544 - DNA, 21 bp, SARS coronavirus 2
  5′ sense SARS-CoV-2 DNA target non-structural proteins (NSPs)
caatgccagattacgtgctaa

SEQ ID NO: 545 - DNA, 21 bp, SARS coronavirus 2
  5′ sense SARS-CoV-2 DNA target non-structural proteins (NSPs)
gtcggcgttgtcctgctgaaa

SEQ ID NO: 546 - DNA, 21 bp, SARS coronavirus 2
  5′ sense SARS-CoV-2 DNA target non-structural proteins (NSPs)
gtcgccgttgtcctgctgaaa

SEQ ID NO: 547 - DNA, 21 bp, SARS coronavirus 2
  5′ sense SARS-CoV-2 DNA target non-structural proteins (NSPs)
cataaagacaaatcagctcaa

SEQ ID NO: 548 - DNA, 21 bp, SARS coronavirus 2
  5′ sense SARS-CoV-2 DNA target non-structural proteins (NSPs)
cacagaatgctgtagcctcaa

SEQ ID NO: 549 - DNA, 21 bp, SARS coronavirus 2
  5′ sense SARS-CoV-2 DNA target non-structural proteins (NSPs)
acagaatgctgtagcctcaaa

SEQ ID NO: 550 - DNA, 21 bp, SARS coronavirus 2
  5′ sense SARS-CoV-2 DNA target non-structural proteins (NSPs)
atattcactcaaaccactgaa

SEQ ID NO: 551 - DNA, 21 bp, SARS coronavirus 2
  5′ sense SARS-CoV-2 DNA target non-structural proteins (NSPs)
tattcactcaaaccactgaaa

SEQ ID NO: 552 - DNA, 21 bp, SARS coronavirus 2
  5′ sense SARS-CoV-2 DNA target non-structural proteins (NSPs)
tgttgctattaccagagcaaa

SEQ ID NO: 553 - DNA, 21 bp, SARS coronavirus 2
  5′ sense SARS-CoV-2 DNA target non-structural proteins (NSPs)
aaatgctgttacgaccatgtc

SEQ ID NO: 554 - DNA, 21 bp, SARS coronavirus 2
  5′ sense SARS-CoV-2 DNA target non-structural proteins (NSPs)
aatgctgttacgaccatgtca

SEQ ID NO: 555 - DNA, 21 bp, SARS coronavirus 2
  5′ sense SARS-CoV-2 DNA target non-structural proteins (NSPs)
aaaaatacatgtgttggtagc

SEQ ID NO: 556 - DNA, 21 bp, SARS coronavirus 2
  5′ sense SARS-CoV-2 DNA target non-structural proteins (NSPs)
aaaatacatgtgttggtagcg

SEQ ID NO: 557 - DNA, 21 bp, SARS coronavirus 2
  5′ sense SARS-CoV-2 DNA target non-structural proteins (NSPs)
aattgcaacatgtgactggac

SEQ ID NO: 558 - DNA, 21 bp, SARS coronavirus 2
  5′ sense SARS-CoV-2 DNA target non-structural proteins (NSPs)
aaacgctcaaagctactgagg

SEQ ID NO: 559 - DNA, 21 bp, SARS coronavirus 2
  5′ sense SARS-CoV-2 DNA target non-structural proteins (NSPs)
aacgctcaaagctactgagga

SEQ ID NO: 560 - DNA, 21 bp, SARS coronavirus 2
  5′ sense SARS-CoV-2 DNA target non-structural proteins (NSPs)
aaactgtcttatggtattgct

SEQ ID NO: 561 - DNA, 21 bp, SARS coronavirus 2
  5′ sense SARS-CoV-2 DNA target non-structural proteins (NSPs)
aagttggtaaacctagaccac

SEQ ID NO: 562 - DNA, 21 bp, SARS coronavirus 2
  5′ sense SARS-CoV-2 DNA target non-structural proteins (NSPs)
aaagtacaaataggagagtac

SEQ ID NO: 563 - DNA, 21 bp, SARS coronavirus 2
  5′ sense SARS-CoV-2 DNA target non-structural proteins (NSPs)
aagtacaaataggagagtaca

SEQ ID NO: 564 - DNA, 21 bp, SARS coronavirus 2
  5′ sense SARS-CoV-2 DNA target non-structural proteins (NSPs)
aaaaggtgactatggtgatgc

SEQ ID NO: 565 - DNA, 21 bp, SARS coronavirus 2
  5′ sense SARS-CoV-2 DNA target non-structural proteins (NSPs)
aaggtgactatggtgatgctg

SEQ ID NO: 566 - DNA, 21 bp, SARS coronavirus 2
  5′ sense SARS-CoV-2 DNA target non-structural proteins (NSPs)
aatgccattaagtgcacctac

SEQ ID NO: 567 - DNA, 21 bp, SARS coronavirus 2
  5′ sense SARS-CoV-2 DNA target non-structural proteins (NSPs)
aagtgcacctacactagtgcc

SEQ ID NO: 568 - DNA, 21 bp, SARS coronavirus 2
  5′ sense SARS-CoV-2 DNA target non-structural proteins (NSPs)
aacactcaatatctcagatga

SEQ ID NO: 569 - DNA, 21 bp, SARS coronavirus 2
  5′ sense SARS-CoV-2 DNA target non-structural proteins (NSPs)
aaattatcaaaaggttggtat

SEQ ID NO: 570 - DNA, 21 bp, SARS coronavirus 2
  5′ sense SARS-CoV-2 DNA target non-structural proteins (NSPs)
aaatgcattgcctgagacgac

SEQ ID NO: 571 - DNA, 21 bp, SARS coronavirus 2
  5′ sense SARS-CoV-2 DNA target non-structural proteins (NSPs)
aatgccagattacgtgctaag

SEQ ID NO: 572 - DNA, 21 bp, SARS coronavirus 2
  5′ sense SARS-CoV-2 DNA target non-structural proteins (NSPs)
aaaactataggtccagacatg

SEQ ID NO: 573 - DNA, 21 bp, SARS coronavirus 2
  5′ sense SARS-CoV-2 DNA target non-structural proteins (NSPs)
aaactataggtccagacatgt

SEQ ID NO: 574 - DNA, 21 bp, SARS coronavirus 2
  5′ sense SARS-CoV-2 DNA target non-structural proteins (NSPs)
aacttgtcggcgttgtcctgc

SEQ ID NO: 575 - DNA, 21 bp, SARS coronavirus 2
  5′ sense SARS-CoV-2 DNA target non-structural proteins (NSPs)
aaattgttgacactgtgagtg

SEQ ID NO: 576 - DNA, 21 bp, SARS coronavirus 2
  5′ sense SARS-CoV-2 DNA target non-structural proteins (NSPs)
aattgttgacactgtgagtgc

SEQ ID NO: 577 - DNA, 21 bp, SARS coronavirus 2
  5′ sense SARS-CoV-2 DNA target non-structural proteins (NSPs)
aaagacaaatcagctcaatgc

SEQ ID NO: 578 - DNA, 21 bp, SARS coronavirus 2
  5′ sense SARS-CoV-2 DNA target non-structural proteins (NSPs)
aagacaaatcagctcaatgct

SEQ ID NO: 579 - DNA, 21 bp, SARS coronavirus 2
  5′ sense SARS-CoV-2 DNA target non-structural proteins (NSPs)
aacaggccacaaataggcgtg

SEQ ID NO: 580 - DNA, 21 bp, SARS coronavirus 2
  5′ sense SARS-CoV-2 DNA target non-structural proteins (NSPs)
aacagacctcaaataggcgtt

SEQ ID NO: 581 - DNA, 21 bp, SARS coronavirus 2
  5′ sense SARS-CoV-2 DNA target non-structural proteins (NSPs)
aaactgttgattcatcacagg

SEQ ID NO: 582 - DNA, 21 bp, SARS coronavirus 2
  5′ sense SARS-CoV-2 DNA target non-structural proteins (NSPs)
aaccactgaaacagctcactc

SEQ ID NO: 583 - DNA, 21 bp, SARS coronavirus 2
  5′ sense SARS-CoV-2 DNA target non-structural proteins (NSPs)
gcacctacactagtgccacaa

SEQ ID NO: 584 - DNA, 21 bp, SARS coronavirus 2
  5′ sense SARS-CoV-2 DNA target non-structural proteins (NSPs)
gtccagacatgttcctcggaa

SEQ ID NO: 585 - DNA, 21 bp, SARS coronavirus 2
  5′ sense SARS-CoV-2 DNA target non-structural proteins (NSPs)
tgtcggcgttgtcctgctgaa

SEQ ID NO: 586 - DNA, 21 bp, SARS coronavirus 2
  5′ sense SARS-CoV-2 DNA target non-structural proteins (NSPs)
tctgcaattaacaggccacaa

SEQ ID NO: 587 - DNA, 21 bp, SARS coronavirus 2
  5′ sense SARS-CoV-2 DNA target non-structural proteins (NSPs)
ctgcaattaacaggccacaaa

SEQ ID NO: 588 - DNA, 21 bp, SARS coronavirus 2
  5′ sense SARS-CoV-2 DNA target non-structural proteins (NSPs)
ggccacaaataggcgtggtaa

SEQ ID NO: 589 - DNA, 21 bp, SARS coronavirus 2
  5′ sense SARS-CoV-2 DNA target non-structural proteins (NSPs)
atgttgctattaccagagcaa

SEQ ID NO: 590 - DNA, 21 bp, SARS coronavirus 2
  5′ sense SARS-CoV-2 DNA target non-structural proteins (NSPs)
aaatacatgtgttggtagcga

SEQ ID NO: 591 - DNA, 21 bp, SARS coronavirus 2
  5′ sense SARS-CoV-2 DNA target non-structural proteins (NSPs)
aatacatgtgttggtagcgat

SEQ ID NO: 592 - DNA, 21 bp, SARS coronavirus 2
  5′ sense SARS-CoV-2 DNA target non-structural proteins (NSPs)
aaaggtgactatggtgatgct

SEQ ID NO: 593 - DNA, 21 bp, SARS coronavirus 2
  5′ sense SARS-CoV-2 DNA target non-structural proteins (NSPs)
aaaagtattctacactccagg

SEQ ID NO: 594 - DNA, 21 bp, SARS coronavirus 2
  5′ sense SARS-CoV-2 DNA target non-structural proteins (NSPs)
aattatacctgcacgtgctcg

SEQ ID NO: 595 - DNA, 21 bp, SARS coronavirus 2
  5′ sense SARS-CoV-2 DNA target non-structural proteins (NSPs)
aatgcattgcctgagacgaca

SEQ ID NO: 596 - DNA, 21 bp, SARS coronavirus 2
  5′ sense SARS-CoV-2 DNA target non-structural proteins (NSPs)
aattacctgcaccacgcacat

SEQ ID NO: 597 - DNA, 21 bp, SARS coronavirus 2
  5′ sense SARS-CoV-2 DNA target non-structural proteins (NSPs)
aattcacagaatgctgtagcc

SEQ ID NO: 598 - DNA, 21 bp, SARS coronavirus 2
  5′ sense SARS-CoV-2 DNA target non-structural proteins (NSPs)
aaaccactgaaacagctcact

SEQ ID NO: 599 - DNA, 21 bp, SARS coronavirus 2
  5′ sense SARS-CoV-2 DNA target non-structural proteins (NSPs)
aatgttgctattaccagagca

SEQ ID NO: 600 - RNA, 21 nt, Artificial Sequence
  5′ sense siRNA sequences non-structural proteins (NSPs)
agcugauguuacuaaaauaaa

SEQ ID NO: 601 - RNA, 21 nt, Artificial Sequence
  5′ sense siRNA sequences non-structural proteins (NSPs)
gcugauguuacuaaaauaaaa

SEQ ID NO: 602 - RNA, 21 nt, Artificial Sequence
  5′ sense siRNA sequences non-structural proteins (NSPs)
auggagcugauguuacuaaaa

SEQ ID NO: 603 - RNA, 21 nt, Artificial Sequence
  5′ sense siRNA sequences non-structural proteins (NSPs)
gagcugauguuacuaaaauaa

SEQ ID NO: 604 - RNA, 21 nt, Artificial Sequence
  5′ sense siRNA sequences non-structural proteins (NSPs)
guggucacuauaaacauauaa

SEQ ID NO: 605 - RNA, 21 nt, Artificial Sequence
  5′ sense siRNA sequences non-structural proteins (NSPs)
aaauaaaaccucauaauucac

SEQ ID NO: 606 - RNA, 21 nt, Artificial Sequence
  5′ sense siRNA sequences non-structural proteins (NSPs)
aaacaagcuacaaaauaucua

SEQ ID NO: 607 - RNA, 21 nt, Artificial Sequence
  5′ sense siRNA sequences non-structural proteins (NSPs)
aacaagcuacaaaauaucuag

SEQ ID NO: 608 - RNA, 21 nt, Artificial Sequence
  5′ sense siRNA sequences non-structural proteins (NSPs)
ggauggagcugauguuacuaa

SEQ ID NO: 609 - RNA, 21 nt, Artificial Sequence
  5′ sense siRNA sequences non-structural proteins (NSPs)
gauggagcugauguuacuaaa

SEQ ID NO: 610 - RNA, 21 nt, Artificial Sequence
  5′ sense siRNA sequences non-structural proteins (NSPs)
auaauucacaugaagguaaaa

SEQ ID NO: 611 - RNA, 21 nt, Artificial Sequence
  5′ sense siRNA sequences non-structural proteins (NSPs)
uagguacaugucagcauuaaa

SEQ ID NO: 612 - RNA, 21 nt, Artificial Sequence
  5′ sense siRNA sequences non-structural proteins (NSPs)
cagcauuaaaucacacuaaaa

SEQ ID NO: 613 - RNA, 21 nt, Artificial Sequence
  5′ sense siRNA sequences non-structural proteins (NSPs)
agcauuaaaucacacuaaaaa

SEQ ID NO: 614 - RNA, 21 nt, Artificial Sequence
  5′ sense siRNA sequences non-structural proteins (NSPs)
aucuugccacugcauuguuaa

SEQ ID NO: 615 - RNA, 21 nt, Artificial Sequence
  5′ sense siRNA sequences non-structural proteins (NSPs)
uaucuuagccuacuguaauaa

SEQ ID NO: 616 - RNA, 21 nt, Artificial Sequence
  5′ sense siRNA sequences non-structural proteins (NSPs)
aguuaggugauguuagagaaa

SEQ ID NO: 617 - RNA, 21 nt, Artificial Sequence
  5′ sense siRNA sequences non-structural proteins (NSPs)
ucuugaacgugguguguaaaa

SEQ ID NO: 618 - RNA, 21 nt, Artificial Sequence
  5′ sense siRNA sequences non-structural proteins (NSPs)
accugcucaguaugaacuuaa

SEQ ID NO: 619 - RNA, 21 nt, Artificial Sequence
  5′ sense siRNA sequences non-structural proteins (NSPs)
uuaccaguguggucacuauaa

SEQ ID NO: 620 - RNA, 21 nt, Artificial Sequence
  5′ sense siRNA sequences non-structural proteins (NSPs)
uaccaguguggucacuauaaa

SEQ ID NO: 621 - RNA, 21 nt, Artificial Sequence
  5′ sense siRNA sequences non-structural proteins (NSPs)
cuauaaacauauaacuucuaa

SEQ ID NO: 622 - RNA, 21 nt, Artificial Sequence
  5′ sense siRNA sequences non-structural proteins (NSPs)
uauaaacauauaacuucuaaa

SEQ ID NO: 623 - RNA, 21 nt, Artificial Sequence
  5′ sense siRNA sequences non-structural proteins (NSPs)
aaacauauaacuucuaaagaa

SEQ ID NO: 624 - RNA, 21 nt, Artificial Sequence
  5′ sense siRNA sequences non-structural proteins (NSPs)
aacauauaacuucuaaagaaa

SEQ ID NO: 625 - RNA, 21 nt, Artificial Sequence
  5′ sense siRNA sequences non-structural proteins (NSPs)
uacaaaguccucagaauacaa

SEQ ID NO: 626 - RNA, 21 nt, Artificial Sequence
  5′ sense siRNA sequences non-structural proteins (NSPs)
acaaaguccucagaauacaaa

SEQ ID NO: 627 - RNA, 21 nt, Artificial Sequence
  5′ sense siRNA sequences non-structural proteins (NSPs)
aaaauaaaaccucauaauuca

SEQ ID NO: 628 - RNA, 21 nt, Artificial Sequence
  5′ sense siRNA sequences non-structural proteins (NSPs)
aauaaaaccucauaauucaca

SEQ ID NO: 629 - RNA, 21 nt, Artificial Sequence
  5′ sense siRNA sequences non-structural proteins (NSPs)
aacacuccaacaaauagaguu

SEQ ID NO: 630 - RNA, 21 nt, Artificial Sequence
  5′ sense siRNA sequences non-structural proteins (NSPs)
aacgugguguguaaaacuugu

SEQ ID NO: 631 - RNA, 21 nt, Artificial Sequence
  5′ sense siRNA sequences non-structural proteins (NSPs)
aagcuacaaaauaucuaguac

SEQ ID NO: 632 - RNA, 21 nt, Artificial Sequence
  5′ sense siRNA sequences non-structural proteins (NSPs)
aaacauauaacuucuaaagaa

SEQ ID NO: 633 - RNA, 21 nt, Artificial Sequence
  5′ sense siRNA sequences non-structural proteins (NSPs)
aacauauaacuucuaaagaaa

SEQ ID NO: 634 - RNA, 21 nt, Artificial Sequence
  5′ sense siRNA sequences non-structural proteins (NSPs)
aaaguccucagaauacaaagg

SEQ ID NO: 635 - RNA, 21 nt, Artificial Sequence
  5′ sense siRNA sequences non-structural proteins (NSPs)
uacuaaaauaaaaccucauaa

SEQ ID NO: 636 - RNA, 21 nt, Artificial Sequence
  5′ sense siRNA sequences non-structural proteins (NSPs)
aaaccucauaauucacaugaa

SEQ ID NO: 637 - RNA, 21 nt, Artificial Sequence
  5′ sense siRNA sequences non-structural proteins (NSPs)
ucauaauucacaugaagguaa

SEQ ID NO: 638 - RNA, 21 nt, Artificial Sequence
  5′ sense siRNA sequences non-structural proteins (NSPs)
cauaauucacaugaagguaaa

SEQ ID NO: 639 - RNA, 21 nt, Artificial Sequence
  5′ sense siRNA sequences non-structural proteins (NSPs)
guagguacaugucagcauuaa

SEQ ID NO: 640 - RNA, 21 nt, Artificial Sequence
  5′ sense siRNA sequences non-structural proteins (NSPs)
gucagcauuaaaucacacuaa

SEQ ID NO: 641 - RNA, 21 nt, Artificial Sequence
  5′ sense siRNA sequences non-structural proteins (NSPs)
ucagcauuaaaucacacuaaa

SEQ ID NO: 642 - RNA, 21 nt, Artificial Sequence
  5′ sense siRNA sequences non-structural proteins (NSPs)
aaaucacacuaaaaaguggaa

SEQ ID NO: 643 - RNA, 21 nt, Artificial Sequence
  5′ sense siRNA sequences non-structural proteins (NSPs)
aaucacacuaaaaaguggaaa

SEQ ID NO: 644 - RNA, 21 nt, Artificial Sequence
  5′ sense siRNA sequences non-structural proteins (NSPs)
cauuguuaacacuccaacaaa

SEQ ID NO: 645 - RNA, 21 nt, Artificial Sequence
  5′ sense siRNA sequences non-structural proteins (NSPs)
acuccaacaaauagaguugaa

SEQ ID NO: 646 - RNA, 21 nt, Artificial Sequence
  5′ sense siRNA sequences non-structural proteins (NSPs)
acuuaucuuagccuacuguaa

SEQ ID NO: 647 - RNA, 21 nt, Artificial Sequence
  5′ sense siRNA sequences non-structural proteins (NSPs)
gaguuaggugauguuagagaa

SEQ ID NO: 648 - RNA, 21 nt, Artificial Sequence
  5′ sense siRNA sequences non-structural proteins (NSPs)
uaggugauguuagagaaacaa

SEQ ID NO: 649 - RNA, 21 nt, Artificial Sequence
  5′ sense siRNA sequences non-structural proteins (NSPs)
uucuugcaaaagagucuugaa

SEQ ID NO: 650 - RNA, 21 nt, Artificial Sequence
  5′ sense siRNA sequences non-structural proteins (NSPs)
gucuugaacgugguguguaaa

SEQ ID NO: 651 - RNA, 21 nt, Artificial Sequence
  5′ sense siRNA sequences non-structural proteins (NSPs)
auaccuuguacgugugguaaa

SEQ ID NO: 652 - RNA, 21 nt, Artificial Sequence
  5′ sense siRNA sequences non-structural proteins (NSPs)
ccuuguacgugugguaaacaa

SEQ ID NO: 653 - RNA, 21 nt, Artificial Sequence
  5′ sense siRNA sequences non-structural proteins (NSPs)
cgugugguaaacaagcuacaa

SEQ ID NO: 654 - RNA, 21 nt, Artificial Sequence
  5′ sense siRNA sequences non-structural proteins (NSPs)
gugugguaaacaagcuacaaa

SEQ ID NO: 655 - RNA, 21 nt, Artificial Sequence
  5′ sense siRNA sequences non-structural proteins (NSPs)
ugugguaaacaagcuacaaaa

SEQ ID NO: 656 - RNA, 21 nt, Artificial Sequence
  5′ sense siRNA sequences non-structural proteins (NSPs)
gcuacaaaauaucuaguacaa

SEQ ID NO: 657 - RNA, 21 nt, Artificial Sequence
  5′ sense siRNA sequences non-structural proteins (NSPs)
gcaccaccugcucaguaugaa

SEQ ID NO: 658 - RNA, 21 nt, Artificial Sequence
  5′ sense siRNA sequences non-structural proteins (NSPs)
gcaccaccugcugaguauaaa

SEQ ID NO: 659 - RNA, 19 nt, Artificial Sequence
  5′ sense siRNA sequences non-structural proteins (NSPs)
accaccugcucaguaugaa

SEQ ID NO: 660 - RNA, 23 nt, Artificial Sequence
  5′ sense siRNA sequences non-structural proteins (NSPs)
cagcaccaccugcucaguaugaa

SEQ ID NO: 661 - RNA, 25 nt, Artificial Sequence
  5′ sense siRNA sequences non-structural proteins (NSPs)
gucagcaccaccugcucaguaugaa

SEQ ID NO: 662 - RNA, 21 nt, Artificial Sequence
  5′ sense siRNA sequences non-structural proteins (NSPs)
acaaagaaaacaguuacacaa

SEQ ID NO: 663 - RNA, 21 nt, Artificial Sequence
  5′ sense siRNA sequences non-structural proteins (NSPs)
aagaaaacaguuacacaacaa

SEQ ID NO: 664 - RNA, 21 nt, Artificial Sequence
  5′ sense siRNA sequences non-structural proteins (NSPs)
aaugacauauggacaacaguu

SEQ ID NO: 665 - RNA, 21 nt, Artificial Sequence
  5′ sense siRNA sequences non-structural proteins (NSPs)
aaaaccucauaauucacauga

SEQ ID NO: 666 - RNA, 21 nt, Artificial Sequence
  5′ sense siRNA sequences non-structural proteins (NSPs)
aaaccucauaauucacaugaa

SEQ ID NO: 667 - RNA, 21 nt, Artificial Sequence
  5′ sense siRNA sequences non-structural proteins (NSPs)
aaccucauaauucacaugaag

SEQ ID NO: 668 - RNA, 21 nt, Artificial Sequence
  5′ sense siRNA sequences non-structural proteins (NSPs)
aauucacaugaagguaaaaca

SEQ ID NO: 669 - RNA, 21 nt, Artificial Sequence
  5′ sense siRNA sequences non-structural proteins (NSPs)
aaaucacacuaaaaaguggaa

SEQ ID NO: 670 - RNA, 21 nt, Artificial Sequence
  5′ sense siRNA sequences non-structural proteins (NSPs)
aaucacacuaaaaaguggaaa

SEQ ID NO: 671 - RNA, 21 nt, Artificial Sequence
  5′ sense siRNA sequences non-structural proteins (NSPs)
aauccaccugcucuacaagau

SEQ ID NO: 672 - RNA, 21 nt, Artificial Sequence
  5′ sense siRNA sequences non-structural proteins (NSPs)
aaagguguucagauaccuugu

SEQ ID NO: 673 - RNA, 21 nt, Artificial Sequence
  5′ sense siRNA sequences non-structural proteins (NSPs)
aagguguucagauaccuugua

SEQ ID NO: 674 - RNA, 21 nt, Artificial Sequence
  5′ sense siRNA sequences non-structural proteins (NSPs)
aaguccucagaauacaaaggu

SEQ ID NO: 675 - RNA, 21 nt, Artificial Sequence
  5′ sense siRNA sequences non-structural proteins (NSPs)
aauacaaagguccuauuacgg

SEQ ID NO: 676 - RNA, 21 nt, Artificial Sequence
  5′ sense siRNA sequences non-structural proteins (NSPs)
aaagaaaacaguuacacaaca

SEQ ID NO: 677 - RNA, 21 nt, Artificial Sequence
  5′ sense siRNA sequences non-structural proteins (NSPs)
aagaaaacaguuacacaacaa

SEQ ID NO: 678 - RNA, 21 nt, Artificial Sequence
  5′ sense siRNA sequences non-structural proteins (NSPs)
aaacaguuacacaacaaccau

SEQ ID NO: 679 - RNA, 21 nt, Artificial Sequence
  5′ sense siRNA sequences non-structural proteins (NSPs)
aacaguuacacaacaaccauu

SEQ ID NO: 680 - RNA, 21 nt, Artificial Sequence
  5′ sense siRNA sequences non-structural proteins (NSPs)
acugcauuguuaacacuccaa

SEQ ID NO: 681 - RNA, 21 nt, Artificial Sequence
  5′ sense siRNA sequences non-structural proteins (NSPs)
gcauuguuaacacuccaacaa

SEQ ID NO: 682 - RNA, 21 nt, Artificial Sequence
  5′ sense siRNA sequences non-structural proteins (NSPs)
aagaugcuuauuacagagcaa

SEQ ID NO: 683 - RNA, 21 nt, Artificial Sequence
  5′ sense siRNA sequences non-structural proteins (NSPs)
agucuugaacgugguguguaa

SEQ ID NO: 684 - RNA, 21 nt, Artificial Sequence
  5′ sense siRNA sequences non-structural proteins (NSPs)
guguguaaaacuuguggacaa

SEQ ID NO: 685 - RNA, 21 nt, Artificial Sequence
  5′ sense siRNA sequences non-structural proteins (NSPs)
cuuguggacaacagcagacaa

SEQ ID NO: 686 - RNA, 21 nt, Artificial Sequence
  5′ sense siRNA sequences non-structural proteins (NSPs)
gauaccuuguacgugugguaa

SEQ ID NO: 687 - RNA, 21 nt, Artificial Sequence
  5′ sense siRNA sequences non-structural proteins (NSPs)
ugcuagugaguacacugguaa

SEQ ID NO: 688 - RNA, 21 nt, Artificial Sequence
  5′ sense siRNA sequences non-structural proteins (NSPs)
uuacuuacaaaguccucagaa

SEQ ID NO: 689 - RNA, 21 nt, Artificial Sequence
  5′ sense siRNA sequences non-structural proteins (NSPs)
aaugaugacacucuacguguu

SEQ ID NO: 690 - RNA, 21 nt, Artificial Sequence
  5′ sense siRNA sequences non-structural proteins (NSPs)
aagaugcuuauuacagagcaa

SEQ ID NO: 691 - RNA, 21 nt, Artificial Sequence
  5′ sense siRNA sequences non-structural proteins (NSPs)
aauaagacaguaggugaguua

SEQ ID NO: 692 - RNA, 21 nt, Artificial Sequence
  5′ sense siRNA sequences non-structural proteins (NSPs)
aagacaguaggugaguuaggu

SEQ ID NO: 693 - RNA, 21 nt, Artificial Sequence
  5′ sense siRNA sequences non-structural proteins (NSPs)
aaaagagucuugaacguggug

SEQ ID NO: 694 - RNA, 21 nt, Artificial Sequence
  5′ sense siRNA sequences non-structural proteins (NSPs)
aaaacuuguggacaacagcag

SEQ ID NO: 695 - RNA, 21 nt, Artificial Sequence
  5′ sense siRNA sequences non-structural proteins (NSPs)
aaacuuguggacaacagcaga

SEQ ID NO: 696 - RNA, 21 nt, Artificial Sequence
  5′ sense siRNA sequences non-structural proteins (NSPs)
aacuuguggacaacagcagac

SEQ ID NO: 697 - RNA, 21 nt, Artificial Sequence
  5′ sense siRNA sequences non-structural proteins (NSPs)
aagaaagguguucagauaccu

SEQ ID NO: 698 - RNA, 21 nt, Artificial Sequence
  5′ sense siRNA sequences non-structural proteins (NSPs)
aaaauaucuaguacaacagga

SEQ ID NO: 699 - RNA, 21 nt, Artificial Sequence
  5′ sense siRNA sequences non-structural proteins (NSPs)
aaauaucuaguacaacaggag

SEQ ID NO: 700 - RNA, 21 nt, Artificial Sequence
  5′ sense siRNA sequences non-structural proteins (NSPs)
aauaucuaguacaacaggagu

SEQ ID NO: 701 - RNA, 21 nt, Artificial Sequence
  5′ sense siRNA sequences non-structural proteins (NSPs)
aauuaccaguguggucacuau

SEQ ID NO: 702 - RNA, 21 nt, Artificial Sequence
  5′ sense siRNA sequences non-structural proteins (NSPs)
aaagguccuauuacggauguu

SEQ ID NO: 703 - RNA, 21 nt, Artificial Sequence
  5′ sense siRNA sequences non-structural proteins (NSPs)
aaaacaguuacacaacaacca

SEQ ID NO: 704 - RNA, 21 nt, Artificial Sequence
  5′ sense siRNA sequences non-structural proteins (NSPs)
aacaacuguuaucuugccacu

SEQ ID NO: 705 - RNA, 21 nt, Artificial Sequence
  5′ sense siRNA sequences non-structural proteins (NSPs)
aaagagucuugaacguggugu

SEQ ID NO: 706 - RNA, 21 nt, Artificial Sequence
  5′ sense siRNA sequences non-structural proteins (NSPs)
aagagucuugaacguggugug

SEQ ID NO: 707 - RNA, 21 nt, Artificial Sequence
  5′ sense siRNA sequences non-structural proteins (NSPs)
aacuguuaucuugccacugca

SEQ ID NO: 708 - RNA, 21 nt, Artificial Sequence
  5′ sense siRNA sequences non-structural proteins (NSPs)
cauucguaagucuaaucauaa

SEQ ID NO: 709 - RNA, 21 nt, Artificial Sequence
  5′ sense siRNA sequences non-structural proteins (NSPs)
guuguacgcugcuguuauaaa

SEQ ID NO: 710 - RNA, 21 nt, Artificial Sequence
  5′ sense siRNA sequences non-structural proteins (NSPs)
gcuaugaaguacaauuaugaa

SEQ ID NO: 711 - RNA, 21 nt, Artificial Sequence
  5′ sense siRNA sequences non-structural proteins (NSPs)
uugugguacaacuacacuuaa

SEQ ID NO: 712 - RNA, 21 nt, Artificial Sequence
  5′ sense siRNA sequences non-structural proteins (NSPs)
guacaggcugguaauguucaa

SEQ ID NO: 713 - RNA, 21 nt, Artificial Sequence
  5′ sense siRNA sequences non-structural proteins (NSPs)
uauuggacauucuaugcaaaa

SEQ ID NO: 714 - RNA, 21 nt, Artificial Sequence
  5′ sense siRNA sequences non-structural proteins (NSPs)
uaugcaaaauuguguacuuaa

SEQ ID NO: 715 - RNA, 21 nt, Artificial Sequence
  5′ sense siRNA sequences non-structural proteins (NSPs)
aaauuguguacuuaagcuuaa

SEQ ID NO: 716 - RNA, 21 nt, Artificial Sequence
  5′ sense siRNA sequences non-structural proteins (NSPs)
auccuaagacaccuaagauaa

SEQ ID NO: 717 - RNA, 21 nt, Artificial Sequence
  5′ sense siRNA sequences non-structural proteins (NSPs)
uucaguguuagcuuguuacaa

SEQ ID NO: 718 - RNA, 21 nt, Artificial Sequence
  5′ sense siRNA sequences non-structural proteins (NSPs)
ugcaccauauggaauuaccaa

SEQ ID NO: 719 - RNA, 21 nt, Artificial Sequence
  5′ sense siRNA sequences non-structural proteins (NSPs)
ggacacaacuauuacaguuaa

SEQ ID NO: 720 - RNA, 21 nt, Artificial Sequence
  5′ sense siRNA sequences non-structural proteins (NSPs)
gguuguacgcugcuguuauaa

SEQ ID NO: 721 - RNA, 21 nt, Artificial Sequence
  5′ sense siRNA sequences non-structural proteins (NSPs)
aaaauuguguacuuaagcuua

SEQ ID NO: 722 - RNA, 21 nt, Artificial Sequence
  5′ sense siRNA sequences non-structural proteins (NSPs)
aaauuguguacuuaagcuuaa

SEQ ID NO: 723 - RNA, 21 nt, Artificial Sequence
  5′ sense siRNA sequences non-structural proteins (NSPs)
aauuguguacuuaagcuuaag

SEQ ID NO: 724 - RNA, 21 nt, Artificial Sequence
  5′ sense siRNA sequences non-structural proteins (NSPs)
aaccuuguggcuaugaaguac

SEQ ID NO: 725 - RNA, 21 nt, Artificial Sequence
  5′ sense siRNA sequences non-structural proteins (NSPs)
aagaccauguugacauacuag

SEQ ID NO: 726 - RNA, 21 nt, Artificial Sequence
  5′ sense siRNA sequences non-structural proteins (NSPs)
gcaccucugaagacaugcuaa

SEQ ID NO: 727 - RNA, 21 nt, Artificial Sequence
  5′ sense siRNA sequences non-structural proteins (NSPs)
guuauuggacauucuaugcaa

SEQ ID NO: 728 - RNA, 21 nt, Artificial Sequence
  5′ sense siRNA sequences non-structural proteins (NSPs)
uuauuggacauucuaugcaaa

SEQ ID NO: 729 - RNA, 21 nt, Artificial Sequence
  5′ sense siRNA sequences non-structural proteins (NSPs)
gguugauacagccaauccuaa

SEQ ID NO: 730 - RNA, 21 nt, Artificial Sequence
  5′ sense siRNA sequences non-structural proteins (NSPs)
uguuacaugcaccauauggaa

SEQ ID NO: 731 - RNA, 21 nt, Artificial Sequence
  5′ sense siRNA sequences non-structural proteins (NSPs)
caugcuggcacagacuuagaa

SEQ ID NO: 732 - RNA, 21 nt, Artificial Sequence
  5′ sense siRNA sequences non-structural proteins (NSPs)
uggcacagacuuagaagguaa

SEQ ID NO: 733 - RNA, 21 nt, Artificial Sequence
  5′ sense siRNA sequences non-structural proteins (NSPs)
ccuuguggcuaugaaguacaa

SEQ ID NO: 734 - RNA, 21 nt, Artificial Sequence
  5′ sense siRNA sequences non-structural proteins (NSPs)
aguacaauuaugaaccucuaa

SEQ ID NO: 735 - RNA, 21 nt, Artificial Sequence
  5′ sense siRNA sequences non-structural proteins (NSPs)
aauuaugaaccucuaacacaa

SEQ ID NO: 736 - RNA, 21 nt, Artificial Sequence
  5′ sense siRNA sequences non-structural proteins (NSPs)
aaguaacuugugguacaacua

SEQ ID NO: 737 - RNA, 21 nt, Artificial Sequence
  5′ sense siRNA sequences non-structural proteins (NSPs)
aacuugugguacaacuacacu

SEQ ID NO: 738 - RNA, 21 nt, Artificial Sequence
  5′ sense siRNA sequences non-structural proteins (NSPs)
aauccuaagacaccuaagaua

SEQ ID NO: 739 - RNA, 21 nt, Artificial Sequence
  5′ sense siRNA sequences non-structural proteins (NSPs)
aauuaccaacuggaguucaug

SEQ ID NO: 740 - RNA, 21 nt, Artificial Sequence
  5′ sense siRNA sequences non-structural proteins (NSPs)
aauuaugaaccucuaacacaa

SEQ ID NO: 741 - RNA, 21 nt, Artificial Sequence
  5′ sense siRNA sequences non-structural proteins (NSPs)
aaccucuaacacaagaccaug

SEQ ID NO: 742 - RNA, 21 nt, Artificial Sequence
  5′ sense siRNA sequences non-structural proteins (NSPs)
aacacaagaccauguugacau

SEQ ID NO: 743 - RNA, 21 nt, Artificial Sequence
  5′ sense siRNA sequences non-structural proteins (NSPs)
uacaaguaacuugugguacaa

SEQ ID NO: 744 - RNA, 21 nt, Artificial Sequence
  5′ sense siRNA sequences non-structural proteins (NSPs)
caugugaucugcaccucugaa

SEQ ID NO: 745 - RNA, 21 nt, Artificial Sequence
  5′ sense siRNA sequences non-structural proteins (NSPs)
gcuuaagguugauacagccaa

SEQ ID NO: 746 - RNA, 21 nt, Artificial Sequence
  5′ sense siRNA sequences non-structural proteins (NSPs)
agccaauccuaagacaccuaa

SEQ ID NO: 747 - RNA, 21 nt, Artificial Sequence
  5′ sense siRNA sequences non-structural proteins (NSPs)
guugacaggcaaacagcacaa

SEQ ID NO: 748 - RNA, 21 nt, Artificial Sequence
  5′ sense siRNA sequences non-structural proteins (NSPs)
aagcuuaagguugauacagcc

SEQ ID NO: 749 - RNA, 21 nt, Artificial Sequence
  5′ sense siRNA sequences non-structural proteins (NSPs)
aaugguucaccaucugguguu

SEQ ID NO: 750 - RNA, 21 nt, Artificial Sequence
  5′ sense siRNA sequences non-structural proteins (NSPs)
aaugguucaugugguaguguu

SEQ ID NO: 751 - RNA, 21 nt, Artificial Sequence
  5′ sense siRNA sequences non-structural proteins (NSPs)
aacagcacaagcagcugguac

SEQ ID NO: 752 - RNA, 21 nt, Artificial Sequence
  5′ sense siRNA sequences non-structural proteins (NSPs)
aacugcacaggcugcagguac

SEQ ID NO: 753 - RNA, 21 nt, Artificial Sequence
  5′ sense siRNA sequences non-structural proteins (NSPs)
aaguacaauuaugaaccucua

SEQ ID NO: 754 - RNA, 21 nt, Artificial Sequence
  5′ sense siRNA sequences non-structural proteins (NSPs)
aagcagcugguacggacacaa

SEQ ID NO: 755 - RNA, 21 nt, Artificial Sequence
  5′ sense siRNA sequences non-structural proteins (NSPs)
aagacaugugaucugcaccuc

SEQ ID NO: 756 - RNA, 21 nt, Artificial Sequence
  5′ sense siRNA sequences non-structural proteins (NSPs)
aagguugauacagccaauccu

SEQ ID NO: 757 - RNA, 21 nt, Artificial Sequence
  5′ sense siRNA sequences non-structural proteins (NSPs)
aacuggaguucaugcuggcac

SEQ ID NO: 758 - RNA, 21 nt, Artificial Sequence
  5′ sense siRNA sequences non-structural proteins (NSPs)
aaacagcacaagcagcuggua

SEQ ID NO: 759 - RNA, 21 nt, Artificial Sequence
  5′ sense siRNA sequences non-structural proteins (NSPs)
aagcagcugguacggacacaa

SEQ ID NO: 760 - RNA, 21 nt, Artificial Sequence
  5′ sense siRNA sequences non-structural proteins (NSPs)
ugcuaaauuccuaaaaacuaa

SEQ ID NO: 761 - RNA, 21 nt, Artificial Sequence
  5′ sense siRNA sequences non-structural proteins (NSPs)
gguaauugugacacauuaaaa

SEQ ID NO: 762 - RNA, 21 nt, Artificial Sequence
  5′ sense siRNA sequences non-structural proteins (NSPs)
aauugugacacauuaaaagaa

SEQ ID NO: 763 - RNA, 21 nt, Artificial Sequence
  5′ sense siRNA sequences non-structural proteins (NSPs)
auugugacacauuaaaagaaa

SEQ ID NO: 764 - RNA, 21 nt, Artificial Sequence
  5′ sense siRNA sequences non-structural proteins (NSPs)
ugguguacugacauuagauaa

SEQ ID NO: 765 - RNA, 21 nt, Artificial Sequence
  5′ sense siRNA sequences non-structural proteins (NSPs)
cauuguuaaugccuauauuaa

SEQ ID NO: 766 - RNA, 21 nt, Artificial Sequence
  5′ sense siRNA sequences non-structural proteins (NSPs)
cuuaacaaagccuuacauuaa

SEQ ID NO: 767 - RNA, 21 nt, Artificial Sequence
  5′ sense siRNA sequences non-structural proteins (NSPs)
aaggaaguucuguugaauuaa

SEQ ID NO: 768 - RNA, 21 nt, Artificial Sequence
  5′ sense siRNA sequences non-structural proteins (NSPs)
aggaaguucuguugaauuaaa

SEQ ID NO: 769 - RNA, 21 nt, Artificial Sequence
  5′ sense siRNA sequences non-structural proteins (NSPs)
ggaaguucuguugaauuaaaa

SEQ ID NO: 770 - RNA, 21 nt, Artificial Sequence
  5′ sense siRNA sequences non-structural proteins (NSPs)
gguuggcacaacauguuaaaa

SEQ ID NO: 771 - RNA, 21 nt, Artificial Sequence
  5′ sense siRNA sequences non-structural proteins (NSPs)
guuggcacaacauguuaaaaa

SEQ ID NO: 772 - RNA, 21 nt, Artificial Sequence
  5′ sense siRNA sequences non-structural proteins (NSPs)
uggcgguucacuauauguuaa

SEQ ID NO: 773 - RNA, 21 nt, Artificial Sequence
  5′ sense siRNA sequences non-structural proteins (NSPs)
ggcgguucacuauauguuaaa

SEQ ID NO: 774 - RNA, 21 nt, Artificial Sequence
  5′ sense siRNA sequences non-structural proteins (NSPs)
aauugugacacauuaaaagaa

SEQ ID NO: 775 - RNA, 21 nt, Artificial Sequence
  5′ sense siRNA sequences non-structural proteins (NSPs)
aauacuugucacauacaauug

SEQ ID NO: 776 - RNA, 21 nt, Artificial Sequence
  5′ sense siRNA sequences non-structural proteins (NSPs)
aaggaaguucuguugaauuaa

SEQ ID NO: 777 - RNA, 21 nt, Artificial Sequence
  5′ sense siRNA sequences non-structural proteins (NSPs)
aaguucuguugaauuaaaaca

SEQ ID NO: 778 - RNA, 21 nt, Artificial Sequence
  5′ sense siRNA sequences non-structural proteins (NSPs)
aaaaauuauugaaaucaauag

SEQ ID NO: 779 - RNA, 21 nt, Artificial Sequence
  5′ sense siRNA sequences non-structural proteins (NSPs)
aacauacaaugcuaguuaaac

SEQ ID NO: 780 - RNA, 21 nt, Artificial Sequence
  5′ sense siRNA sequences non-structural proteins (NSPs)
cuaacuaccaacaugaagaaa

SEQ ID NO: 781 - RNA, 21 nt, Artificial Sequence
  5′ sense siRNA sequences non-structural proteins (NSPs)
acuaccaacaugaagaaacaa

SEQ ID NO: 782 - RNA, 21 nt, Artificial Sequence
  5′ sense siRNA sequences non-structural proteins (NSPs)
aucacgucaacgucuuacuaa

SEQ ID NO: 783 - RNA, 21 nt, Artificial Sequence
  5′ sense siRNA sequences non-structural proteins (NSPs)
ucacgucaacgucuuacuaaa

SEQ ID NO: 784 - RNA, 21 nt, Artificial Sequence
  5′ sense siRNA sequences non-structural proteins (NSPs)
aacgucuuacuaaauacacaa

SEQ ID NO: 785 - RNA, 21 nt, Artificial Sequence
  5′ sense siRNA sequences non-structural proteins (NSPs)
aagguaauugugacacauuaa

SEQ ID NO: 786 - RNA, 21 nt, Artificial Sequence
  5′ sense siRNA sequences non-structural proteins (NSPs)
agguaauugugacacauuaaa

SEQ ID NO: 787 - RNA, 21 nt, Artificial Sequence
  5′ sense siRNA sequences non-structural proteins (NSPs)
guacugacauuagauaaucaa

SEQ ID NO: 788 - RNA, 21 nt, Artificial Sequence
  5′ sense siRNA sequences non-structural proteins (NSPs)
auucuuauuauucauuguuaa

SEQ ID NO: 789 - RNA, 21 nt, Artificial Sequence
  5′ sense siRNA sequences non-structural proteins (NSPs)
auguugacacugacuuaacaa

SEQ ID NO: 790 - RNA, 21 nt, Artificial Sequence
  5′ sense siRNA sequences non-structural proteins (NSPs)
uguugacacugacuuaacaaa

SEQ ID NO: 791 - RNA, 21 nt, Artificial Sequence
  5′ sense siRNA sequences non-structural proteins (NSPs)
uucacggaagagagguuaaaa

SEQ ID NO: 792 - RNA, 21 nt, Artificial Sequence
  5′ sense siRNA sequences non-structural proteins (NSPs)
uuggaccacuagugagaaaaa

SEQ ID NO: 793 - RNA, 21 nt, Artificial Sequence
  5′ sense siRNA sequences non-structural proteins (NSPs)
agagcuagguguuguacauaa

SEQ ID NO: 794 - RNA, 21 nt, Artificial Sequence
  5′ sense siRNA sequences non-structural proteins (NSPs)
ugguaaucuauuacuagauaa

SEQ ID NO: 795 - RNA, 21 nt, Artificial Sequence
  5′ sense siRNA sequences non-structural proteins (NSPs)
gguaaucuauuacuagauaaa

SEQ ID NO: 796 - RNA, 21 nt, Artificial Sequence
  5′ sense siRNA sequences non-structural proteins (NSPs)
uucaguagcugcacuuacuaa

SEQ ID NO: 797 - RNA, 21 nt, Artificial Sequence
  5′ sense siRNA sequences non-structural proteins (NSPs)
aguagcugcacuuacuaacaa

SEQ ID NO: 798 - RNA, 21 nt, Artificial Sequence
  5′ sense siRNA sequences non-structural proteins (NSPs)
uuaugacuacuaucguuauaa

SEQ ID NO: 799 - RNA, 21 nt, Artificial Sequence
  5′ sense siRNA sequences non-structural proteins (NSPs)
uguaguugaaguuguugauaa

SEQ ID NO: 800 - RNA, 21 nt, Artificial Sequence
  5′ sense siRNA sequences non-structural proteins (NSPs)
uuacgaugguggcuguauuaa

SEQ ID NO: 801 - RNA, 21 nt, Artificial Sequence
  5′ sense siRNA sequences non-structural proteins (NSPs)
ugguggcuguauuaaugcuaa

SEQ ID NO: 802 - RNA, 21 nt, Artificial Sequence
  5′ sense siRNA sequences non-structural proteins (NSPs)
uauaacucaaaugaaucuuaa

SEQ ID NO: 803 - RNA, 21 nt, Artificial Sequence
  5′ sense siRNA sequences non-structural proteins (NSPs)
aucaaaaauuauugaaaucaa

SEQ ID NO: 804 - RNA, 21 nt, Artificial Sequence
  5′ sense siRNA sequences non-structural proteins (NSPs)
gagcuacuguaguaauuggaa

SEQ ID NO: 805 - RNA, 21 nt, Artificial Sequence
  5′ sense siRNA sequences non-structural proteins (NSPs)
gugguuggcacaacauguuaa

SEQ ID NO: 806 - RNA, 21 nt, Artificial Sequence
  5′ sense siRNA sequences non-structural proteins (NSPs)
ugguuggcacaacauguuaaa

SEQ ID NO: 807 - RNA, 21 nt, Artificial Sequence
  5′ sense siRNA sequences non-structural proteins (NSPs)
uucuugcucgcaaacauacaa

SEQ ID NO: 808 - RNA, 21 nt, Artificial Sequence
  5′ sense siRNA sequences non-structural proteins (NSPs)
ugccacaacugcuuaugcuaa

SEQ ID NO: 809 - RNA, 21 nt, Artificial Sequence
  5′ sense siRNA sequences non-structural proteins (NSPs)
agcugucacggccaauguuaa

SEQ ID NO: 810 - RNA, 21 nt, Artificial Sequence
  5′ sense siRNA sequences non-structural proteins (NSPs)
uaucuacugaugguaacaaaa

SEQ ID NO: 811 - RNA, 21 nt, Artificial Sequence
  5′ sense siRNA sequences non-structural proteins (NSPs)
uuaugagugucucuauagaaa

SEQ ID NO: 812 - RNA, 21 nt, Artificial Sequence
  5′ sense siRNA sequences non-structural proteins (NSPs)
aggucuaguggcuagcauaaa

SEQ ID NO: 813 - RNA, 21 nt, Artificial Sequence
  5′ sense siRNA sequences non-structural proteins (NSPs)
ucuaguggcuagcauaaagaa

SEQ ID NO: 814 - RNA, 21 nt, Artificial Sequence
  5′ sense siRNA sequences non-structural proteins (NSPs)
acugagacugaccuuacuaaa

SEQ ID NO: 815 - RNA, 21 nt, Artificial Sequence
  5′ sense siRNA sequences non-structural proteins (NSPs)
caacauacaaugcuaguuaaa

SEQ ID NO: 816 - RNA, 21 nt, Artificial Sequence
  5′ sense siRNA sequences non-structural proteins (NSPs)
uuguagaugauaucguaaaaa

SEQ ID NO: 817 - RNA, 21 nt, Artificial Sequence
  5′ sense siRNA sequences non-structural proteins (NSPs)
gaugguacacuuaugauugaa

SEQ ID NO: 818 - RNA, 21 nt, Artificial Sequence
  5′ sense siRNA sequences non-structural proteins (NSPs)
uguacuuacaauacauaagaa

SEQ ID NO: 819 - RNA, 21 nt, Artificial Sequence
  5′ sense siRNA sequences non-structural proteins (NSPs)
guacuuacaauacauaagaaa

SEQ ID NO: 820 - RNA, 21 nt, Artificial Sequence
  5′ sense siRNA sequences non-structural proteins (NSPs)
gaaagcuacaugaugaguuaa

SEQ ID NO: 821 - RNA, 21 nt, Artificial Sequence
  5′ sense siRNA sequences non-structural proteins (NSPs)
aaauuccuaaaaacuaauugu

SEQ ID NO: 822 - RNA, 21 nt, Artificial Sequence
  5′ sense siRNA sequences non-structural proteins (NSPs)
aauuccuaaaaacuaauuguu

SEQ ID NO: 823 - RNA, 21 nt, Artificial Sequence
  5′ sense siRNA sequences non-structural proteins (NSPs)
aaaaggacgaagaugacaauu

SEQ ID NO: 824 - RNA, 21 nt, Artificial Sequence
  5′ sense siRNA sequences non-structural proteins (NSPs)
aacgucuuacuaaauacacaa

SEQ ID NO: 825 - RNA, 21 nt, Artificial Sequence
  5′ sense siRNA sequences non-structural proteins (NSPs)
aagguaauugugacacauuaa

SEQ ID NO: 826 - RNA, 21 nt, Artificial Sequence
  5′ sense siRNA sequences non-structural proteins (NSPs)
aaauacuugucacauacaauu

SEQ ID NO: 827 - RNA, 21 nt, Artificial Sequence
  5′ sense siRNA sequences non-structural proteins (NSPs)
aauuguugugaugaugauuau

SEQ ID NO: 828 - RNA, 21 nt, Artificial Sequence
  5′ sense siRNA sequences non-structural proteins (NSPs)
aaucaagaucucaaugguaac

SEQ ID NO: 829 - RNA, 21 nt, Artificial Sequence
  5′ sense siRNA sequences non-structural proteins (NSPs)
aacaaagccuuacauuaagug

SEQ ID NO: 830 - RNA, 21 nt, Artificial Sequence
  5′ sense siRNA sequences non-structural proteins (NSPs)
aaucaggauguaaacuuacau

SEQ ID NO: 831 - RNA, 21 nt, Artificial Sequence
  5′ sense siRNA sequences non-structural proteins (NSPs)
aacucaaaugaaucuuaagua

SEQ ID NO: 832 - RNA, 21 nt, Artificial Sequence
  5′ sense siRNA sequences non-structural proteins (NSPs)
aaaauuauugaaaucaauagc

SEQ ID NO: 833 - RNA, 21 nt, Artificial Sequence
  5′ sense siRNA sequences non-structural proteins (NSPs)
aauuggaacaagcaaauucua

SEQ ID NO: 834 - RNA, 21 nt, Artificial Sequence
  5′ sense siRNA sequences non-structural proteins (NSPs)
aaugagugugcucaaguauug

SEQ ID NO: 835 - RNA, 21 nt, Artificial Sequence
  5′ sense siRNA sequences non-structural proteins (NSPs)
aacugcuuaugcuaauagugu

SEQ ID NO: 836 - RNA, 21 nt, Artificial Sequence
  5′ sense siRNA sequences non-structural proteins (NSPs)
aacaaaauugccgauaaguau

SEQ ID NO: 837 - RNA, 21 nt, Artificial Sequence
  5′ sense siRNA sequences non-structural proteins (NSPs)
aaaauugccgauaaguauguc

SEQ ID NO: 838 - RNA, 21 nt, Artificial Sequence
  5′ sense siRNA sequences non-structural proteins (NSPs)
aaaaacagaugguacacuuau

SEQ ID NO: 839 - RNA, 21 nt, Artificial Sequence
  5′ sense siRNA sequences non-structural proteins (NSPs)
aaaacagaugguacacuuaug

SEQ ID NO: 840 - RNA, 21 nt, Artificial Sequence
  5′ sense siRNA sequences non-structural proteins (NSPs)
aacagaugguacacuuaugau

SEQ ID NO: 841 - RNA, 21 nt, Artificial Sequence
  5′ sense siRNA sequences non-structural proteins (NSPs)
aaagcuacaugaugaguuaac

SEQ ID NO: 842 - RNA, 21 nt, Artificial Sequence
  5′ sense siRNA sequences non-structural proteins (NSPs)
uauggcuguaguugugaucaa

SEQ ID NO: 843 - RNA, 21 nt, Artificial Sequence
  5′ sense siRNA sequences non-structural proteins (NSPs)
aauuguugucgcuuccaagaa

SEQ ID NO: 844 - RNA, 21 nt, Artificial Sequence
  5′ sense siRNA sequences non-structural proteins (NSPs)
auuguugucgcuuccaagaaa

SEQ ID NO: 845 - RNA, 21 nt, Artificial Sequence
  5′ sense siRNA sequences non-structural proteins (NSPs)
uuguugucgcuuccaagaaaa

SEQ ID NO: 846 - RNA, 21 nt, Artificial Sequence
  5′ sense siRNA sequences non-structural proteins (NSPs)
agaaaaggacgaagaugacaa

SEQ ID NO: 847 - RNA, 21 nt, Artificial Sequence
  5′ sense siRNA sequences non-structural proteins (NSPs)
uucucuaacuaccaacaugaa

SEQ ID NO: 848 - RNA, 21 nt, Artificial Sequence
  5′ sense siRNA sequences non-structural proteins (NSPs)
ucuaacuaccaacaugaagaa

SEQ ID NO: 849 - RNA, 21 nt, Artificial Sequence
  5′ sense siRNA sequences non-structural proteins (NSPs)
ggauuguccagcuguugcuaa

SEQ ID NO: 850 - RNA, 21 nt, Artificial Sequence
  5′ sense siRNA sequences non-structural proteins (NSPs)
gauuguccagcuguugcuaaa

SEQ ID NO: 851 - RNA, 21 nt, Artificial Sequence
  5′ sense siRNA sequences non-structural proteins (NSPs)
agaaauacuugucacauacaa

SEQ ID NO: 852 - RNA, 21 nt, Artificial Sequence
  5′ sense siRNA sequences non-structural proteins (NSPs)
guauacgccaacuuaggugaa

SEQ ID NO: 853 - RNA, 21 nt, Artificial Sequence
  5′ sense siRNA sequences non-structural proteins (NSPs)
auuagauaaucaagaucucaa

SEQ ID NO: 854 - RNA, 21 nt, Artificial Sequence
  5′ sense siRNA sequences non-structural proteins (NSPs)
uaaucaagaucucaaugguaa

SEQ ID NO: 855 - RNA, 21 nt, Artificial Sequence
  5′ sense siRNA sequences non-structural proteins (NSPs)
cacauguugacacugacuuaa

SEQ ID NO: 856 - RNA, 21 nt, Artificial Sequence
  5′ sense siRNA sequences non-structural proteins (NSPs)
acuucacggaagagagguuaa

SEQ ID NO: 857 - RNA, 21 nt, Artificial Sequence
  5′ sense siRNA sequences non-structural proteins (NSPs)
cuucacggaagagagguuaaa

SEQ ID NO: 858 - RNA, 21 nt, Artificial Sequence
  5′ sense siRNA sequences non-structural proteins (NSPs)
gaugcauucugcauugugcaa

SEQ ID NO: 859 - RNA, 21 nt, Artificial Sequence
  5′ sense siRNA sequences non-structural proteins (NSPs)
augcauucugcauugugcaaa

SEQ ID NO: 860 - RNA, 21 nt, Artificial Sequence
  5′ sense siRNA sequences non-structural proteins (NSPs)
uuguacauaaucaggauguaa

SEQ ID NO: 861 - RNA, 21 nt, Artificial Sequence
  5′ sense siRNA sequences non-structural proteins (NSPs)
uguacauaaucaggauguaaa

SEQ ID NO: 862 - RNA, 21 nt, Artificial Sequence
  5′ sense siRNA sequences non-structural proteins (NSPs)
uaugcacgcugcuucugguaa

SEQ ID NO: 863 - RNA, 21 nt, Artificial Sequence
  5′ sense siRNA sequences non-structural proteins (NSPs)
aaggaaggaaguucuguugaa

SEQ ID NO: 864 - RNA, 21 nt, Artificial Sequence
  5′ sense siRNA sequences non-structural proteins (NSPs)
acuaucguuauaaucuaccaa

SEQ ID NO: 865 - RNA, 21 nt, Artificial Sequence
  5′ sense siRNA sequences non-structural proteins (NSPs)
aucguuauaaucuaccaacaa

SEQ ID NO: 866 - RNA, 21 nt, Artificial Sequence
  5′ sense siRNA sequences non-structural proteins (NSPs)
acaaugugugauaucagacaa

SEQ ID NO: 867 - RNA, 21 nt, Artificial Sequence
  5′ sense siRNA sequences non-structural proteins (NSPs)
aucgucaacaaccuagacaaa

SEQ ID NO: 868 - RNA, 21 nt, Artificial Sequence
  5′ sense siRNA sequences non-structural proteins (NSPs)
uuaaguaugccauuagugcaa

SEQ ID NO: 869 - RNA, 21 nt, Artificial Sequence
  5′ sense siRNA sequences non-structural proteins (NSPs)
uaaguaugccauuagugcaaa

SEQ ID NO: 870 - RNA, 21 nt, Artificial Sequence
  5′ sense siRNA sequences non-structural proteins (NSPs)
guaugccauuagugcaaagaa

SEQ ID NO: 871 - RNA, 21 nt, Artificial Sequence
  5′ sense siRNA sequences non-structural proteins (NSPs)
uaucuguaguacuaugaccaa

SEQ ID NO: 872 - RNA, 21 nt, Artificial Sequence
  5′ sense siRNA sequences non-structural proteins (NSPs)
cuagaggagcuacuguaguaa

SEQ ID NO: 873 - RNA, 21 nt, Artificial Sequence
  5′ sense siRNA sequences non-structural proteins (NSPs)
cuacuguaguaauuggaacaa

SEQ ID NO: 874 - RNA, 21 nt, Artificial Sequence
  5′ sense siRNA sequences non-structural proteins (NSPs)
uguaguaauuggaacaagcaa

SEQ ID NO: 875 - RNA, 21 nt, Artificial Sequence
  5′ sense siRNA sequences non-structural proteins (NSPs)
guaguaauuggaacaagcaaa

SEQ ID NO: 876 - RNA, 21 nt, Artificial Sequence
  5′ sense siRNA sequences non-structural proteins (NSPs)
ccaugccuaacaugcuuagaa

SEQ ID NO: 877 - RNA, 21 nt, Artificial Sequence
  5′ sense siRNA sequences non-structural proteins (NSPs)
ugugcucaaguauugagugaa

SEQ ID NO: 878 - RNA, 21 nt, Artificial Sequence
  5′ sense siRNA sequences non-structural proteins (NSPs)
gugcucaaguauugagugaaa

SEQ ID NO: 879 - RNA, 21 nt, Artificial Sequence
  5′ sense siRNA sequences non-structural proteins (NSPs)
uuaucuacugaugguaacaaa

SEQ ID NO: 880 - RNA, 21 nt, Artificial Sequence
  5′ sense siRNA sequences non-structural proteins (NSPs)
aaggucuaguggcuagcauaa

SEQ ID NO: 881 - RNA, 21 nt, Artificial Sequence
  5′ sense siRNA sequences non-structural proteins (NSPs)
gacugagacugaccuuacuaa

SEQ ID NO: 882 - RNA, 21 nt, Artificial Sequence
  5′ sense siRNA sequences non-structural proteins (NSPs)
ucaacauacaaugcuaguuaa

SEQ ID NO: 883 - RNA, 21 nt, Artificial Sequence
  5′ sense siRNA sequences non-structural proteins (NSPs)
guauucuguuaugcuuacuaa

SEQ ID NO: 884 - RNA, 21 nt, Artificial Sequence
  5′ sense siRNA sequences non-structural proteins (NSPs)
aaagguuauggcuguaguugu

SEQ ID NO: 885 - RNA, 21 nt, Artificial Sequence
  5′ sense siRNA sequences non-structural proteins (NSPs)
aagguuauggcuguaguugug

SEQ ID NO: 886 - RNA, 21 nt, Artificial Sequence
  5′ sense siRNA sequences non-structural proteins (NSPs)
aauuguugucgcuuccaagaa

SEQ ID NO: 887 - RNA, 21 nt, Artificial Sequence
  5′ sense siRNA sequences non-structural proteins (NSPs)
aagaaaaggacgaagaugaca

SEQ ID NO: 888 - RNA, 21 nt, Artificial Sequence
  5′ sense siRNA sequences non-structural proteins (NSPs)
aacuaccaacaugaagaaaca

SEQ ID NO: 889 - RNA, 21 nt, Artificial Sequence
  5′ sense siRNA sequences non-structural proteins (NSPs)
aaaagaaauacuugucacaua

SEQ ID NO: 890 - RNA, 21 nt, Artificial Sequence
  5′ sense siRNA sequences non-structural proteins (NSPs)
aaagaaauacuugucacauac

SEQ ID NO: 891 - RNA, 21 nt, Artificial Sequence
  5′ sense siRNA sequences non-structural proteins (NSPs)
aagaaauacuugucacauaca

SEQ ID NO: 892 - RNA, 21 nt, Artificial Sequence
  5′ sense siRNA sequences non-structural proteins (NSPs)
aauaaaaaggacugguaugau

SEQ ID NO: 893 - RNA, 21 nt, Artificial Sequence
  5′ sense siRNA sequences non-structural proteins (NSPs)
aaaaacaguacaauucuguga

SEQ ID NO: 894 - RNA, 21 nt, Artificial Sequence
  5′ sense siRNA sequences non-structural proteins (NSPs)
aaaacaguacaauucugugau

SEQ ID NO: 895 - RNA, 21 nt, Artificial Sequence
  5′ sense siRNA sequences non-structural proteins (NSPs)
aaacaguacaauucugugaug

SEQ ID NO: 896 - RNA, 21 nt, Artificial Sequence
  5′ sense siRNA sequences non-structural proteins (NSPs)
aaugccuauauuaaccuugac

SEQ ID NO: 897 - RNA, 21 nt, Artificial Sequence
  5′ sense siRNA sequences non-structural proteins (NSPs)
aacugcagagucacauguuga

SEQ ID NO: 898 - RNA, 21 nt, Artificial Sequence
  5′ sense siRNA sequences non-structural proteins (NSPs)
aacuuacauagcucuagacuu

SEQ ID NO: 899 - RNA, 21 nt, Artificial Sequence
  5′ sense siRNA sequences non-structural proteins (NSPs)
aaucuauuacuagauaaacgc

SEQ ID NO: 900 - RNA, 21 nt, Artificial Sequence
  5′ sense siRNA sequences non-structural proteins (NSPs)
aaggaaggaaguucuguugaa

SEQ ID NO: 901 - RNA, 21 nt, Artificial Sequence
  5′ sense siRNA sequences non-structural proteins (NSPs)
aaugcugcuaucagcgauuau

SEQ ID NO: 902 - RNA, 21 nt, Artificial Sequence
  5′ sense siRNA sequences non-structural proteins (NSPs)
aaucuaccaacaaugugugau

SEQ ID NO: 903 - RNA, 21 nt, Artificial Sequence
  5′ sense siRNA sequences non-structural proteins (NSPs)
aacaaugugugauaucagaca

SEQ ID NO: 904 - RNA, 21 nt, Artificial Sequence
  5′ sense siRNA sequences non-structural proteins (NSPs)
aaccaagucaucgucaacaac

SEQ ID NO: 905 - RNA, 21 nt, Artificial Sequence
  5′ sense siRNA sequences non-structural proteins (NSPs)
aagucaucgucaacaaccuag

SEQ ID NO: 906 - RNA, 21 nt, Artificial Sequence
  5′ sense siRNA sequences non-structural proteins (NSPs)
aacaaccuagacaaaucagcu

SEQ ID NO: 907 - RNA, 21 nt, Artificial Sequence
  5′ sense siRNA sequences non-structural proteins (NSPs)
aaguaugccauuagugcaaag

SEQ ID NO: 908 - RNA, 21 nt, Artificial Sequence
  5′ sense siRNA sequences non-structural proteins (NSPs)
aaauuauugaaaucaauagcc

SEQ ID NO: 909 - RNA, 21 nt, Artificial Sequence
  5′ sense siRNA sequences non-structural proteins (NSPs)
aacaugcuuagaauuauggcc

SEQ ID NO: 910 - RNA, 21 nt, Artificial Sequence
  5′ sense siRNA sequences non-structural proteins (NSPs)
aauuauggccucacuuguucu

SEQ ID NO: 911 - RNA, 21 nt, Artificial Sequence
  5′ sense siRNA sequences non-structural proteins (NSPs)
aaacauacaacguguuguagc

SEQ ID NO: 912 - RNA, 21 nt, Artificial Sequence
  5′ sense siRNA sequences non-structural proteins (NSPs)
aaguauugagugaaaugguca

SEQ ID NO: 913 - RNA, 21 nt, Artificial Sequence
  5′ sense siRNA sequences non-structural proteins (NSPs)
aagcugucacggccaauguua

SEQ ID NO: 914 - RNA, 21 nt, Artificial Sequence
  5′ sense siRNA sequences non-structural proteins (NSPs)
aaauugccgauaaguaugucc

SEQ ID NO: 915 - RNA, 21 nt, Artificial Sequence
  5′ sense siRNA sequences non-structural proteins (NSPs)
aaggucuaguggcuagcauaa

SEQ ID NO: 916 - RNA, 21 nt, Artificial Sequence
  5′ sense siRNA sequences non-structural proteins (NSPs)
aaacagaugguacacuuauga

SEQ ID NO: 917 - RNA, 21 nt, Artificial Sequence
  5′ sense siRNA sequences non-structural proteins (NSPs)
aacauccuaaucaggaguaug

SEQ ID NO: 918 - RNA, 21 nt, Artificial Sequence
  5′ sense siRNA sequences non-structural proteins (NSPs)
aauacauaagaaagcuacaug

SEQ ID NO: 919 - RNA, 21 nt, Artificial Sequence
  5′ sense siRNA sequences non-structural proteins (NSPs)
aagaaagcuacaugaugaguu

SEQ ID NO: 920 - RNA, 21 nt, Artificial Sequence
  5′ sense siRNA sequences non-structural proteins (NSPs)
aagcuacaugaugaguuaaca

SEQ ID NO: 921 - RNA, 21 nt, Artificial Sequence
  5′ sense siRNA sequences non-structural proteins (NSPs)
aacaggacacauguuagacau

SEQ ID NO: 922 - RNA, 21 nt, Artificial Sequence
  5′ sense siRNA sequences non-structural proteins (NSPs)
aaugauaacacuucaagguau

SEQ ID NO: 923 - RNA, 21 nt, Artificial Sequence
  5′ sense siRNA sequences non-structural proteins (NSPs)
cuucagucagcugaugcacaa

SEQ ID NO: 924 - RNA, 21 nt, Artificial Sequence
  5′ sense siRNA sequences non-structural proteins (NSPs)
cgcuuccaagaaaaggacgaa

SEQ ID NO: 925 - RNA, 21 nt, Artificial Sequence
  5′ sense siRNA sequences non-structural proteins (NSPs)
guaccacauauaucacgucaa

SEQ ID NO: 926 - RNA, 21 nt, Artificial Sequence
  5′ sense siRNA sequences non-structural proteins (NSPs)
uauauuacgcguauacgccaa

SEQ ID NO: 927 - RNA, 21 nt, Artificial Sequence
  5′ sense siRNA sequences non-structural proteins (NSPs)
auucugugaugccaugcgaaa

SEQ ID NO: 928 - RNA, 21 nt, Artificial Sequence
  5′ sense siRNA sequences non-structural proteins (NSPs)
uuaaaauaugacuucacggaa

SEQ ID NO: 929 - RNA, 21 nt, Artificial Sequence
  5′ sense siRNA sequences non-structural proteins (NSPs)
ggcuguauuaaugcuaaccaa

SEQ ID NO: 930 - RNA, 21 nt, Artificial Sequence
  5′ sense siRNA sequences non-structural proteins (NSPs)
ugcuaaccaagucaucgucaa

SEQ ID NO: 931 - RNA, 21 nt, Artificial Sequence
  5′ sense siRNA sequences non-structural proteins (NSPs)
uaaccaagucaucgucaacaa

SEQ ID NO: 932 - RNA, 21 nt, Artificial Sequence
  5′ sense siRNA sequences non-structural proteins (NSPs)
caucgucaacaaccuagacaa

SEQ ID NO: 933 - RNA, 21 nt, Artificial Sequence
  5′ sense siRNA sequences non-structural proteins (NSPs)
ucaaugaguuaugaggaucaa

SEQ ID NO: 934 - RNA, 21 nt, Artificial Sequence
  5′ sense siRNA sequences non-structural proteins (NSPs)
auucuauggugguuggcacaa

SEQ ID NO: 935 - RNA, 21 nt, Artificial Sequence
  5′ sense siRNA sequences non-structural proteins (NSPs)
augugauagagccaugccuaa

SEQ ID NO: 936 - RNA, 21 nt, Artificial Sequence
  5′ sense siRNA sequences non-structural proteins (NSPs)
uuagcuaaugagugugcucaa

SEQ ID NO: 937 - RNA, 21 nt, Artificial Sequence
  5′ sense siRNA sequences non-structural proteins (NSPs)
ccucaucaggagaugccacaa

SEQ ID NO: 938 - RNA, 21 nt, Artificial Sequence
  5′ sense siRNA sequences non-structural proteins (NSPs)
caucauccggugaugcuacaa

SEQ ID NO: 939 - RNA, 21 nt, Artificial Sequence
  5′ sense siRNA sequences non-structural proteins (NSPs)
ugguaacaaaauugccgauaa

SEQ ID NO: 940 - RNA, 21 nt, Artificial Sequence
  5′ sense siRNA sequences non-structural proteins (NSPs)
aauagcacuuaugcaucucaa

SEQ ID NO: 941 - RNA, 21 nt, Artificial Sequence
  5′ sense siRNA sequences non-structural proteins (NSPs)
cuuacuaaaggaccucaugaa

SEQ ID NO: 942 - RNA, 21 nt, Artificial Sequence
  5′ sense siRNA sequences non-structural proteins (NSPs)
aaaaacuaauuguugucgcuu

SEQ ID NO: 943 - RNA, 21 nt, Artificial Sequence
  5′ sense siRNA sequences non-structural proteins (NSPs)
aaaacuaauuguugucgcuuc

SEQ ID NO: 944 - RNA, 21 nt, Artificial Sequence
  5′ sense siRNA sequences non-structural proteins (NSPs)
aauagacggugacaugguacc

SEQ ID NO: 945 - RNA, 21 nt, Artificial Sequence
  5′ sense siRNA sequences non-structural proteins (NSPs)
aauggcagaccucgucuaugc

SEQ ID NO: 946 - RNA, 21 nt, Artificial Sequence
  5′ sense siRNA sequences non-structural proteins (NSPs)
aauggcugauuuagucuaugc

SEQ ID NO: 947 - RNA, 21 nt, Artificial Sequence
  5′ sense siRNA sequences non-structural proteins (NSPs)
aacaguacaauucugugaugc

SEQ ID NO: 948 - RNA, 21 nt, Artificial Sequence
  5′ sense siRNA sequences non-structural proteins (NSPs)
aaaugcugguauuguuggugu

SEQ ID NO: 949 - RNA, 21 nt, Artificial Sequence
  5′ sense siRNA sequences non-structural proteins (NSPs)
aaugcugguauuguuggugua

SEQ ID NO: 950 - RNA, 21 nt, Artificial Sequence
  5′ sense siRNA sequences non-structural proteins (NSPs)
aagaucucaaugguaacuggu

SEQ ID NO: 951 - RNA, 21 nt, Artificial Sequence
  5′ sense siRNA sequences non-structural proteins (NSPs)
aaaccacgccagguaguggag

SEQ ID NO: 952 - RNA, 21 nt, Artificial Sequence
  5′ sense siRNA sequences non-structural proteins (NSPs)
aaguagcaccaggcugcggag

SEQ ID NO: 953 - RNA, 21 nt, Artificial Sequence
  5′ sense siRNA sequences non-structural proteins (NSPs)
aaccacgccagguaguggagu

SEQ ID NO: 954 - RNA, 21 nt, Artificial Sequence
  5′ sense siRNA sequences non-structural proteins (NSPs)
aaauaugacuucacggaagag

SEQ ID NO: 955 - RNA, 21 nt, Artificial Sequence
  5′ sense siRNA sequences non-structural proteins (NSPs)
aacuggauaccacuucagaga

SEQ ID NO: 956 - RNA, 21 nt, Artificial Sequence
  5′ sense siRNA sequences non-structural proteins (NSPs)
aaacuuacauagcucuagacu

SEQ ID NO: 957 - RNA, 21 nt, Artificial Sequence
  5′ sense siRNA sequences non-structural proteins (NSPs)
aaggaauuacuuguguaugcu

SEQ ID NO: 958 - RNA, 21 nt, Artificial Sequence
  5′ sense siRNA sequences non-structural proteins (NSPs)
aauuacuuguguaugcugcug

SEQ ID NO: 959 - RNA, 21 nt, Artificial Sequence
  5′ sense siRNA sequences non-structural proteins (NSPs)
aaugugugauaucagacaacu

SEQ ID NO: 960 - RNA, 21 nt, Artificial Sequence
  5′ sense siRNA sequences non-structural proteins (NSPs)
aaugcuaaccaagucaucguc

SEQ ID NO: 961 - RNA, 21 nt, Artificial Sequence
  5′ sense siRNA sequences non-structural proteins (NSPs)
aaugaguuaugaggaucaaga

SEQ ID NO: 962 - RNA, 21 nt, Artificial Sequence
  5′ sense siRNA sequences non-structural proteins (NSPs)
aaaugaaucuuaaguaugcca

SEQ ID NO: 963 - RNA, 21 nt, Artificial Sequence
  5′ sense siRNA sequences non-structural proteins (NSPs)
aaugaaucuuaaguaugccau

SEQ ID NO: 964 - RNA, 21 nt, Artificial Sequence
  5′ sense siRNA sequences non-structural proteins (NSPs)
aaucuuaaguaugccauuagu

SEQ ID NO: 965 - RNA, 21 nt, Artificial Sequence
  5′ sense siRNA sequences non-structural proteins (NSPs)
aauuauugaaaucaauagccg

SEQ ID NO: 966 - RNA, 21 nt, Artificial Sequence
  5′ sense siRNA sequences non-structural proteins (NSPs)
aaucaauagccgccacuagag

SEQ ID NO: 967 - RNA, 21 nt, Artificial Sequence
  5′ sense siRNA sequences non-structural proteins (NSPs)
aauagccgccacuagaggagc

SEQ ID NO: 968 - RNA, 21 nt, Artificial Sequence
  5′ sense siRNA sequences non-structural proteins (NSPs)
aacaagcaaauucuauggugg

SEQ ID NO: 969 - RNA, 21 nt, Artificial Sequence
  5′ sense siRNA sequences non-structural proteins (NSPs)
aagcaaauucuauggugguug

SEQ ID NO: 970 - RNA, 21 nt, Artificial Sequence
  5′ sense siRNA sequences non-structural proteins (NSPs)
aacauacaacguguuguagcu

SEQ ID NO: 971 - RNA, 21 nt, Artificial Sequence
  5′ sense siRNA sequences non-structural proteins (NSPs)
aacguguuguagcuugucaca

SEQ ID NO: 972 - RNA, 21 nt, Artificial Sequence
  5′ sense siRNA sequences non-structural proteins (NSPs)
aaaccagguggaaccucauca

SEQ ID NO: 973 - RNA, 21 nt, Artificial Sequence
  5′ sense siRNA sequences non-structural proteins (NSPs)
aaccagguggaaccucaucag

SEQ ID NO: 974 - RNA, 21 nt, Artificial Sequence
  5′ sense siRNA sequences non-structural proteins (NSPs)
aaccagguggaacaucauccg

SEQ ID NO: 975 - RNA, 21 nt, Artificial Sequence
  5′ sense siRNA sequences non-structural proteins (NSPs)
aaccucaucaggagaugccac

SEQ ID NO: 976 - RNA, 21 nt, Artificial Sequence
  5′ sense siRNA sequences non-structural proteins (NSPs)
aacaucauccggugaugcuac

SEQ ID NO: 977 - RNA, 21 nt, Artificial Sequence
  5′ sense siRNA sequences non-structural proteins (NSPs)
aauugccgauaaguauguccg

SEQ ID NO: 978 - RNA, 21 nt, Artificial Sequence
  5′ sense siRNA sequences non-structural proteins (NSPs)
aaauagagauguugacacaga

SEQ ID NO: 979 - RNA, 21 nt, Artificial Sequence
  5′ sense siRNA sequences non-structural proteins (NSPs)
aauagagauguugacacagac

SEQ ID NO: 980 - RNA, 21 nt, Artificial Sequence
  5′ sense siRNA sequences non-structural proteins (NSPs)
aauagcacuuaugcaucucaa

SEQ ID NO: 981 - RNA, 21 nt, Artificial Sequence
  5′ sense siRNA sequences non-structural proteins (NSPs)
aaaauguuggacugagacuga

SEQ ID NO: 982 - RNA, 21 nt, Artificial Sequence
  5′ sense siRNA sequences non-structural proteins (NSPs)
aaauguuggacugagacugac

SEQ ID NO: 983 - RNA, 21 nt, Artificial Sequence
  5′ sense siRNA sequences non-structural proteins (NSPs)
aauguuggacugagacugacc

SEQ ID NO: 984 - RNA, 21 nt, Artificial Sequence
  5′ sense siRNA sequences non-structural proteins (NSPs)
aaacauccuaaucaggaguau

SEQ ID NO: 985 - RNA, 21 nt, Artificial Sequence
  5′ sense siRNA sequences non-structural proteins (NSPs)
aaucaggaguaugcugauguc

SEQ ID NO: 986 - RNA, 21 nt, Artificial Sequence
  5′ sense siRNA sequences non-structural proteins (NSPs)
acuaauuguugucgcuuccaa

SEQ ID NO: 987 - RNA, 21 nt, Artificial Sequence
  5′ sense siRNA sequences non-structural proteins (NSPs)
uuaggugaacguguacgccaa

SEQ ID NO: 988 - RNA, 21 nt, Artificial Sequence
  5′ sense siRNA sequences non-structural proteins (NSPs)
aauucugugaugccaugcgaa

SEQ ID NO: 989 - RNA, 21 nt, Artificial Sequence
  5′ sense siRNA sequences non-structural proteins (NSPs)
cucacuuguucuugcucgcaa

SEQ ID NO: 990 - RNA, 21 nt, Artificial Sequence
  5′ sense siRNA sequences non-structural proteins (NSPs)
ucacuuguucuugcucgcaaa

SEQ ID NO: 991 - RNA, 21 nt, Artificial Sequence
  5′ sense siRNA sequences non-structural proteins (NSPs)
uauauguuaaaccagguggaa

SEQ ID NO: 992 - RNA, 21 nt, Artificial Sequence
  5′ sense siRNA sequences non-structural proteins (NSPs)
ugccgauaaguauguccgcaa

SEQ ID NO: 993 - RNA, 21 nt, Artificial Sequence
  5′ sense siRNA sequences non-structural proteins (NSPs)
aacaccgugcggcacaggcac

SEQ ID NO: 994 - RNA, 21 nt, Artificial Sequence
  5′ sense siRNA sequences non-structural proteins (NSPs)
aaacuaauuguugucgcuucc

SEQ ID NO: 995 - RNA, 21 nt, Artificial Sequence
  5′ sense siRNA sequences non-structural proteins (NSPs)
aacuaauuguugucgcuucca

SEQ ID NO: 996 - RNA, 21 nt, Artificial Sequence
  5′ sense siRNA sequences non-structural proteins (NSPs)
aaggauuguccagcuguugcu

SEQ ID NO: 997 - RNA, 21 nt, Artificial Sequence
  5′ sense siRNA sequences non-structural proteins (NSPs)
aaauacacaauggcagaccuc

SEQ ID NO: 998 - RNA, 21 nt, Artificial Sequence
  5′ sense siRNA sequences non-structural proteins (NSPs)
aauacacaauggcagaccucg

SEQ ID NO: 999 - RNA, 21 nt, Artificial Sequence
  5′ sense siRNA sequences non-structural proteins (NSPs)
aacuuaggugaacguguacgc

SEQ ID NO: 1000 - RNA, 21 nt, Artificial Sequence
  5′ sense siRNA sequences non-structural proteins (NSPs)
aauucugugaugccaugcgaa

SEQ ID NO: 1001 - RNA, 21 nt, Artificial Sequence
  5′ sense siRNA sequences non-structural proteins (NSPs)
aaaauaugacuucacggaaga

SEQ ID NO: 1002 - RNA, 21 nt, Artificial Sequence
  5′ sense siRNA sequences non-structural proteins (NSPs)
aauaugacuucacggaagaga

SEQ ID NO: 1003 - RNA, 21 nt, Artificial Sequence
  5′ sense siRNA sequences non-structural proteins (NSPs)
aaccuagacaaaucagcuggu

SEQ ID NO: 1004 - RNA, 21 nt, Artificial Sequence
  5′ sense siRNA sequences non-structural proteins (NSPs)
aauagagcucgcaccguagcu

SEQ ID NO: 1005 - RNA, 21 nt, Artificial Sequence
  5′ sense siRNA sequences non-structural proteins (NSPs)
aaaucaauagccgccacuaga

SEQ ID NO: 1006 - RNA, 21 nt, Artificial Sequence
  5′ sense siRNA sequences non-structural proteins (NSPs)
aaauucuauggugguuggcac

SEQ ID NO: 1007 - RNA, 21 nt, Artificial Sequence
  5′ sense siRNA sequences non-structural proteins (NSPs)
aauucuauggugguuggcaca

SEQ ID NO: 1008 - RNA, 21 nt, Artificial Sequence
  5′ sense siRNA sequences non-structural proteins (NSPs)
aaugugauagagccaugccua

SEQ ID NO: 1009 - RNA, 21 nt, Artificial Sequence
  5′ sense siRNA sequences non-structural proteins (NSPs)
aauggucauguguggcgguuc

SEQ ID NO: 1010 - RNA, 21 nt, Artificial Sequence
  5′ sense siRNA sequences non-structural proteins (NSPs)
aaugaugauacucucugacga

SEQ ID NO: 1011 - RNA, 21 nt, Artificial Sequence
  5′ sense siRNA sequences non-structural proteins (NSPs)
aagcaaaauguuggacugaga

SEQ ID NO: 1012 - RNA, 21 nt, Artificial Sequence
  5′ sense siRNA sequences non-structural proteins (NSPs)
aguugugaucaacuccgcgaa

SEQ ID NO: 1013 - RNA, 21 nt, Artificial Sequence
  5′ sense siRNA sequences non-structural proteins (NSPs)
uugucaagcugucacggccaa

SEQ ID NO: 1014 - RNA, 21 nt, Artificial Sequence
  5′ sense siRNA sequences non-structural proteins (NSPs)
aaacaccgugcggcacaggca

SEQ ID NO: 1015 - RNA, 21 nt, Artificial Sequence
  5′ sense siRNA sequences non-structural proteins (NSPs)
aaagaauagagcucgcaccgu

SEQ ID NO: 1016 - RNA, 21 nt, Artificial Sequence
  5′ sense siRNA sequences non-structural proteins (NSPs)
aagaauagagcucgcaccgua

SEQ ID NO: 1017 - RNA, 21 nt, Artificial Sequence
  5′ sense siRNA sequences non-structural proteins (NSPs)
aaaugugauagagccaugccu

SEQ ID NO: 1018 - RNA, 21 nt, Artificial Sequence
  5′ sense siRNA sequences non-structural proteins (NSPs)
aaauggucauguguggcgguu

SEQ ID NO: 1019 - RNA, 21 nt, Artificial Sequence
  5′ sense siRNA sequences non-structural proteins (NSPs)
agguaugagcuauuauuguaa

SEQ ID NO: 1020 - RNA, 21 nt, Artificial Sequence
  5′ sense siRNA sequences non-structural proteins (NSPs)
gguaugagcuauuauuguaaa

SEQ ID NO: 1021 - RNA, 21 nt, Artificial Sequence
  5′ sense siRNA sequences non-structural proteins (NSPs)
cugguuaucguguaacuaaaa

SEQ ID NO: 1022 - RNA, 21 nt, Artificial Sequence
  5′ sense siRNA sequences non-structural proteins (NSPs)
ugguuaucguguaacuaaaaa

SEQ ID NO: 1023 - RNA, 21 nt, Artificial Sequence
  5′ sense siRNA sequences non-structural proteins (NSPs)
guacaacaacuuacaaauuaa

SEQ ID NO: 1024 - RNA, 21 nt, Artificial Sequence
  5′ sense siRNA sequences non-structural proteins (NSPs)
uacaacaacuuacaaauuaaa

SEQ ID NO: 1025 - RNA, 21 nt, Artificial Sequence
  5′ sense siRNA sequences non-structural proteins (NSPs)
agcaauguugcaaauuaucaa

SEQ ID NO: 1026 - RNA, 21 nt, Artificial Sequence
  5′ sense siRNA sequences non-structural proteins (NSPs)
gcaauguugcaaauuaucaaa

SEQ ID NO: 1027 - RNA, 21 nt, Artificial Sequence
  5′ sense siRNA sequences non-structural proteins (NSPs)
caauguugcaaauuaucaaaa

SEQ ID NO: 1028 - RNA, 21 nt, Artificial Sequence
  5′ sense siRNA sequences non-structural proteins (NSPs)
caguguguagacuuaugaaaa

SEQ ID NO: 1029 - RNA, 21 nt, Artificial Sequence
  5′ sense siRNA sequences non-structural proteins (NSPs)
agcucacucuuguaauguaaa

SEQ ID NO: 1030 - RNA, 21 nt, Artificial Sequence
  5′ sense siRNA sequences non-structural proteins (NSPs)
aaaacaguaaaguacaaauag

SEQ ID NO: 1031 - RNA, 21 nt, Artificial Sequence
  5′ sense siRNA sequences non-structural proteins (NSPs)
aacaacuuacaaauuaaaugu

SEQ ID NO: 1032 - RNA, 21 nt, Artificial Sequence
  5′ sense siRNA sequences non-structural proteins (NSPs)
aagagcacuauguuagaauua

SEQ ID NO: 1033 - RNA, 21 nt, Artificial Sequence
  5′ sense siRNA sequences non-structural proteins (NSPs)
aauguugcaaauuaucaaaag

SEQ ID NO: 1034 - RNA, 21 nt, Artificial Sequence
  5′ sense siRNA sequences non-structural proteins (NSPs)
uagaccauucuuauguuguaa

SEQ ID NO: 1035 - RNA, 21 nt, Artificial Sequence
  5′ sense siRNA sequences non-structural proteins (NSPs)
agaccauucuuauguuguaaa

SEQ ID NO: 1036 - RNA, 21 nt, Artificial Sequence
  5′ sense siRNA sequences non-structural proteins (NSPs)
guuacgaccaugucauaucaa

SEQ ID NO: 1037 - RNA, 21 nt, Artificial Sequence
  5′ sense siRNA sequences non-structural proteins (NSPs)
ugucauaucaacaucacauaa

SEQ ID NO: 1038 - RNA, 21 nt, Artificial Sequence
  5′ sense siRNA sequences non-structural proteins (NSPs)
gucauaucaacaucacauaaa

SEQ ID NO: 1039 - RNA, 21 nt, Artificial Sequence
  5′ sense siRNA sequences non-structural proteins (NSPs)
cuauuauuguaaaucacauaa

SEQ ID NO: 1040 - RNA, 21 nt, Artificial Sequence
  5′ sense siRNA sequences non-structural proteins (NSPs)
uauuauuguaaaucacauaaa

SEQ ID NO: 1041 - RNA, 21 nt, Artificial Sequence
  5′ sense siRNA sequences non-structural proteins (NSPs)
uacugguuaucguguaacuaa

SEQ ID NO: 1042 - RNA, 21 nt, Artificial Sequence
  5′ sense siRNA sequences non-structural proteins (NSPs)
acugguuaucguguaacuaaa

SEQ ID NO: 1043 - RNA, 21 nt, Artificial Sequence
  5′ sense siRNA sequences non-structural proteins (NSPs)
ucguguaacuaaaaacaguaa

SEQ ID NO: 1044 - RNA, 21 nt, Artificial Sequence
  5′ sense siRNA sequences non-structural proteins (NSPs)
cguguaacuaaaaacaguaaa

SEQ ID NO: 1045 - RNA, 21 nt, Artificial Sequence
  5′ sense siRNA sequences non-structural proteins (NSPs)
acuaaaaacaguaaaguacaa

SEQ ID NO: 1046 - RNA, 21 nt, Artificial Sequence
  5′ sense siRNA sequences non-structural proteins (NSPs)
cuaaaaacaguaaaguacaaa

SEQ ID NO: 1047 - RNA, 21 nt, Artificial Sequence
  5′ sense siRNA sequences non-structural proteins (NSPs)
ccgagguacaacaacuuacaa

SEQ ID NO: 1048 - RNA, 21 nt, Artificial Sequence
  5′ sense siRNA sequences non-structural proteins (NSPs)
cgagguacaacaacuuacaaa

SEQ ID NO: 1049 - RNA, 21 nt, Artificial Sequence
  5′ sense siRNA sequences non-structural proteins (NSPs)
ugcugacaucacauacaguaa

SEQ ID NO: 1050 - RNA, 21 nt, Artificial Sequence
  5′ sense siRNA sequences non-structural proteins (NSPs)
cacaagagcacuauguuagaa

SEQ ID NO: 1051 - RNA, 21 nt, Artificial Sequence
  5′ sense siRNA sequences non-structural proteins (NSPs)
ucaaaagguugguaugcaaaa

SEQ ID NO: 1052 - RNA, 21 nt, Artificial Sequence
  5′ sense siRNA sequences non-structural proteins (NSPs)
cuaugugagaaggcauuaaaa

SEQ ID NO: 1053 - RNA, 21 nt, Artificial Sequence
  5′ sense siRNA sequences non-structural proteins (NSPs)
auaaauucaaagugaauucaa

SEQ ID NO: 1054 - RNA, 21 nt, Artificial Sequence
  5′ sense siRNA sequences non-structural proteins (NSPs)
aaagugaauucaacauuagaa

SEQ ID NO: 1055 - RNA, 21 nt, Artificial Sequence
  5′ sense siRNA sequences non-structural proteins (NSPs)
uucaguguguagacuuaugaa

SEQ ID NO: 1056 - RNA, 21 nt, Artificial Sequence
  5′ sense siRNA sequences non-structural proteins (NSPs)
ucaguguguagacuuaugaaa

SEQ ID NO: 1057 - RNA, 21 nt, Artificial Sequence
  5′ sense siRNA sequences non-structural proteins (NSPs)
gcuuaaagcacauaaagacaa

SEQ ID NO: 1058 - RNA, 21 nt, Artificial Sequence
  5′ sense siRNA sequences non-structural proteins (NSPs)
cuuaaagcacauaaagacaaa

SEQ ID NO: 1059 - RNA, 21 nt, Artificial Sequence
  5′ sense siRNA sequences non-structural proteins (NSPs)
cagcucacucuuguaauguaa

SEQ ID NO: 1060 - RNA, 21 nt, Artificial Sequence
  5′ sense siRNA sequences non-structural proteins (NSPs)
aacaucacauaaauuagucuu

SEQ ID NO: 1061 - RNA, 21 nt, Artificial Sequence
  5′ sense siRNA sequences non-structural proteins (NSPs)
aaauuagucuugucuguuaau

SEQ ID NO: 1062 - RNA, 21 nt, Artificial Sequence
  5′ sense siRNA sequences non-structural proteins (NSPs)
aauuagucuugucuguuaauc

SEQ ID NO: 1063 - RNA, 21 nt, Artificial Sequence
  5′ sense siRNA sequences non-structural proteins (NSPs)
aacuaaaaacaguaaaguaca

SEQ ID NO: 1064 - RNA, 21 nt, Artificial Sequence
  5′ sense siRNA sequences non-structural proteins (NSPs)
aaaaacaguaaaguacaaaua

SEQ ID NO: 1065 - RNA, 21 nt, Artificial Sequence
  5′ sense siRNA sequences non-structural proteins (NSPs)
aaacaguaaaguacaaauagg

SEQ ID NO: 1066 - RNA, 21 nt, Artificial Sequence
  5′ sense siRNA sequences non-structural proteins (NSPs)
aacuuacaaauuaaauguugg

SEQ ID NO: 1067 - RNA, 21 nt, Artificial Sequence
  5′ sense siRNA sequences non-structural proteins (NSPs)
aauuaaauguuggugauuauu

SEQ ID NO: 1068 - RNA, 21 nt, Artificial Sequence
  5′ sense siRNA sequences non-structural proteins (NSPs)
aagguugguaugcaaaaguau

SEQ ID NO: 1069 - RNA, 21 nt, Artificial Sequence
  5′ sense siRNA sequences non-structural proteins (NSPs)
aaauguaguagaauuauaccu

SEQ ID NO: 1070 - RNA, 21 nt, Artificial Sequence
  5′ sense siRNA sequences non-structural proteins (NSPs)
aauguaguagaauuauaccug

SEQ ID NO: 1071 - RNA, 21 nt, Artificial Sequence
  5′ sense siRNA sequences non-structural proteins (NSPs)
aaauucaaagugaauucaaca

SEQ ID NO: 1072 - RNA, 21 nt, Artificial Sequence
  5′ sense siRNA sequences non-structural proteins (NSPs)
aauucaaagugaauucaacau

SEQ ID NO: 1073 - RNA, 21 nt, Artificial Sequence
  5′ sense siRNA sequences non-structural proteins (NSPs)
aaagugaauucaacauuagaa

SEQ ID NO: 1074 - RNA, 21 nt, Artificial Sequence
  5′ sense siRNA sequences non-structural proteins (NSPs)
aagugaauucaacauuagaac

SEQ ID NO: 1075 - RNA, 21 nt, Artificial Sequence
  5′ sense siRNA sequences non-structural proteins (NSPs)
aauucaguguguagacuuaug

SEQ ID NO: 1076 - RNA, 21 nt, Artificial Sequence
  5′ sense siRNA sequences non-structural proteins (NSPs)
aagcuuaaagcacauaaagac

SEQ ID NO: 1077 - RNA, 21 nt, Artificial Sequence
  5′ sense siRNA sequences non-structural proteins (NSPs)
aagcacauaaagacaaaucag

SEQ ID NO: 1078 - RNA, 21 nt, Artificial Sequence
  5′ sense siRNA sequences non-structural proteins (NSPs)
aauuaacaggccacaaauagg

SEQ ID NO: 1079 - RNA, 21 nt, Artificial Sequence
  5′ sense siRNA sequences non-structural proteins (NSPs)
aaauaggcgugguaagagaau

SEQ ID NO: 1080 - RNA, 21 nt, Artificial Sequence
  5′ sense siRNA sequences non-structural proteins (NSPs)
aauaggcgugguaagagaauu

SEQ ID NO: 1081 - RNA, 21 nt, Artificial Sequence
  5′ sense siRNA sequences non-structural proteins (NSPs)
aaugcuguagccucaaagauu

SEQ ID NO: 1082 - RNA, 21 nt, Artificial Sequence
  5′ sense siRNA sequences non-structural proteins (NSPs)
aacucaaacuguugauucauc

SEQ ID NO: 1083 - RNA, 21 nt, Artificial Sequence
  5′ sense siRNA sequences non-structural proteins (NSPs)
aauaugacuaugucauauuca

SEQ ID NO: 1084 - RNA, 21 nt, Artificial Sequence
  5′ sense siRNA sequences non-structural proteins (NSPs)
aaugugacuaugucauauuca

SEQ ID NO: 1085 - RNA, 21 nt, Artificial Sequence
  5′ sense siRNA sequences non-structural proteins (NSPs)
aaacagcucacucuuguaaug

SEQ ID NO: 1086 - RNA, 21 nt, Artificial Sequence
  5′ sense siRNA sequences non-structural proteins (NSPs)
aacagcucacucuuguaaugu

SEQ ID NO: 1087 - RNA, 21 nt, Artificial Sequence
  5′ sense siRNA sequences non-structural proteins (NSPs)
uaaauuagucuugucuguuaa

SEQ ID NO: 1088 - RNA, 21 nt, Artificial Sequence
  5′ sense siRNA sequences non-structural proteins (NSPs)
ccauugugugcuaauggacaa

SEQ ID NO: 1089 - RNA, 21 nt, Artificial Sequence
  5′ sense siRNA sequences non-structural proteins (NSPs)
uacauguguugguagcgauaa

SEQ ID NO: 1090 - RNA, 21 nt, Artificial Sequence
  5′ sense siRNA sequences non-structural proteins (NSPs)
uuagcuaacaccuguacugaa

SEQ ID NO: 1091 - RNA, 21 nt, Artificial Sequence
  5′ sense siRNA sequences non-structural proteins (NSPs)
uagcuaacaccuguacugaaa

SEQ ID NO: 1092 - RNA, 21 nt, Artificial Sequence
  5′ sense siRNA sequences non-structural proteins (NSPs)
caccuguacugaaagacucaa

SEQ ID NO: 1093 - RNA, 21 nt, Artificial Sequence
  5′ sense siRNA sequences non-structural proteins (NSPs)
uaaaccuagaccaccacuuaa

SEQ ID NO: 1094 - RNA, 21 nt, Artificial Sequence
  5′ sense siRNA sequences non-structural proteins (NSPs)
cuagaccaccacuuaaccgaa

SEQ ID NO: 1095 - RNA, 19 nt, Artificial Sequence
  5′ sense siRNA sequences non-structural proteins (NSPs)
gcaccacgcacauugcuaa

SEQ ID NO: 1096 - RNA, 23 nt, Artificial Sequence
  5′ sense siRNA sequences non-structural proteins (NSPs)
accugcaccacgcacauugcuaa

SEQ ID NO: 1097 - RNA, 25 nt, Artificial Sequence
  5′ sense siRNA sequences non-structural proteins (NSPs)
uuaccugcaccacgcacauugcuaa

SEQ ID NO: 1098 - RNA, 21 nt, Artificial Sequence
  5′ sense siRNA sequences non-structural proteins (NSPs)
uagaccaccacuuaaccgaaa

SEQ ID NO: 1099 - RNA, 21 nt, Artificial Sequence
  5′ sense siRNA sequences non-structural proteins (NSPs)
cacauacaguaaugccauuaa

SEQ ID NO: 1100 - RNA, 21 nt, Artificial Sequence
  5′ sense siRNA sequences non-structural proteins (NSPs)
aucaaaagguugguaugcaaa

SEQ ID NO: 1101 - RNA, 21 nt, Artificial Sequence
  5′ sense siRNA sequences non-structural proteins (NSPs)
uguugaugcacuaugugagaa

SEQ ID NO: 1102 - RNA, 21 nt, Artificial Sequence
  5′ sense siRNA sequences non-structural proteins (NSPs)
cacuaugugagaaggcauuaa

SEQ ID NO: 1103 - RNA, 21 nt, Artificial Sequence
  5′ sense siRNA sequences non-structural proteins (NSPs)
acuaugugagaaggcauuaaa

SEQ ID NO: 1104 - RNA, 21 nt, Artificial Sequence
  5′ sense siRNA sequences non-structural proteins (NSPs)
cuauagauaaauguaguagaa

SEQ ID NO: 1105 - RNA, 21 nt, Artificial Sequence
  5′ sense siRNA sequences non-structural proteins (NSPs)
cugcaccacgcacauugcuaa

SEQ ID NO: 1106 - RNA, 21 nt, Artificial Sequence
  5′ sense siRNA sequences non-structural proteins (NSPs)
accacgcacauugcuaacuaa

SEQ ID NO: 1107 - RNA, 21 nt, Artificial Sequence
  5′ sense siRNA sequences non-structural proteins (NSPs)
uaauaagcuuaaagcacauaa

SEQ ID NO: 1108 - RNA, 21 nt, Artificial Sequence
  5′ sense siRNA sequences non-structural proteins (NSPs)
aauaagcuuaaagcacauaaa

SEQ ID NO: 1109 - RNA, 21 nt, Artificial Sequence
  5′ sense siRNA sequences non-structural proteins (NSPs)
caaauaggcgugguaagagaa

SEQ ID NO: 1110 - RNA, 21 nt, Artificial Sequence
  5′ sense siRNA sequences non-structural proteins (NSPs)
aagagaauuccuuacacguaa

SEQ ID NO: 1111 - RNA, 21 nt, Artificial Sequence
  5′ sense siRNA sequences non-structural proteins (NSPs)
uucaccuuauaauucacagaa

SEQ ID NO: 1112 - RNA, 21 nt, Artificial Sequence
  5′ sense siRNA sequences non-structural proteins (NSPs)
gacuaugucauauucacucaa

SEQ ID NO: 1113 - RNA, 21 nt, Artificial Sequence
  5′ sense siRNA sequences non-structural proteins (NSPs)
acuaugucauauucacucaaa

SEQ ID NO: 1114 - RNA, 21 nt, Artificial Sequence
  5′ sense siRNA sequences non-structural proteins (NSPs)
ugaaacagcucacucuuguaa

SEQ ID NO: 1115 - RNA, 21 nt, Artificial Sequence
  5′ sense siRNA sequences non-structural proteins (NSPs)
guugcuauuaccagagcaaaa

SEQ ID NO: 1116 - RNA, 21 nt, Artificial Sequence
  5′ sense siRNA sequences non-structural proteins (NSPs)
aaugcuccagguugugauguc

SEQ ID NO: 1117 - RNA, 21 nt, Artificial Sequence
  5′ sense siRNA sequences non-structural proteins (NSPs)
aaugcaauugcaacaugugac

SEQ ID NO: 1118 - RNA, 21 nt, Artificial Sequence
  5′ sense siRNA sequences non-structural proteins (NSPs)
aacaugugacuggacaaaugc

SEQ ID NO: 1119 - RNA, 21 nt, Artificial Sequence
  5′ sense siRNA sequences non-structural proteins (NSPs)
aacaccuguacugaaagacuc

SEQ ID NO: 1120 - RNA, 21 nt, Artificial Sequence
  5′ sense siRNA sequences non-structural proteins (NSPs)
aacugucuuaugguauugcua

SEQ ID NO: 1121 - RNA, 21 nt, Artificial Sequence
  5′ sense siRNA sequences non-structural proteins (NSPs)
aagugcugucugacagagaau

SEQ ID NO: 1122 - RNA, 21 nt, Artificial Sequence
  5′ sense siRNA sequences non-structural proteins (NSPs)
aaaccuagaccaccacuuaac

SEQ ID NO: 1123 - RNA, 21 nt, Artificial Sequence
  5′ sense siRNA sequences non-structural proteins (NSPs)
aaccuagaccaccacuuaacc

SEQ ID NO: 1124 - RNA, 21 nt, Artificial Sequence
  5′ sense siRNA sequences non-structural proteins (NSPs)
aacaguaaaguacaaauagga

SEQ ID NO: 1125 - RNA, 21 nt, Artificial Sequence
  5′ sense siRNA sequences non-structural proteins (NSPs)
aaaaaggugacuauggugaug

SEQ ID NO: 1126 - RNA, 21 nt, Artificial Sequence
  5′ sense siRNA sequences non-structural proteins (NSPs)
aaauuaaauguuggugauuau

SEQ ID NO: 1127 - RNA, 21 nt, Artificial Sequence
  5′ sense siRNA sequences non-structural proteins (NSPs)
aauuaucaaaagguugguaug

SEQ ID NO: 1128 - RNA, 21 nt, Artificial Sequence
  5′ sense siRNA sequences non-structural proteins (NSPs)
aaaagguugguaugcaaaagu

SEQ ID NO: 1129 - RNA, 21 nt, Artificial Sequence
  5′ sense siRNA sequences non-structural proteins (NSPs)
aaagguugguaugcaaaagua

SEQ ID NO: 1130 - RNA, 21 nt, Artificial Sequence
  5′ sense siRNA sequences non-structural proteins (NSPs)
aauucaacauuagaacaguau

SEQ ID NO: 1131 - RNA, 21 nt, Artificial Sequence
  5′ sense siRNA sequences non-structural proteins (NSPs)
aacauuagaacaguaugucuu

SEQ ID NO: 1132 - RNA, 21 nt, Artificial Sequence
  5′ sense siRNA sequences non-structural proteins (NSPs)
aagcacuauguguacauuggc

SEQ ID NO: 1133 - RNA, 21 nt, Artificial Sequence
  5′ sense siRNA sequences non-structural proteins (NSPs)
aacuauagguccagacauguu

SEQ ID NO: 1134 - RNA, 21 nt, Artificial Sequence
  5′ sense siRNA sequences non-structural proteins (NSPs)
aauaagcuuaaagcacauaaa

SEQ ID NO: 1135 - RNA, 21 nt, Artificial Sequence
  5′ sense siRNA sequences non-structural proteins (NSPs)
aaagcacauaaagacaaauca

SEQ ID NO: 1136 - RNA, 21 nt, Artificial Sequence
  5′ sense siRNA sequences non-structural proteins (NSPs)
aagagaauuccuuacacguaa

SEQ ID NO: 1137 - RNA, 21 nt, Artificial Sequence
  5′ sense siRNA sequences non-structural proteins (NSPs)
gaugucacagaugugacucaa

SEQ ID NO: 1138 - RNA, 21 nt, Artificial Sequence
  5′ sense siRNA sequences non-structural proteins (NSPs)
uugcaacaugugacuggacaa

SEQ ID NO: 1139 - RNA, 21 nt, Artificial Sequence
  5′ sense siRNA sequences non-structural proteins (NSPs)
ugcaacaugugacuggacaaa

SEQ ID NO: 1140 - RNA, 21 nt, Artificial Sequence
  5′ sense siRNA sequences non-structural proteins (NSPs)
gguauugcuacuguacgugaa

SEQ ID NO: 1141 - RNA, 21 nt, Artificial Sequence
  5′ sense siRNA sequences non-structural proteins (NSPs)
gaagugcugucugacagagaa

SEQ ID NO: 1142 - RNA, 21 nt, Artificial Sequence
  5′ sense siRNA sequences non-structural proteins (NSPs)
uaucaaaagguugguaugcaa

SEQ ID NO: 1143 - RNA, 21 nt, Artificial Sequence
  5′ sense siRNA sequences non-structural proteins (NSPs)
caaugccagauuacgugcuaa

SEQ ID NO: 1144 - RNA, 21 nt, Artificial Sequence
  5′ sense siRNA sequences non-structural proteins (NSPs)
gucggcguuguccugcugaaa

SEQ ID NO: 1145 - RNA, 21 nt, Artificial Sequence
  5′ sense siRNA sequences non-structural proteins (NSPs)
gucgccguuguccugcugaaa

SEQ ID NO: 1146 - RNA, 21 nt, Artificial Sequence
  5′ sense siRNA sequences non-structural proteins (NSPs)
cauaaagacaaaucagcucaa

SEQ ID NO: 1147 - RNA, 21 nt, Artificial Sequence
  5′ sense siRNA sequences non-structural proteins (NSPs)
cacagaaugcuguagccucaa

SEQ ID NO: 1148 - RNA, 21 nt, Artificial Sequence
  5′ sense siRNA sequences non-structural proteins (NSPs)
acagaaugcuguagccucaaa

SEQ ID NO: 1149 - RNA, 21 nt, Artificial Sequence
  5′ sense siRNA sequences non-structural proteins (NSPs)
auauucacucaaaccacugaa

SEQ ID NO: 1150 - RNA, 21 nt, Artificial Sequence
  5′ sense siRNA sequences non-structural proteins (NSPs)
uauucacucaaaccacugaaa

SEQ ID NO: 1151 - RNA, 21 nt, Artificial Sequence
  5′ sense siRNA sequences non-structural proteins (NSPs)
uguugcuauuaccagagcaaa

SEQ ID NO: 1152 - RNA, 21 nt, Artificial Sequence
  5′ sense siRNA sequences non-structural proteins (NSPs)
aaaugcuguuacgaccauguc

SEQ ID NO: 1153 - RNA, 21 nt, Artificial Sequence
  5′ sense siRNA sequences non-structural proteins (NSPs)
aaugcuguuacgaccauguca

SEQ ID NO: 1154 - RNA, 21 nt, Artificial Sequence
  5′ sense siRNA sequences non-structural proteins (NSPs)
aaaaauacauguguugguagc

SEQ ID NO: 1155 - RNA, 21 nt, Artificial Sequence
  5′ sense siRNA sequences non-structural proteins (NSPs)
aaaauacauguguugguagcg

SEQ ID NO: 1156 - RNA, 21 nt, Artificial Sequence
  5′ sense siRNA sequences non-structural proteins (NSPs)
aauugcaacaugugacuggac

SEQ ID NO: 1157 - RNA, 21 nt, Artificial Sequence
  5′ sense siRNA sequences non-structural proteins (NSPs)
aaacgcucaaagcuacugagg

SEQ ID NO: 1158 - RNA, 21 nt, Artificial Sequence
  5′ sense siRNA sequences non-structural proteins (NSPs)
aacgcucaaagcuacugagga

SEQ ID NO: 1159 - RNA, 21 nt, Artificial Sequence
  5′ sense siRNA sequences non-structural proteins (NSPs)
aaacugucuuaugguauugcu

SEQ ID NO: 1160 - RNA, 21 nt, Artificial Sequence
  5′ sense siRNA sequences non-structural proteins (NSPs)
aaguugguaaaccuagaccac

SEQ ID NO: 1161 - RNA, 21 nt, Artificial Sequence
  5′ sense siRNA sequences non-structural proteins (NSPs)
aaaguacaaauaggagaguac

SEQ ID NO: 1162 - RNA, 21 nt, Artificial Sequence
  5′ sense siRNA sequences non-structural proteins (NSPs)
aaguacaaauaggagaguaca

SEQ ID NO: 1163 - RNA, 21 nt, Artificial Sequence
  5′ sense siRNA sequences non-structural proteins (NSPs)
aaaaggugacuauggugaugc

SEQ ID NO: 1164 - RNA, 21 nt, Artificial Sequence
  5′ sense siRNA sequences non-structural proteins (NSPs)
aaggugacuauggugaugcug

SEQ ID NO: 1165 - RNA, 21 nt, Artificial Sequence
  5′ sense siRNA sequences non-structural proteins (NSPs)
aaugccauuaagugcaccuac

SEQ ID NO: 1166 - RNA, 21 nt, Artificial Sequence
  5′ sense siRNA sequences non-structural proteins (NSPs)
aagugcaccuacacuagugcc

SEQ ID NO: 1167 - RNA, 21 nt, Artificial Sequence
  5′ sense siRNA sequences non-structural proteins (NSPs)
aacacucaauaucucagauga

SEQ ID NO: 1168 - RNA, 21 nt, Artificial Sequence
  5′ sense siRNA sequences non-structural proteins (NSPs)
aaauuaucaaaagguugguau

SEQ ID NO: 1169 - RNA, 21 nt, Artificial Sequence
  5′ sense siRNA sequences non-structural proteins (NSPs)
aaaugcauugccugagacgac

SEQ ID NO: 1170 - RNA, 21 nt, Artificial Sequence
  5′ sense siRNA sequences non-structural proteins (NSPs)
aaugccagauuacgugcuaag

SEQ ID NO: 1171 - RNA, 21 nt, Artificial Sequence
  5′ sense siRNA sequences non-structural proteins (NSPs)
aaaacuauagguccagacaug

SEQ ID NO: 1172 - RNA, 21 nt, Artificial Sequence
  5′ sense siRNA sequences non-structural proteins (NSPs)
aaacuauagguccagacaugu

SEQ ID NO: 1173 - RNA, 21 nt, Artificial Sequence
  5′ sense siRNA sequences non-structural proteins (NSPs)
aacuugucggcguuguccugc

SEQ ID NO: 1174 - RNA, 21 nt, Artificial Sequence
  5′ sense siRNA sequences non-structural proteins (NSPs)
aaauuguugacacugugagug

SEQ ID NO: 1175 - RNA, 21 nt, Artificial Sequence
  5′ sense siRNA sequences non-structural proteins (NSPs)
aauuguugacacugugagugc

SEQ ID NO: 1176 - RNA, 21 nt, Artificial Sequence
  5′ sense siRNA sequences non-structural proteins (NSPs)
aaagacaaaucagcucaaugc

SEQ ID NO: 1177 - RNA, 21 nt, Artificial Sequence
  5′ sense siRNA sequences non-structural proteins (NSPs)
aagacaaaucagcucaaugcu

SEQ ID NO: 1178 - RNA, 21 nt, Artificial Sequence
  5′ sense siRNA sequences non-structural proteins (NSPs)
aacaggccacaaauaggcgug

SEQ ID NO: 1179 - RNA, 21 nt, Artificial Sequence
  5′ sense siRNA sequences non-structural proteins (NSPs)
aacagaccucaaauaggcguu

SEQ ID NO: 1180 - RNA, 21 nt, Artificial Sequence
  5′ sense siRNA sequences non-structural proteins (NSPs)
aaacuguugauucaucacagg

SEQ ID NO: 1181 - RNA, 21 nt, Artificial Sequence
  5′ sense siRNA sequences non-structural proteins (NSPs)
aaccacugaaacagcucacuc

SEQ ID NO: 1182 - RNA, 21 nt, Artificial Sequence
  5′ sense siRNA sequences non-structural proteins (NSPs)
gcaccuacacuagugccacaa

SEQ ID NO: 1183 - RNA, 21 nt, Artificial Sequence
  5′ sense siRNA sequences non-structural proteins (NSPs)
guccagacauguuccucggaa

SEQ ID NO: 1184 - RNA, 21 nt, Artificial Sequence
  5′ sense siRNA sequences non-structural proteins (NSPs)
ugucggcguuguccugcugaa

SEQ ID NO: 1185 - RNA, 21 nt, Artificial Sequence
  5′ sense siRNA sequences non-structural proteins (NSPs)
ucugcaauuaacaggccacaa

SEQ ID NO: 1186 - RNA, 21 nt, Artificial Sequence
  5′ sense siRNA sequences non-structural proteins (NSPs)
cugcaauuaacaggccacaaa

SEQ ID NO: 1187 - RNA, 21 nt, Artificial Sequence
  5′ sense siRNA sequences non-structural proteins (NSPs)
ggccacaaauaggcgugguaa

SEQ ID NO: 1188 - RNA, 21 nt, Artificial Sequence
  5′ sense siRNA sequences non-structural proteins (NSPs)
auguugcuauuaccagagcaa

SEQ ID NO: 1189 - RNA, 21 nt, Artificial Sequence
  5′ sense siRNA sequences non-structural proteins (NSPs)
aaauacauguguugguagcga

SEQ ID NO: 1190 - RNA, 21 nt, Artificial Sequence
  5′ sense siRNA sequences non-structural proteins (NSPs)
aauacauguguugguagcgau

SEQ ID NO: 1191 - RNA, 21 nt, Artificial Sequence
  5′ sense siRNA sequences non-structural proteins (NSPs)
aaaggugacuauggugaugcu

SEQ ID NO: 1192 - RNA, 21 nt, Artificial Sequence
  5′ sense siRNA sequences non-structural proteins (NSPs)
aaaaguauucuacacuccagg

SEQ ID NO: 1193 - RNA, 21 nt, Artificial Sequence
  5′ sense siRNA sequences non-structural proteins (NSPs)
aauuauaccugcacgugcucg

SEQ ID NO: 1194 - RNA, 21 nt, Artificial Sequence
  5′ sense siRNA sequences non-structural proteins (NSPs)
aaugcauugccugagacgaca

SEQ ID NO: 1195 - RNA, 21 nt, Artificial Sequence
  5′ sense siRNA sequences non-structural proteins (NSPs)
aauuaccugcaccacgcacau

SEQ ID NO: 1196 - RNA, 21 nt, Artificial Sequence
  5′ sense siRNA sequences non-structural proteins (NSPs)
aauucacagaaugcuguagcc

SEQ ID NO: 1197 - RNA, 21 nt, Artificial Sequence
  5′ sense siRNA sequences non-structural proteins (NSPs)
aaaccacugaaacagcucacu

SEQ ID NO: 1198 - RNA, 21 nt, Artificial Sequence
  5′ sense siRNA sequences non-structural proteins (NSPs)
aauguugcuauuaccagagca

SEQ ID NO: 1199 - RNA, 21 nt, Artificial Sequence
  5′ anti-sense siRNA sequences non-structural proteins (NSPs)
uuuauuuuaguaacaucagcu

SEQ ID NO: 1200 - RNA, 21 nt, Artificial Sequence
  5′ anti-sense siRNA sequences non-structural proteins (NSPs)
uuuuauuuuaguaacaucagc

SEQ ID NO: 1201 - RNA, 21 nt, Artificial Sequence
  5′ anti-sense siRNA sequences non-structural proteins (NSPs)
uuuuaguaacaucagcuccau

SEQ ID NO: 1202 - RNA, 21 nt, Artificial Sequence
  5′ anti-sense siRNA sequences non-structural proteins (NSPs)
uuauuuuaguaacaucagcuc

SEQ ID NO: 1203 - RNA, 21 nt, Artificial Sequence
  5′ anti-sense siRNA sequences non-structural proteins (NSPs)
uuauauguuuauagugaccac

SEQ ID NO: 1204 - RNA, 21 nt, Artificial Sequence
  5′ anti-sense siRNA sequences non-structural proteins (NSPs)
gugaauuaugagguuuuauuu

SEQ ID NO: 1205 - RNA, 21 nt, Artificial Sequence
  5′ anti-sense siRNA sequences non-structural proteins (NSPs)
uagauauuuuguagcuuguuu

SEQ ID NO: 1206 - RNA, 21 nt, Artificial Sequence
  5′ anti-sense siRNA sequences non-structural proteins (NSPs)
cuagauauuuuguagcuuguu

SEQ ID NO: 1207 - RNA, 21 nt, Artificial Sequence
  5′ anti-sense siRNA sequences non-structural proteins (NSPs)
uuaguaacaucagcuccaucc

SEQ ID NO: 1208 - RNA, 21 nt, Artificial Sequence
  5′ anti-sense siRNA sequences non-structural proteins (NSPs)
uuuaguaacaucagcuccauc

SEQ ID NO: 1209 - RNA, 21 nt, Artificial Sequence
  5′ anti-sense siRNA sequences non-structural proteins (NSPs)
uuuuaccuucaugugaauuau

SEQ ID NO: 1210 - RNA, 21 nt, Artificial Sequence
  5′ anti-sense siRNA sequences non-structural proteins (NSPs)
uuuaaugcugacauguaccua

SEQ ID NO: 1211 - RNA, 21 nt, Artificial Sequence
  5′ anti-sense siRNA sequences non-structural proteins (NSPs)
uuuuagugugauuuaaugcug

SEQ ID NO: 1212 - RNA, 21 nt, Artificial Sequence
  5′ anti-sense siRNA sequences non-structural proteins (NSPs)
uuuuuagugugauuuaaugcu

SEQ ID NO: 1213 - RNA, 21 nt, Artificial Sequence
  5′ anti-sense siRNA sequences non-structural proteins (NSPs)
uuaacaaugcaguggcaagau

SEQ ID NO: 1214 - RNA, 21 nt, Artificial Sequence
  5′ anti-sense siRNA sequences non-structural proteins (NSPs)
uuauuacaguaggcuaagaua

SEQ ID NO: 1215 - RNA, 21 nt, Artificial Sequence
  5′ anti-sense siRNA sequences non-structural proteins (NSPs)
uuucucuaacaucaccuaacu

SEQ ID NO: 1216 - RNA, 21 nt, Artificial Sequence
  5′ anti-sense siRNA sequences non-structural proteins (NSPs)
uuuuacacaccacguucaaga

SEQ ID NO: 1217 - RNA, 21 nt, Artificial Sequence
  5′ anti-sense siRNA sequences non-structural proteins (NSPs)
uuaaguucauacugagcaggu

SEQ ID NO: 1218 - RNA, 21 nt, Artificial Sequence
  5′ anti-sense siRNA sequences non-structural proteins (NSPs)
uuauagugaccacacugguaa

SEQ ID NO: 1219 - RNA, 21 nt, Artificial Sequence
  5′ anti-sense siRNA sequences non-structural proteins (NSPs)
uuuauagugaccacacuggua

SEQ ID NO: 1220 - RNA, 21 nt, Artificial Sequence
  5′ anti-sense siRNA sequences non-structural proteins (NSPs)
uuagaaguuauauguuuauag

SEQ ID NO: 1221 - RNA, 21 nt, Artificial Sequence
  5′ anti-sense siRNA sequences non-structural proteins (NSPs)
uuuagaaguuauauguuuaua

SEQ ID NO: 1222 - RNA, 21 nt, Artificial Sequence
  5′ anti-sense siRNA sequences non-structural proteins (NSPs)
uucuuuagaaguuauauguuu

SEQ ID NO: 1223 - RNA, 21 nt, Artificial Sequence
  5′ anti-sense siRNA sequences non-structural proteins (NSPs)
uuucuuuagaaguuauauguu

SEQ ID NO: 1224 - RNA, 21 nt, Artificial Sequence
  5′ anti-sense siRNA sequences non-structural proteins (NSPs)
uuguauucugaggacuuugua

SEQ ID NO: 1225 - RNA, 21 nt, Artificial Sequence
  5′ anti-sense siRNA sequences non-structural proteins (NSPs)
uuuguauucugaggacuuugu

SEQ ID NO: 1226 - RNA, 21 nt, Artificial Sequence
  5′ anti-sense siRNA sequences non-structural proteins (NSPs)
ugaauuaugagguuuuauuuu

SEQ ID NO: 1227 - RNA, 21 nt, Artificial Sequence
  5′ anti-sense siRNA sequences non-structural proteins (NSPs)
ugugaauuaugagguuuuauu

SEQ ID NO: 1228 - RNA, 21 nt, Artificial Sequence
  5′ anti-sense siRNA sequences non-structural proteins (NSPs)
aacucuauuuguuggaguguu

SEQ ID NO: 1229 - RNA, 21 nt, Artificial Sequence
  5′ anti-sense siRNA sequences non-structural proteins (NSPs)
acaaguuuuacacaccacguu

SEQ ID NO: 1230 - RNA, 21 nt, Artificial Sequence
  5′ anti-sense siRNA sequences non-structural proteins (NSPs)
guacuagauauuuuguagcuu

SEQ ID NO: 1231 - RNA, 21 nt, Artificial Sequence
  5′ anti-sense siRNA sequences non-structural proteins (NSPs)
uucuuuagaaguuauauguuu

SEQ ID NO: 1232 - RNA, 21 nt, Artificial Sequence
  5′ anti-sense siRNA sequences non-structural proteins (NSPs)
uuucuuuagaaguuauauguu

SEQ ID NO: 1233 - RNA, 21 nt, Artificial Sequence
  5′ anti-sense siRNA sequences non-structural proteins (NSPs)
ccuuuguauucugaggacuuu

SEQ ID NO: 1234 - RNA, 21 nt, Artificial Sequence
  5′ anti-sense siRNA sequences non-structural proteins (NSPs)
uuaugagguuuuauuuuagua

SEQ ID NO: 1235 - RNA, 21 nt, Artificial Sequence
  5′ anti-sense siRNA sequences non-structural proteins (NSPs)
uucaugugaauuaugagguuu

SEQ ID NO: 1236 - RNA, 21 nt, Artificial Sequence
  5′ anti-sense siRNA sequences non-structural proteins (NSPs)
uuaccuucaugugaauuauga

SEQ ID NO: 1237 - RNA, 21 nt, Artificial Sequence
  5′ anti-sense siRNA sequences non-structural proteins (NSPs)
uuuaccuucaugugaauuaug

SEQ ID NO: 1238 - RNA, 21 nt, Artificial Sequence
  5′ anti-sense siRNA sequences non-structural proteins (NSPs)
uuaaugcugacauguaccuac

SEQ ID NO: 1239 - RNA, 21 nt, Artificial Sequence
  5′ anti-sense siRNA sequences non-structural proteins (NSPs)
uuagugugauuuaaugcugac

SEQ ID NO: 1240 - RNA, 21 nt, Artificial Sequence
  5′ anti-sense siRNA sequences non-structural proteins (NSPs)
uuuagugugauuuaaugcuga

SEQ ID NO: 1241 - RNA, 21 nt, Artificial Sequence
  5′ anti-sense siRNA sequences non-structural proteins (NSPs)
uuccacuuuuuagugugauuu

SEQ ID NO: 1242 - RNA, 21 nt, Artificial Sequence
  5′ anti-sense siRNA sequences non-structural proteins (NSPs)
uuuccacuuuuuagugugauu

SEQ ID NO: 1243 - RNA, 21 nt, Artificial Sequence
  5′ anti-sense siRNA sequences non-structural proteins (NSPs)
uuuguuggaguguuaacaaug

SEQ ID NO: 1244 - RNA, 21 nt, Artificial Sequence
  5′ anti-sense siRNA sequences non-structural proteins (NSPs)
uucaacucuauuuguuggagu

SEQ ID NO: 1245 - RNA, 21 nt, Artificial Sequence
  5′ anti-sense siRNA sequences non-structural proteins (NSPs)
uuacaguaggcuaagauaagu

SEQ ID NO: 1246 - RNA, 21 nt, Artificial Sequence
  5′ anti-sense siRNA sequences non-structural proteins (NSPs)
uucucuaacaucaccuaacuc

SEQ ID NO: 1247 - RNA, 21 nt, Artificial Sequence
  5′ anti-sense siRNA sequences non-structural proteins (NSPs)
uuguuucucuaacaucaccua

SEQ ID NO: 1248 - RNA, 21 nt, Artificial Sequence
  5′ anti-sense siRNA sequences non-structural proteins (NSPs)
uucaagacucuuuugcaagaa

SEQ ID NO: 1249 - RNA, 21 nt, Artificial Sequence
  5′ anti-sense siRNA sequences non-structural proteins (NSPs)
uuuacacaccacguucaagac

SEQ ID NO: 1250 - RNA, 21 nt, Artificial Sequence
  5′ anti-sense siRNA sequences non-structural proteins (NSPs)
uuuaccacacguacaagguau

SEQ ID NO: 1251 - RNA, 21 nt, Artificial Sequence
  5′ anti-sense siRNA sequences non-structural proteins (NSPs)
uuguuuaccacacguacaagg

SEQ ID NO: 1252 - RNA, 21 nt, Artificial Sequence
  5′ anti-sense siRNA sequences non-structural proteins (NSPs)
uuguagcuuguuuaccacacg

SEQ ID NO: 1253 - RNA, 21 nt, Artificial Sequence
  5′ anti-sense siRNA sequences non-structural proteins (NSPs)
uuuguagcuuguuuaccacac

SEQ ID NO: 1254 - RNA, 21 nt, Artificial Sequence
  5′ anti-sense siRNA sequences non-structural proteins (NSPs)
uuuuguagcuuguuuaccaca

SEQ ID NO: 1255 - RNA, 21 nt, Artificial Sequence
  5′ anti-sense siRNA sequences non-structural proteins (NSPs)
uuguacuagauauuuuguagc

SEQ ID NO: 1256 - RNA, 21 nt, Artificial Sequence
  5′ anti-sense siRNA sequences non-structural proteins (NSPs)
uucauacugagcagguggugc

SEQ ID NO: 1257 - RNA, 21 nt, Artificial Sequence
  5′ anti-sense siRNA sequences non-structural proteins (NSPs)
uuuauacucagcagguggugc

SEQ ID NO: 1258 - RNA, 19 nt, Artificial Sequence
  5′ anti-sense siRNA sequences non-structural proteins (NSPs)
uucauacugagcagguggu

SEQ ID NO: 1259 - RNA, 23 nt, Artificial Sequence
  5′ anti-sense siRNA sequences non-structural proteins (NSPs)
uucauacugagcagguggugcug

SEQ ID NO: 1260 - RNA, 25 nt, Artificial Sequence
  5′ anti-sense siRNA sequences non-structural proteins (NSPs)
uucauacugagcagguggugcugac

SEQ ID NO: 1261 - RNA, 21 nt, Artificial Sequence
  5′ anti-sense siRNA sequences non-structural proteins (NSPs)
uuguguaacuguuuucuuugu

SEQ ID NO: 1262 - RNA, 21 nt, Artificial Sequence
  5′ anti-sense siRNA sequences non-structural proteins (NSPs)
uuguuguguaacuguuuucuu

SEQ ID NO: 1263 - RNA, 21 nt, Artificial Sequence
  5′ anti-sense siRNA sequences non-structural proteins (NSPs)
aacuguuguccauaugucauu

SEQ ID NO: 1264 - RNA, 21 nt, Artificial Sequence
  5′ anti-sense siRNA sequences non-structural proteins (NSPs)
ucaugugaauuaugagguuuu

SEQ ID NO: 1265 - RNA, 21 nt, Artificial Sequence
  5′ anti-sense siRNA sequences non-structural proteins (NSPs)
uucaugugaauuaugagguuu

SEQ ID NO: 1266 - RNA, 21 nt, Artificial Sequence
  5′ anti-sense siRNA sequences non-structural proteins (NSPs)
cuucaugugaauuaugagguu

SEQ ID NO: 1267 - RNA, 21 nt, Artificial Sequence
  5′ anti-sense siRNA sequences non-structural proteins (NSPs)
uguuuuaccuucaugugaauu

SEQ ID NO: 1268 - RNA, 21 nt, Artificial Sequence
  5′ anti-sense siRNA sequences non-structural proteins (NSPs)
uuccacuuuuuagugugauuu

SEQ ID NO: 1269 - RNA, 21 nt, Artificial Sequence
  5′ anti-sense siRNA sequences non-structural proteins (NSPs)
uuuccacuuuuuagugugauu

SEQ ID NO: 1270 - RNA, 21 nt, Artificial Sequence
  5′ anti-sense siRNA sequences non-structural proteins (NSPs)
aucuuguagagcagguggauu

SEQ ID NO: 1271 - RNA, 21 nt, Artificial Sequence
  5′ anti-sense siRNA sequences non-structural proteins (NSPs)
acaagguaucugaacaccuuu

SEQ ID NO: 1272 - RNA, 21 nt, Artificial Sequence
  5′ anti-sense siRNA sequences non-structural proteins (NSPs)
uacaagguaucugaacaccuu

SEQ ID NO: 1273 - RNA, 21 nt, Artificial Sequence
  5′ anti-sense siRNA sequences non-structural proteins (NSPs)
accuuuguauucugaggacuu

SEQ ID NO: 1274 - RNA, 21 nt, Artificial Sequence
  5′ anti-sense siRNA sequences non-structural proteins (NSPs)
ccguaauaggaccuuuguauu

SEQ ID NO: 1275 - RNA, 21 nt, Artificial Sequence
  5′ anti-sense siRNA sequences non-structural proteins (NSPs)
uguuguguaacuguuuucuuu

SEQ ID NO: 1276 - RNA, 21 nt, Artificial Sequence
  5′ anti-sense siRNA sequences non-structural proteins (NSPs)
uuguuguguaacuguuuucuu

SEQ ID NO: 1277 - RNA, 21 nt, Artificial Sequence
  5′ anti-sense siRNA sequences non-structural proteins (NSPs)
augguuguuguguaacuguuu

SEQ ID NO: 1278 - RNA, 21 nt, Artificial Sequence
  5′ anti-sense siRNA sequences non-structural proteins (NSPs)
aaugguuguuguguaacuguu

SEQ ID NO: 1279 - RNA, 21 nt, Artificial Sequence
  5′ anti-sense siRNA sequences non-structural proteins (NSPs)
uuggaguguuaacaaugcagu

SEQ ID NO: 1280 - RNA, 21 nt, Artificial Sequence
  5′ anti-sense siRNA sequences non-structural proteins (NSPs)
uuguuggaguguuaacaaugc

SEQ ID NO: 1281 - RNA, 21 nt, Artificial Sequence
  5′ anti-sense siRNA sequences non-structural proteins (NSPs)
uugcucuguaauaagcaucuu

SEQ ID NO: 1282 - RNA, 21 nt, Artificial Sequence
  5′ anti-sense siRNA sequences non-structural proteins (NSPs)
uuacacaccacguucaagacu

SEQ ID NO: 1283 - RNA, 21 nt, Artificial Sequence
  5′ anti-sense siRNA sequences non-structural proteins (NSPs)
uuguccacaaguuuuacacac

SEQ ID NO: 1284 - RNA, 21 nt, Artificial Sequence
  5′ anti-sense siRNA sequences non-structural proteins (NSPs)
uugucugcuguuguccacaag

SEQ ID NO: 1285 - RNA, 21 nt, Artificial Sequence
  5′ anti-sense siRNA sequences non-structural proteins (NSPs)
uuaccacacguacaagguauc

SEQ ID NO: 1286 - RNA, 21 nt, Artificial Sequence
  5′ anti-sense siRNA sequences non-structural proteins (NSPs)
uuaccaguguacucacuagca

SEQ ID NO: 1287 - RNA, 21 nt, Artificial Sequence
  5′ anti-sense siRNA sequences non-structural proteins (NSPs)
uucugaggacuuuguaaguaa

SEQ ID NO: 1288 - RNA, 21 nt, Artificial Sequence
  5′ anti-sense siRNA sequences non-structural proteins (NSPs)
aacacguagagugucaucauu

SEQ ID NO: 1289 - RNA, 21 nt, Artificial Sequence
  5′ anti-sense siRNA sequences non-structural proteins (NSPs)
uugcucuguaauaagcaucuu

SEQ ID NO: 1290 - RNA, 21 nt, Artificial Sequence
  5′ anti-sense siRNA sequences non-structural proteins (NSPs)
uaacucaccuacugucuuauu

SEQ ID NO: 1291 - RNA, 21 nt, Artificial Sequence
  5′ anti-sense siRNA sequences non-structural proteins (NSPs)
accuaacucaccuacugucuu

SEQ ID NO: 1292 - RNA, 21 nt, Artificial Sequence
  5′ anti-sense siRNA sequences non-structural proteins (NSPs)
caccacguucaagacucuuuu

SEQ ID NO: 1293 - RNA, 21 nt, Artificial Sequence
  5′ anti-sense siRNA sequences non-structural proteins (NSPs)
cugcuguuguccacaaguuuu

SEQ ID NO: 1294 - RNA, 21 nt, Artificial Sequence
  5′ anti-sense siRNA sequences non-structural proteins (NSPs)
ucugcuguuguccacaaguuu

SEQ ID NO: 1295 - RNA, 21 nt, Artificial Sequence
  5′ anti-sense siRNA sequences non-structural proteins (NSPs)
gucugcuguuguccacaaguu

SEQ ID NO: 1296 - RNA, 21 nt, Artificial Sequence
  5′ anti-sense siRNA sequences non-structural proteins (NSPs)
agguaucugaacaccuuucuu

SEQ ID NO: 1297 - RNA, 21 nt, Artificial Sequence
  5′ anti-sense siRNA sequences non-structural proteins (NSPs)
uccuguuguacuagauauuuu

SEQ ID NO: 1298 - RNA, 21 nt, Artificial Sequence
  5′ anti-sense siRNA sequences non-structural proteins (NSPs)
cuccuguuguacuagauauuu

SEQ ID NO: 1299 - RNA, 21 nt, Artificial Sequence
  5′ anti-sense siRNA sequences non-structural proteins (NSPs)
acuccuguuguacuagauauu

SEQ ID NO: 1300 - RNA, 21 nt, Artificial Sequence
  5′ anti-sense siRNA sequences non-structural proteins (NSPs)
auagugaccacacugguaauu

SEQ ID NO: 1301 - RNA, 21 nt, Artificial Sequence
  5′ anti-sense siRNA sequences non-structural proteins (NSPs)
aacauccguaauaggaccuuu

SEQ ID NO: 1302 - RNA, 21 nt, Artificial Sequence
  5′ anti-sense siRNA sequences non-structural proteins (NSPs)
ugguuguuguguaacuguuuu

SEQ ID NO: 1303 - RNA, 21 nt, Artificial Sequence
  5′ anti-sense siRNA sequences non-structural proteins (NSPs)
aguggcaagauaacaguuguu

SEQ ID NO: 1304 - RNA, 21 nt, Artificial Sequence
  5′ anti-sense siRNA sequences non-structural proteins (NSPs)
acaccacguucaagacucuuu

SEQ ID NO: 1305 - RNA, 21 nt, Artificial Sequence
  5′ anti-sense siRNA sequences non-structural proteins (NSPs)
cacaccacguucaagacucuu

SEQ ID NO: 1306 - RNA, 21 nt, Artificial Sequence
  5′ anti-sense siRNA sequences non-structural proteins (NSPs)
ugcaguggcaagauaacaguu

SEQ ID NO: 1307 - RNA, 21 nt, Artificial Sequence
  5′ anti-sense siRNA sequences non-structural proteins (NSPs)
uuaugauuagacuuacgaaug

SEQ ID NO: 1308 - RNA, 21 nt, Artificial Sequence
  5′ anti-sense siRNA sequences non-structural proteins (NSPs)
uuuauaacagcagcguacaac

SEQ ID NO: 1309 - RNA, 21 nt, Artificial Sequence
  5′ anti-sense siRNA sequences non-structural proteins (NSPs)
uucauaauuguacuucauagc

SEQ ID NO: 1310 - RNA, 21 nt, Artificial Sequence
  5′ anti-sense siRNA sequences non-structural proteins (NSPs)
uuaaguguaguuguaccacaa

SEQ ID NO: 1311 - RNA, 21 nt, Artificial Sequence
  5′ anti-sense siRNA sequences non-structural proteins (NSPs)
uugaacauuaccagccuguac

SEQ ID NO: 1312 - RNA, 21 nt, Artificial Sequence
  5′ anti-sense siRNA sequences non-structural proteins (NSPs)
uuuugcauagaauguccaaua

SEQ ID NO: 1313 - RNA, 21 nt, Artificial Sequence
  5′ anti-sense siRNA sequences non-structural proteins (NSPs)
uuaaguacacaauuuugcaua

SEQ ID NO: 1314 - RNA, 21 nt, Artificial Sequence
  5′ anti-sense siRNA sequences non-structural proteins (NSPs)
uuaagcuuaaguacacaauuu

SEQ ID NO: 1315 - RNA, 21 nt, Artificial Sequence
  5′ anti-sense siRNA sequences non-structural proteins (NSPs)
uuaucuuaggugucuuaggau

SEQ ID NO: 1316 - RNA, 21 nt, Artificial Sequence
  5′ anti-sense siRNA sequences non-structural proteins (NSPs)
uuguaacaagcuaacacugaa

SEQ ID NO: 1317 - RNA, 21 nt, Artificial Sequence
  5′ anti-sense siRNA sequences non-structural proteins (NSPs)
uugguaauuccauauggugca

SEQ ID NO: 1318 - RNA, 21 nt, Artificial Sequence
  5′ anti-sense siRNA sequences non-structural proteins (NSPs)
uuaacuguaauaguugugucc

SEQ ID NO: 1319 - RNA, 21 nt, Artificial Sequence
  5′ anti-sense siRNA sequences non-structural proteins (NSPs)
uuauaacagcagcguacaacc

SEQ ID NO: 1320 - RNA, 21 nt, Artificial Sequence
  5′ anti-sense siRNA sequences non-structural proteins (NSPs)
uaagcuuaaguacacaauuuu

SEQ ID NO: 1321 - RNA, 21 nt, Artificial Sequence
  5′ anti-sense siRNA sequences non-structural proteins (NSPs)
uuaagcuuaaguacacaauuu

SEQ ID NO: 1322 - RNA, 21 nt, Artificial Sequence
  5′ anti-sense siRNA sequences non-structural proteins (NSPs)
cuuaagcuuaaguacacaauu

SEQ ID NO: 1323 - RNA, 21 nt, Artificial Sequence
  5′ anti-sense siRNA sequences non-structural proteins (NSPs)
guacuucauagccacaagguu

SEQ ID NO: 1324 - RNA, 21 nt, Artificial Sequence
  5′ anti-sense siRNA sequences non-structural proteins (NSPs)
cuaguaugucaacauggucuu

SEQ ID NO: 1325 - RNA, 21 nt, Artificial Sequence
  5′ anti-sense siRNA sequences non-structural proteins (NSPs)
uuagcaugucuucagaggugc

SEQ ID NO: 1326 - RNA, 21 nt, Artificial Sequence
  5′ anti-sense siRNA sequences non-structural proteins (NSPs)
uugcauagaauguccaauaac

SEQ ID NO: 1327 - RNA, 21 nt, Artificial Sequence
  5′ anti-sense siRNA sequences non-structural proteins (NSPs)
uuugcauagaauguccaauaa

SEQ ID NO: 1328 - RNA, 21 nt, Artificial Sequence
  5′ anti-sense siRNA sequences non-structural proteins (NSPs)
uuaggauuggcuguaucaacc

SEQ ID NO: 1329 - RNA, 21 nt, Artificial Sequence
  5′ anti-sense siRNA sequences non-structural proteins (NSPs)
uuccauauggugcauguaaca

SEQ ID NO: 1330 - RNA, 21 nt, Artificial Sequence
  5′ anti-sense siRNA sequences non-structural proteins (NSPs)
uucuaagucugugccagcaug

SEQ ID NO: 1331 - RNA, 21 nt, Artificial Sequence
  5′ anti-sense siRNA sequences non-structural proteins (NSPs)
uuaccuucuaagucugugcca

SEQ ID NO: 1332 - RNA, 21 nt, Artificial Sequence
  5′ anti-sense siRNA sequences non-structural proteins (NSPs)
uuguacuucauagccacaagg

SEQ ID NO: 1333 - RNA, 21 nt, Artificial Sequence
  5′ anti-sense siRNA sequences non-structural proteins (NSPs)
uuagagguucauaauuguacu

SEQ ID NO: 1334 - RNA, 21 nt, Artificial Sequence
  5′ anti-sense siRNA sequences non-structural proteins (NSPs)
uuguguuagagguucauaauu

SEQ ID NO: 1335 - RNA, 21 nt, Artificial Sequence
  5′ anti-sense siRNA sequences non-structural proteins (NSPs)
uaguuguaccacaaguuacuu

SEQ ID NO: 1336 - RNA, 21 nt, Artificial Sequence
  5′ anti-sense siRNA sequences non-structural proteins (NSPs)
aguguaguuguaccacaaguu

SEQ ID NO: 1337 - RNA, 21 nt, Artificial Sequence
  5′ anti-sense siRNA sequences non-structural proteins (NSPs)
uaucuuaggugucuuaggauu

SEQ ID NO: 1338 - RNA, 21 nt, Artificial Sequence
  5′ anti-sense siRNA sequences non-structural proteins (NSPs)
caugaacuccaguugguaauu

SEQ ID NO: 1339 - RNA, 21 nt, Artificial Sequence
  5′ anti-sense siRNA sequences non-structural proteins (NSPs)
uuguguuagagguucauaauu

SEQ ID NO: 1340 - RNA, 21 nt, Artificial Sequence
  5′ anti-sense siRNA sequences non-structural proteins (NSPs)
cauggucuuguguuagagguu

SEQ ID NO: 1341 - RNA, 21 nt, Artificial Sequence
  5′ anti-sense siRNA sequences non-structural proteins (NSPs)
augucaacauggucuuguguu

SEQ ID NO: 1342 - RNA, 21 nt, Artificial Sequence
  5′ anti-sense siRNA sequences non-structural proteins (NSPs)
uuguaccacaaguuacuugua

SEQ ID NO: 1343 - RNA, 21 nt, Artificial Sequence
  5′ anti-sense siRNA sequences non-structural proteins (NSPs)
uucagaggugcagaucacaug

SEQ ID NO: 1344 - RNA, 21 nt, Artificial Sequence
  5′ anti-sense siRNA sequences non-structural proteins (NSPs)
uuggcuguaucaaccuuaagc

SEQ ID NO: 1345 - RNA, 21 nt, Artificial Sequence
  5′ anti-sense siRNA sequences non-structural proteins (NSPs)
uuaggugucuuaggauuggcu

SEQ ID NO: 1346 - RNA, 21 nt, Artificial Sequence
  5′ anti-sense siRNA sequences non-structural proteins (NSPs)
uugugcuguuugccugucaac

SEQ ID NO: 1347 - RNA, 21 nt, Artificial Sequence
  5′ anti-sense siRNA sequences non-structural proteins (NSPs)
ggcuguaucaaccuuaagcuu

SEQ ID NO: 1348 - RNA, 21 nt, Artificial Sequence
  5′ anti-sense siRNA sequences non-structural proteins (NSPs)
aacaccagauggugaaccauu

SEQ ID NO: 1349 - RNA, 21 nt, Artificial Sequence
  5′ anti-sense siRNA sequences non-structural proteins (NSPs)
aacacuaccacaugaaccauu

SEQ ID NO: 1350 - RNA, 21 nt, Artificial Sequence
  5′ anti-sense siRNA sequences non-structural proteins (NSPs)
guaccagcugcuugugcuguu

SEQ ID NO: 1351 - RNA, 21 nt, Artificial Sequence
  5′ anti-sense siRNA sequences non-structural proteins (NSPs)
guaccugcagccugugcaguu

SEQ ID NO: 1352 - RNA, 21 nt, Artificial Sequence
  5′ anti-sense siRNA sequences non-structural proteins (NSPs)
uagagguucauaauuguacuu

SEQ ID NO: 1353 - RNA, 21 nt, Artificial Sequence
  5′ anti-sense siRNA sequences non-structural proteins (NSPs)
uuguguccguaccagcugcuu

SEQ ID NO: 1354 - RNA, 21 nt, Artificial Sequence
  5′ anti-sense siRNA sequences non-structural proteins (NSPs)
gaggugcagaucacaugucuu

SEQ ID NO: 1355 - RNA, 21 nt, Artificial Sequence
  5′ anti-sense siRNA sequences non-structural proteins (NSPs)
aggauuggcuguaucaaccuu

SEQ ID NO: 1356 - RNA, 21 nt, Artificial Sequence
  5′ anti-sense siRNA sequences non-structural proteins (NSPs)
gugccagcaugaacuccaguu

SEQ ID NO: 1357 - RNA, 21 nt, Artificial Sequence
  5′ anti-sense siRNA sequences non-structural proteins (NSPs)
uaccagcugcuugugcuguuu

SEQ ID NO: 1358 - RNA, 21 nt, Artificial Sequence
  5′ anti-sense siRNA sequences non-structural proteins (NSPs)
uuguguccguaccagcugcuu

SEQ ID NO: 1359 - RNA, 21 nt, Artificial Sequence
  5′ anti-sense siRNA sequences non-structural proteins (NSPs)
uuaguuuuuaggaauuuagca

SEQ ID NO: 1360 - RNA, 21 nt, Artificial Sequence
  5′ anti-sense siRNA sequences non-structural proteins (NSPs)
uuuuaaugugucacaauuacc

SEQ ID NO: 1361 - RNA, 21 nt, Artificial Sequence
  5′ anti-sense siRNA sequences non-structural proteins (NSPs)
uucuuuuaaugugucacaauu

SEQ ID NO: 1362 - RNA, 21 nt, Artificial Sequence
  5′ anti-sense siRNA sequences non-structural proteins (NSPs)
uuucuuuuaaugugucacaau

SEQ ID NO: 1363 - RNA, 21 nt, Artificial Sequence
  5′ anti-sense siRNA sequences non-structural proteins (NSPs)
uuaucuaaugucaguacacca

SEQ ID NO: 1364 - RNA, 21 nt, Artificial Sequence
  5′ anti-sense siRNA sequences non-structural proteins (NSPs)
uuaauauaggcauuaacaaug

SEQ ID NO: 1365 - RNA, 21 nt, Artificial Sequence
  5′ anti-sense siRNA sequences non-structural proteins (NSPs)
uuaauguaaggcuuuguuaag

SEQ ID NO: 1366 - RNA, 21 nt, Artificial Sequence
  5′ anti-sense siRNA sequences non-structural proteins (NSPs)
uuaauucaacagaacuuccuu

SEQ ID NO: 1367 - RNA, 21 nt, Artificial Sequence
  5′ anti-sense siRNA sequences non-structural proteins (NSPs)
uuuaauucaacagaacuuccu

SEQ ID NO: 1368 - RNA, 21 nt, Artificial Sequence
  5′ anti-sense siRNA sequences non-structural proteins (NSPs)
uuuuaauucaacagaacuucc

SEQ ID NO: 1369 - RNA, 21 nt, Artificial Sequence
  5′ anti-sense siRNA sequences non-structural proteins (NSPs)
uuuuaacauguugugccaacc

SEQ ID NO: 1370 - RNA, 21 nt, Artificial Sequence
  5′ anti-sense siRNA sequences non-structural proteins (NSPs)
uuuuuaacauguugugccaac

SEQ ID NO: 1371 - RNA, 21 nt, Artificial Sequence
  5′ anti-sense siRNA sequences non-structural proteins (NSPs)
uuaacauauagugaaccgcca

SEQ ID NO: 1372 - RNA, 21 nt, Artificial Sequence
  5′ anti-sense siRNA sequences non-structural proteins (NSPs)
uuuaacauauagugaaccgcc

SEQ ID NO: 1373 - RNA, 21 nt, Artificial Sequence
  5′ anti-sense siRNA sequences non-structural proteins (NSPs)
uucuuuuaaugugucacaauu

SEQ ID NO: 1374 - RNA, 21 nt, Artificial Sequence
  5′ anti-sense siRNA sequences non-structural proteins (NSPs)
caauuguaugugacaaguauu

SEQ ID NO: 1375 - RNA, 21 nt, Artificial Sequence
  5′ anti-sense siRNA sequences non-structural proteins (NSPs)
uuaauucaacagaacuuccuu

SEQ ID NO: 1376 - RNA, 21 nt, Artificial Sequence
  5′ anti-sense siRNA sequences non-structural proteins (NSPs)
uguuuuaauucaacagaacuu

SEQ ID NO: 1377 - RNA, 21 nt, Artificial Sequence
  5′ anti-sense siRNA sequences non-structural proteins (NSPs)
cuauugauuucaauaauuuuu

SEQ ID NO: 1378 - RNA, 21 nt, Artificial Sequence
  5′ anti-sense siRNA sequences non-structural proteins (NSPs)
guuuaacuagcauuguauguu

SEQ ID NO: 1379 - RNA, 21 nt, Artificial Sequence
  5′ anti-sense siRNA sequences non-structural proteins (NSPs)
uuucuucauguugguaguuag

SEQ ID NO: 1380 - RNA, 21 nt, Artificial Sequence
  5′ anti-sense siRNA sequences non-structural proteins (NSPs)
uuguuucuucauguugguagu

SEQ ID NO: 1381 - RNA, 21 nt, Artificial Sequence
  5′ anti-sense siRNA sequences non-structural proteins (NSPs)
uuaguaagacguugacgugau

SEQ ID NO: 1382 - RNA, 21 nt, Artificial Sequence
  5′ anti-sense siRNA sequences non-structural proteins (NSPs)
uuuaguaagacguugacguga

SEQ ID NO: 1383 - RNA, 21 nt, Artificial Sequence
  5′ anti-sense siRNA sequences non-structural proteins (NSPs)
uuguguauuuaguaagacguu

SEQ ID NO: 1384 - RNA, 21 nt, Artificial Sequence
  5′ anti-sense siRNA sequences non-structural proteins (NSPs)
uuaaugugucacaauuaccuu

SEQ ID NO: 1385 - RNA, 21 nt, Artificial Sequence
  5′ anti-sense siRNA sequences non-structural proteins (NSPs)
uuuaaugugucacaauuaccu

SEQ ID NO: 1386 - RNA, 21 nt, Artificial Sequence
  5′ anti-sense siRNA sequences non-structural proteins (NSPs)
uugauuaucuaaugucaguac

SEQ ID NO: 1387 - RNA, 21 nt, Artificial Sequence
  5′ anti-sense siRNA sequences non-structural proteins (NSPs)
uuaacaaugaauaauaagaau

SEQ ID NO: 1388 - RNA, 21 nt, Artificial Sequence
  5′ anti-sense siRNA sequences non-structural proteins (NSPs)
uuguuaagucagugucaacau

SEQ ID NO: 1389 - RNA, 21 nt, Artificial Sequence
  5′ anti-sense siRNA sequences non-structural proteins (NSPs)
uuuguuaagucagugucaaca

SEQ ID NO: 1390 - RNA, 21 nt, Artificial Sequence
  5′ anti-sense siRNA sequences non-structural proteins (NSPs)
uuuuaaccucucuuccgugaa

SEQ ID NO: 1391 - RNA, 21 nt, Artificial Sequence
  5′ anti-sense siRNA sequences non-structural proteins (NSPs)
uuuuucucacuagugguccaa

SEQ ID NO: 1392 - RNA, 21 nt, Artificial Sequence
  5′ anti-sense siRNA sequences non-structural proteins (NSPs)
uuauguacaacaccuagcucu

SEQ ID NO: 1393 - RNA, 21 nt, Artificial Sequence
  5′ anti-sense siRNA sequences non-structural proteins (NSPs)
uuaucuaguaauagauuacca

SEQ ID NO: 1394 - RNA, 21 nt, Artificial Sequence
  5′ anti-sense siRNA sequences non-structural proteins (NSPs)
uuuaucuaguaauagauuacc

SEQ ID NO: 1395 - RNA, 21 nt, Artificial Sequence
  5′ anti-sense siRNA sequences non-structural proteins (NSPs)
uuaguaagugcagcuacugaa

SEQ ID NO: 1396 - RNA, 21 nt, Artificial Sequence
  5′ anti-sense siRNA sequences non-structural proteins (NSPs)
uuguuaguaagugcagcuacu

SEQ ID NO: 1397 - RNA, 21 nt, Artificial Sequence
  5′ anti-sense siRNA sequences non-structural proteins (NSPs)
uuauaacgauaguagucauaa

SEQ ID NO: 1398 - RNA, 21 nt, Artificial Sequence
  5′ anti-sense siRNA sequences non-structural proteins (NSPs)
uuaucaacaacuucaacuaca

SEQ ID NO: 1399 - RNA, 21 nt, Artificial Sequence
  5′ anti-sense siRNA sequences non-structural proteins (NSPs)
uuaauacagccaccaucguaa

SEQ ID NO: 1400 - RNA, 21 nt, Artificial Sequence
  5′ anti-sense siRNA sequences non-structural proteins (NSPs)
uuagcauuaauacagccacca

SEQ ID NO: 1401 - RNA, 21 nt, Artificial Sequence
  5′ anti-sense siRNA sequences non-structural proteins (NSPs)
uuaagauucauuugaguuaua

SEQ ID NO: 1402 - RNA, 21 nt, Artificial Sequence
  5′ anti-sense siRNA sequences non-structural proteins (NSPs)
uugauuucaauaauuuuugau

SEQ ID NO: 1403 - RNA, 21 nt, Artificial Sequence
  5′ anti-sense siRNA sequences non-structural proteins (NSPs)
uuccaauuacuacaguagcuc

SEQ ID NO: 1404 - RNA, 21 nt, Artificial Sequence
  5′ anti-sense siRNA sequences non-structural proteins (NSPs)
uuaacauguugugccaaccac

SEQ ID NO: 1405 - RNA, 21 nt, Artificial Sequence
  5′ anti-sense siRNA sequences non-structural proteins (NSPs)
uuuaacauguugugccaacca

SEQ ID NO: 1406 - RNA, 21 nt, Artificial Sequence
  5′ anti-sense siRNA sequences non-structural proteins (NSPs)
uuguauguuugcgagcaagaa

SEQ ID NO: 1407 - RNA, 21 nt, Artificial Sequence
  5′ anti-sense siRNA sequences non-structural proteins (NSPs)
uuagcauaagcaguuguggca

SEQ ID NO: 1408 - RNA, 21 nt, Artificial Sequence
  5′ anti-sense siRNA sequences non-structural proteins (NSPs)
uuaacauuggccgugacagcu

SEQ ID NO: 1409 - RNA, 21 nt, Artificial Sequence
  5′ anti-sense siRNA sequences non-structural proteins (NSPs)
uuuuguuaccaucaguagaua

SEQ ID NO: 1410 - RNA, 21 nt, Artificial Sequence
  5′ anti-sense siRNA sequences non-structural proteins (NSPs)
uuucuauagagacacucauaa

SEQ ID NO: 1411 - RNA, 21 nt, Artificial Sequence
  5′ anti-sense siRNA sequences non-structural proteins (NSPs)
uuuaugcuagccacuagaccu

SEQ ID NO: 1412 - RNA, 21 nt, Artificial Sequence
  5′ anti-sense siRNA sequences non-structural proteins (NSPs)
uucuuuaugcuagccacuaga

SEQ ID NO: 1413 - RNA, 21 nt, Artificial Sequence
  5′ anti-sense siRNA sequences non-structural proteins (NSPs)
uuuaguaaggucagucucagu

SEQ ID NO: 1414 - RNA, 21 nt, Artificial Sequence
  5′ anti-sense siRNA sequences non-structural proteins (NSPs)
uuuaacuagcauuguauguug

SEQ ID NO: 1415 - RNA, 21 nt, Artificial Sequence
  5′ anti-sense siRNA sequences non-structural proteins (NSPs)
uuuuuacgauaucaucuacaa

SEQ ID NO: 1416 - RNA, 21 nt, Artificial Sequence
  5′ anti-sense siRNA sequences non-structural proteins (NSPs)
uucaaucauaaguguaccauc

SEQ ID NO: 1417 - RNA, 21 nt, Artificial Sequence
  5′ anti-sense siRNA sequences non-structural proteins (NSPs)
uucuuauguauuguaaguaca

SEQ ID NO: 1418 - RNA, 21 nt, Artificial Sequence
  5′ anti-sense siRNA sequences non-structural proteins (NSPs)
uuucuuauguauuguaaguac

SEQ ID NO: 1419 - RNA, 21 nt, Artificial Sequence
  5′ anti-sense siRNA sequences non-structural proteins (NSPs)
uuaacucaucauguagcuuuc

SEQ ID NO: 1420 - RNA, 21 nt, Artificial Sequence
  5′ anti-sense siRNA sequences non-structural proteins (NSPs)
acaauuaguuuuuaggaauuu

SEQ ID NO: 1421 - RNA, 21 nt, Artificial Sequence
  5′ anti-sense siRNA sequences non-structural proteins (NSPs)
aacaauuaguuuuuaggaauu

SEQ ID NO: 1422 - RNA, 21 nt, Artificial Sequence
  5′ anti-sense siRNA sequences non-structural proteins (NSPs)
aauugucaucuucguccuuuu

SEQ ID NO: 1423 - RNA, 21 nt, Artificial Sequence
  5′ anti-sense siRNA sequences non-structural proteins (NSPs)
uuguguauuuaguaagacguu

SEQ ID NO: 1424 - RNA, 21 nt, Artificial Sequence
  5′ anti-sense siRNA sequences non-structural proteins (NSPs)
uuaaugugucacaauuaccuu

SEQ ID NO: 1425 - RNA, 21 nt, Artificial Sequence
  5′ anti-sense siRNA sequences non-structural proteins (NSPs)
aauuguaugugacaaguauuu

SEQ ID NO: 1426 - RNA, 21 nt, Artificial Sequence
  5′ anti-sense siRNA sequences non-structural proteins (NSPs)
auaaucaucaucacaacaauu

SEQ ID NO: 1427 - RNA, 21 nt, Artificial Sequence
  5′ anti-sense siRNA sequences non-structural proteins (NSPs)
guuaccauugagaucuugauu

SEQ ID NO: 1428 - RNA, 21 nt, Artificial Sequence
  5′ anti-sense siRNA sequences non-structural proteins (NSPs)
cacuuaauguaaggcuuuguu

SEQ ID NO: 1429 - RNA, 21 nt, Artificial Sequence
  5′ anti-sense siRNA sequences non-structural proteins (NSPs)
auguaaguuuacauccugauu

SEQ ID NO: 1430 - RNA, 21 nt, Artificial Sequence
  5′ anti-sense siRNA sequences non-structural proteins (NSPs)
uacuuaagauucauuugaguu

SEQ ID NO: 1431 - RNA, 21 nt, Artificial Sequence
  5′ anti-sense siRNA sequences non-structural proteins (NSPs)
gcuauugauuucaauaauuuu

SEQ ID NO: 1432 - RNA, 21 nt, Artificial Sequence
  5′ anti-sense siRNA sequences non-structural proteins (NSPs)
uagaauuugcuuguuccaauu

SEQ ID NO: 1433 - RNA, 21 nt, Artificial Sequence
  5′ anti-sense siRNA sequences non-structural proteins (NSPs)
caauacuugagcacacucauu

SEQ ID NO: 1434 - RNA, 21 nt, Artificial Sequence
  5′ anti-sense siRNA sequences non-structural proteins (NSPs)
acacuauuagcauaagcaguu

SEQ ID NO: 1435 - RNA, 21 nt, Artificial Sequence
  5′ anti-sense siRNA sequences non-structural proteins (NSPs)
auacuuaucggcaauuuuguu

SEQ ID NO: 1436 - RNA, 21 nt, Artificial Sequence
  5′ anti-sense siRNA sequences non-structural proteins (NSPs)
gacauacuuaucggcaauuuu

SEQ ID NO: 1437 - RNA, 21 nt, Artificial Sequence
  5′ anti-sense siRNA sequences non-structural proteins (NSPs)
auaaguguaccaucuguuuuu

SEQ ID NO: 1438 - RNA, 21 nt, Artificial Sequence
  5′ anti-sense siRNA sequences non-structural proteins (NSPs)
cauaaguguaccaucuguuuu

SEQ ID NO: 1439 - RNA, 21 nt, Artificial Sequence
  5′ anti-sense siRNA sequences non-structural proteins (NSPs)
aucauaaguguaccaucuguu

SEQ ID NO: 1440 - RNA, 21 nt, Artificial Sequence
  5′ anti-sense siRNA sequences non-structural proteins (NSPs)
guuaacucaucauguagcuuu

SEQ ID NO: 1441 - RNA, 21 nt, Artificial Sequence
  5′ anti-sense siRNA sequences non-structural proteins (NSPs)
uugaucacaacuacagccaua

SEQ ID NO: 1442 - RNA, 21 nt, Artificial Sequence
  5′ anti-sense siRNA sequences non-structural proteins (NSPs)
uucuuggaagcgacaacaauu

SEQ ID NO: 1443 - RNA, 21 nt, Artificial Sequence
  5′ anti-sense siRNA sequences non-structural proteins (NSPs)
uuucuuggaagcgacaacaau

SEQ ID NO: 1444 - RNA, 21 nt, Artificial Sequence
  5′ anti-sense siRNA sequences non-structural proteins (NSPs)
uuuucuuggaagcgacaacaa

SEQ ID NO: 1445 - RNA, 21 nt, Artificial Sequence
  5′ anti-sense siRNA sequences non-structural proteins (NSPs)
uugucaucuucguccuuuucu

SEQ ID NO: 1446 - RNA, 21 nt, Artificial Sequence
  5′ anti-sense siRNA sequences non-structural proteins (NSPs)
uucauguugguaguuagagaa

SEQ ID NO: 1447 - RNA, 21 nt, Artificial Sequence
  5′ anti-sense siRNA sequences non-structural proteins (NSPs)
uucuucauguugguaguuaga

SEQ ID NO: 1448 - RNA, 21 nt, Artificial Sequence
  5′ anti-sense siRNA sequences non-structural proteins (NSPs)
uuagcaacagcuggacaaucc

SEQ ID NO: 1449 - RNA, 21 nt, Artificial Sequence
  5′ anti-sense siRNA sequences non-structural proteins (NSPs)
uuuagcaacagcuggacaauc

SEQ ID NO: 1450 - RNA, 21 nt, Artificial Sequence
  5′ anti-sense siRNA sequences non-structural proteins (NSPs)
uuguaugugacaaguauuucu

SEQ ID NO: 1451 - RNA, 21 nt, Artificial Sequence
  5′ anti-sense siRNA sequences non-structural proteins (NSPs)
uucaccuaaguuggcguauac

SEQ ID NO: 1452 - RNA, 21 nt, Artificial Sequence
  5′ anti-sense siRNA sequences non-structural proteins (NSPs)
uugagaucuugauuaucuaau

SEQ ID NO: 1453 - RNA, 21 nt, Artificial Sequence
  5′ anti-sense siRNA sequences non-structural proteins (NSPs)
uuaccauugagaucuugauua

SEQ ID NO: 1454 - RNA, 21 nt, Artificial Sequence
  5′ anti-sense siRNA sequences non-structural proteins (NSPs)
uuaagucagugucaacaugug

SEQ ID NO: 1455 - RNA, 21 nt, Artificial Sequence
  5′ anti-sense siRNA sequences non-structural proteins (NSPs)
uuaaccucucuuccgugaagu

SEQ ID NO: 1456 - RNA, 21 nt, Artificial Sequence
  5′ anti-sense siRNA sequences non-structural proteins (NSPs)
uuuaaccucucuuccgugaag

SEQ ID NO: 1457 - RNA, 21 nt, Artificial Sequence
  5′ anti-sense siRNA sequences non-structural proteins (NSPs)
uugcacaaugcagaaugcauc

SEQ ID NO: 1458 - RNA, 21 nt, Artificial Sequence
  5′ anti-sense siRNA sequences non-structural proteins (NSPs)
uuugcacaaugcagaaugcau

SEQ ID NO: 1459 - RNA, 21 nt, Artificial Sequence
  5′ anti-sense siRNA sequences non-structural proteins (NSPs)
uuacauccugauuauguacaa

SEQ ID NO: 1460 - RNA, 21 nt, Artificial Sequence
  5′ anti-sense siRNA sequences non-structural proteins (NSPs)
uuuacauccugauuauguaca

SEQ ID NO: 1461 - RNA, 21 nt, Artificial Sequence
  5′ anti-sense siRNA sequences non-structural proteins (NSPs)
uuaccagaagcagcgugcaua

SEQ ID NO: 1462 - RNA, 21 nt, Artificial Sequence
  5′ anti-sense siRNA sequences non-structural proteins (NSPs)
uucaacagaacuuccuuccuu

SEQ ID NO: 1463 - RNA, 21 nt, Artificial Sequence
  5′ anti-sense siRNA sequences non-structural proteins (NSPs)
uugguagauuauaacgauagu

SEQ ID NO: 1464 - RNA, 21 nt, Artificial Sequence
  5′ anti-sense siRNA sequences non-structural proteins (NSPs)
uuguugguagauuauaacgau

SEQ ID NO: 1465 - RNA, 21 nt, Artificial Sequence
  5′ anti-sense siRNA sequences non-structural proteins (NSPs)
uugucugauaucacacauugu

SEQ ID NO: 1466 - RNA, 21 nt, Artificial Sequence
  5′ anti-sense siRNA sequences non-structural proteins (NSPs)
uuugucuagguuguugacgau

SEQ ID NO: 1467 - RNA, 21 nt, Artificial Sequence
  5′ anti-sense siRNA sequences non-structural proteins (NSPs)
uugcacuaauggcauacuuaa

SEQ ID NO: 1468 - RNA, 21 nt, Artificial Sequence
  5′ anti-sense siRNA sequences non-structural proteins (NSPs)
uuugcacuaauggcauacuua

SEQ ID NO: 1469 - RNA, 21 nt, Artificial Sequence
  5′ anti-sense siRNA sequences non-structural proteins (NSPs)
uucuuugcacuaauggcauac

SEQ ID NO: 1470 - RNA, 21 nt, Artificial Sequence
  5′ anti-sense siRNA sequences non-structural proteins (NSPs)
uuggucauaguacuacagaua

SEQ ID NO: 1471 - RNA, 21 nt, Artificial Sequence
  5′ anti-sense siRNA sequences non-structural proteins (NSPs)
uuacuacaguagcuccucuag

SEQ ID NO: 1472 - RNA, 21 nt, Artificial Sequence
  5′ anti-sense siRNA sequences non-structural proteins (NSPs)
uuguuccaauuacuacaguag

SEQ ID NO: 1473 - RNA, 21 nt, Artificial Sequence
  5′ anti-sense siRNA sequences non-structural proteins (NSPs)
uugcuuguuccaauuacuaca

SEQ ID NO: 1474 - RNA, 21 nt, Artificial Sequence
  5′ anti-sense siRNA sequences non-structural proteins (NSPs)
uuugcuuguuccaauuacuac

SEQ ID NO: 1475 - RNA, 21 nt, Artificial Sequence
  5′ anti-sense siRNA sequences non-structural proteins (NSPs)
uucuaagcauguuaggcaugg

SEQ ID NO: 1476 - RNA, 21 nt, Artificial Sequence
  5′ anti-sense siRNA sequences non-structural proteins (NSPs)
uucacucaauacuugagcaca

SEQ ID NO: 1477 - RNA, 21 nt, Artificial Sequence
  5′ anti-sense siRNA sequences non-structural proteins (NSPs)
uuucacucaauacuugagcac

SEQ ID NO: 1478 - RNA, 21 nt, Artificial Sequence
  5′ anti-sense siRNA sequences non-structural proteins (NSPs)
uuuguuaccaucaguagauaa

SEQ ID NO: 1479 - RNA, 21 nt, Artificial Sequence
  5′ anti-sense siRNA sequences non-structural proteins (NSPs)
uuaugcuagccacuagaccuu

SEQ ID NO: 1480 - RNA, 21 nt, Artificial Sequence
  5′ anti-sense siRNA sequences non-structural proteins (NSPs)
uuaguaaggucagucucaguc

SEQ ID NO: 1481 - RNA, 21 nt, Artificial Sequence
  5′ anti-sense siRNA sequences non-structural proteins (NSPs)
uuaacuagcauuguauguuga

SEQ ID NO: 1482 - RNA, 21 nt, Artificial Sequence
  5′ anti-sense siRNA sequences non-structural proteins (NSPs)
uuaguaagcauaacagaauac

SEQ ID NO: 1483 - RNA, 21 nt, Artificial Sequence
  5′ anti-sense siRNA sequences non-structural proteins (NSPs)
acaacuacagccauaaccuuu

SEQ ID NO: 1484 - RNA, 21 nt, Artificial Sequence
  5′ anti-sense siRNA sequences non-structural proteins (NSPs)
cacaacuacagccauaaccuu

SEQ ID NO: 1485 - RNA, 21 nt, Artificial Sequence
  5′ anti-sense siRNA sequences non-structural proteins (NSPs)
uucuuggaagcgacaacaauu

SEQ ID NO: 1486 - RNA, 21 nt, Artificial Sequence
  5′ anti-sense siRNA sequences non-structural proteins (NSPs)
ugucaucuucguccuuuucuu

SEQ ID NO: 1487 - RNA, 21 nt, Artificial Sequence
  5′ anti-sense siRNA sequences non-structural proteins (NSPs)
uguuucuucauguugguaguu

SEQ ID NO: 1488 - RNA, 21 nt, Artificial Sequence
  5′ anti-sense siRNA sequences non-structural proteins (NSPs)
uaugugacaaguauuucuuuu

SEQ ID NO: 1489 - RNA, 21 nt, Artificial Sequence
  5′ anti-sense siRNA sequences non-structural proteins (NSPs)
guaugugacaaguauuucuuu

SEQ ID NO: 1490 - RNA, 21 nt, Artificial Sequence
  5′ anti-sense siRNA sequences non-structural proteins (NSPs)
uguaugugacaaguauuucuu

SEQ ID NO: 1491 - RNA, 21 nt, Artificial Sequence
  5′ anti-sense siRNA sequences non-structural proteins (NSPs)
aucauaccaguccuuuuuauu

SEQ ID NO: 1492 - RNA, 21 nt, Artificial Sequence
  5′ anti-sense siRNA sequences non-structural proteins (NSPs)
ucacagaauuguacuguuuuu

SEQ ID NO: 1493 - RNA, 21 nt, Artificial Sequence
  5′ anti-sense siRNA sequences non-structural proteins (NSPs)
aucacagaauuguacuguuuu

SEQ ID NO: 1494 - RNA, 21 nt, Artificial Sequence
  5′ anti-sense siRNA sequences non-structural proteins (NSPs)
caucacagaauuguacuguuu

SEQ ID NO: 1495 - RNA, 21 nt, Artificial Sequence
  5′ anti-sense siRNA sequences non-structural proteins (NSPs)
gucaagguuaauauaggcauu

SEQ ID NO: 1496 - RNA, 21 nt, Artificial Sequence
  5′ anti-sense siRNA sequences non-structural proteins (NSPs)
ucaacaugugacucugcaguu

SEQ ID NO: 1497 - RNA, 21 nt, Artificial Sequence
  5′ anti-sense siRNA sequences non-structural proteins (NSPs)
aagucuagagcuauguaaguu

SEQ ID NO: 1498 - RNA, 21 nt, Artificial Sequence
  5′ anti-sense siRNA sequences non-structural proteins (NSPs)
gcguuuaucuaguaauagauu

SEQ ID NO: 1499 - RNA, 21 nt, Artificial Sequence
  5′ anti-sense siRNA sequences non-structural proteins (NSPs)
uucaacagaacuuccuuccuu

SEQ ID NO: 1500 - RNA, 21 nt, Artificial Sequence
  5′ anti-sense siRNA sequences non-structural proteins (NSPs)
auaaucgcugauagcagcauu

SEQ ID NO: 1501 - RNA, 21 nt, Artificial Sequence
  5′ anti-sense siRNA sequences non-structural proteins (NSPs)
aucacacauuguugguagauu

SEQ ID NO: 1502 - RNA, 21 nt, Artificial Sequence
  5′ anti-sense siRNA sequences non-structural proteins (NSPs)
ugucugauaucacacauuguu

SEQ ID NO: 1503 - RNA, 21 nt, Artificial Sequence
  5′ anti-sense siRNA sequences non-structural proteins (NSPs)
guuguugacgaugacuugguu

SEQ ID NO: 1504 - RNA, 21 nt, Artificial Sequence
  5′ anti-sense siRNA sequences non-structural proteins (NSPs)
cuagguuguugacgaugacuu

SEQ ID NO: 1505 - RNA, 21 nt, Artificial Sequence
  5′ anti-sense siRNA sequences non-structural proteins (NSPs)
agcugauuugucuagguuguu

SEQ ID NO: 1506 - RNA, 21 nt, Artificial Sequence
  5′ anti-sense siRNA sequences non-structural proteins (NSPs)
cuuugcacuaauggcauacuu

SEQ ID NO: 1507 - RNA, 21 nt, Artificial Sequence
  5′ anti-sense siRNA sequences non-structural proteins (NSPs)
ggcuauugauuucaauaauuu

SEQ ID NO: 1508 - RNA, 21 nt, Artificial Sequence
  5′ anti-sense siRNA sequences non-structural proteins (NSPs)
ggccauaauucuaagcauguu

SEQ ID NO: 1509 - RNA, 21 nt, Artificial Sequence
  5′ anti-sense siRNA sequences non-structural proteins (NSPs)
agaacaagugaggccauaauu

SEQ ID NO: 1510 - RNA, 21 nt, Artificial Sequence
  5′ anti-sense siRNA sequences non-structural proteins (NSPs)
gcuacaacacguuguauguuu

SEQ ID NO: 1511 - RNA, 21 nt, Artificial Sequence
  5′ anti-sense siRNA sequences non-structural proteins (NSPs)
ugaccauuucacucaauacuu

SEQ ID NO: 1512 - RNA, 21 nt, Artificial Sequence
  5′ anti-sense siRNA sequences non-structural proteins (NSPs)
uaacauuggccgugacagcuu

SEQ ID NO: 1513 - RNA, 21 nt, Artificial Sequence
  5′ anti-sense siRNA sequences non-structural proteins (NSPs)
ggacauacuuaucggcaauuu

SEQ ID NO: 1514 - RNA, 21 nt, Artificial Sequence
  5′ anti-sense siRNA sequences non-structural proteins (NSPs)
uuaugcuagccacuagaccuu

SEQ ID NO: 1515 - RNA, 21 nt, Artificial Sequence
  5′ anti-sense siRNA sequences non-structural proteins (NSPs)
ucauaaguguaccaucuguuu

SEQ ID NO: 1516 - RNA, 21 nt, Artificial Sequence
  5′ anti-sense siRNA sequences non-structural proteins (NSPs)
cauacuccugauuaggauguu

SEQ ID NO: 1517 - RNA, 21 nt, Artificial Sequence
  5′ anti-sense siRNA sequences non-structural proteins (NSPs)
cauguagcuuucuuauguauu

SEQ ID NO: 1518 - RNA, 21 nt, Artificial Sequence
  5′ anti-sense siRNA sequences non-structural proteins (NSPs)
aacucaucauguagcuuucuu

SEQ ID NO: 1519 - RNA, 21 nt, Artificial Sequence
  5′ anti-sense siRNA sequences non-structural proteins (NSPs)
uguuaacucaucauguagcuu

SEQ ID NO: 1520 - RNA, 21 nt, Artificial Sequence
  5′ anti-sense siRNA sequences non-structural proteins (NSPs)
augucuaacauguguccuguu

SEQ ID NO: 1521 - RNA, 21 nt, Artificial Sequence
  5′ anti-sense siRNA sequences non-structural proteins (NSPs)
auaccuugaaguguuaucauu

SEQ ID NO: 1522 - RNA, 21 nt, Artificial Sequence
  5′ anti-sense siRNA sequences non-structural proteins (NSPs)
uugugcaucagcugacugaag

SEQ ID NO: 1523 - RNA, 21 nt, Artificial Sequence
  5′ anti-sense siRNA sequences non-structural proteins (NSPs)
uucguccuuuucuuggaagcg

SEQ ID NO: 1524 - RNA, 21 nt, Artificial Sequence
  5′ anti-sense siRNA sequences non-structural proteins (NSPs)
uugacgugauauaugugguac

SEQ ID NO: 1525 - RNA, 21 nt, Artificial Sequence
  5′ anti-sense siRNA sequences non-structural proteins (NSPs)
uuggcguauacgcguaauaua

SEQ ID NO: 1526 - RNA, 21 nt, Artificial Sequence
  5′ anti-sense siRNA sequences non-structural proteins (NSPs)
uuucgcauggcaucacagaau

SEQ ID NO: 1527 - RNA, 21 nt, Artificial Sequence
  5′ anti-sense siRNA sequences non-structural proteins (NSPs)
uuccgugaagucauauuuuaa

SEQ ID NO: 1528 - RNA, 21 nt, Artificial Sequence
  5′ anti-sense siRNA sequences non-structural proteins (NSPs)
uugguuagcauuaauacagcc

SEQ ID NO: 1529 - RNA, 21 nt, Artificial Sequence
  5′ anti-sense siRNA sequences non-structural proteins (NSPs)
uugacgaugacuugguuagca

SEQ ID NO: 1530 - RNA, 21 nt, Artificial Sequence
  5′ anti-sense siRNA sequences non-structural proteins (NSPs)
uuguugacgaugacuugguua

SEQ ID NO: 1531 - RNA, 21 nt, Artificial Sequence
  5′ anti-sense siRNA sequences non-structural proteins (NSPs)
uugucuagguuguugacgaug

SEQ ID NO: 1532 - RNA, 21 nt, Artificial Sequence
  5′ anti-sense siRNA sequences non-structural proteins (NSPs)
uugauccucauaacucauuga

SEQ ID NO: 1533 - RNA, 21 nt, Artificial Sequence
  5′ anti-sense siRNA sequences non-structural proteins (NSPs)
uugugccaaccaccauagaau

SEQ ID NO: 1534 - RNA, 21 nt, Artificial Sequence
  5′ anti-sense siRNA sequences non-structural proteins (NSPs)
uuaggcauggcucuaucacau

SEQ ID NO: 1535 - RNA, 21 nt, Artificial Sequence
  5′ anti-sense siRNA sequences non-structural proteins (NSPs)
uugagcacacucauuagcuaa

SEQ ID NO: 1536 - RNA, 21 nt, Artificial Sequence
  5′ anti-sense siRNA sequences non-structural proteins (NSPs)
uuguggcaucuccugaugagg

SEQ ID NO: 1537 - RNA, 21 nt, Artificial Sequence
  5′ anti-sense siRNA sequences non-structural proteins (NSPs)
uuguagcaucaccggaugaug

SEQ ID NO: 1538 - RNA, 21 nt, Artificial Sequence
  5′ anti-sense siRNA sequences non-structural proteins (NSPs)
uuaucggcaauuuuguuacca

SEQ ID NO: 1539 - RNA, 21 nt, Artificial Sequence
  5′ anti-sense siRNA sequences non-structural proteins (NSPs)
uugagaugcauaagugcuauu

SEQ ID NO: 1540 - RNA, 21 nt, Artificial Sequence
  5′ anti-sense siRNA sequences non-structural proteins (NSPs)
uucaugagguccuuuaguaag

SEQ ID NO: 1541 - RNA, 21 nt, Artificial Sequence
  5′ anti-sense siRNA sequences non-structural proteins (NSPs)
aagcgacaacaauuaguuuuu

SEQ ID NO: 1542 - RNA, 21 nt, Artificial Sequence
  5′ anti-sense siRNA sequences non-structural proteins (NSPs)
gaagcgacaacaauuaguuuu

SEQ ID NO: 1543 - RNA, 21 nt, Artificial Sequence
  5′ anti-sense siRNA sequences non-structural proteins (NSPs)
gguaccaugucaccgucuauu

SEQ ID NO: 1544 - RNA, 21 nt, Artificial Sequence
  5′ anti-sense siRNA sequences non-structural proteins (NSPs)
gcauagacgaggucugccauu

SEQ ID NO: 1545 - RNA, 21 nt, Artificial Sequence
  5′ anti-sense siRNA sequences non-structural proteins (NSPs)
gcauagacuaaaucagccauu

SEQ ID NO: 1546 - RNA, 21 nt, Artificial Sequence
  5′ anti-sense siRNA sequences non-structural proteins (NSPs)
gcaucacagaauuguacuguu

SEQ ID NO: 1547 - RNA, 21 nt, Artificial Sequence
  5′ anti-sense siRNA sequences non-structural proteins (NSPs)
acaccaacaauaccagcauuu

SEQ ID NO: 1548 - RNA, 21 nt, Artificial Sequence
  5′ anti-sense siRNA sequences non-structural proteins (NSPs)
uacaccaacaauaccagcauu

SEQ ID NO: 1549 - RNA, 21 nt, Artificial Sequence
  5′ anti-sense siRNA sequences non-structural proteins (NSPs)
accaguuaccauugagaucuu

SEQ ID NO: 1550 - RNA, 21 nt, Artificial Sequence
  5′ anti-sense siRNA sequences non-structural proteins (NSPs)
cuccacuaccuggcgugguuu

SEQ ID NO: 1551 - RNA, 21 nt, Artificial Sequence
  5′ anti-sense siRNA sequences non-structural proteins (NSPs)
cuccgcagccuggugcuacuu

SEQ ID NO: 1552 - RNA, 21 nt, Artificial Sequence
  5′ anti-sense siRNA sequences non-structural proteins (NSPs)
acuccacuaccuggcgugguu

SEQ ID NO: 1553 - RNA, 21 nt, Artificial Sequence
  5′ anti-sense siRNA sequences non-structural proteins (NSPs)
cucuuccgugaagucauauuu

SEQ ID NO: 1554 - RNA, 21 nt, Artificial Sequence
  5′ anti-sense siRNA sequences non-structural proteins (NSPs)
ucucugaagugguauccaguu

SEQ ID NO: 1555 - RNA, 21 nt, Artificial Sequence
  5′ anti-sense siRNA sequences non-structural proteins (NSPs)
agucuagagcuauguaaguuu

SEQ ID NO: 1556 - RNA, 21 nt, Artificial Sequence
  5′ anti-sense siRNA sequences non-structural proteins (NSPs)
agcauacacaaguaauuccuu

SEQ ID NO: 1557 - RNA, 21 nt, Artificial Sequence
  5′ anti-sense siRNA sequences non-structural proteins (NSPs)
cagcagcauacacaaguaauu

SEQ ID NO: 1558 - RNA, 21 nt, Artificial Sequence
  5′ anti-sense siRNA sequences non-structural proteins (NSPs)
aguugucugauaucacacauu

SEQ ID NO: 1559 - RNA, 21 nt, Artificial Sequence
  5′ anti-sense siRNA sequences non-structural proteins (NSPs)
gacgaugacuugguuagcauu

SEQ ID NO: 1560 - RNA, 21 nt, Artificial Sequence
  5′ anti-sense siRNA sequences non-structural proteins (NSPs)
ucuugauccucauaacucauu

SEQ ID NO: 1561 - RNA, 21 nt, Artificial Sequence
  5′ anti-sense siRNA sequences non-structural proteins (NSPs)
uggcauacuuaagauucauuu

SEQ ID NO: 1562 - RNA, 21 nt, Artificial Sequence
  5′ anti-sense siRNA sequences non-structural proteins (NSPs)
auggcauacuuaagauucauu

SEQ ID NO: 1563 - RNA, 21 nt, Artificial Sequence
  5′ anti-sense siRNA sequences non-structural proteins (NSPs)
acuaauggcauacuuaagauu

SEQ ID NO: 1564 - RNA, 21 nt, Artificial Sequence
  5′ anti-sense siRNA sequences non-structural proteins (NSPs)
cggcuauugauuucaauaauu

SEQ ID NO: 1565 - RNA, 21 nt, Artificial Sequence
  5′ anti-sense siRNA sequences non-structural proteins (NSPs)
cucuaguggcggcuauugauu

SEQ ID NO: 1566 - RNA, 21 nt, Artificial Sequence
  5′ anti-sense siRNA sequences non-structural proteins (NSPs)
gcuccucuaguggcggcuauu

SEQ ID NO: 1567 - RNA, 21 nt, Artificial Sequence
  5′ anti-sense siRNA sequences non-structural proteins (NSPs)
ccaccauagaauuugcuuguu

SEQ ID NO: 1568 - RNA, 21 nt, Artificial Sequence
  5′ anti-sense siRNA sequences non-structural proteins (NSPs)
caaccaccauagaauuugcuu

SEQ ID NO: 1569 - RNA, 21 nt, Artificial Sequence
  5′ anti-sense siRNA sequences non-structural proteins (NSPs)
agcuacaacacguuguauguu

SEQ ID NO: 1570 - RNA, 21 nt, Artificial Sequence
  5′ anti-sense siRNA sequences non-structural proteins (NSPs)
ugugacaagcuacaacacguu

SEQ ID NO: 1571 - RNA, 21 nt, Artificial Sequence
  5′ anti-sense siRNA sequences non-structural proteins (NSPs)
ugaugagguuccaccugguuu

SEQ ID NO: 1572 - RNA, 21 nt, Artificial Sequence
  5′ anti-sense siRNA sequences non-structural proteins (NSPs)
cugaugagguuccaccugguu

SEQ ID NO: 1573 - RNA, 21 nt, Artificial Sequence
  5′ anti-sense siRNA sequences non-structural proteins (NSPs)
cggaugauguuccaccugguu

SEQ ID NO: 1574 - RNA, 21 nt, Artificial Sequence
  5′ anti-sense siRNA sequences non-structural proteins (NSPs)
guggcaucuccugaugagguu

SEQ ID NO: 1575 - RNA, 21 nt, Artificial Sequence
  5′ anti-sense siRNA sequences non-structural proteins (NSPs)
guagcaucaccggaugauguu

SEQ ID NO: 1576 - RNA, 21 nt, Artificial Sequence
  5′ anti-sense siRNA sequences non-structural proteins (NSPs)
cggacauacuuaucggcaauu

SEQ ID NO: 1577 - RNA, 21 nt, Artificial Sequence
  5′ anti-sense siRNA sequences non-structural proteins (NSPs)
ucugugucaacaucucuauuu

SEQ ID NO: 1578 - RNA, 21 nt, Artificial Sequence
  5′ anti-sense siRNA sequences non-structural proteins (NSPs)
gucugugucaacaucucuauu

SEQ ID NO: 1579 - RNA, 21 nt, Artificial Sequence
  5′ anti-sense siRNA sequences non-structural proteins (NSPs)
uugagaugcauaagugcuauu

SEQ ID NO: 1580 - RNA, 21 nt, Artificial Sequence
  5′ anti-sense siRNA sequences non-structural proteins (NSPs)
ucagucucaguccaacauuuu

SEQ ID NO: 1581 - RNA, 21 nt, Artificial Sequence
  5′ anti-sense siRNA sequences non-structural proteins (NSPs)
gucagucucaguccaacauuu

SEQ ID NO: 1582 - RNA, 21 nt, Artificial Sequence
  5′ anti-sense siRNA sequences non-structural proteins (NSPs)
ggucagucucaguccaacauu

SEQ ID NO: 1583 - RNA, 21 nt, Artificial Sequence
  5′ anti-sense siRNA sequences non-structural proteins (NSPs)
auacuccugauuaggauguuu

SEQ ID NO: 1584 - RNA, 21 nt, Artificial Sequence
  5′ anti-sense siRNA sequences non-structural proteins (NSPs)
gacaucagcauacuccugauu

SEQ ID NO: 1585 - RNA, 21 nt, Artificial Sequence
  5′ anti-sense siRNA sequences non-structural proteins (NSPs)
uuggaagcgacaacaauuagu

SEQ ID NO: 1586 - RNA, 21 nt, Artificial Sequence
  5′ anti-sense siRNA sequences non-structural proteins (NSPs)
uuggcguacacguucaccuaa

SEQ ID NO: 1587 - RNA, 21 nt, Artificial Sequence
  5′ anti-sense siRNA sequences non-structural proteins (NSPs)
uucgcauggcaucacagaauu

SEQ ID NO: 1588 - RNA, 21 nt, Artificial Sequence
  5′ anti-sense siRNA sequences non-structural proteins (NSPs)
uugcgagcaagaacaagugag

SEQ ID NO: 1589 - RNA, 21 nt, Artificial Sequence
  5′ anti-sense siRNA sequences non-structural proteins (NSPs)
uuugcgagcaagaacaaguga

SEQ ID NO: 1590 - RNA, 21 nt, Artificial Sequence
  5′ anti-sense siRNA sequences non-structural proteins (NSPs)
uuccaccugguuuaacauaua

SEQ ID NO: 1591 - RNA, 21 nt, Artificial Sequence
  5′ anti-sense siRNA sequences non-structural proteins (NSPs)
uugcggacauacuuaucggca

SEQ ID NO: 1592 - RNA, 21 nt, Artificial Sequence
  5′ anti-sense siRNA sequences non-structural proteins (NSPs)
gugccugugccgcacgguguu

SEQ ID NO: 1593 - RNA, 21 nt, Artificial Sequence
  5′ anti-sense siRNA sequences non-structural proteins (NSPs)
ggaagcgacaacaauuaguuu

SEQ ID NO: 1594 - RNA, 21 nt, Artificial Sequence
  5′ anti-sense siRNA sequences non-structural proteins (NSPs)
uggaagcgacaacaauuaguu

SEQ ID NO: 1595 - RNA, 21 nt, Artificial Sequence
  5′ anti-sense siRNA sequences non-structural proteins (NSPs)
agcaacagcuggacaauccuu

SEQ ID NO: 1596 - RNA, 21 nt, Artificial Sequence
  5′ anti-sense siRNA sequences non-structural proteins (NSPs)
gaggucugccauuguguauuu

SEQ ID NO: 1597 - RNA, 21 nt, Artificial Sequence
  5′ anti-sense siRNA sequences non-structural proteins (NSPs)
cgaggucugccauuguguauu

SEQ ID NO: 1598 - RNA, 21 nt, Artificial Sequence
  5′ anti-sense siRNA sequences non-structural proteins (NSPs)
gcguacacguucaccuaaguu

SEQ ID NO: 1599 - RNA, 21 nt, Artificial Sequence
  5′ anti-sense siRNA sequences non-structural proteins (NSPs)
uucgcauggcaucacagaauu

SEQ ID NO: 1600 - RNA, 21 nt, Artificial Sequence
  5′ anti-sense siRNA sequences non-structural proteins (NSPs)
ucuuccgugaagucauauuuu

SEQ ID NO: 1601 - RNA, 21 nt, Artificial Sequence
  5′ anti-sense siRNA sequences non-structural proteins (NSPs)
ucucuuccgugaagucauauu

SEQ ID NO: 1602 - RNA, 21 nt, Artificial Sequence
  5′ anti-sense siRNA sequences non-structural proteins (NSPs)
accagcugauuugucuagguu

SEQ ID NO: 1603 - RNA, 21 nt, Artificial Sequence
  5′ anti-sense siRNA sequences non-structural proteins (NSPs)
agcuacggugcgagcucuauu

SEQ ID NO: 1604 - RNA, 21 nt, Artificial Sequence
  5′ anti-sense siRNA sequences non-structural proteins (NSPs)
ucuaguggcggcuauugauuu

SEQ ID NO: 1605 - RNA, 21 nt, Artificial Sequence
  5′ anti-sense siRNA sequences non-structural proteins (NSPs)
gugccaaccaccauagaauuu

SEQ ID NO: 1606 - RNA, 21 nt, Artificial Sequence
  5′ anti-sense siRNA sequences non-structural proteins (NSPs)
ugugccaaccaccauagaauu

SEQ ID NO: 1607 - RNA, 21 nt, Artificial Sequence
  5′ anti-sense siRNA sequences non-structural proteins (NSPs)
uaggcauggcucuaucacauu

SEQ ID NO: 1608 - RNA, 21 nt, Artificial Sequence
  5′ anti-sense siRNA sequences non-structural proteins (NSPs)
gaaccgccacacaugaccauu

SEQ ID NO: 1609 - RNA, 21 nt, Artificial Sequence
  5′ anti-sense siRNA sequences non-structural proteins (NSPs)
ucgucagagaguaucaucauu

SEQ ID NO: 1610 - RNA, 21 nt, Artificial Sequence
  5′ anti-sense siRNA sequences non-structural proteins (NSPs)
ucucaguccaacauuuugcuu

SEQ ID NO: 1611 - RNA, 21 nt, Artificial Sequence
  5′ anti-sense siRNA sequences non-structural proteins (NSPs)
uucgcggaguugaucacaacu

SEQ ID NO: 1612 - RNA, 21 nt, Artificial Sequence
  5′ anti-sense siRNA sequences non-structural proteins (NSPs)
uuggccgugacagcuugacaa

SEQ ID NO: 1613 - RNA, 21 nt, Artificial Sequence
  5′ anti-sense siRNA sequences non-structural proteins (NSPs)
ugccugugccgcacgguguuu

SEQ ID NO: 1614 - RNA, 21 nt, Artificial Sequence
  5′ anti-sense siRNA sequences non-structural proteins (NSPs)
acggugcgagcucuauucuuu

SEQ ID NO: 1615 - RNA, 21 nt, Artificial Sequence
  5′ anti-sense siRNA sequences non-structural proteins (NSPs)
uacggugcgagcucuauucuu

SEQ ID NO: 1616 - RNA, 21 nt, Artificial Sequence
  5′ anti-sense siRNA sequences non-structural proteins (NSPs)
aggcauggcucuaucacauuu

SEQ ID NO: 1617 - RNA, 21 nt, Artificial Sequence
  5′ anti-sense siRNA sequences non-structural proteins (NSPs)
aaccgccacacaugaccauuu

SEQ ID NO: 1618 - RNA, 21 nt, Artificial Sequence
  5′ anti-sense siRNA sequences non-structural proteins (NSPs)
uuacaauaauagcucauaccu

SEQ ID NO: 1619 - RNA, 21 nt, Artificial Sequence
  5′ anti-sense siRNA sequences non-structural proteins (NSPs)
uuuacaauaauagcucauacc

SEQ ID NO: 1620 - RNA, 21 nt, Artificial Sequence
  5′ anti-sense siRNA sequences non-structural proteins (NSPs)
uuuuaguuacacgauaaccag

SEQ ID NO: 1621 - RNA, 21 nt, Artificial Sequence
  5′ anti-sense siRNA sequences non-structural proteins (NSPs)
uuuuuaguuacacgauaacca

SEQ ID NO: 1622 - RNA, 21 nt, Artificial Sequence
  5′ anti-sense siRNA sequences non-structural proteins (NSPs)
uuaauuuguaaguuguuguac

SEQ ID NO: 1623 - RNA, 21 nt, Artificial Sequence
  5′ anti-sense siRNA sequences non-structural proteins (NSPs)
uuuaauuuguaaguuguugua

SEQ ID NO: 1624 - RNA, 21 nt, Artificial Sequence
  5′ anti-sense siRNA sequences non-structural proteins (NSPs)
uugauaauuugcaacauugcu

SEQ ID NO: 1625 - RNA, 21 nt, Artificial Sequence
  5′ anti-sense siRNA sequences non-structural proteins (NSPs)
uuugauaauuugcaacauugc

SEQ ID NO: 1626 - RNA, 21 nt, Artificial Sequence
  5′ anti-sense siRNA sequences non-structural proteins (NSPs)
uuuugauaauuugcaacauug

SEQ ID NO: 1627 - RNA, 21 nt, Artificial Sequence
  5′ anti-sense siRNA sequences non-structural proteins (NSPs)
uuuucauaagucuacacacug

SEQ ID NO: 1628 - RNA, 21 nt, Artificial Sequence
  5′ anti-sense siRNA sequences non-structural proteins (NSPs)
uuuacauuacaagagugagcu

SEQ ID NO: 1629 - RNA, 21 nt, Artificial Sequence
  5′ anti-sense siRNA sequences non-structural proteins (NSPs)
cuauuuguacuuuacuguuuu

SEQ ID NO: 1630 - RNA, 21 nt, Artificial Sequence
  5′ anti-sense siRNA sequences non-structural proteins (NSPs)
acauuuaauuuguaaguuguu

SEQ ID NO: 1631 - RNA, 21 nt, Artificial Sequence
  5′ anti-sense siRNA sequences non-structural proteins (NSPs)
uaauucuaacauagugcucuu

SEQ ID NO: 1632 - RNA, 21 nt, Artificial Sequence
  5′ anti-sense siRNA sequences non-structural proteins (NSPs)
cuuuugauaauuugcaacauu

SEQ ID NO: 1633 - RNA, 21 nt, Artificial Sequence
  5′ anti-sense siRNA sequences non-structural proteins (NSPs)
uuacaacauaagaauggucua

SEQ ID NO: 1634 - RNA, 21 nt, Artificial Sequence
  5′ anti-sense siRNA sequences non-structural proteins (NSPs)
uuuacaacauaagaauggucu

SEQ ID NO: 1635 - RNA, 21 nt, Artificial Sequence
  5′ anti-sense siRNA sequences non-structural proteins (NSPs)
uugauaugacauggucguaac

SEQ ID NO: 1636 - RNA, 21 nt, Artificial Sequence
  5′ anti-sense siRNA sequences non-structural proteins (NSPs)
uuaugugauguugauaugaca

SEQ ID NO: 1637 - RNA, 21 nt, Artificial Sequence
  5′ anti-sense siRNA sequences non-structural proteins (NSPs)
uuuaugugauguugauaugac

SEQ ID NO: 1638 - RNA, 21 nt, Artificial Sequence
  5′ anti-sense siRNA sequences non-structural proteins (NSPs)
uuaugugauuuacaauaauag

SEQ ID NO: 1639 - RNA, 21 nt, Artificial Sequence
  5′ anti-sense siRNA sequences non-structural proteins (NSPs)
uuuaugugauuuacaauaaua

SEQ ID NO: 1640 - RNA, 21 nt, Artificial Sequence
  5′ anti-sense siRNA sequences non-structural proteins (NSPs)
uuaguuacacgauaaccagua

SEQ ID NO: 1641 - RNA, 21 nt, Artificial Sequence
  5′ anti-sense siRNA sequences non-structural proteins (NSPs)
uuuaguuacacgauaaccagu

SEQ ID NO: 1642 - RNA, 21 nt, Artificial Sequence
  5′ anti-sense siRNA sequences non-structural proteins (NSPs)
uuacuguuuuuaguuacacga

SEQ ID NO: 1643 - RNA, 21 nt, Artificial Sequence
  5′ anti-sense siRNA sequences non-structural proteins (NSPs)
uuuacuguuuuuaguuacacg

SEQ ID NO: 1644 - RNA, 21 nt, Artificial Sequence
  5′ anti-sense siRNA sequences non-structural proteins (NSPs)
uuguacuuuacuguuuuuagu

SEQ ID NO: 1645 - RNA, 21 nt, Artificial Sequence
  5′ anti-sense siRNA sequences non-structural proteins (NSPs)
uuuguacuuuacuguuuuuag

SEQ ID NO: 1646 - RNA, 21 nt, Artificial Sequence
  5′ anti-sense siRNA sequences non-structural proteins (NSPs)
uuguaaguuguuguaccucgg

SEQ ID NO: 1647 - RNA, 21 nt, Artificial Sequence
  5′ anti-sense siRNA sequences non-structural proteins (NSPs)
uuuguaaguuguuguaccucg

SEQ ID NO: 1648 - RNA, 21 nt, Artificial Sequence
  5′ anti-sense siRNA sequences non-structural proteins (NSPs)
uuacuguaugugaugucagca

SEQ ID NO: 1649 - RNA, 21 nt, Artificial Sequence
  5′ anti-sense siRNA sequences non-structural proteins (NSPs)
uucuaacauagugcucuugug

SEQ ID NO: 1650 - RNA, 21 nt, Artificial Sequence
  5′ anti-sense siRNA sequences non-structural proteins (NSPs)
uuuugcauaccaaccuuuuga

SEQ ID NO: 1651 - RNA, 21 nt, Artificial Sequence
  5′ anti-sense siRNA sequences non-structural proteins (NSPs)
uuuuaaugccuucucacauag

SEQ ID NO: 1652 - RNA, 21 nt, Artificial Sequence
  5′ anti-sense siRNA sequences non-structural proteins (NSPs)
uugaauucacuuugaauuuau

SEQ ID NO: 1653 - RNA, 21 nt, Artificial Sequence
  5′ anti-sense siRNA sequences non-structural proteins (NSPs)
uucuaauguugaauucacuuu

SEQ ID NO: 1654 - RNA, 21 nt, Artificial Sequence
  5′ anti-sense siRNA sequences non-structural proteins (NSPs)
uucauaagucuacacacugaa

SEQ ID NO: 1655 - RNA, 21 nt, Artificial Sequence
  5′ anti-sense siRNA sequences non-structural proteins (NSPs)
uuucauaagucuacacacuga

SEQ ID NO: 1656 - RNA, 21 nt, Artificial Sequence
  5′ anti-sense siRNA sequences non-structural proteins (NSPs)
uugucuuuaugugcuuuaagc

SEQ ID NO: 1657 - RNA, 21 nt, Artificial Sequence
  5′ anti-sense siRNA sequences non-structural proteins (NSPs)
uuugucuuuaugugcuuuaag

SEQ ID NO: 1658 - RNA, 21 nt, Artificial Sequence
  5′ anti-sense siRNA sequences non-structural proteins (NSPs)
uuacauuacaagagugagcug

SEQ ID NO: 1659 - RNA, 21 nt, Artificial Sequence
  5′ anti-sense siRNA sequences non-structural proteins (NSPs)
aagacuaauuuaugugauguu

SEQ ID NO: 1660 - RNA, 21 nt, Artificial Sequence
  5′ anti-sense siRNA sequences non-structural proteins (NSPs)
auuaacagacaagacuaauuu

SEQ ID NO: 1661 - RNA, 21 nt, Artificial Sequence
  5′ anti-sense siRNA sequences non-structural proteins (NSPs)
gauuaacagacaagacuaauu

SEQ ID NO: 1662 - RNA, 21 nt, Artificial Sequence
  5′ anti-sense siRNA sequences non-structural proteins (NSPs)
uguacuuuacuguuuuuaguu

SEQ ID NO: 1663 - RNA, 21 nt, Artificial Sequence
  5′ anti-sense siRNA sequences non-structural proteins (NSPs)
uauuuguacuuuacuguuuuu

SEQ ID NO: 1664 - RNA, 21 nt, Artificial Sequence
  5′ anti-sense siRNA sequences non-structural proteins (NSPs)
ccuauuuguacuuuacuguuu

SEQ ID NO: 1665 - RNA, 21 nt, Artificial Sequence
  5′ anti-sense siRNA sequences non-structural proteins (NSPs)
ccaacauuuaauuuguaaguu

SEQ ID NO: 1666 - RNA, 21 nt, Artificial Sequence
  5′ anti-sense siRNA sequences non-structural proteins (NSPs)
aauaaucaccaacauuuaauu

SEQ ID NO: 1667 - RNA, 21 nt, Artificial Sequence
  5′ anti-sense siRNA sequences non-structural proteins (NSPs)
auacuuuugcauaccaaccuu

SEQ ID NO: 1668 - RNA, 21 nt, Artificial Sequence
  5′ anti-sense siRNA sequences non-structural proteins (NSPs)
agguauaauucuacuacauuu

SEQ ID NO: 1669 - RNA, 21 nt, Artificial Sequence
  5′ anti-sense siRNA sequences non-structural proteins (NSPs)
cagguauaauucuacuacauu

SEQ ID NO: 1670 - RNA, 21 nt, Artificial Sequence
  5′ anti-sense siRNA sequences non-structural proteins (NSPs)
uguugaauucacuuugaauuu

SEQ ID NO: 1671 - RNA, 21 nt, Artificial Sequence
  5′ anti-sense siRNA sequences non-structural proteins (NSPs)
auguugaauucacuuugaauu

SEQ ID NO: 1672 - RNA, 21 nt, Artificial Sequence
  5′ anti-sense siRNA sequences non-structural proteins (NSPs)
uucuaauguugaauucacuuu

SEQ ID NO: 1673 - RNA, 21 nt, Artificial Sequence
  5′ anti-sense siRNA sequences non-structural proteins (NSPs)
guucuaauguugaauucacuu

SEQ ID NO: 1674 - RNA, 21 nt, Artificial Sequence
  5′ anti-sense siRNA sequences non-structural proteins (NSPs)
cauaagucuacacacugaauu

SEQ ID NO: 1675 - RNA, 21 nt, Artificial Sequence
  5′ anti-sense siRNA sequences non-structural proteins (NSPs)
gucuuuaugugcuuuaagcuu

SEQ ID NO: 1676 - RNA, 21 nt, Artificial Sequence
  5′ anti-sense siRNA sequences non-structural proteins (NSPs)
cugauuugucuuuaugugcuu

SEQ ID NO: 1677 - RNA, 21 nt, Artificial Sequence
  5′ anti-sense siRNA sequences non-structural proteins (NSPs)
ccuauuuguggccuguuaauu

SEQ ID NO: 1678 - RNA, 21 nt, Artificial Sequence
  5′ anti-sense siRNA sequences non-structural proteins (NSPs)
auucucuuaccacgccuauuu

SEQ ID NO: 1679 - RNA, 21 nt, Artificial Sequence
  5′ anti-sense siRNA sequences non-structural proteins (NSPs)
aauucucuuaccacgccuauu

SEQ ID NO: 1680 - RNA, 21 nt, Artificial Sequence
  5′ anti-sense siRNA sequences non-structural proteins (NSPs)
aaucuuugaggcuacagcauu

SEQ ID NO: 1681 - RNA, 21 nt, Artificial Sequence
  5′ anti-sense siRNA sequences non-structural proteins (NSPs)
gaugaaucaacaguuugaguu

SEQ ID NO: 1682 - RNA, 21 nt, Artificial Sequence
  5′ anti-sense siRNA sequences non-structural proteins (NSPs)
ugaauaugacauagucauauu

SEQ ID NO: 1683 - RNA, 21 nt, Artificial Sequence
  5′ anti-sense siRNA sequences non-structural proteins (NSPs)
uuacacugauacaguauaagu

SEQ ID NO: 1684 - RNA, 21 nt, Artificial Sequence
  5′ anti-sense siRNA sequences non-structural proteins (NSPs)
cauuacaagagugagcuguuu

SEQ ID NO: 1685 - RNA, 21 nt, Artificial Sequence
  5′ anti-sense siRNA sequences non-structural proteins (NSPs)
acauuacaagagugagcuguu

SEQ ID NO: 1686 - RNA, 21 nt, Artificial Sequence
  5′ anti-sense siRNA sequences non-structural proteins (NSPs)
uuaacagacaagacuaauuua

SEQ ID NO: 1687 - RNA, 21 nt, Artificial Sequence
  5′ anti-sense siRNA sequences non-structural proteins (NSPs)
uuguccauuagcacacaaugg

SEQ ID NO: 1688 - RNA, 21 nt, Artificial Sequence
  5′ anti-sense siRNA sequences non-structural proteins (NSPs)
uuaucgcuaccaacacaugua

SEQ ID NO: 1689 - RNA, 21 nt, Artificial Sequence
  5′ anti-sense siRNA sequences non-structural proteins (NSPs)
uucaguacagguguuagcuaa

SEQ ID NO: 1690 - RNA, 21 nt, Artificial Sequence
  5′ anti-sense siRNA sequences non-structural proteins (NSPs)
uuucaguacagguguuagcua

SEQ ID NO: 1691 - RNA, 21 nt, Artificial Sequence
  5′ anti-sense siRNA sequences non-structural proteins (NSPs)
uugagucuuucaguacaggug

SEQ ID NO: 1692 - RNA, 21 nt, Artificial Sequence
  5′ anti-sense siRNA sequences non-structural proteins (NSPs)
uuaagugguggucuagguuua

SEQ ID NO: 1693 - RNA, 21 nt, Artificial Sequence
  5′ anti-sense siRNA sequences non-structural proteins (NSPs)
uucgguuaagugguggucuag

SEQ ID NO: 1694 - RNA, 19 nt, Artificial Sequence
  5′ anti-sense siRNA sequences non-structural proteins (NSPs)
uuagcaaugugcguggugc

SEQ ID NO: 1695 - RNA, 23 nt, Artificial Sequence
  5′ anti-sense siRNA sequences non-structural proteins (NSPs)
uuagcaaugugcguggugcaggu

SEQ ID NO: 1696 - RNA, 25 nt, Artificial Sequence
  5′ anti-sense siRNA sequences non-structural proteins (NSPs)
uuagcaaugugcguggugcagguaa

SEQ ID NO: 1697 - RNA, 21 nt, Artificial Sequence
  5′ anti-sense siRNA sequences non-structural proteins (NSPs)
uuucgguuaagugguggucua

SEQ ID NO: 1698 - RNA, 21 nt, Artificial Sequence
  5′ anti-sense siRNA sequences non-structural proteins (NSPs)
uuaauggcauuacuguaugug

SEQ ID NO: 1699 - RNA, 21 nt, Artificial Sequence
  5′ anti-sense siRNA sequences non-structural proteins (NSPs)
uuugcauaccaaccuuuugau

SEQ ID NO: 1700 - RNA, 21 nt, Artificial Sequence
  5′ anti-sense siRNA sequences non-structural proteins (NSPs)
uucucacauagugcaucaaca

SEQ ID NO: 1701 - RNA, 21 nt, Artificial Sequence
  5′ anti-sense siRNA sequences non-structural proteins (NSPs)
uuaaugccuucucacauagug

SEQ ID NO: 1702 - RNA, 21 nt, Artificial Sequence
  5′ anti-sense siRNA sequences non-structural proteins (NSPs)
uuuaaugccuucucacauagu

SEQ ID NO: 1703 - RNA, 21 nt, Artificial Sequence
  5′ anti-sense siRNA sequences non-structural proteins (NSPs)
uucuacuacauuuaucuauag

SEQ ID NO: 1704 - RNA, 21 nt, Artificial Sequence
  5′ anti-sense siRNA sequences non-structural proteins (NSPs)
uuagcaaugugcguggugcag

SEQ ID NO: 1705 - RNA, 21 nt, Artificial Sequence
  5′ anti-sense siRNA sequences non-structural proteins (NSPs)
uuaguuagcaaugugcguggu

SEQ ID NO: 1706 - RNA, 21 nt, Artificial Sequence
  5′ anti-sense siRNA sequences non-structural proteins (NSPs)
uuaugugcuuuaagcuuauua

SEQ ID NO: 1707 - RNA, 21 nt, Artificial Sequence
  5′ anti-sense siRNA sequences non-structural proteins (NSPs)
uuuaugugcuuuaagcuuauu

SEQ ID NO: 1708 - RNA, 21 nt, Artificial Sequence
  5′ anti-sense siRNA sequences non-structural proteins (NSPs)
uucucuuaccacgccuauuug

SEQ ID NO: 1709 - RNA, 21 nt, Artificial Sequence
  5′ anti-sense siRNA sequences non-structural proteins (NSPs)
uuacguguaaggaauucucuu

SEQ ID NO: 1710 - RNA, 21 nt, Artificial Sequence
  5′ anti-sense siRNA sequences non-structural proteins (NSPs)
uucugugaauuauaaggugaa

SEQ ID NO: 1711 - RNA, 21 nt, Artificial Sequence
  5′ anti-sense siRNA sequences non-structural proteins (NSPs)
uugagugaauaugacauaguc

SEQ ID NO: 1712 - RNA, 21 nt, Artificial Sequence
  5′ anti-sense siRNA sequences non-structural proteins (NSPs)
uuugagugaauaugacauagu

SEQ ID NO: 1713 - RNA, 21 nt, Artificial Sequence
  5′ anti-sense siRNA sequences non-structural proteins (NSPs)
uuacaagagugagcuguuuca

SEQ ID NO: 1714 - RNA, 21 nt, Artificial Sequence
  5′ anti-sense siRNA sequences non-structural proteins (NSPs)
uuuugcucugguaauagcaac

SEQ ID NO: 1715 - RNA, 21 nt, Artificial Sequence
  5′ anti-sense siRNA sequences non-structural proteins (NSPs)
gacaucacaaccuggagcauu

SEQ ID NO: 1716 - RNA, 21 nt, Artificial Sequence
  5′ anti-sense siRNA sequences non-structural proteins (NSPs)
gucacauguugcaauugcauu

SEQ ID NO: 1717 - RNA, 21 nt, Artificial Sequence
  5′ anti-sense siRNA sequences non-structural proteins (NSPs)
gcauuuguccagucacauguu

SEQ ID NO: 1718 - RNA, 21 nt, Artificial Sequence
  5′ anti-sense siRNA sequences non-structural proteins (NSPs)
gagucuuucaguacagguguu

SEQ ID NO: 1719 - RNA, 21 nt, Artificial Sequence
  5′ anti-sense siRNA sequences non-structural proteins (NSPs)
uagcaauaccauaagacaguu

SEQ ID NO: 1720 - RNA, 21 nt, Artificial Sequence
  5′ anti-sense siRNA sequences non-structural proteins (NSPs)
auucucugucagacagcacuu

SEQ ID NO: 1721 - RNA, 21 nt, Artificial Sequence
  5′ anti-sense siRNA sequences non-structural proteins (NSPs)
guuaagugguggucuagguuu

SEQ ID NO: 1722 - RNA, 21 nt, Artificial Sequence
  5′ anti-sense siRNA sequences non-structural proteins (NSPs)
gguuaagugguggucuagguu

SEQ ID NO: 1723 - RNA, 21 nt, Artificial Sequence
  5′ anti-sense siRNA sequences non-structural proteins (NSPs)
uccuauuuguacuuuacuguu

SEQ ID NO: 1724 - RNA, 21 nt, Artificial Sequence
  5′ anti-sense siRNA sequences non-structural proteins (NSPs)
caucaccauagucaccuuuuu

SEQ ID NO: 1725 - RNA, 21 nt, Artificial Sequence
  5′ anti-sense siRNA sequences non-structural proteins (NSPs)
auaaucaccaacauuuaauuu

SEQ ID NO: 1726 - RNA, 21 nt, Artificial Sequence
  5′ anti-sense siRNA sequences non-structural proteins (NSPs)
cauaccaaccuuuugauaauu

SEQ ID NO: 1727 - RNA, 21 nt, Artificial Sequence
  5′ anti-sense siRNA sequences non-structural proteins (NSPs)
acuuuugcauaccaaccuuuu

SEQ ID NO: 1728 - RNA, 21 nt, Artificial Sequence
  5′ anti-sense siRNA sequences non-structural proteins (NSPs)
uacuuuugcauaccaaccuuu

SEQ ID NO: 1729 - RNA, 21 nt, Artificial Sequence
  5′ anti-sense siRNA sequences non-structural proteins (NSPs)
auacuguucuaauguugaauu

SEQ ID NO: 1730 - RNA, 21 nt, Artificial Sequence
  5′ anti-sense siRNA sequences non-structural proteins (NSPs)
aagacauacuguucuaauguu

SEQ ID NO: 1731 - RNA, 21 nt, Artificial Sequence
  5′ anti-sense siRNA sequences non-structural proteins (NSPs)
gccaauguacacauagugcuu

SEQ ID NO: 1732 - RNA, 21 nt, Artificial Sequence
  5′ anti-sense siRNA sequences non-structural proteins (NSPs)
aacaugucuggaccuauaguu

SEQ ID NO: 1733 - RNA, 21 nt, Artificial Sequence
  5′ anti-sense siRNA sequences non-structural proteins (NSPs)
uuuaugugcuuuaagcuuauu

SEQ ID NO: 1734 - RNA, 21 nt, Artificial Sequence
  5′ anti-sense siRNA sequences non-structural proteins (NSPs)
ugauuugucuuuaugugcuuu

SEQ ID NO: 1735 - RNA, 21 nt, Artificial Sequence
  5′ anti-sense siRNA sequences non-structural proteins (NSPs)
uuacguguaaggaauucucuu

SEQ ID NO: 1736 - RNA, 21 nt, Artificial Sequence
  5′ anti-sense siRNA sequences non-structural proteins (NSPs)
uugagucacaucugugacauc

SEQ ID NO: 1737 - RNA, 21 nt, Artificial Sequence
  5′ anti-sense siRNA sequences non-structural proteins (NSPs)
uuguccagucacauguugcaa

SEQ ID NO: 1738 - RNA, 21 nt, Artificial Sequence
  5′ anti-sense siRNA sequences non-structural proteins (NSPs)
uuuguccagucacauguugca

SEQ ID NO: 1739 - RNA, 21 nt, Artificial Sequence
  5′ anti-sense siRNA sequences non-structural proteins (NSPs)
uucacguacaguagcaauacc

SEQ ID NO: 1740 - RNA, 21 nt, Artificial Sequence
  5′ anti-sense siRNA sequences non-structural proteins (NSPs)
uucucugucagacagcacuuc

SEQ ID NO: 1741 - RNA, 21 nt, Artificial Sequence
  5′ anti-sense siRNA sequences non-structural proteins (NSPs)
uugcauaccaaccuuuugaua

SEQ ID NO: 1742 - RNA, 21 nt, Artificial Sequence
  5′ anti-sense siRNA sequences non-structural proteins (NSPs)
uuagcacguaaucuggcauug

SEQ ID NO: 1743 - RNA, 21 nt, Artificial Sequence
  5′ anti-sense siRNA sequences non-structural proteins (NSPs)
uuucagcaggacaacgccgac

SEQ ID NO: 1744 - RNA, 21 nt, Artificial Sequence
  5′ anti-sense siRNA sequences non-structural proteins (NSPs)
uuucagcaggacaacggcgac

SEQ ID NO: 1745 - RNA, 21 nt, Artificial Sequence
  5′ anti-sense siRNA sequences non-structural proteins (NSPs)
uugagcugauuugucuuuaug

SEQ ID NO: 1746 - RNA, 21 nt, Artificial Sequence
  5′ anti-sense siRNA sequences non-structural proteins (NSPs)
uugaggcuacagcauucugug

SEQ ID NO: 1747 - RNA, 21 nt, Artificial Sequence
  5′ anti-sense siRNA sequences non-structural proteins (NSPs)
uuugaggcuacagcauucugu

SEQ ID NO: 1748 - RNA, 21 nt, Artificial Sequence
  5′ anti-sense siRNA sequences non-structural proteins (NSPs)
uucagugguuugagugaauau

SEQ ID NO: 1749 - RNA, 21 nt, Artificial Sequence
  5′ anti-sense siRNA sequences non-structural proteins (NSPs)
uuucagugguuugagugaaua

SEQ ID NO: 1750 - RNA, 21 nt, Artificial Sequence
  5′ anti-sense siRNA sequences non-structural proteins (NSPs)
uuugcucugguaauagcaaca

SEQ ID NO: 1751 - RNA, 21 nt, Artificial Sequence
  5′ anti-sense siRNA sequences non-structural proteins (NSPs)
gacauggucguaacagcauuu

SEQ ID NO: 1752 - RNA, 21 nt, Artificial Sequence
  5′ anti-sense siRNA sequences non-structural proteins (NSPs)
ugacauggucguaacagcauu

SEQ ID NO: 1753 - RNA, 21 nt, Artificial Sequence
  5′ anti-sense siRNA sequences non-structural proteins (NSPs)
gcuaccaacacauguauuuuu

SEQ ID NO: 1754 - RNA, 21 nt, Artificial Sequence
  5′ anti-sense siRNA sequences non-structural proteins (NSPs)
cgcuaccaacacauguauuuu

SEQ ID NO: 1755 - RNA, 21 nt, Artificial Sequence
  5′ anti-sense siRNA sequences non-structural proteins (NSPs)
guccagucacauguugcaauu

SEQ ID NO: 1756 - RNA, 21 nt, Artificial Sequence
  5′ anti-sense siRNA sequences non-structural proteins (NSPs)
ccucaguagcuuugagcguuu

SEQ ID NO: 1757 - RNA, 21 nt, Artificial Sequence
  5′ anti-sense siRNA sequences non-structural proteins (NSPs)
uccucaguagcuuugagcguu

SEQ ID NO: 1758 - RNA, 21 nt, Artificial Sequence
  5′ anti-sense siRNA sequences non-structural proteins (NSPs)
agcaauaccauaagacaguuu

SEQ ID NO: 1759 - RNA, 21 nt, Artificial Sequence
  5′ anti-sense siRNA sequences non-structural proteins (NSPs)
guggucuagguuuaccaacuu

SEQ ID NO: 1760 - RNA, 21 nt, Artificial Sequence
  5′ anti-sense siRNA sequences non-structural proteins (NSPs)
guacucuccuauuuguacuuu

SEQ ID NO: 1761 - RNA, 21 nt, Artificial Sequence
  5′ anti-sense siRNA sequences non-structural proteins (NSPs)
uguacucuccuauuuguacuu

SEQ ID NO: 1762 - RNA, 21 nt, Artificial Sequence
  5′ anti-sense siRNA sequences non-structural proteins (NSPs)
gcaucaccauagucaccuuuu

SEQ ID NO: 1763 - RNA, 21 nt, Artificial Sequence
  5′ anti-sense siRNA sequences non-structural proteins (NSPs)
cagcaucaccauagucaccuu

SEQ ID NO: 1764 - RNA, 21 nt, Artificial Sequence
  5′ anti-sense siRNA sequences non-structural proteins (NSPs)
guaggugcacuuaauggcauu

SEQ ID NO: 1765 - RNA, 21 nt, Artificial Sequence
  5′ anti-sense siRNA sequences non-structural proteins (NSPs)
ggcacuaguguaggugcacuu

SEQ ID NO: 1766 - RNA, 21 nt, Artificial Sequence
  5′ anti-sense siRNA sequences non-structural proteins (NSPs)
ucaucugagauauugaguguu

SEQ ID NO: 1767 - RNA, 21 nt, Artificial Sequence
  5′ anti-sense siRNA sequences non-structural proteins (NSPs)
auaccaaccuuuugauaauuu

SEQ ID NO: 1768 - RNA, 21 nt, Artificial Sequence
  5′ anti-sense siRNA sequences non-structural proteins (NSPs)
gucgucucaggcaaugcauuu

SEQ ID NO: 1769 - RNA, 21 nt, Artificial Sequence
  5′ anti-sense siRNA sequences non-structural proteins (NSPs)
cuuagcacguaaucuggcauu

SEQ ID NO: 1770 - RNA, 21 nt, Artificial Sequence
  5′ anti-sense siRNA sequences non-structural proteins (NSPs)
caugucuggaccuauaguuuu

SEQ ID NO: 1771 - RNA, 21 nt, Artificial Sequence
  5′ anti-sense siRNA sequences non-structural proteins (NSPs)
acaugucuggaccuauaguuu

SEQ ID NO: 1772 - RNA, 21 nt, Artificial Sequence
  5′ anti-sense siRNA sequences non-structural proteins (NSPs)
gcaggacaacgccgacaaguu

SEQ ID NO: 1773 - RNA, 21 nt, Artificial Sequence
  5′ anti-sense siRNA sequences non-structural proteins (NSPs)
cacucacagugucaacaauuu

SEQ ID NO: 1774 - RNA, 21 nt, Artificial Sequence
  5′ anti-sense siRNA sequences non-structural proteins (NSPs)
gcacucacagugucaacaauu

SEQ ID NO: 1775 - RNA, 21 nt, Artificial Sequence
  5′ anti-sense siRNA sequences non-structural proteins (NSPs)
gcauugagcugauuugucuuu

SEQ ID NO: 1776 - RNA, 21 nt, Artificial Sequence
  5′ anti-sense siRNA sequences non-structural proteins (NSPs)
agcauugagcugauuugucuu

SEQ ID NO: 1777 - RNA, 21 nt, Artificial Sequence
  5′ anti-sense siRNA sequences non-structural proteins (NSPs)
cacgccuauuuguggccuguu

SEQ ID NO: 1778 - RNA, 21 nt, Artificial Sequence
  5′ anti-sense siRNA sequences non-structural proteins (NSPs)
aacgccuauuugaggucuguu

SEQ ID NO: 1779 - RNA, 21 nt, Artificial Sequence
  5′ anti-sense siRNA sequences non-structural proteins (NSPs)
ccugugaugaaucaacaguuu

SEQ ID NO: 1780 - RNA, 21 nt, Artificial Sequence
  5′ anti-sense siRNA sequences non-structural proteins (NSPs)
gagugagcuguuucagugguu

SEQ ID NO: 1781 - RNA, 21 nt, Artificial Sequence
  5′ anti-sense siRNA sequences non-structural proteins (NSPs)
uuguggcacuaguguaggugc

SEQ ID NO: 1782 - RNA, 21 nt, Artificial Sequence
  5′ anti-sense siRNA sequences non-structural proteins (NSPs)
uuccgaggaacaugucuggac

SEQ ID NO: 1783 - RNA, 21 nt, Artificial Sequence
  5′ anti-sense siRNA sequences non-structural proteins (NSPs)
uucagcaggacaacgccgaca

SEQ ID NO: 1784 - RNA, 21 nt, Artificial Sequence
  5′ anti-sense siRNA sequences non-structural proteins (NSPs)
uuguggccuguuaauugcaga

SEQ ID NO: 1785 - RNA, 21 nt, Artificial Sequence
  5′ anti-sense siRNA sequences non-structural proteins (NSPs)
uuuguggccuguuaauugcag

SEQ ID NO: 1786 - RNA, 21 nt, Artificial Sequence
  5′ anti-sense siRNA sequences non-structural proteins (NSPs)
uuaccacgccuauuuguggcc

SEQ ID NO: 1787 - RNA, 21 nt, Artificial Sequence
  5′ anti-sense siRNA sequences non-structural proteins (NSPs)
uugcucugguaauagcaacau

SEQ ID NO: 1788 - RNA, 21 nt, Artificial Sequence
  5′ anti-sense siRNA sequences non-structural proteins (NSPs)
ucgcuaccaacacauguauuu

SEQ ID NO: 1789 - RNA, 21 nt, Artificial Sequence
  5′ anti-sense siRNA sequences non-structural proteins (NSPs)
aucgcuaccaacacauguauu

SEQ ID NO: 1790 - RNA, 21 nt, Artificial Sequence
  5′ anti-sense siRNA sequences non-structural proteins (NSPs)
agcaucaccauagucaccuuu

SEQ ID NO: 1791 - RNA, 21 nt, Artificial Sequence
  5′ anti-sense siRNA sequences non-structural proteins (NSPs)
ccuggaguguagaauacuuuu

SEQ ID NO: 1792 - RNA, 21 nt, Artificial Sequence
  5′ anti-sense siRNA sequences non-structural proteins (NSPs)
cgagcacgugcagguauaauu

SEQ ID NO: 1793 - RNA, 21 nt, Artificial Sequence
  5′ anti-sense siRNA sequences non-structural proteins (NSPs)
ugucgucucaggcaaugcauu

SEQ ID NO: 1794 - RNA, 21 nt, Artificial Sequence
  5′ anti-sense siRNA sequences non-structural proteins (NSPs)
augugcguggugcagguaauu

SEQ ID NO: 1795 - RNA, 21 nt, Artificial Sequence
  5′ anti-sense siRNA sequences non-structural proteins (NSPs)
ggcuacagcauucugugaauu

SEQ ID NO: 1796 - RNA, 21 nt, Artificial Sequence
  5′ anti-sense siRNA sequences non-structural proteins (NSPs)
agugagcuguuucagugguuu

SEQ ID NO: 1797 - RNA, 21 nt, Artificial Sequence
  5′ anti-sense siRNA sequences non-structural proteins (NSPs)
ugcucugguaauagcaacauu